(19) United States
(12) Patent Application Publication
(10) Pub. No.: US 2007/0099199 A1
(43) Pub. Date: May 3, 2007

(54) Methods and compositions for treating cervical cancer

(75) Inventors: Peter S. Lu, Mountain View, CA (US); Christoph Peter Bagowski, Palo Alto, CA (US); Johannes Schweizer, Mountain View, CA (US); Chamorro Somoza Diaz-Sarmiento, Palo Alto, CA (US); Jonathan David Garman, San Jose, CA (US); Michael P. Belmares, San Jose, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Arbor Vita Corporation

(21) Appl. No.: 11/403,141

(22) Filed: Apr. 11, 2006

Related U.S. Application Data
(63) Continuation of application No. 10/789,102, filed on Feb. 27, 2004, now abandoned.
Application No. 10/789,102 is a continuation-in-part of application No. 10/630,590, filed on Jul. 29, 2003.
Application No. 10/789,102 is a continuation-in-part of application No. PCT/US02/24655, filed on Aug. 2, 2002.
Application No. 10/789,102 is a continuation-in-part of application No. 10/080,273, filed on Feb. 19, 2002.
Application No. 10/789,102 is a continuation-in-part of application No. 09/710,059, filed on Nov. 10, 2000, now abandoned.
(60) Provisional application No. 60/490,094, filed on Jul. 25, 2003.
(60) Provisional application No. 60/450,464, filed on Feb. 27, 2003.
(60) Provisional application No. 60/309,841, filed on Aug. 3, 2001.
(60) Provisional application No. 60/360,061, filed on Feb. 25, 2002.
(60) Provisional application No. 60/269,523, filed on Feb. 16, 2001.

(51) Int. Cl. C12Q 1/68; C12N 5/06; C12N 5/16

(52) U.S. Cl. 435/6; 435/345

(57) ABSTRACT

The invention provides methods and compositions for treating pathogen infections, particularly human papillomavirus infections. Specifically, the invention provides a method of screening that involves determining an effect of a candidate agent on binding of an E6 protein from an oncogenic strain of HPV to a polypeptide containing the amino acid sequence of a particular PDZ domain from the cellular protein MAGI-1. The invention provides methods to treat diseases associated with expression of pathogen proteins by modulating their interactions with MAGI-1, and a number of isolated peptides useful in such methods. Also provided are kits for performing the subject methods.

### CROSS-REFERENCE

This application: a) claims the benefit of: U.S. patent application Ser. No. 10/630,590, filed Jul. 29, 2003; U.S. Provisional Application No. 60/490,094, filed Jul. 25, 2003; and U.S. Provisional Application No. 60/450,464, filed Feb. 27, 2003; b) is a CIP of of PCT Application No. US02/24655, filed Aug. 2, 2002, which application claims the benefit of U.S. Provisional Application No. 60/309841, filed Aug. 3, 2001, and U.S. Provisional Application No. 60/360061, filed Feb. 25, 2002; c) is a CIP of U.S. Non-Provisional Application Ser. No. 10/080,273, filed Feb. 19, 2002, which application claims the benefit of U.S. Provisional Application No. 60/269,523, filed Feb. 16, 2001; and d) is a CIP of U.S. Non-Provisional Application Ser. No. 09/710,059, filed Nov. 10, 2000, all of which applications are incorporated herein by reference in their entirety for all purposes.

### FIELD OF THE INVENTION

The present invention relates to therapeutics for the treatment of pathogenic infections such as human Papillomavirus (HPV) infections, and methods for using such therapeutics to treat cells, tissues, or patients that are infected and may develop cancerous growth or other disorders.

### BACKGROUND

Cervical cancer is the second most common cancer diagnosis in women and is linked to high-risk human papillomavirus infection 99.7% of the time. Currently, 12,000 new cases of invasive cervical cancer are diagnosed in US women annually, resulting in 5,000 deaths each year. Furthermore, there are approximately 400,000 cases of cervical cancer and close to 200,000 deaths annually worldwide. Human papillomaviruses (HPVs) are one of the most common causes of sexually transmitted disease in the world. Overall, 50-75% of sexually active men and women acquire genital HPV infections at some point in their lives. An estimated 5.5 million people become infected with HPV each year in the US alone, and at least 20 million are currently infected. The more than 100 different isolates of HPV have been broadly subdivided into high-risk and low-risk subtypes based on their association with cervical carcinomas or with benign cervical lesions or dysplasias.

A number of lines of evidence point to HPV infections as the etiological agents of cervical cancers. Multiple studies in the 1980's reported the presence of HPV variants in cervical dysplasias, cancer, and in cell lines derived from cervical cancer. Further research demonstrated that the E6-E7 region of the genome from oncogenic HPV 18 is selectively retained in cervical cancer cells, suggesting that HPV infection could be causative and that continued expression of the E6-E7 region is required for maintenance of the immortalized or cancerous state. The following year, Sedman et al demonstrated that the E6-E7 genes from HPV 16 were sufficient to immortalize human keratinocytes in culture. Barbosa et al demonstrated that although E6-E7 genes from high risk HPVs could transform cell lines, the E6-E7 regions from low risk, or non-oncogenic variants such as HPV 6 and HPV 11 were unable to transform human keratinocytes. More recently, Pillai et al examined HPV 16 and 18 infection by in situ hybridization and E6 protein expression by immunocytochemistry in 623 cervical tissue samples at various stages of tumor progression and found a significant correlation between histological abnormality and HPV infection.

Human papillomaviruses characterized to date are associated with lesions confined to the epithelial layers of skin, or oral, pharyngeal, respiratory, and, most importantly, anogenital mucosae. Specific human papillomavirus types, including HPV 6 and 11, frequently cause benign mucosal lesions, whereas other types such as HPV 16, 18, and a host of other strains, are predominantly found in high-grade lesions and cancer. Individual types of human papillomaviruses (HPV) which infect mucosal surfaces have been implicated as the causative agents for carcinomas of the cervix, anus, penis, larynx and the buccal cavity, occasional periungal carcinomas, as well as benign anogenital warts. The identification of particular HPV types is used for identifying patients with premalignant lesions who are at risk of progression to malignancy. Although visible anogenital lesions are present in some persons infected with human papillomavirus, the majority of individuals with HPV genital tract infection do not have clinically apparent disease, but analysis of cytomorphological traits present in cervical smears can be used to detect HPV infection. Papanicolaou tests are a valuable screening tool, but they miss a large proportion of HPV-infected persons due to the unfortunate false positive and false negative test results. In addition, they are not amenable to worldwide testing because interpretation of results requires trained pathologists. Because of the limited use and success rate of the Papanicolaou test, many HPV-infected individuals fail to receive timely diagnosis, a problem that precludes efforts to administer treatment prior to the appearance of clinical symptoms. A significant unmet need exists for early and accurate diagnosis of oncogenic HPV infection as well as for treatments directed at the causative HPV infection, preventing the development of cervical cancer by intervening earlier in disease progression.

Because treatments are usually administered after the onset of clinical symptoms, current treatment paradigms are focused on the actual cervical dysplasia rather than the underlying infection with HPV. Women are screened by physicians annually for cervical dysplasia and are treated with superficial ablative techniques, including cryosurgery, laser ablation and excision. As the disease progresses, treatment options become more aggressive, including partial or radical hysterectomy, radiation or chemotherapy. All of these treatments are invasive and carry the possibility or guarantee of permanent infertility. In addition, surgical removal of tissue may not guarantee that all infected cells have been eliminated due to the fact that some transformed cells may not yet be displaying the morphological changes associated with HPV infection.

More recently, research has focused on nonsurgical alternatives for the treatment of HPV infection and cervical cancer. Various DNA and protein treatments designed to induce apoptosis in cells may reduce the number of cancerous cells, but may also induce apoptosis in healthy cells. Topoisomerase inhibitors such as irinotecan (Camptosar®) and inhibitors of thymine production such as fluorouracil (Fluoroplex®, Efudex®, Adrucil®) nonspecifically prevent cell division. While these treatments are beneficial therapies for the treatment of a variety of cancers, they pose significant risk to healthy cells and fail to specifically target HPV infected cells.

Because the oncogenicity of HPV has been shown to be protein based, treatments that specifically block the activity of oncogenic strains of HPV protein may provide more effective and less invasive treatments than those currently in use. Administration of antagonistic compounds specific for oncogenic strains of HPV may eliminate the need for expensive surgical procedures by treating the causative HPV infection prior to the appearance of clinical symptoms or early in the disease progression. In addition, the specificity of an oncogenic HPV antagonist significantly reduces risk of damage to healthy cells, thereby minimizing side effects.

### SUMMARY

The invention provides methods and compositions for treating pathogen infections, particularly human papillomavirus infections. Specifically, the invention provides a method of screening for modulators of protein-protein interactions that involves determining an effect of a candidate agent on binding of an E6 protein from an oncogenic strain of HPV to a polypeptide containing the amino acid sequence of a particular PDZ domain from the cellular protein MAGI-1. The invention provides methods to treat diseases associated with expression of pathogen proteins by modulating their interactions with MAGI-1, and a number of isolated peptides useful in such methods. Also provided are kits for performing the subject methods.

Accordingly, in one embodiment, the invention provides a method of screening. In general, the subject screening methods generally involve determining an effect of a candidate agent on binding of an oncogenic E6 protein to a polypeptide comprising the amino acid sequence of a second PDZ domain from MAGI-1. In certain embodiments, such a polypeptide comprises the sequence of SEQ ID NO:320, or a oncogenic E6 protein-binding variant thereof, examples of which are set forth as SEQ ID NOS:321-357. In many embodiments, therefore, the candidate agent is contacted with such a MAGI-1 PDZ polypeptide, and the effect of binding of the polypeptide to an oncogenic E6 protein in the presence of the agent is determined.

In most embodiments, the screening methods are done in both the presence and absence of the candidate agent, and any agent that reduces binding between the two molecules may be used as an anti-HPV agent. Usually, a library of candidate agents is screened for anti-HPV activity.

Binding of the MAGI-1 PDZ domain and the oncogenic E6 protein may be assayed using assays that are well known in the art. For example, binding may be assayed biochemically, or, in other embodiments, the MAGI-1 PDZ domain and the oncogenic E6 protein may produce a signal when bound together. In testing candidate agents, such a signal can be assayed in order to assess binding between the two proteins. For example, as used in the subject assays, the MAGI-1 PDZ domain and the oncogenic E6 protein may form a fluorescence resonance energy transfer (FRET), bioluminescence resonance energy transfer (BRET), or colorimetric signal producing system, that could be assayed.

The screening assays may be extracellular (i.e., biochemical) assays using isolated polypeptides, or, in some embodiments, cellular assays, where binding of the two proteins is assayed in a cell contacted with a candidate agent.

Once identified, agents that disrupt interactions between the two proteins may be tested in HPV oncogenicity assays in vitro, which assays are well known in the art.

The invention also provides isolated peptides that can effectively inhibit binding between the second MAGI-1 PDZ domain and an E6 protein from oncogenic strains of HPV. In general, the peptides contain at least two (e.g. 3, 4, 5, 6, 7 or more, usually up to about 10 or 15), contiguous amino acids of the C-terminus of an E6 protein from oncogenic strain of HPV. In certain embodiments, the peptides contain a sequence that is at the immediate C-terminus (i.e., containing the terminal amino acid) of such an E6 protein, whereas in other embodiments, the peptides contain a sequence that is spaced from the terminu of the E6 protein by 1, 2, or 3 or more amino acids. In certain embodiments, the at least three contiguous amino acids, when present in a subject peptide, are typically, although not always, at the C-terminus of the isolated peptide.

In certain embodiments, a subject peptide may be linked to a cell permeable peptide carrier moiety that provides for internalization of a subject peptide. Such moieties are well known in the art, and described in greater below.

The subject peptides may be used to modulate an interaction between a MAGI-1 protein and an oncogenic HPV E6 protein. In general, this method involves contacting the MAGI-1 protein a subject isolated peptide.

Accordingly, the invention also provides a method of reducing the oncogenicity of an oncogenic strain of HPV in a cell. In general, this method involves reducing binding of an E6 protein of said HPV to a MAGI-1 protein of the-cell. The cell may be present in vitro, e.g., as a cultured cell or the like, or as a cell in vivo, i.e., in a subject. In most embodiments, binding between the two polypeptides can be reduced by contacting at least one of the components, usually the MAGI-1 protein, with a subject peptide, or an agent discovered using the subject screening assays.

A subject isolated peptide may be present in a pharmaceutical composition containing the peptide and a pharmaceutically acceptable carrier, and such a composition may be used in a method of treating a cancer associated with HPV infection. In general, this method involves administering to a subject in need thereof such a pharmaceutical composition. In particular embodiments, the subject has one or more of the following HPV-related cancers: cervical cancer, uterine cancer, anal cancer, colorectal cancer, penile cancer, oral cancer, skin cancer or esophageal cancer.

Finally, a kit containing a subject peptide is provided. In most embodiments, such a kit also contains instructions for using the peptide to treat a cancer associated with HPV infection.

The present inventors have identified methods for treating diseases associated with HPV, including but not limited to cervical cancer, anal cancer, penile cancer, throat cancer and skin cancers. The methods of the invention involve modulation of interactions between PDZ proteins and HPV PL proteins as listed in Table 3, interactions that play a significant role in the biological function and morphology associated with HPV infection. Methods for determining PDZ-PL interactions are disclosed herein, as well as methods for identifying modulators of those interactions in vitro and in vivo. Administration and optimization of treatment is also disclosed.

The methods of the invention provide treatment that is highly specific, targeting cells that are infected with HPV. This specificity significantly reduces or eliminates the negative effects of treatment of uninfected, healthy cells, thereby minimizing side effects. Because the treatments of the invention can be administered prior to the appearance of clinical symptoms, HPV infection can be effectively treated before life-threatening diseases (e.g. cervical cancer) develop. In addition, early and specific treatment eliminates the need for invasive and costly surgical procedures that cause significant damage to healthy tissue and often fail to eliminate all infected cells.

The invention provides methods of screening for anti-cancer agents, methods of reducing the oncogenicity of an oncogenic HPV, methods for reducing a cancerous phenotype of a cell infected with an oncogenic HPV, and methods for treating HPV infection or cancer, e.g., cervical cancer. In general, the methods involve disrupting the interaction between a PDZ protein, particularly MAGI-1, and the PDZ ligand found in the E6 proteins of oncogenic strains of HPV.

In certain embodiments, the subject invention involves modulating (i.e., increasing or decreasing) interactions between PTEN and PDZ proteins, e.g., MAGI-1, in order to modulate downstream molecular events that involve cell division.

In certain other embodiment, the subject invention involves blocking JNK, FAK or the transcription factor AP-1 to reduce the oncogenicity of an oncogenic HPV, reduce a cancerous phenotype of a cell infected with an oncogenic HPV, and treat HPV infection or cancer.

The invention also provides assays for identifying agents for reducing the oncogenicity of an oncogenic HPV, methods for reducing a cancerous phenotype of a cell infected with an oncogenic HPV, and methods for treating HPV infection or cancer. In general, these methods involve providing a cell that produces MAGI-1 and oncogenic HPV E6 proteins, and testing the ability of agents to E6 activation of FAK, JNK or AP 1, or any other downstream event activated by binding of the E6 protein to MAGI-1. Methods for assessing activity of FAK, JNK and AP1 are well known in the art or are described herein. For example, AP1 activity can be measured using a promoter-reporter fusion, where the promoter is an AP1 promoter or a promoter from a gene activated by AP1, or a JNK assay, a method for which is provided herein.

Also provided are screening methods using transgenic mice that recombinantly express an oncogenic E6 protein, such as a mouse that is known in the art. Such methods may use a mouse with reduced MAGI-1 expression (e.g., a MAGI-1 “knockout” mouse). Such E6 and MAGI-1 mice may be crossed with each other, and may be in genetic backgrounds that have altered FAK, JNK or AP1 activity (e.g., they have a knockout in or overexpress on of these genes).

The methods of the invention provide a more specific, effective, and cost-efficient alternative to current treatments for oncogenic HPV infection.

### BRIEF DESCRIPTION OF THE DRAWINGS

FIG. 1A: Northern blot analysis of HPV16 E6 and HPV18 E6 expression in various-cell lines. Lanes: 1 B-cell (Ramos); 2 No HPV (HTB32); 3 1550 HPV 16+18; 4 1595 HPV18; 5 1594 HPV18; 6 HTB 35 (HPV 16); 7 RNA marker. HPV18 E6 and HPV16 E6 refer to the radiolabeled probe used to detect expression in each of the cell lines.

FIG. 1B : Northern blot analysis of Magi-1 and TIP-1 expression in various cervical cell lines. The expected size for Magi-1 mRNA is 4.5 kb, although alternative splice forms are noted in Genbank. The expected size for Tip-1 mRNA is 1.4 kb. For Magi-1, we found that a probe encompassing PDZ domain 2 gave a high background on total RNA blots, so polyA+ RNA was isolated using the mRNA purification kit (Amersham-Pharmacia).

FIG. 2: PDZ proteins can specifically recognize oncogenic E6 proteins from human papillomavirus. An ELISA assay was used to demonstrate that a PDZ protein (TIP-1) could specifically recognize full length E6 protein from an oncogenic strain (HPV18) but did not show any reactivity with a non-oncogenic strain (HPV11). Series 1 and Series 2 represent independent trials. E6 ab indicates that an antibody against E6 from HPV18 was used for detection instead of the PDZ protein.

FIG. 3: Inhibition of the interaction between HPV E6 16 and TIP1 by Tax peptide. OD (A450) is shown on the y-axis, and titrating concentrations of Tax inhibitor (uM) are shown on the x-axis. HPV E6 16 peptide was used at a concentration of 10 uM, and TIP1 fusion protein was used at a concentration of 5 ug/mL. See Example 7 for further details.

FIG. 4: is a complation of four panels of autoradiographs, A), B) and C). A) Oncogenic HPV E6 16, but not non-oncogenic HPV E6 11, activates c-JUN N-terminal kinase (JNK), a kinase known to be involved in numerous oncogenic pathways. B) HPV E6 16-dependent activation of JNK can be inhibited by co-injection of peptide corresponding to the C-terminus of oncogenic Tax, but not with the peptide representing the C-terminus of non-oncogenic HPV E6 11. C) HPV E6 16 dependent activation of JNK can be inhibited by peptide representing HPV E6 16 oncoprotein, but not by peptide representing the C-terminus of nononcogenic HPV E6 11.

FIGS. 5A, 5B, 5C and 5D: show results of mammalian cell migration assays. Cells were transfected with a construct that expresses the E6 protein from HPV 16 or the same protein with a deletion of 3 amino acids at the carboxyl-terminus that abolishes the ability to interact with PDZ domains. E6-transfected cells migrate through a scratch, indicative of cell transformation, while E6 cells with a c-terminal deletion do not migrate to fill in the scratch.

FIG. 6: Examination of cJUN N-terminal Kinase (JNK) activity using a kinase assay for it's ability to phosphorylate a GST-cJUN protein. 293 HEK cells were transfected with pmKIT vectors encoding proteins listed above the first six lanes or stimulated with EGF or Sorbitol as controls for JNK activation. HA—hemagglutinin tag (vector control), E6-E6 from HPV 16, ΔPL-E6 from HPV 16 with deleted PDZ Ligand, E7-E7 protein from HPV 16, E6/E7—co-transfection with both proteins, ΔPL/E7—co-transfected with PL-deleted E6 and wild type E7. Brackets indicate the sizes of phosphorylated GST-Jun fusions used to assess JNK activity.

FIG. 7: Titration curve showing binding of a 20 amino acid peptide corresponding to the C-terminus of the E6 protein from HPV 16 to a PDZ domain containing protein TIP-1. Assay was performed as described in the specification (G assay). Numbers on the X-axis are micromolar units.

FIG. 8: displays four panels of graphs, A-E, showing effect of small molecule inhibitors on the interaction between E6 protein from HPV 16 and TIP-1.

FIG. 9A, 9B, 9C and 9D: HPV E6 activates JNK in epithelial cells. (A) HEK293 cells were transiently transfected with indicated Ha-tagged constructs. Lysates were used for immunoprecipitation and immunoblot detection with anti-HA antibodies (upper). Lysates from the same experiment were investigated in a GST-Jun pull down in vitro kinase assay for their JNK activity. Shown is the autoradiogram of the JNK assay (lower) (B) Xenopus oocytes were microinjected with bacterial expressed proteins of GST HPV16E6, GST HPV18E6 and GST HPV11E6 at 100 nM final concentration calculated per oocyte. After 3 h cells were lysed and lysates were tested for JNK activity (Upper). Oocytes were coinjected with GST HPV16E6 (100 nM) and a 20 mer peptide corresponding to the C-terminus of HPVE616. The peptide concentrations are indicated and are calculated as final concentration per oocyte. The control is the 20 mer C-terminal peptide of HPV11E6 at 10 μM. (C) Basal JNK activities in one HPV-negative (C33A) and six HPV-positive cervical cancer cell lines were tested. Shown is the quantification by PhosphorImager of three independent experiments. Differences in JNK expression were not significant and could not account for the observed differences in JNK activity between HPV positive and HPV negative cell lines (data not shown). (D) Expression of small interfering RNAs for MAGI 1 led to JNK activation. HEK293 cells were transfected with pSilencer vectors encoding small interfering RNA's for sequences not present in the human genome (si-control), present in GAPDH (si-GAPDH) (as an additional control) and for a sequence present in MAGI 1 (si-MAGI). Protein expression levels of MAGI 1 were significantly reduced compared to the two controls (Upper). JNK activity was measured from lysates of these transfection. Sorbitol treated 293 cells were used for positive control (Similar results were obtained in three independent experiments).

FIG. 10A, 10B, 10C and 10D: Regulation of MAGI 1 expression by HPV16 E6 PL (A) MAGI 1 and Dlg1 protein levels in HPV positive or negative cervical cancer cells. Total cell lysates analyzed by western blot with anti-Magi1 and anti-Dlg1 antibodies (B) Relative levels of Magi 1 and Dlg1 RNA levels in cervical cancer cell lines, as determined by real time PCR # (C) MAGI 1 and Dlg1 protein expression in HEK293 cells expressing E6 and E6ΔPL. Cells were transiently transfected with pmkit-HA-E6, pmkit-HA-E6ΔPL or the control pmkit-HA expression vector. Shown are the MAGI 1 protein expression levels. E6 protein expression levels were determined with anti-HA antibody and were comparable for E6 and -E6ΔPL (not shown) (D) Magi1 and Dlg1 RNA levels in 293 cells transfected with E6 and E6ΔPL analyzed by real time PCR.

FIGS. 11A, 11B, 11C: show the structures of various chemical groups used in the subject compositions and methods in panels A through O.

### DESCRIPTION

I. Definitions

As used herein, the term “biological function” in the context of a cell, refers to a detectable biological activity normally carried out by the cell, e.g., a phenotypic change such as cell proliferation, cell activation (e.g., T cell activation, B cell activation, T-B cell conjugate formation), cytokine release, degranulation, tyrosine phosphorylation, ion (e.g., calcium) flux, metabolic activity, apoptosis, changes in gene expression, maintenance of cell structure, cell migration, adherence to a substrate, signal transduction, cell-cell interactions, and others described herein or known in the art.

A ‘marker” or “biological marker” as used herein refers to a measurable or detectable entity in a biological sample. Examples or markers include nucleic acids, proteins, or chemicals that are present in biological samples. One example of a marker is the presence of viral or pathogen proteins or nucleic acids in a biological sample from a human source. As used herein the term “isolated” refers to a polynucleotide, a polypeptide, an antibody, or a host cell that is in an environment different from that in which the polynucleotide, the polypeptide, the antibody, or the host cell naturally occurs. A polynucleotide, a polypeptide, an antibody, or a host cell which is isolated is generally substantially purified.

A subject “infected” with HPV is a subject having cells that contain HPV. The HPV in the cells may not exhibit any other phenotype (i.e., cells infected with HPV do not have to be cancerous). In other words, cells infected with HPV may be pre-cancerous (i.e., not exhibiting any abnormal phenotype, other than those that may be associated with viral infection), or cancerous cells.

As used herein, the term “substantially purified” refers to a compound (e.g., either a polynucleotide or a polypeptide or an antibody) that is removed from its natural environment and is at least 60% free, preferably 75% free, and most preferably 90% free from other components with which it is naturally associated. Thus, for example, a composition containing A is “substantially free of” B when at least 85% by weight of the total A+B in the composition is A. Preferably, A comprises at least about 90% by weight of the total of A+B in the composition, more preferably at least about 95% or even 99% by weight.

The terms “polypeptide” and “protein” are used interchangeably throughout the application and mean at least two covalently attached amino acids, which includes proteins, polypeptides, oligopeptides and peptides. The protein may be made up of naturally occurring amino acids and peptide bonds, or synthetic peptidomimetic structures. Peptidomimetics will be discussed in greater detail below. Thus “amino acid”, or “peptide residue”, as used herein means both naturally occurring and synthetic amino acids. For example, homo-phenylalanine, citrulline and norleucine are considered amino acids for the purposes of the invention. “Amino acid” also includes imino acid residues such as proline and hydroxyproline. The side chains may be in either the (R) or the (S) configuration. Normally, the amino acids are in the (S) or L-configuration. If non-naturally occurring side chains are used, non-amino acid substituents may be used, for example to prevent or retard in vivo degradation. Naturally occurring amino acids are normally used and the protein is a cellular protein that is either endogenous or expressed recombinantly.

In general, polypeptides may be of any length, e.g., greater than 2 amino acids, greater than 4 amino acids, greater than about 10 amino acids, greater than about 20 amino acids, greater than about 50 amino acids, greater than about 100 amino acids, greater than about 300 amino acids, usually up to about 500 or 1000 or more amino acids. “Peptides” are generally greater than 2 amino acids, greater than 4 amino acids, greater than about 10 amino acids, greater than about 20 amino acids, usually up to about 3, 4, 5, 10, 30 or 50 amino acids. In some embodiments, peptides are between 5 and 30 amino acids in length.

A recombinant protein is distinguished from naturally occurring protein by at least one or more characteristics. For example, the protein may be isolated or purified away from some or all of the proteins and compounds with which it is normally associated in its wild type host, and thus may be substantially pure. For example, an isolated protein is unaccompanied by at least some of the material with which it is normally associated in its natural state, preferably constituting at least about 0.5%, more preferably at least about 5% by weight of the total protein in a given sample. A substantially pure protein comprises at least about 75% by weight of the total protein, with at least about 80% being preferred, and at least about 90% being particularly preferred. The definition includes, but is not limited to, the production of a protein from one organism in a different organism or host cell. Alternatively, the protein may be made at a significantly higher concentration than is normally seen, through the use of an inducible promoter or high expression promoter, such that the protein is made at increased concentration levels. Alternatively, the protein may be in a form not normally found in nature, as in the addition of an epitope tag or amino acid substitutions, insertions and deletions, as discussed below. A “fusion protein” or “fusion polypeptide” as used herein refers to a composite protein, i.e., a single contiguous amino acid sequence, made up of two (or more) distinct, heterologous polypeptides that are not normally fused together in a single amino acid sequence. Thus, a fusion protein can include a single amino acid sequence that contains two entirely distinct amino acid sequences or two similar or identical polypeptide sequences, provided that these sequences are not normally found together in the same configuration in a single amino acid sequence found in nature. Fusion proteins can generally be prepared using either recombinant nucleic acid methods, i.e., as a result of transcription and translation of a recombinant gene fusion product, which fusion comprises a segment encoding a polypeptide of the invention and a segment encoding a heterologous protein, or by chemical synthesis methods well known in the art. A “fusion protein construct” as used herein is a polynucleotide encoding a fusion protein.

An “oncogenic HPV strain” is an HPV strain that is known to cause cervical cancer as determined by the National Cancer Institute (NCI,2001). “Oncogenic E6 proteins” are E6 proteins encoded by the above oncogenic HPV strains. Exemplary oncogenic strains are shown in Table 3. Oncogenic strains of HPV not specifically listed here, are known in the art, and may be found at the world wide website of the National Center for Biotechnology Information (NCBI).

An “oncogenic E6 protein binding partner” is any molecule that specifically binds to an oncogenic E6 protein. Suitable oncogenic E6 protein binding partners include a PDZ domain (as described below), an antibody against an oncogenic E6 protein; other proteins that recognize oncogenic E6 protein (e.g., p53, E6-AP or E6-BP); DNA (i.e., cruciform DNA); and other partners such as aptamers or single chain antibodies from phage display). In general, binding partner bind E6 with an binding affinity of 10⁻⁵ M or more, e.g., 10⁻⁶ or more, 10⁻⁷ or more, 10⁻⁸ M or more (e.g., 10⁻⁹ M, 10⁻¹⁰, 10⁻¹¹, etc.).

As used herein, the term “PDZ domain” refers to protein sequence (i.e., modular protein domain) of less than approximately 90 amino acids, (i.e., about 80-90, about 70-80, about 60-70 or about 50-60 amino acids), characterized by homology to the brain synaptic protein PSD-95, the Drosophila septate junction protein Discs-Large (DLG), and the epithelial tight junction protein ZO1 (ZO1). PDZ domains are also known as Discs-Large homology repeats (“DHRs”) and GLGF repeats. PDZ domains generally appear to maintain a core consensus sequence (Doyle, D. A., 1996, Cell 85: 1067-76).

PDZ domains are found in diverse membrane-associated proteins including members of the MAGUK family of guanylate kinase homologs, several protein phosphatases and kinases, neuronal nitric oxide synthase, tumor suppressor proteins, and several dystrophin-associated proteins, collectively known as syntrophins.

Exemplary PDZ domain-containing proteins and PDZ domain sequences are shown in TABLE 2 and EXAMPLE 4. The term “PDZ domain” also encompasses variants (e.g., naturally occurring variants) of the sequences (e.g., polymorphic variants, variants with conservative substitutions, and the like) and domains from alternative species (e.g. mouse, rat). Typically, PDZ domains are substantially identical to those shown in U.S. patent application Ser. No. 09/724553, e.g., at least about 70%, at least about 80%, or at least about 90% amino acid residue identity when compared and aligned for maximum correspondence. It is appreciated in the art that PDZ domains can be mutated to give amino acid changes that can strengthen or weaken binding and to alter specificity, yet they remain PDZ domains (Schneider et al,., 1998, Nat. Biotech. 17:170-5). Unless otherwise indicated, a reference to a particular PDZ domain (e.g. a MAGI-1 domain 2) is intended to encompass the particular PDZ domain and HPV E6-binding variants thereof. In other words, if a reference is made to a particular PDZ domain, a reference is also made to variants of that PDZ domain that bind oncogenic E6 protein of HPV, as described below. In this respect it is noted that the numbering of PDZ domains in a protein may change. For example, the MAGI-1 domain 2, as referenced herein, may be referenced as MAGI-1 domain. 1 in other literature. As such, when a particular PDZ domain of a protein is referenced in this application, this reference should be understood in view of the sequence of that domain, as described herein, particularly in the sequence listing.

As used herein, the term “PDZ protein” refers to a naturally occurring protein containing a PDZ domain. Exemplary PDZ proteins include CASK, MPP1, DLG1, DLG2, PSD95, NeDLG, TIP-33, SYN1a, TIP-43, LDP, LIM, LIMK1, LIMK2, MPP2, NOS1, AF6, PTN-4, prIL16, 41.8 kD, KIAA0559, RGS12, KIAA0316, DVL1, TIP-40, TIAM1, MINT1, MAGI-1, MAGI-2, MAGI-3, KIAA0303, CBP, MINT3, TIP-2, KIAA0561, and TIP-1.

As used herein, the term “PDZ-domain polypeptide” refers to a polypeptide containing a PDZ domain, such as a fusion protein including a PDZ domain sequence, a naturally occurring PDZ protein, or an isolated PDZ domain peptide. A PDZ-domain polypeptide may therefore be about 60 amino acids or more in length, about 70 amino acids or more in length, about 80 amino acids or more in length, about 90 amino acids or more in length, about 100 amino acids or more in length, about 200 amino acids or more in length, about 300 amino acids or more in length, about 500 amino acids or more in length, about 800 amino acids or more in length, about 1000 amino acids or more in length, usually up to about 2000 amino acids or more in length. PDZ domain peptides are usually no more than about 100 amino acids (e.g. 50-60 amino acids, 60-70 amino acids, 80-90 amino acids, or 90-100 amino acids), and encode a PDZ domain.

As used herein, the term “PL protein” or “PDZ Ligand protein” refers to a polypeptide that may be a naturally-occurring or non-naturally occurring peptide, that forms a molecular complex with a PDZ-domain, or to a protein whose carboxy-terminus, when expressed separately from the full length protein (e.g., as a peptide of 4-25 residues, e.g., 8, 10, 12, 14 or 16 residues), forms such a molecular complex. The molecular complex can be observed in vitro using the “A assay” or “G assay” described infra, or in vivo. Exemplary PL proteins listed in TABLES 2 and 3 are demonstrated to bind specific PDZ proteins. This definition is not intended to include anti-PDZ antibodies and the like.

As used herein, a “PDZ ligand sequence” refers to the amino acid sequence of the C-terminus of a PL protein (e.g., the C-terminal 2, 3, 4, 5, 6, 7, 8, 9, 10, 12, 14, 16, 20 or 25 residues) (“C-terminal PL sequence”) or to an internal sequence known to bind a PDZ domain (“internal PL sequence”), or variant thereof.

As used herein, a “PDZ ligand peptide” is a peptide of having a sequence from, or based on, the sequence of the C-terminus of a PL protein. Exemplary PL peptides (biotinylated) are listed in TABLE 2.

As used herein, a “PL detector” is a protein that can specifically recognize and bind to a PL sequence.

As used herein, a “PL fusion protein” is a fusion protein that has a PL sequence as one domain, typically as the C-terminal domain of the fusion protein. An exemplary PL fusion protein is a tat-PL sequence fusion.

As used herein, the term “PL inhibitor peptide sequence” refers to PL peptide amino acid sequence that (in the form of a peptide or PL fusion protein) inhibits the interaction between a PDZ domain polypeptide and a PL peptide (e.g., in an A assay or a G assay).

As used herein, a “PDZ-domain encoding sequence” means a segment of a polynucleotide encoding a PDZ domain. In various embodiments, the polynucleotide is DNA, RNA, single stranded or double stranded.

As used herein, the terms “antagonist” and “inhibitor,” when used in the context of modulating a binding interaction (such as the binding of a PDZ domain sequence to a PL sequence), are used interchangeably and refer to an agent that reduces the binding of the, e.g., PL sequence (e.g., PL peptide) and the, e.g., PDZ domain sequence (e.g., PDZ protein, PDZ domain peptide).

As used herein, the terms “agonist” and “enhancer,” when used in the context of modulating a binding interaction (such as the binding of a PDZ domain sequence to a PL sequence), are used interchangeably and refer to an agent that increases the binding of the, e.g., PL sequence (e.g., PL peptide) and the, e.g., PDZ domain sequence (e.g., PDZ protein, PDZ domain peptide).

As used herein, the terms “peptide mimetic,” “peptidomimetic,” and “peptide analog” are used interchangeably and refer to a synthetic chemical compound that has substantially the same structural and/or functional characteristics of a PL inhibitory or PL binding peptide of the invention. The mimetic can be either entirely composed of synthetic, non-natural analogues of amino acids, or, is a chimeric molecule of partly natural peptide amino acids and partly non-natural analogs of amino acids. The mimetic can also incorporate any amount of natural amino acid conservative substitutions as long as such substitutions also do not substantially alter the mimetic's structure and/or inhibitory or binding activity. As with polypeptides of the invention which are conservative variants, routine experimentation will determine whether a mimetic is within the scope of the invention, i.e., that its structure and/or function is not substantially altered. Thus, a mimetic composition is within the scope of the invention if it is capable of binding to a PDZ domain and/or inhibiting a PL-PDZ interaction.

Polypeptide mimetic compositions can contain any combination of nonnatural structural components, which are typically from three structural groups: a) residue linkage groups other than the natural amide bond (“peptide bond”) linkages; b) non-natural residues in place of naturally occurring amino acid residues; or c) residues which induce secondary structural mimicry, i.e., to induce or stabilize a secondary structure, e.g., a beta turn, gamma turn, beta sheet, alpha helix conformation, and the like.

A polypeptide can be characterized as a mimetic when all or some of its residues are joined by chemical means other than natural peptide bonds. Individual peptidomimetic residues can be joined by peptide bonds, other chemical bonds or coupling means, such as, e.g., glutaraldehyde, N-hydroxysuccinimide esters, bifunctional maleimides, N,N═-dicyclohexylcarbodiimide (DCC) or N,N═-diisopropylcarbodiimide (DIC). Linking groups that can be an alternative to the traditional amide bond (“peptide bond”) linkages include, e.g., ketomethylene (e.g., —C(═O)—CH₂— for —C(═O)—NH—), aminomethylene (CH₂—NH), ethylene, olefin (CH═CH), ether (CH₂—O), thioether (CH₂—S), tetrazole (CN₄—), thiazole, retroamide, thioamide, or ester (see, e.g., Spatola (1983) in Chemistry and Biochemistry of Amino Acids, Peptides and Proteins, Vol. 7, pp 267-357, A Peptide Backbone Modifications, Marcell Dekker, NY).

A polypeptide can also be characterized as a mimetic by containing all or some non-natural residues in place of naturally occurring amino acid residues. Nonnatural residues are well described in the scientific and patent literature; a few exemplary nonnatural compositions useful as mimetics of natural amino acid residues and guidelines are described below.

Mimetics of aromatic amino acids can be generated by replacing by, e.g., D- or L-naphylalanine; D- or L-phenylglycine; D- or L-2 thieneylalanine; D- or L-1, -2, 3-, or 4-pyreneylalanine; D- or L-3 thieneylalanine; D- or L-(2-pyridinyl)-alanine; D- or L-(3-pyridinyl)-alanine; D- or L-(2-pyrazinyl)-alanine; D- or L-(4-isopropyl)-phenylglycine; D-(trifluoromethyl)-phenylglycine; D-(trifluoromethyl)-phenylalanine; D-p-fluorophenylalanine; D- or L-p-biphenylphenylalanine; K- or L-p-methoxybiphenylphenylalanine; D- or L-2-indole(alkyl)alanines; and, D- or L-alkylainines, where alkyl can be substituted or unsubstituted methyl, ethyl, propyl, hexyl, butyl, pentyl, isopropyl, iso-butyl, sec-isotyl, iso-pentyl, or a non-acidic amino acids. Aromatic rings of a nonnatural amino acid include, e.g., thiazolyl, thiophenyl, pyrazolyl, benzimidazolyl, naphthyl, furanyl, pyrrolyl, and pyridyl aromatic rings.

Mimetics of acidic amino acids can be generated by substitution by, e.g., non-carboxylate amino acids while maintaining a negative charge; (phosphono)alanine; sulfated threonine. Carboxyl side groups (e.g., aspartyl or glutamyl) can also be selectively modified by reaction with carbodiimides (R═—N—C—N—R═) such as, e.g., 1-cyclohexyl-3(2-morpholinyl-(4-ethyl) carbodiimide or 1-ethyl-3(4-azonia-4,4-dimetholpentyl) carbodiimide. Aspartyl or glutamyl can also be converted to asparaginyl and glutaminyl residues by reaction with ammonium ions.

Mimetics of basic amino acids can be generated by substitution with, e.g., (in addition to lysine and arginine) the amino acids ornithine, citrulline, or (guanidino)-acetic acid, or (guanidino)alkyl-acetic acid, where alkyl is defined above. Nitrile derivative (e.g., containing the CN-moiety in place of COOH) can be substituted for asparagine or glutamine. Asparaginyl and glutaminyl residues can be deaminated to the corresponding aspartyl or glutamyl residues.

Arginine residue mimetics can be generated by reacting arginyl with, e.g., one or more conventional reagents, including, e.g., phenylglyoxal, 2,3-butanedione, 1,2-cyclohexanedione, or ninhydrin, preferably under alkaline conditions.

Tyrosine residue mimetics can be generated by reacting tyrosyl with, e.g., aromatic diazonium compounds or tetranitromethane. N-acetylimidizol and tetranitromethane can be used to form O-acetyl tyrosyl species and 3-nitro derivatives, respectively.

Cysteine residue mimetics can be generated by reacting cysteinyl residues with, e.g., alpha-haloacetates such as 2-chloroacetic acid or chloroacetamide and corresponding amines, to give carboxymethyl or carboxyamidomethyl derivatives. Cysteine residue mimetics can also be generated by reacting cysteinyl residues with, e.g., bromo-trifluoroacetone, alpha-bromo-beta-(5-imidozoyl) propionic acid; chloroacetyl phosphate, N-alkylmaleimides, 3-nitro-2-pyridyl disulfide; methyl 2-pyridyl disulfide; p-chloromercuribenzoate; 2-chloromercuri-4 nitrophenol; or, chloro-7-nitrobenzo-oxa-1,3-diazole.

Lysine mimetics can be generated (and amino terminal residues can be altered) by reacting lysinyl with, e.g., succinic or other carboxylic acid anhydrides. Lysine and other alpha-amino-containing residue mimetics can also be generated by reaction with imidoesters, such as methyl picolinimidate, pyridoxal phosphate, pyridoxal, chloroborohydride, trinitrobenzenesulfonic acid, O-methylisourea, 2,4, pentanedione, and transamidase-catalyzed reactions with glyoxylate.

Mimetics of methionine can be generated by reaction with, e.g., methionine sulfoxide. Mimetics of proline include, e.g., pipecolic acid, thiazolidine carboxylic acid, 3- or 4-hydroxy proline, dehydroproline, 3- or 4-methylproline, or 3,3,-dimethylproline. Histidine residue mimetics can be generated by reacting histidyl with, e.g., diethylprocarbonate or para-bromophenacyl bromide.

Other mimetics include, e.g., those generated by hydroxylation of proline and lysine; phosphorylation of the hydroxyl groups of seryl or threonyl residues; methylation of the alpha-amino groups of lysine, arginine and histidine; acetylation of the N-terminal amine; methylation of main chain amide residues or substitution with N-methyl amino acids; or amidation of C-terminal carboxyl groups.

A component of a natural polypeptide (e.g., a PL polypeptide or PDZ polypeptide) can also be replaced by an amino acid (or peptidomimetic residue) of the opposite chirality. Thus, any amino acid naturally occurring in the L-configuration (which can also be referred to as the R or S, depending upon the structure of the chemical entity) can be replaced with the amino acid of the same chemical structural type or a peptidomimetic, but of the opposite chirality, generally referred to as the D- amino acid, but which can additionally be referred to as the R- or S-form. The mimetics of the invention can also include compositions that contain a structural mimetic residue, particularly a residue that induces or mimics secondary structures, such as a beta turn, beta sheet, alpha helix structures, gamma turns, and the like. For example, substitution of natural amino acid residues with D-amino acids; N-alpha-methyl amino acids; C-alpha-methyl amino acids; or dehydroamino acids within a peptide can induce or stabilize beta turns, gamma turns, beta sheets or alpha helix conformations. Beta turn mimetic structures have been described, e.g., by Nagai (1985) Tet. Lett. 26:647-650; Feigl (1986) J. Amer. Chem. Soc. 108:181-182; Kahn (1988) J. Amer. Chem. Soc. 110:1638-1639; Kemp (1988) Tet. Lett. 29:5057-5060; Kahn (1988) J. Molec. Recognition 1:75-79. Beta sheet mimetic structures have been described, e.g., by Smith (1992) J. Amer. Chem. Soc. 114:10672-10674. For example, a type IV beta turn induced by a cis amide surrogate, 1,5-disubstituted tetrazol, is described by Beusen (1995) Biopolymers 36:181-200. Incorporation of achiral omega-amino acid residues to generate polymethylene units as a substitution for amide bonds is described by Banerjee (1996) Biopolymers 39:769-777. Secondary structures of polypeptides can be analyzed by, e.g., high-field 1H NMR or 2D NMR spectroscopy, see, e.g., Higgins (1997) J. Pept. Res. 50:421-435. See also, Hruby (1997) Biopolymers 43:219-266, Balaji, et al., U.S. Pat. No. 5,612,895.

As used herein, “peptide variants” and “conservative amino acid substitutions” refer to peptides that differ from a reference peptide (e.g., a peptide having the sequence of the carboxy-terminus of a specified PL protein) by substitution of an amino acid residue having similar properties (based on size, polarity, hydrophobicity, and the like). Thus, insofar as the compounds that are encompassed within the scope of the invention are partially defined in terms of amino acid residues of designated classes, the amino acids may be generally categorized into three main classes: hydrophilic amino acids, hydrophobic amino acids and cysteine-like amino acids, depending primarily on the characteristics of the amino acid side chain. These main classes may be further divided into subclasses. Hydrophilic amino acids include amino acids having acidic, basic or polar side chains and hydrophobic amino acids include amino acids having aromatic or apolar side chains. Apolar amino acids may be further subdivided to include, among others, aliphatic amino acids. The definitions of the classes of amino acids as used herein are as follows:

“Hydrophobic Amino Acid” refers to an amino acid having a side chain that is uncharged at physiological pH and that is repelled by aqueous solution. Examples of genetically encoded hydrophobic amino acids include Ile, Leu and Val. Examples of non-genetically encoded hydrophobic amino acids include t-BuA.

“Aromatic Amino Acid” refers to a hydrophobic amino acid having a side chain containing at least one ring having a conjugated n-electron system (aromatic group). The aromatic group may be further substituted with groups such as alkyl, alkenyl, alkynyl, hydroxyl, sulfanyl, nitro and amino groups, as well as others. Examples of genetically encoded aromatic amino acids include Phe, Tyr and Trp. Commonly encountered non-genetically encoded aromatic amino acids include phenylglycine, 2-naphthylalanine, β-2-thienylalanine, 1,2,3,4-tetrahydroisoquinoline-3-carboxylic acid, 4-chloro-phenylalanine, 2-fluorophenyl-alanine, 3-fluorophenylalanine and 4-fluorophenylalanine.

“Anolar Amino Acid” refers to a hydrophobic amino acid having a side chain that is generally uncharged at physiological pH and that is not polar. Examples of genetically encoded apolar amino acids include Gly, Pro and Met. Examples of non-encoded apolar amino acids include Cha.

“Aliphatic Amino Acid” refers to an apolar amino acid having a saturated or unsaturated straight chain, branched or cyclic hydrocarbon side chain. Examples of genetically encoded aliphatic amino acids include Ala, Leu, Val and Ile. Examples of non-encoded aliphatic amino acids include Nle.

“Hydrophilic Amino Acid” refers to an amino acid having a side chain that is attracted by aqueous solution. Examples of genetically encoded hydrophilic amino acids include Ser and Lys. Examples of non-encoded hydrophilic amino acids include Cit and hCys.

“Acidic Amino Acid” refers to a hydrophilic amino acid having a side chain pK value of less than 7. Acidic amino acids typically have negatively charged side chains at physiological pH due to loss of a hydrogen ion. Examples of genetically encoded acidic amino acids include Asp and Glu.

“Basic Amino Acid” refers to a hydrophilic amino acid having a side chain pK value of greater than 7. Basic amino acids typically have positively charged side chains at physiological pH due to association with hydronium ion. Examples of genetically encoded basic amino acids include Arg, Lys and His. Examples of non-genetically encoded basic amino acids include the non-cyclic amino acids ornithine, 2,3-diaminopropionic acid, 2,4-diaminobutyric acid and homoarginine.

“Polar Amino Acid” refers to a hydrophilic amino acid having a side chain that is uncharged at physiological pH, but which has a bond in which the pair of electrons shared in common by two atoms is held more closely by one of the atoms. Examples of genetically encoded polar amino acids include Asx and Glx. Examples of non-genetically encoded polar amino acids include citrulline, N-acetyl lysine and methionine sulfoxide.

“Cysteine-Like Amino Acid” refers to an amino acid having a side chain capable of forming a covalent linkage with a side chain of another amino acid residue, such as a disulfide linkage. Typically, cysteine-like amino acids generally have a side chain containing at least one thiol (SH) group. Examples of genetically encoded cysteine-like amino acids include Cys. Examples of non-genetically encoded cysteine-like amino acids include homocysteine and penicillamine.

As will be appreciated by those having skill in the art, the above classification are not absolute—several amino acids exhibit more than one characteristic property, and can therefore be included in more than one category. For example, tyrosine has both an aromatic ring and a polar hydroxyl group. Thus, tyrosine has dual properties and can be included in both the aromatic and polar categories. Similarly, in addition to being able to form disulfide linkages, cysteine also has apolar character. Thus, while not strictly classified as a hydrophobic or apolar amino acid, in many instances cysteine can be used to confer hydrophobicity to a peptide.

Certain commonly encountered amino acids which are not genetically encoded of which the peptides and peptide analogues of the invention may be composed include, but are not limited to, β-alanine (b-Ala) and other omega-amino acids such as 3-aminopropionic acid (Dap), 2,3-diaminopropionic acid (Dpr), 4-aminobutyric acid and so forth; α-aminoisobutyric acid (Aib); ε-aminohexanoic acid (Aha); δ-aminovaleric acid (Ava); N-methylglycine or sarcosine (MeGly); ornithine (Orn); citrulline (Cit); t-butylalanine (t-BuA); t-butylglycine (t-BuG); N-methylisoleucine (MeIle); phenylglycine (Phg); cyclohexylalanine (Cha); norleucine (Nle); 2-naphthylalanine (2-Nal); 4-chlorophenylalanine (Phe(4-Cl)); 2-fluorophenylalanine (Phe(2-F)); 3-fluorophenylalanine (Phe(3-F)); 4-fluorophenylalanine (Phe(4-F)); penicillamine (Pen); 1,2,3,4-tetrahydroisoquinoline-3-carboxylic acid (Tic); β-2-thienylalanine (Thi); methionine sulfoxide (MSO); homoarginine (hArg); N-acetyl lysine (AcLys); 2,3-diaminobutyric acid (Dab); 2,3-diaminobutyric acid (Dbu); p-aminophenylalanine (Phe(pNH₂)); N-methyl valine (MeVal); homocysteine (hCys) and homoserine (hSer). These amino acids also fall conveniently into the categories defined above.

The classifications of the above-described genetically encoded and non-encoded amino acids are summarized in TABLE 1, below. It is to be understood that TABLE 1 is for illustrative purposes only and does not purport to be an exhaustive list of amino acid residues which may comprise the peptides and peptide analogues described herein. Other amino acid residues which are useful for making the peptides and peptide analogues described herein can be found, e.g., in Fasman, 1989, CRC Practical Handbook of Biochemistry and Molecular Biology, CRC Press, Inc., and the references cited therein. Amino acids not specifically mentioned herein can be conveniently classified into the above-described categories on the basis of known behavior and/or their characteristic chemical and/or physical properties as compared with amino acids specifically identified.

TABLE 1

| | Genetically | |
|---|---|---|
| Classification | Encoded | Genetically Non-Encoded |
| Hydrophobic | | |
| Aromatic | F, Y, W | Phg, Nal, Thi, Tic, Phe(4-Cl), Phe(2-F), |
| | | Phe(3-F), Phe(4-F), Pyridyl Ala, |
| | | Benzothienyl Ala |
| Apolar | M, G, P | |
| Aliphatic | A, V, L, I | t-BuA, t-BuG, MeIle, Nle, MeVal, Cha, |
| | | bAla, MeGly, Aib |
| Hydrophilic | | |
| Acidic | D, E | |
| Basic | H, K, R | Dpr, Orn, hArg, Phe(p-NH₂), DBU, |
| | | A₂BU |
| Polar | Q, N, S, T, Y | Cit, AcLys, MSO, hSer |
| Cysteine-Like | C | Pen, hCys, p-methyl Cys |

In the case of the PDZ domains described herein, a “HPV E6-binding variant” of a particular PDZ domain is a PDZ domain variant that retains HPV E6 PDZ ligand binding activity. Assays for determining whether a PDZ domain variant binds HPV E6 are described in great detail below, and guidance for identifying which amino acids to change in a specific PDZ domain to make it into a variant may be found in a variety of sources. In one example, a PDZ domain may be compared to other PDZ domains described herein and amino acids at corresponding positions may be substituted, for example. In another example, the sequence a PDZ domain of a particular PDZ protein may be compared to the sequence of an equivalent PDZ domain in an equivalent PDZ protein from another species. For example, the sequence a PDZ domain from a human PDZ protein may be compared to the sequence of other known and equivalent PDZ domains from other species (e.g., mouse, rat, etc.) and any amino acids that are variant between the two sequences may be substituted into the human PDZ domain to make a variant of the PDZ domain. For example, the sequence of the human MAGI-1 PDZ domain 2 maybe compared to equivalent MAGI-1 PDZ domains from other species (e.g. mouse Genbank gi numbers 7513782 and 28526157 or other homologous sequences) to identify amino acids that may be substituted into the human MAGI-1-PDZ domain to make a variant thereof. Such method may be applied to any of the MAGI-1 PDZ domains described herein. Minimal MAGI-PDZ domain 2 sequence is provided as SEQ ID NOS:320-328. Particular variants may have 1, up to 5, up to about 10, up to about 15, up to about 20 or up to about 30 or more, usually up to about 50 amino acid changes as compared to a sequence set forth in the sequence listing. Exemplary MAGI-1 PDZ variants include the sequences set forth in SEQ ID NOS: 329-357. In making a variant, if a GFG motif is present in a PDZ domain, in general, it should not be altered in sequence.

In general, variant PDZ domain polypeptides have a PDZ domain that has at least about 70 or 80%, usually at least about 90%, and more usually at least about 98% sequence identity with a variant PDZ domain polypeptide described herein, as measured by BLAST 2.0 using default parameters, over a region extending over the entire PDZ domain.

As used herein, a “detectable label” has the ordinary meaning in the art and refers to an atom (e.g., radionuclide), molecule (e.g., fluorescein), or complex, that is or can be used to detect (e.g., due to a physical or chemical property), indicate the presence of a molecule or to enable binding of another molecule to which it is covalently bound or otherwise associated. The term “label” also refers to covalently bound or otherwise associated molecules (e.g., a biomolecule such as an enzyme) that act on a substrate to produce a detectable atom, molecule or complex. Detectable labels suitable for use in the present invention include any composition detectable by spectroscopic, photochemical, biochemical, immunochemical, electrical, optical or chemical means. Labels useful in the present invention include biotin for staining with labeled streptavidin conjugate, magnetic beads (e.g., Dynabeads™), fluorescent dyes (e.g., fluorescein, Texas red, rhodamine, green fluorescent protein, enhanced green fluorescent protein, and the like), radiolabels (e.g., ³H, ¹²⁵I, ³⁵S, ¹⁴C, or ³²P), enzymes ( e.g., hydrolases, particularly phosphatases such as alkaline phosphatase, esterases and glycosidases, or oxidoreductases, particularly peroxidases such as horse radish peroxidase, and others commonly used in ELISAS), substrates, cofactors, inhibitors, chemiluminescent groups, chromogenic agents, and colorimetric labels such as colloidal gold or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241. Means of detecting such labels are well known to those of skill in the art. Thus, for example, radiolabels and chemiluminescent labels may be detected using photographic film or scintillation counters, fluorescent markers may be detected using a photodetector to detect emitted light (e.g., as in fluorescence-activated cell sorting). Enzymatic labels are typically detected by providing the enzyme with a substrate and detecting the reaction product produced by the action of the enzyme on the substrate, and colorimetric labels are detected by simply visualizing the colored label. Thus, a label is any composition detectable by spectroscopic, photochemical, biochemical, immunochemical, electrical, optical or chemical means. The label may be coupled directly or indirectly to the desired component of the assay according to methods well known in the art. Non-radioactive labels are often attached by indirect means. Generally, a ligand molecule (e.g., biotin) is covalently bound to the molecule. The ligand then binds to an anti-ligand (e.g., streptavidin) molecule which is either inherently detectable or covalently bound to a signal generating system, such as a detectable enzyme, a fluorescent compound, or a chemiluminescent compound. A number of ligands and anti-ligands can be used. Where a ligand has a natural anti-ligand, for example, biotin, thyroxine, and cortisol, it can be used in conjunction with the labeled, naturally occurring anti-ligands. Alternatively, any haptenic or antigenic compound can be used in combination with an antibody. The molecules can also be conjugated directly to signal generating compounds, e.g., by conjugation with an enzyme or fluorophore. Means of detecting labels are well known to those of skill in the art. Thus, for example, where the label is a radioactive label, means for detection include a scintillation counter, photographic film as in autoradiography, or storage phosphor imaging. Where the label is a fluorescent label, it may be detected by exciting the fluorochrome with the appropriate wavelength of light and detecting the resulting fluorescence. The fluorescence may be detected visually, by means of photographic film, by the use of electronic detectors such as charge coupled devices (CCDs) or photomultipliers and the like. Similarly, enzymatic labels may be detected by providing the appropriate substrates for the enzyme and detecting the resulting reaction product. Also, simple colorimetric labels may be detected by observing the color associated with the label. It will be appreciated that when pairs of fluorophores are used in an assay, it is often preferred that they have distinct emission patterns (wavelengths) so that they can be easily distinguished.

As used herein, the term “substantially identical” in the context of comparing amino acid sequences, means that the sequences have at least about 70%, at least about 80%, or at least about 90% amino acid residue identity when compared and aligned for maximum correspondence. An algorithm that is suitable for determining percent sequence identity and sequence similarity is the FASTA algorithm, which is described in Pearson, W. R. & Lipman, D. J., 1988, Proc. Natl. Acad. Sci. U.S.A. 85: 2444. See also W. R. Pearson, 1996, Methods Enzymol. 266: 227-258. Preferred parameters used in a FASTA alignment of DNA sequences to calculate percent identity are optimized, BL50 Matrix 15: −5, k-tuple=2; joining penalty=40, optimization=28; gap penalty −12, gap length penalty=−2; and width=16.

As used herein, the terms “sandwich”, “sandwich ELISA”, “Sandwich diagnostic” and “capture ELISA” all refer to the concept of detecting a biological polypeptide with two different test agents. For example, a PDZ protein could be attached to a solid support. Test sample could be passed over the surface and the PDZ protein could bind it's cognate PL protein(s). An antibody with detection reagent could then be used to determine whether a specific PL protein had bound the PDZ protein.

As used herein, the terms “test compound” or “test agent” are used interchangeably and refer to a candidate agent that may have enhancer/agonist, or inhibitor/antagonist activity, e.g., inhibiting or enhancing an interaction such as PDZ-PL binding. The candidate agents or test compounds may be any of a large variety of compounds, both naturally occurring and synthetic, organic and inorganic, and including polymers (e.g., oligopeptides, polypeptides, oligonucleotides, and polynucleotides), small molecules, antibodies (as broadly defined herein), sugars, fatty acids, nucleotides and nucleotide analogs, analogs of naturally occurring structures (e.g., peptide mimetics, nucleic acid analogs, and the like), and numerous other compounds. In certain embodiment, test agents are prepared from diversity libraries, such as random or combinatorial peptide or non-peptide libraries. Many libraries are known in the art that can be used, e.g., chemically synthesized libraries, recombinant (e.g., phage display libraries), and in vitro translation-based libraries. Examples of chemically synthesized libraries are described in Fodor et al., 1991, Science 251:767-773; Houghten et al., 1991, Nature 354:84-86; Lam et al., 1991, Nature 354:82-84; Medynski, 1994, Bio/Technology 12:709-710; Gallop et al., 1994, J. Medicinal Chemistry 37(9):1233-1251; Ohlmeyer et al., 1993, Proc. Natl. Acad. Sci. USA 90:10922-10926; Erb et al., 1994, Proc. Natl. Acad. Sci. USA 91:11422-11426; Houghten et al., 1992, Biotechniques 13:412; Jayawickreme et al., 1994, Proc. Natl. Acad. Sci. USA 91:1614-1618; Salmon et al., 1993, Proc. Natl. Acad. Sci. USA 90:11708-11712; PCT Publication No. WO 93/20242; and Brenner and Lerner, 1992, Proc. Natl. Acad. Sci. USA 89:5381-5383. Examples of phage display libraries are described in Scott and Smith, 1990, Science 249:386-390; Devlin et al., 1990, Science, 249:404-406; Christian, R. B., et al., 1992, J. Mol. Biol. 227:711-718); Lenstra, 1992, J. Immunol. Meth. 152:149-157; Kay et al., 1993, Gene 128:59-65; and PCT Publication No. WO 94/18318 dated Aug. 18, 1994. In vitro translation-based libraries include but are not limited to those described in PCT Publication No. WO 91/05058 dated Apr. 18, 1991; and Mattheakis et al., 1994, Proc. Natl. Acad. Sci. USA 91:9022-9026. By way of examples of nonpeptide libraries, a benzodiazepine library (see e.g., Bunin et al., 1994, Proc. Natl. Acad. Sci. USA 91:4708-4712) can be adapted for use. Peptoid libraries (Simon et al., 1992, Proc. Natl. Acad. Sci. USA 89:9367-9371) can also be used. Another example of a library that can be used, in which the amide functionalities in peptides have been permethylated to generate a chemically transformed combinatorial library, is described by Ostresh et al. (1994, Proc. Natl. Acad. Sci. USA 91:11138-11142). In certain embodiments, peptides containing at least two of the three C-terminal amino acids, of E6 proteins from oncogenic strains of HPV, or mimetics thereof.

The term “specific binding” refers to binding between two molecules, for example, a ligand and a receptor, characterized by the ability of a molecule (ligand) to associate with another specific molecule (receptor) even in the presence of many other diverse molecules, i.e., to show preferential binding of one molecule for another in a heterogeneous mixture of molecules. Specific binding of a ligand to a receptor is also evidenced by reduced binding of a detectably labeled ligand to the receptor in the presence of excess unlabeled ligand (i.e., a binding competition assay).

As used herein, a “plurality” of PDZ proteins (or corresponding PDZ domains or PDZ fusion polypeptides) has its usual meaning. In some embodiments, the plurality is at least 5, and often at least 25, at least 40, or at least 60 different PDZ proteins. In some embodiments, the plurality is selected from the list of PDZ polypeptides listed in TABLE 8. In some embodiments, the plurality of different PDZ proteins are from (i.e., expressed in) a particular specified tissue or a particular class or type of cell. In some embodiments, the plurality of different PDZ proteins represents a substantial fraction (e.g., typically at least 50%, more often at least 80%) of all of the PDZ proteins known to be, or suspected of being, expressed in the tissue or cell(s), e.g., all of the PDZ proteins known to be present in lymphocytes or hematopoetic cells. In some embodiments, the plurality is at least 50%, usually at least 80%, at least 90% or all of the PDZ proteins disclosed herein as being expressed in a particular cell.

When referring to PL peptides (or the corresponding proteins, e.g., corresponding to those listed in TABLE 2, or elsewhere herein) a “plurality” may refer to at least 5, at least 10, and often at least 16 PLs such as those specifically listed herein, or to the classes and percentages set forth supra for PDZ domains.

As used herein, “HPV PL protein” refers to a protein in the family of human papillomavirus proteins that displays a PDZ-ligand motif on the C-terminus of the protein.

II. Overview

Methods and compositions for treating a disease correlated with binding between a PDZ protein and a HPV protein containing a PL motif are also disclosed herein, the method comprising administering a therapeutically effective amount of a modulator as provided herein, wherein the PDZ protein and the PL protein are a binding pair as specified in Table 3. As indicated supra, such methods can be used to treat a variety of diseases associated with HPV infection, including, but not limited to, cervical cancer, penile cancer, anal cancer, throat cancer, skin cancer and genital warts.

Certain methods involve introducing into the cell an agent that alters binding between a PDZ protein and a HPV PL protein in the cell, whereby the biological function is modulated in the cell, and wherein the PDZ protein and PL protein are a binding pair as specified in Table 3. In some of these methods, the agent is a polypeptide comprising at least the two, three or four carboxy-terminal residues of the PL protein.

Screening methods to identify compounds that modulate binding between PDZ proteins and PL peptides or proteins are also provided. Some screening methods involve contacting under suitable binding conditions (i) a PDZ-domain polypeptide having a sequence from a PDZ protein, and (ii) a PL peptide, wherein the PL peptide comprises a C-terminal sequence of the PL protein, the PDZ -domain polypeptide and the PL peptide are a binding pair as specified in Table 3; and contacting is performed in the presence of the test compound. Presence or absence of complex is then detected. The presence of the complex at a level that is statistically significantly higher in the presence of the test compound than in the absence of test compound is an indication that the test compound is an agonist, whereas, the presence of the complex at a level that is statistically significantly lower in the presence of the test compound than in the absence of test compound is an indication that the test compound is an antagonist.

Modulators of binding between a PDZ protein and a PL protein are also described herein. In certain instances, the modulator is (a) a peptide comprising at least 3 residues of a C-terminal sequence of a PL protein, and wherein the PDZ protein and the PL protein are a binding pair as specified in Table 3; or (b) a peptide mimetic of the peptide of section (a); or (c) a small molecule having similar functional activity with respect to the PDZ and PL protein binding pair as the peptide of section (a). The modulator can be either an agonist or antagonist. Such modulators can be formulated as a pharmaceutical composition.

Routes of administration of modulators and effective dosages are also described herein. In certain instances, the modulator is administered topically, in the form of a cream.

III. PDZ Protein and PL Protein Interactions

TABLE 3 lists PDZ proteins and HPV PL proteins which the current inventors have identified as binding to one another. TABLE 3 is organized into four columns. The columns from left to right show the HPV E6 strain and terminal 4 amino acids of the PL that was tested in the G assay (generally 20 amino acids), followed by the PDZ domains that bound that ligand at high affinity, and then a repetition of additional HPV strains and PDZ domains that bind the E6 PL immediately to the left of the domains. Thus, the first column in each section is labeled “HPV Strain” and lists the names of the various E6 proteins and the carboxy-terminal 4 amino acids (potential PLs) that were examined. The second column, labeled “PDZ binding partner” lists PDZ domains that bind the biotinylated peptide at relatively high strength. All ligands are biotinylated at the amino-terminus and partial sequences are presented in TABLE 3.

The PDZ protein (or proteins) that interact(s) with HPV E6-PL peptides are listed in the third column labeled “PDZ binding partner”. This column provides the gene name for the PDZ portion of the GST-PDZ fusion that interacts with the PDZ-ligand to the left. For PDZ domain-containing proteins with multiple domains the domain number is listed to the right of the PDZ (i.e., in column 4 labeled “PDZ Domain”), and indicates the PDZ domain number when numbered from the amino-terminus to the carboxy-terminus. Column 5, labeled “Classification,” lists a measure of the level of binding, as determined in the “G” Assay. In particular, it provides an absorbance value at 450 nm which indicates the amount of PL peptide bound to the PDZ protein. The following numerical values have the following meanings: ‘1’—A₄₅₀ nm 0-1; ‘2’—A₄₅₀ nm 1-2; ‘3’—A₄₅₀ nm 2-3; ‘4’—A₄₅₀ nm 3-4; ‘5’—A₄₅₀ nm of 4 more than 2× repeated; ‘0’—A₄₅₀ nm 0, i.e., not found to interact.

Further information regarding these PL proteins and PDZ proteins is provided in TABLES 2 and 8. In particular, TABLE 2 provides a listing of the amino acid sequences of peptides used in the assays. When numbered from left to right, the first column labeled “HPV strain” provides the name of the HPV strain, corresponding to the name listed in column 1 of Table 2. The second column labeled “E6 C-terminal sequence” provides the predicted sequence of the carboxy-terminal 10 amino acids of the E6 protein. The third column labeled “PL yes/no” designates whether the E6-PL sequence contains sequence elements predicted by the inventors to bind to PDZ domains. The final column labeled “oncogenic” indicates that this HPV strain is known to cause cervical cancer as determined by the National Cancer Institute (NCI, 2001) or published reports in the literature.

TABLE 8 lists the sequences of the PDZ domains cloned into a vector (PGEX-3X vector) for production of GST-PDZ fusion proteins (Pharmacia). More specifically, the first column (left to right) entitled “Gene Name” lists the name of the gene containing the PDZ domain. The second column labeled “GI or Acc#” is a unique Genbank identifier for the gene used to design primers for PCR amplification of the listed sequence. The next column labeled “PDZ#” indicates the Pfam-predicted PDZ domain number, as numbered from the amino-terminus of the gene to the carboxy-terminus. The last column entitled “Sequence fused to GST construct” provides the actual amino acid sequence inserted into the GST-PDZ expression vector as determined by DNA sequencing of the constructs.

As discussed in detail herein, the PDZ proteins listed in TABLE 3 are naturally occurring proteins containing a PDZ domain. Only significant interactions are presented in this table. Thus, the present invention is particularly directed to the modulation of interactions between a PDZ protein and a HPV PL protein.

In another embodiment of the invention, cellular abnormalities or diseases can be treated through the correction of imbalances in the expression levels of cellular PDZ proteins or PL proteins. Using either the PL protein or the PDZ protein in an assay derived from the ‘A assay’ or ‘G assay’ one can determine the protein expression levels of binding partners in a normal or abnormal cell. Differences in protein expression levels have been correlated with a number of diseases.

In certain embodiments of the invention, a PDZ protein is used to treat diseases associated with the presence of a PL protein from a pathogenic organism, such as diseases associated with HPV infection, including but not limited to cervical cancer, genital warts, penile cancer, and anal cancer.

In a preferred embodiment of the invention, an antagonist of the interaction is used to block the interaction between a PDZ protein and a PL protein from a pathogenic organism, thus providing treatment for diseases associated with that pathogen. An antagonist may be in the form of a PL peptide, a PL protein, a peptide mimetic, a small molecule, or any other antagonist compound known in the art.

IV. Detecting PDZ-PL Interactions

The present inventors were able in part to identify the interactions summarized in TABLE 3 by developing new high throughput screening assays which are described in greater detail infra. Various other assay formats known in the art can be used to select ligands that are specifically reactive with a particular protein. For example, solid-phase ELISA immunoassays, immunoprecipitation, Biacore, and Western blot assays can be used to identify peptides that specifically bind PDZ-domain polypeptides. As discussed supra, two different, complementary assays were developed to detect PDZ-PL interactions. In each, one binding partner of a PDZ-PL pair is immobilized, and the ability of the second binding partner to bind is determined. These assays, which are described infra, can be readily used to screen for hundreds to thousands of potential PDZ-ligand interactions in a few hours. Thus these assays can be used to identify yet more novel PDZ-PL interactions in cells. In addition, they can be used to identify antagonists of PDZ-PL interactions (see infra).

In various embodiments, fusion proteins are used in the assays and devices of the invention. Methods for constructing and expressing fusion proteins are well known. Fusion proteins generally are described in Ausubel et al., supra, Kroll et al., 1993, DNA Cell. Biol. 12:441, and Imai et al., 1997, Cell 91:521-30. Usually, the fusion protein includes a domain to facilitate immobilization of the protein to a solid substrate (“an immobilization domain”). Often, the immobilization domain includes an epitope tag (i.e., a sequence recognized by an antibody, typically a monoclonal antibody) such as polyhistidine (Bush et al, 1991, J. Biol Chem 266:13811-14), SEAP (Berger et al, 1988, Gene 66:1-10), or M1 and M2 flag (see, e.g, U.S. Pat. Nos. 5,011,912; 4,851,341; 4,703,004; 4,782,137). In an embodiment, the immobilization domain is a GST coding region. It will be recognized that, in addition to the PDZ-domain and the particular residues bound by an immobilized antibody, protein A, or otherwise contacted with the surface, the protein (e.g., fusion protein), will contain additional residues. In some embodiments these are residues naturally associated with the PDZ-domain (i.e., in a particular PDZ-protein) but they may include residues of synthetic (e.g., poly(alanine)) or heterologous origin (e.g., spacers of, e.g., between 10 and 300 residues).

PDZ domain-containing polypeptide used in the methods of the invention (e.g., PDZ fusion proteins) of the invention are typically made by (1) constructing a vector (e.g., plasmid, phage or phagemid) comprising a polynucleotide sequence encoding the desired polypeptide, (2) introducing the vector into an suitable expression system (e.g., a prokaryotic, insect, mammalian, or cell free expression system), (3) expressing the fusion protein and (4) optionally purifying the fusion protein.

(1) In one embodiment, expression of the protein comprises inserting the coding sequence into an appropriate expression vector (i.e., a vector that contains the necessary elements for the transcription and translation of the inserted coding sequence required for the expression system employed, e.g., control elements including enhancers, promoters, transcription terminators, origins of replication, a suitable initiation codon (e.g., methionine), open reading frame, and translational regulatory signals (e.g., a ribosome binding site, a termination codon and a polyadenylation sequence. Depending on the vector system and host utilized, any number of suitable transcription and translation elements, including constitutive and inducible promoters, can be used.

The coding sequence of the fusion protein includes a PDZ domain and an immobilization domain as described elsewhere herein. Polynucleotides encoding the amino acid sequence for each domain can be obtained in a variety of ways known in the art; typically the polynucleotides are obtained by PCR amplification of cloned plasmids, cDNA libraries, and cDNA generated by reverse transcription of RNA, using primers designed based on sequences determined by the practitioner or, more often, publicly available (e.g., through GenBank). The primers include linker regions (e.g., sequences including restriction sites) to facilitate cloning and manipulation in production of the fusion construct. The polynucleotides corresponding to the PDZ and immobilization regions are joined in-frame to produce the fusion protein-encoding sequence.

The fusion proteins of the invention may be expressed as secreted proteins (e.g., by including the signal sequence encoding DNA in the fusion gene; see, e.g., Lui et al, 1993, PNAS USA, 90:8957-61) or as nonsecreted proteins.

A. Production of Fusion Proteins Containing PDZ-Domains

GST-PDZ domain fusion proteins were prepared for use in the assays of the invention. PCR products containing PDZ encoding domains (as described supra) were subcloned into an expression vector to permit expression of fusion proteins containing a PDZ domain and a heterologous domain (i.e., a glutathione-S transferase sequence, “GST”). PCR products (i.e., DNA fragments) representing PDZ domain encoding DNA were extracted from agarose gels using the “Sephaglas” gel extraction system (Pharmacia) according to the manufacturer's recommendations. Amino acid sequences for all of the PDZ domains used in the assays of the invention are listed in Table 8.

As noted supra, PCR primers were designed to include endonuclease restriction sites to facilitate ligation of PCR fragments into a GST gene fusion vector (pGEX-3X; Pharmacia, GenBank accession no. XXU13852) in-frame with the glutathione-S transferase coding sequence. This vector contains an IPTG inducible lacZ promoter. The pGEX-3X vector was linearized using Bam HI and Eco RI or, in some cases, Eco RI or Sma I, and dephosphorylated. For most cloning approaches, double digestion with Bain HI and Eco RI was performed, so that the ends of the PCR fragments to clone were Bam HI and Eco RI. In some cases, restriction endonuclease combinations used were Bgl II and Eco RI, Bam HI and Mfe I, or Eco RI only, Sma I only, or BamHI only. When more than one PDZ domain was cloned, the DNA portion cloned represents the PDZ domains and the cDNA portion located between individual domains. Precise locations of cloned fragments used in the assays are indicated in U.S. Patent Application (60/360061). DNA linker sequences between the GST portion and the PDZ domain containing DNA portion vary slightly, dependent on which of the above described cloning sites and approaches were used. As a consequence, the amino acid sequence of the GST-PDZ fusion protein varies in the linker region between GST and PDZ domain. Protein linker sequences corresponding to different cloning sites/approaches are shown below. Linker sequences (vector DNA encoded) are bold, PDZ domain containing gene derived sequences are in italics.
1) GST-BamHI/BamHI- PDZ domain insert
   Gly-Ile-PDZ domain insert
2) GST-BamHI/BglII-PDZ domain insert
   Gly-Ile-PDZ domain insert
3) GST-EcoRI/EcoI-PDZ domain insert
   Gly-Ile-Pro-Gly-Asn-PDZ domain insert
4) GST-SmaI/SmaI-PDZ domain insert
   Gly-Ile-Pro-PDZ domain insert

The PDZ-encoding PCR fragment and linearized pGEX-3X vector were ethanol precipitated and resuspended in 10 ul standard ligation buffer. Ligation was performed for 4-10 hours at 7° C. using T4 DNA ligase. It will be understood that some of the resulting constructs include very short linker sequences and that, when multiple PDZ domains were cloned, the constructs included some DNA located between individual PDZ domains.

The ligation products were transformed in DH5alpha or BL-21 E. coli bacteria strains. Colonies were screened for presence and identity of the cloned PDZ domain containing DNA as well as for correct fusion with the glutathione S-transferase encoding DNA portion by PCR and by sequence analysis. Positive clones were tested in a small-scale assay for expression of the GST/PDZ domain fusion protein and, if expressing, these clones were subsequently grown up for large scale preparations of GST/PDZ fusion protein.

GST-PDZ domain fusion protein was overexpressed following addition of IPTG to the culture medium and purified. Detailed procedure of small scale and large-scale fusion protein expression and purification are described in “GST Gene Fusion System” (second edition, revision 2; published by Pharmacia). In brief, a small culture (50 mls) containing a bacterial strain (DH5α, BL21 or JM109) with the fusion protein construct was grown overnight in 2×YT media at 37° C. with the appropriate antibiotic selection (100 ug/ml ampicillin; a.k.a. 2×YT-amp). The overnight culture was poured into a fresh preparation of 2×YT-amp (typically 1 liter) and grown until the optical density (OD) of the culture was between 0.5 and 0.9 (approximately 2.5 hours). IPTG (isopropyl β-D-thiogalactopyranoside) was added to a final concentration of 1.0 mM to induce production of GST fusion protein, and culture was grown an additional 1 hour. All following steps, including centrifugation, were performed on ice or at 4° C. Bacteria were collected by centrifugation (4500×g) and resuspended in Buffer A− (50 nmM Tris, pH 8.0, 50 mM dextrose, 1 mM EDTA, 200 uM phenylmethylsulfonylfluoride). An equal volume of Buffer A+ (Buffer A−, 4 mg/ml lysozyme) was added and incubated on ice for 3 min to lyse bacteria, or until lysis had begun. An equal volume of Buffer B (10 mM Tris, pH 8.0, 50 mM KCl, 1 mM EDTA. 0.5% Tween-20, 0.5% NP40 (a.k.a. IGEPAL CA-630), 200 uM phenylmethylsulfonylfluoride) was added and incubated for an additional 20 min on ice. The bacterial cell lysate was centrifuged (×20,000 g), and supernatant was run over a column containing 20 ml Sepharose CL-4B (Pharmacia) “precolumn beads,” i.e., sepharose beads without conjugated glutathione that had been previously washed with 3 bed volumes PBS. The flow-through was added to glutathione Sepharose 4B (Pharmacia, cat no.17-0765-01) previously swelled (rehydrated) in 1×phosphate-buffered saline (PBS) and incubated while rotating for 30 min-1 hr. The supernatant-Sepharose slurry was poured into a column and washed with at least 20 bed volumes of 1×PBS. GST fusion protein was eluted off the glutathione sepharose by applying 0.5-1.0 ml aliquots of 5 mM glutathione and collected as separate fractions. Concentrations of fractions were determined by reading absorbance at 280 nm and calculating concentration using the absorbance and extinction coefficient. Those fractions containing the highest concentration of fusion protein were pooled and an equal volume of 70% glycerol was added to a final concentration of 35% glycerol. Fusion proteins were assayed for size and quality by SDS gel electrophoresis (PAGE) as described in “Sambrook.” Fusion protein aliquots were stored at minus 80° C. and at minus 20° C.

B. Identification of Candidate PL Proteins and Synthesis of Peptides

Certain PDZ domains are bound by the C-terminal residues of PDZ-binding proteins. To identify PL proteins the C-terminal residues of sequences were visually inspected for sequences that one might predict would bind to PDZ-domain containing proteins (see, e.g., Doyle et al., 1996, Cell 85, 1067; Songyang et al., 1997, Science 275, 73), including the additional consenses for PLs identified at Arbor Vita Corporation (U.S. Patent Application 60/360061). TABLE 2 lists some of these proteins, and provides corresponding C-terminal sequences.

Synthetic peptides of defined sequence (e.g., corresponding to the carboxyl-termini of the indicated proteins) can be synthesized by any standard resin-based method (see, e.g., U.S. Pat. No. 4,108,846; see also, Caruthers et al., 1980, Nucleic Acids Res. Symp. Ser., 215-223; Horn et al., 1980, Nucleic Acids Res. Symp. Ser., 225-232; Roberge, et al., 1995, Science 269:202). The peptides used in the assays described herein were prepared by the FMOC (see, e.g., Guy and Fields, 1997, Meth. Enz. 289:67-83; Wellings and Atherton, 1997, Meth. Enz. 289:44-67). In some cases (e.g., for use in the A and G assays of the invention), peptides were labeled with biotin at the amino-terminus by reaction with a four-fold excess of biotin methyl ester in dimethylsulfoxide with a catalytic amount of base. The peptides were cleaved from the resin using a halide containing acid (e.g. trifluoroacetic acid) in the presence of appropriate antioxidants (e.g. ethanedithiol) and excess solvent lyophilized.

Following lyophilization, peptides can be redissolved and purified by reverse phase high performance liquid chromatography (HPLC). One appropriate HPLC solvent system involves a Vydac C-18 semi-preparative column running at 5 mL per minute with increasing quantities of acetonitrile plus 0.1% trifluoroacetic acid in a base solvent of water plus 0.1% trifluoroacetic acid. After HPLC purification, the identities of the peptides are confirmed by MALDI cation-mode mass spectrometry.

C. Assays for Detection of PDZ-PL Interactions

Two complementary assays, termed “A” and “G”, were developed to detect binding between a PDZ-domain polypeptide and candidate PDZ ligand. In each of the two different assays, binding is detected between a peptide having a sequence corresponding to the C-terminus of a HPV protein anticipated to bind to one or more PDZ domains (i.e. a candidate HPV PL peptide) and a PDZ-domain polypeptide (typically a fusion protein containing a PDZ domain). In the “A” assay, the candidate PL peptide is immobilized and binding of a soluble PDZ-domain polypeptide to the immobilized peptide is detected (the “A′” assay is named for the fact that in one embodiment an avidin surface is used to immobilize the peptide). In the “G” assay, the PDZ-domain polypeptide is immobilized and binding of a soluble PL peptide is detected (The “G” assay is named for the fact that in one embodiment a GST-binding surface is used to immobilize the PDZ-domain polypeptide). Preferred embodiments of these assays are described in detail infra. However, it will be appreciated by ordinarily skilled practitioners that these assays can be modified in numerous ways while remaining useful for the purposes of the present invention. In some embodiments, the PDZ-containing proteins or PL polypeptides are immobilized on a solid surface. The substrate to which the polypeptide is bound may in any of a variety of forms, e.g., a microtiter dish, a test tube, a dipstick, a microcentrifuge tube, a bead, a spinnable disk, a permeable or semi-permeable membrane, and the like. Suitable materials include glass, plastic (e.g., polyethylene, PVC, polypropylene, polystyrene, and the like), protein, paper, carbohydrate, lipid monolayer or supported lipid bilayer, films and other solid supports. Other materials that may be employed include ceramics, metals, metalloids, semiconductive materials, cements and the like.

In some embodiments, the PDZ and/or PL fusion proteins are organized as an array. The term “array,” as used herein, refers to an ordered arrangement of immobilized fusion proteins, in which particular different fusion proteins (i.e., having different PDZ domains) are located at different predetermined sites on the substrate. Because the location of particular fusion proteins on the array is known, binding at that location can be correlated with binding to the PDZ domain situated at that location. Immobilization of fusion proteins on beads (individually or in groups) is another particularly useful approach. In one embodiment, individual fusion proteins are immobilized on beads. In one embodiment, mixtures of distinguishable beads are used. Distinguishable beads are beads that can be separated from each other on the basis of a property such as size, magnetic property, color (e.g., using FACS) or affinity tag (e.g., a bead coated with protein A can be separated from a bead not coated with protein A by using IgG affinity methods). Binding to particular PDZ domain may be determined.

Methods for immobilizing proteins are known, and include covalent and non-covalent methods. One suitable immobilization method is antibody-mediated immobilization. According to this method, an antibody specific for the sequence of an “immobilization domain” of the PDZ-domain containing protein is itself immobilized on the substrate (e.g., by adsorption). One advantage of this approach is that a single antibody may be adhered to the substrate and used for immobilization of a number of polypeptides (sharing the same immobilization domain). For example, an immobilization domain consisting of poly-histidine (Bush et al, 1991, J. Biol Chem 266:13811-14) can be bound by an anti-histidine monoclonal antibody (R&D Systems, Minneapolis, Minn.); an immobilization domain consisting of secreted alkaline phosphatase (“SEAP”) (Berger et al, 1988, Gene 66:1-10) can be bound by anti-SEAP (Sigma Chemical Company, St. Louis, Mo.); an immobilization domain consisting of a FLAG epitope can be bound by anti-FLAG. Other ligand-antiligand immobilization methods are also suitable (e.g., an immobilization domain consisting of protein A sequences (Harlow and Lane, 1988, Antibodies A Laboratory Manual, Cold Spring Harbor Laboratory; Sigma Chemical Co., St. Louis, Mo.) can be bound by IgG; and an immobilization domain consisting of streptavidin can be bound by biotin (Harlow & Lane, supra; Sigma Chemical Co., St. Louis, Mo.). In a preferred embodiment, the immobilization domain is a GST moiety, as described herein.

When antibody-mediated immobilization methods are used, glass and plastic are especially useful substrates. The substrates may be printed with a hydrophobic (e.g., Teflon) mask to form wells. Preprinted glass slides with 3, 10 and 21 wells per 14.5 cm² slide “working area” are available from, e.g., SPI Supplies, West Chester, Pa.; also see U.S. Pat. No. 4,011,350). In certain applications, a large format (12.4 cm×8.3 cm) glass slide is printed in a 96 well format is used; this format facilitates the use of automated liquid handling equipment and utilization of 96 well format plate readers of various types (fluorescent, colorimetric, scintillation). However, higher densities maybe used (e.g., more than 10 or 100 polypeptides per cm²). See, e.g., MacBeath et al, 2000, Science 289:1760-63.

Typically, antibodies are bound to substrates (e.g., glass substrates) by adsorption. Suitable adsorption conditions are well known in the art and include incubation of 0.5-50 ug/ml (e.g., 10 ug/ml) mAb in buffer (e.g., PBS, or 50 to 300 mM Tris, MOPS, HEPES, PIPES, acetate buffers, pHs 6.5 to 8, at 4° C.) to 37° C. and from 1 hr to more than 24 hours.

Proteins may be covalently bound or noncovalently attached through nonspecific bonding. If covalent bonding between the fusion protein and the surface is desired, the surface will usually be polyfunctional or be capable of being polyfunctionalized. Functional groups which may be present on the surface and used for linking can include carboxylic acids, aldehydes, amino groups, cyano groups, ethylenic groups, hydroxyl groups, mercapto groups and the like. The manner of linking a wide variety of compounds to various surfaces is well known and is amply illustrated in the literature.

i. “A Assay” Detection of PDZ-Ligand Binding Using Immobilized PL Peptide.

In one aspect, the invention provides an assay in which biotinylated candidate PL peptides are immobilized on an avidin-coated surface. The binding of PDZ-domain fusion protein to this surface is then measured. In a preferred embodiment, the PDZ-domain fusion protein is a GST/PDZ fusion protein and the assay is carried out as follows:

(1) Avidin is bound to a surface, e.g. a protein binding surface. In one embodiment, avidin is bound to a polystyrene 96 well plate (e.g., Nunc Polysorb (cat #475094) by addition of 100 uL per well of 20 ug/mL of avidin (Pierce) in phosphate buffered saline without calcium and magnesium, pH 7.4 (“PBS”, GibcoBRL) at 4° C. for 12 hours. The plate is then treated to block nonspecific interactions by addition of 200 uL per well of PBS containing 2 g per 100 mL protease-free bovine serum albumin (“PBS/BSA”) for 2 hours at 4° C. The plate is then washed 3 times with PBS by repeatedly adding 200 uL per well of PBS to each well of the, plate and then dumping the contents of the plate into a waste container and tapping the plate gently on a dry surface.

(2) Biotinylated PL peptides (or candidate PL peptides, e.g. see TABLE 2) are immobilized on the surface of wells of the plate by addition of 50 uL per well of 0.4 uM peptide in PBS/BSA for 30 minutes at 4° C. Usually, each different peptide is added to at least eight different wells so that multiple measurements (e.g. duplicates and also measurements using different (GST/PDZ-domain fusion proteins and a GST alone negative control) can be made, and also additional negative control wells are prepared in which no peptide is immobilized. Following immobilization of the PL peptide on the surface, the plate is washed 3 times with PBS.

(3) GST/PDZ-domain fusion protein (prepared as described supra) is allowed to react with the surface by addition of 50 uL per well of a solution containing 5 ug/mL GST/PDZ-domain fusion protein in PBS/BSA for 2 hours at 4° C. As a negative control, GST alone (i.e. not a fusion protein) is added to specified wells, generally at least 2 wells (i.e. duplicate measurements) for each immobilized peptide. After the 2 hour reaction, the plate is washed 3 times with PBS to remove unbound fusion protein.

(4) The binding of the GST/PDZ-domain fusion protein to the avidin-biotinylated peptide surface can be detected using a variety of methods, and detectors known in the art. In one embodiment, 50 uL per well of an anti-GST antibody in PBS/BSA (e.g. 2.5 ug/mL of polyclonal goat-anti-GST antibody, Pierce) is. added to the plate and allowed to react for 20 minutes at 4° C. The plate is washed 3 times with PBS and a second, detectably labeled antibody is added. In one embodiment, 50 uL per well of 2.5 ug/mL of horseradish peroxidase (HRP)-conjugated polyclonal rabbit anti-goat immunoglobulin antibody is added to the plate and allowed to react for 20 minutes at 4° C. The plate is washed 5 times with 50 mM Tris pH 8.0 containing 0.2% Tween 20, and developed by addition of 100 uL per well of HRP-substrate solution (TMB, Dako) for 20 minutes at room temperature (RT). The reaction of the HRP and its substrate is terminated by the addition of 100 uL per well of 1M sulfuric acid and the absorbance (A) of each well of the plate is read at 450 nm.

(5) Specific binding of a PL peptide and a PDZ-domain polypeptide is detected by comparing the signal from the well(s) in which the PL peptide and PDZ domain polypeptide are combined with the background signal(s). The background signal is the signal found in the negative controls. Typically a specific or selective reaction will be at least twice background signal, more typically more than 5 times background, and most typically 10 or more times the background signal. In addition, a statistically significant reaction will involve multiple measurements of the reaction with the signal and the background differing by at least two standard errors, more typically four standard errors, and most typically six or more standard errors. Correspondingly, a statistical test (e.g. a T-test) comparing repeated measurements of the signal with repeated measurements of the background will result in a p-value<0.05, more typically a p-value<0.01, and most typically a p-value<0.001 or less.

As noted, in an embodiment of the “A” assay, the signal from binding of a GST/PDZ-domain fusion protein to an avidin surface not exposed to (i.e. not covered with) the PL peptide is one suitable negative control (sometimes referred to as “B”). The signal from binding of GST polypeptide alone (i.e. not a fusion protein) to an avidin-coated surface that has been exposed to (i.e. covered with) the PL peptide is a second suitable negative control (sometimes referred to as “B2”). Because all measurements are done in multiples (i.e. at least duplicate) the arithmetic mean (or, equivalently, average) of several measurements is used in determining the binding, and the standard error of the mean is used in determining the probable error in the measurement of the binding. The standard error of the mean of N measurements equals the square root of the following: the sum of the squares of the difference between each measurement and the mean, divided by the product of (N) and (N−1). Thus, in one embodiment, specific binding of the PDZ protein to the plate-bound PL peptide is determined by comparing the mean signal (“mean S”) and standard error of the signal (“SE”) for a particular PL-PDZ combination with the mean B1 and/or mean B2.

ii. “G Assay”—Detection of PDZ-Ligand Binding Using Immobilized PDZ-Domain Fusion Polypeptide

In one aspect, the invention provides an assay in which a GST/PDZ fusion protein is immobilized on a surface (“G” assay). The binding of labeled PL peptide (e.g., as listed in TABLE 2) to this surface is then measured. In a preferred embodiment, the assay is carried out as follows:

(1) APDZ-domain polypeptide is bound to a surface, e.g. a protein binding surface. In a preferred embodiment, a GST/PDZ fusion protein containing one or more PDZ domains is bound to a polystyrene 96-well plate. The GST/PDZ fusion protein can be bound to the plate by any of a variety of standard methods known to one of skill in the art, although some care must be taken that the process of binding the fusion protein to the plate does not alter the ligand-binding properties of the PDZ domain. In one embodiment, the GST/PDZ fusion protein is bound via an anti-GST antibody that is coated onto the 96-well plate. Adequate binding to the plate can be achieved when:
a. 100 uL per well of 5 ug/mL goat anti-GST polyclonal antibody (Pierce) in PBS is added to a polystyrene 96-well plate (e.g., Nunc Polysorb) at 4° C. for 12 hours.
b. The plate is blocked by addition of 200 uL per well of PBS/BSA for 2 hours at 4° C.
c. The plate is washed 3 times with PBS.
d. 50 uL per well of 5 ug/mL GST/PDZ fusion protein) or, as a negative control, GST polypeptide alone (i.e. not a fusion protein) in PBS/BSA is added to the plate for 2 hours at 4° C.
e. The plate is again washed 3 times with PBS.

(2) Biotinylated PL peptides are allowed to react with the surface by addition of 50 uL per well of 20 uM solution of the biotinylated peptide in PBS/BSA for 10 minutes at 4° C., followed by an additional 20 minute incubation at 25° C. The plate is washed 3 times with ice cold PBS.

(3) The binding of the biotinylated peptide to the GST/PDZ fusion protein surface can be detected using a variety of methods and detectors known to one of skill in the art. In one embodiment, 100 uL per well of 0.5 ug/mL streptavidin-horse radish peroxidase (HRP) conjugate dissolved in BSA/PBS is added and allowed to react for 20 minutes at 4° C. The plate is then washed 5 times with 50 mM Tris pH 8.0 containing 0.2% Tween 20, and developed by addition of 100 uL per well of HRP-substrate solution (TMB, Dako) for 20 minutes at room temperature (RT). The reaction of the HRP and its substrate is terminated by addition of 100 uL per well of 1M sulfuric acid, and the absorbance of each well of the plate is read at 450 nm.

(4) Specific binding of a PL peptide and a PDZ domain polypeptide is determined by comparing the signal from the well(s) in which the PL peptide and PDZ domain polypeptide are combined, with the background signal(s). The background signal is the signal found in the negative control(s). Typically a specific or selective reaction will be at least twice background signal, more typically more than 5 times background, and most typically 10 or more times the background signal. In addition, a statistically significant reaction will involve multiple measurements of the reaction with the signal and the background differing by at least two standard errors, more typically four standard errors, and most typically six or more standard errors. Correspondingly, a statistical test (e.g. a T-test) comparing repeated measurements of the signal with -repeated measurements of the background will result in a p-value<0.05, more typically a p-value<0.01, and most typically a p-value<0.001 or less. As noted, in an embodiment of the “G” assay, the signal from binding of a given PL peptide to immobilized (surface bound) GST polypeptide alone is one suitable negative control (sometimes referred to as “B 1”). Because all measurement are done in multiples (i.e. at least duplicate) the arithmetic mean (or, equivalently, average.) of several measurements is used in determining the binding, and the standard error of the mean is used in determining the probable error in the measurement of the binding. The standard error of the mean of N measurements equals the square root of the following: the sum of the squares of the difference between each measurement and the mean, divided by the product of (N) and (N−1). Thus, in one embodiment, specific binding of the PDZ protein to the platebound peptide is determined by comparing the mean signal (“mean S”) and standard error of the signal (“SE”) for a particular PL-PDZ combination with the mean B1.

iii. “G′ assay” and “G″ assay”

Two specific modifications of the specific conditions described supra for the “G assay” are particularly useful. The modified assays use lesser quantities of labeled PL peptide and have slightly different biochemical requirements for detection of PDZ-ligand binding compared to the specific assay conditions described supra.

For convenience, the assay conditions described in this section are referred to as the “G′ assay” and the “G″ assay,” with the specific conditions described in the preceding section on G assays being referred to as the “G⁰ assay.” The “G′ assay” is identical to the “G⁰ assay” except at step (2) the peptide concentration is 10 uM instead of 20 uM. This results in slightly lower sensitivity for detection of interactions with low affinity and/or rapid dissociation rate. Correspondingly, it slightly increases the certainty that detected interactions are of sufficient affinity and half-life to be of biological importance and useful therapeutic targets.

The “G″ assay” is identical to the “G⁰ assay” except that at step (2) the peptide concentration is 1 uM instead of 20 uM and the incubation is performed for 60 minutes at 25° C. (rather than, e.g., 10 minutes at 4° C. followed by 20 minutes at 25° C.). This results in lower sensitivity for interactions of low affinity, rapid dissociation rate, and/or affinity that is less at 25° C. than at 4° C. Interactions will have lower affinity at 25° C. than at 4° C. if (as we have to be generally true for PDZ-ligand binding) the reaction entropy is negative (i.e. the entropy of the products is less than the entropy of the reactants). In contrast, the PDZ-PL binding signal may be similar in the “G″ assay” and the “G⁰ assay” for interactions of slow association and dissociation rate, as the PDZ-PL complex will accumulate during the longer incubation of the “G″ assay.” Thus comparison of results of the “G″ assay” and the “G⁰ assay” can be used to estimate the relative entropies, enthalpies, and kinetics of different PDZ-PL interactions. (Entropies and enthalpies are related to binding affinity by the equations delta G=RT In (Kd)=delta H-T delta S where delta G, H, and S are the reaction free energy, enthalpy, and entropy respectively, T is the temperature in degrees Kelvin, R is the gas constant, and Kd is the equilibrium dissociation constant). In particular, interactions that are detected only or much more strongly in the “Go assay” generally have a rapid dissociation rate at 25° C. (t1/2<10 minutes) and a negative reaction entropy, while interactions that are detected similarly strongly in the “G″ assay” generally have a slower dissociation rate at 25° C. (t1/2>10 minutes). Rough estimation of the thermodynamics and kinetics of PDZ-PL interactions (as can be achieved via comparison of results of the “G⁰ assay” versus the “G″ assay” as outlined supra) can be used in the design of efficient inhibitors of the interactions. For example, a small molecule inhibitor based on the chemical structure of a PL that dissociates slowly from a given PDZ domain (as evidenced by similar binding in the “G″ assay” as in the “G⁰ assay”) may itself dissociate slowly and thus be of high affinity.

In this manner, variation of the temperature and duration of step (2) of the “G assay” can be used to provide insight into the kinetics and thermodynamics of the PDZ-ligand binding reaction and into design of inhibitors of the reaction.

iv. Assay Variations

As discussed supra, it will be appreciated that many of the steps in the above-described assays can be varied, for example, various substrates can be used for binding the PL and PDZ-containing proteins; different types of PDZ containing fusion proteins can be used; different labels for detecting PDZ/PL interactions can be employed; and different ways of detection can be used.

The PDZ-PL detection assays can employ a variety of surfaces to bind the PL and/or PDZ-containing proteins. For example, a surface can be an “assay plate” which is formed from a material (e.g. polystyrene) which optimizes adherence of either the PL protein or PDZ-containing protein thereto. Generally, the individual wells of the assay plate will have a high surface area to volume ratio and therefore a suitable shape is a flat bottom well (where the proteins of the assays are adherent). Other surfaces include, but are not limited to, polystyrene or glass beads, polystyrene or glass slides, papers, dipsticks, plastics, films and the like.

For example, the assay plate can be a “microtiter” plate. The term “microtiter” plate when used herein refers to a multiwell assay plate, e.g., having between about 30 to 200 individual wells, usually 96 wells. Alternatively, high-density arrays can be used. Often, the individual wells of the microtiter plate will hold a maximum volume of about 250 ul. Conveniently, the assay plate is a 96 well polystyrene plate (such as that sold by Becton Dickinson Labware, Lincoln Park, N.J.), which allows for automation and high throughput screening. Other surfaces include polystyrene microtiter ELISA plates such as that sold by Nunc Maxisorp, Inter Med, Denmark. Often, about 50 ul to 300 ul, more preferably 100 ul to 200 ul, of an aqueous sample comprising buffers suspended therein will be added to each well of the assay plate.

The detectable labels of the invention can be any detectable compound or composition which is conjugated directly or indirectly with a molecule (such as described above). The label can be detectable by itself (e.g., radioisotope labels or fluorescent labels) or, in the case of an enzymatic label, can catalyze a chemical alteration of a substrate compound or composition which is detectable. The preferred label is an enzymatic one which catalyzes a color change of a non-radioactive color reagent.

Sometimes, the label is indirectly conjugated with the antibody. One of skill is aware of various techniques for direct and indirect conjugation. For example, the antibody can be conjugated with biotin and any of the categories of labels mentioned above can be conjugated with avidin, or vice versa (see also “A” and “G” assay above). Biotin binds selectively to avidin and thus, the label can be conjugated with the antibody in this indirect manner. See, Ausubel, supra, for a review of techniques involving biotin-avidin conjugation and similar assays. Alternatively, to achieve indirect conjugation of the label with the antibody, the antibody is conjugated with a small hapten (e.g. digoxin) and one of the different types of labels mentioned above is conjugated with an anti-hapten antibody (e.g. anti-digoxin antibody). Thus, indirect conjugation of the label with the antibody can be achieved.

Assay variations can include different washing steps. By “washing” is meant exposing the solid phase to an aqueous solution (usually a buffer or cell culture media) in such a way that unbound material (e.g., non-adhering cells, non-adhering capture agent, unbound ligand, receptor, receptor construct, cell lysate, or HRP antibody) is removed therefrom. To reduce background noise, it is convenient to include a detergent (e.g., Triton X) in the washing solution. Usually, the aqueous washing solution is decanted from the wells of the assay plate following washing. Conveniently, washing can be achieved using an automated washing device. Sometimes, several washing steps (e.g., between about 1 to 10 washing steps) can be required.

Various buffers can also be used in PDZ-PL detection assays. For example, various blocking buffers can be used to reduce assay background. The term “blocking buffer” refers to an aqueous, pH buffered solution containing at least one blocking compound which is able to bind to exposed surfaces of the substrate which are not coated with a PL or PDZ-containing protein. The blocking compound is normally a protein such as bovine serum albumin (BSA), gelatin, casein or milk powder and does not cross-react with any of the reagents in the assay. The block buffer is generally provided at a pH between about 7 to 7.5 and suitable buffering agents include phosphate and TRIS.

Various enzyme-substrate combinations can also be utilized in detecting PDZ-PL interactions. Examples of enzyme-substrate combinations include, for example:

(i) Horseradish peroxidase (HRP or HRPO) with hydrogen peroxidase as a substrate, wherein the hydrogen peroxidase oxidizes a dye precursor (e.g. orthophenylene diamine [OPD] or 3,3′,5,5′-tetramethyl benzidine hydrochloride [TMB]) (as described above).

(ii) alkaline phosphatase (AP) with para-Nitrophenyl phosphate as chromogenic substrate.

(iii) Beta-D-galactosidase (Beta D-Gal) with a chromogenic substrate (e.g. p-nitrophenyl-Beta-D-galactosidase) or fluorogenic substrate 4-methylumbelliferyl-Beta-D-galactosidase.

Numerous other enzyme-substrate combinations are available to those skilled in the art. For a general review of these, see U.S. Pat. Nos. 4,275,149 and 4,318,980, both of which are herein incorporated by reference.

Further, it will be appreciated that, although, for convenience, the present discussion primarily refers to detection of PDZ-PL interactions, agonists or antagonists of PDZ-PL interactions can be used to treat cellular abnormalities.

V. Measurements of PDZ-Ligand Binding Affinity

The “A” and “G” assays of the invention can be used to determine the “apparent affinity” of binding of a PDZ ligand peptide to a PDZ-domain polypeptide. Apparent affinity is determined based on the concentration of one molecule required to saturate the binding of a second molecule (e.g., the binding of a ligand to a receptor). Two particularly useful approaches for quantitation of apparent affinity of PDZ-ligand binding are provided infra. These methods can be used to compare the sensitivity and affinity of differing PL constructs. Understanding the sensitivity of the PDZ for pathogen PLs is essential because it helps in the design of a modulator with the appropriate specificity for the interaction, PL, or PDZ.

(1) A GST/PDZ fusion protein, as well as GST alone as a negative control, are bound to a surface (e.g., a 96-well plate) and the surface blocked and washed as described supra for the “G” assay.

(2) 50 uL per well of a solution of biotinylated PL peptide (e.g. as shown in TABLE 2) is added to the surface in increasing concentrations in PBS/BSA (e.g. at 0.1 uM, 0.33 uM, 1 uM, 3.3 uM, 10 uM, 33 uM, and 100 uM). In one embodiment, the PL peptide is allowed to react with the bound GST/PDZ fusion protein (as well as the GST alone negative control) for 10 minutes at 4° C. followed by 20 minutes at 25° C. The plate is washed 3 times with ice cold PBS to remove unbound labeled peptide.

(3) The binding of the PL peptide to the inunobilized PDZ-domain polypeptide is detected as described supra for the “G” assay.

(4) For each concentration of peptide, the net binding signal is determined by subtracting the binding of the peptide to GST alone from the binding of the peptide to the GST/PDZ fusion protein. The net binding signal is then plotted as a function of ligand concentration and the plot is fit (e.g. by using the Kaleidagraph software package curve fitting algorithm; Synergy Software) to the following equation, where “Signal_{[ligand]}” is the net binding signal at PL peptide concentration “[ligand],” “Kd” is the apparent affinity of the binding event, and “Saturation Binding” is a constant determined by the curve fitting algorithm to optimize the fit to the experimental data:
Signal_{[ligand]}=Saturation Binding×([ligand]/([ligand]+Kd))

For reliable application of the above equation it is necessary that the highest peptide ligand concentration successfully tested experimentally be greater than, or at least similar to, the calculated Kd (equivalently, the maximum observed binding should be similar to the calculated saturation binding). In cases where satisfying the above criteria proves difficult, an alternative approach (infra) can be used.

Approach 2:

(1) A fixed concentration of a PDZ-domain polypeptide and increasing concentrations of a labeled PL peptide (labeled with, for example, biotin or fluorescein, see TABLE 2 for representative peptide amino acid sequences) are mixed together in solution and allowed to react. In one embodiment, preferred peptide concentrations are 0.1 uM, 1 uM, 10 uM, 100 uM, 1 mM. In various embodiments, appropriate reaction times can range from 10 minutes to 2 days at temperatures ranging from 4° C. to 37° C. In some embodiments, the identical reaction can also be carried out using a non-PDZ domain-containing protein as a control (e.g., if the PDZ-domain polypeptide is fusion protein, the fusion partner can be used).

(2) PDZ-ligand complexes can be separated from unbound labeled peptide using a variety of methods known in the art. For example, the complexes can be separated using high performance size-exclusion chromatography (HPSEC, gel filtration) (Rabinowitz et al., 1998, Immunity 9:699), affinity chromatography(e.g. using glutathione Sepharose beads), and affinity absorption (e.g., by binding to an anti-GST-coated plate as described supra).

(3) The PDZ-ligand complex is detected based on presence of the label on the peptide ligand using a variety of methods and detectors known to one of skill in the art. For example, if the label is fluorescein and the separation is achieved using HPSEC, an in-line fluorescence detector can be used. The binding can also be detected as described supra for the G assay.

(4) The PDZ-ligand binding signal is plotted as a function of ligand concentration and the plot is fit. (e.g., by using the Kaleidagraph software package curve fitting algorithm) to the following equation, where “Signal_{[ligand]}” is the binding signal at PL peptide concentration “[ligand],” “Kd” is the apparent affinity of the binding event, and “Saturation Binding” is a constant determined by the curve fitting algorithm to optimize the fit to the experimental data:
Signal_{[Ligand]}=Saturation Binding×([ligand]/([ligand+Kd])

Measurement of the affinity of a labeled peptide ligand binding to a PDZ-domain polypeptide is useful because knowledge of the affinity (or apparent affinity) of this interaction allows rational design of inhibitors of the interaction with known potency. The potency of inhibitors in inhibition would be similar to (i.e. within one-order of magnitude of) the apparent affinity of the labeled peptide ligand binding to the PDZ-domain.

Thus, in one aspect, the invention provides a method of determining the apparent affinity of binding between a PDZ domain and a ligand by immobilizing a polypeptide comprising the PDZ domain and a non-PDZ domain on a surface, contacting the immobilized polypeptide with a plurality of different concentrations of the ligand, determining the amount of binding of the ligand to the immobilized polypeptide at each of the concentrations of ligand, and calculating the apparent affinity of the binding based on that data. Typically, the polypeptide comprising the PDZ domain and a non-PDZ domain is a fusion protein. In one embodiment, the e.g., fusion protein is GST-PDZ fusion protein, but other polypeptides can also be used (e.g., a fusion protein including a PDZ domain and any of a variety of epitope tags, biotinylation signals and the like) so long as the polypeptide can be immobilized In an orientation that does not abolish the ligand binding properties of the PDZ domain, e.g, by tethering the polypeptide to the surface via the non-PDZ domain via an anti-domain antibody and leaving the PDZ domain as the free end. It was discovered, for example, reacting a PDZ-GST fusion polypeptide directly to a plastic plate provided suboptimal results. The calculation of binding affinity itself can be determined using any suitable equation (e.g., as shown supra; also see Cantor and Schimmel (1980) BIOPHYSICAL CHEMISTRY WH Freeman & Co., San Francisco) or software.

Thus, in a preferred embodiment, the polypeptide is immobilized by binding the polypeptide to an immobilized immunoglobulin that binds the non-PDZ domain (e.g., an anti-GST antibody when a GST-PDZ fusion polypeptide is used). In a preferred embodiment, the step of contacting the ligand and PDZ-domain polypeptide is carried out under the conditions provided supra in the description of the “G” assay. It will be appreciated that binding assays are conveniently carried out in multiwell plates (e.g., 24-well, 96-well plates, or 384 well plates).

The present method has considerable advantages over other methods for measuring binding affinities PDZ-PL affinities, which typically involve contacting varying concentrations of a GST-PDZ fusion protein to a ligand-coated surface. For example, some previously described methods for determining affinity (e.g., using immobilized ligand and GST-PDZ protein in solution) did not account for oligomerization state of the fusion proteins used, resulting in potential errors of more than an order of magnitude.

Although not sufficient for quantitative measurement of PDZ-PL binding affinity, an estimate of the relative strength of binding of different PDZ-PL pairs can be made based on the absolute magnitude of the signals observed in the “G assay.” This estimate will reflect several factors, including biologically relevant aspects of the interaction, including the affinity and the dissociation rate. For comparisons of different ligands binding to a given PDZ domain-containing protein, differences in absolute binding signal likely relate primarily to the affinity and/or dissociation rate of the interactions of interest.

Another method of increasing the specificity or sensitivity of a PDZ-PL interaction is through mutagenesis and selection of high affinity or high specificity variants. Methods such as UV, chemical (e.g., EMS) or biological mutagenesis (e.g. Molecular shuffling or DNA polymerase mutagenesis) can be applied to create mutations in DNA encoding PDZ domains or PL domains. Proteins can then be made from variants and tested using a number of methods described herein (e.g., ‘A’ assay, ‘G’ assay or yeast two hybrid). In general, one would assay mutants for high affinity binding between the mutated PDZ domain and a test sample (such as an oncogenic E6 PL) that have reduced affinity for other cellular PLs (as described in section IX). These methods are known to those skilled in the art and examples herein are not intended to be limiting.

VI. Measurements of PDZ or PL Specificity

As described supra, the present invention provides powerful methods for analysis of PDZ-ligand interactions, including high-throughput methods such as the “G” assay and affinity assays described supra. In one embodiment of the invention, the affinity is determined for a particular ligand and a plurality of PDZ proteins. Typically the plurality is at least 5, and often at least 25, or at least 40 different PDZ proteins. In a preferred embodiment, the plurality of different PDZ proteins are from a particular tissue (e.g., reproductive system) or a particular class or type of cell, (e.g., a cervical cell, a muscular cell, an epithelial cell) and the like. In a most preferred embodiment, the plurality of different PDZ proteins represents a substantial fraction (e.g., typically a majority, more often at least 80%) of all of the PDZ proteins known to be, or suspected of being, expressed in the tissue or cell(s), e.g., all of the PDZ proteins known to be present in cervical cells. In an embodiment, the plurality is at least 50%, usually at least 80%, at least 90% or all of the PDZ proteins disclosed herein as being expressed in cervical cells.

In one embodiment of the invention, the binding of a ligand to the plurality of PDZ proteins is determined. Using this method, it is possible to identify a particular PDZ domain bound with particular specificity by the ligand. The binding may be designated as “specific” if the affinity of the ligand to the particular PDZ domain is at least 2-fold that of the binding to other PDZ domains in the plurality (e.g., present in that cell type). The binding is deemed “very specific” if the affinity is at least 10-fold higher than to any other PDZ in the plurality or, alternatively, at least 10-fold higher than to at least 90%, more often 95% of the other PDZs in a defined plurality. Similarly, the binding is deemed “exceedingly specific” if it is at least 100-fold higher. For example, a ligand could bind to 2 different PDZs with an affinity of 1 uM and to no other PDZs out of a set 40 with an affinity of less than 100 uM. This would constitute specific binding to those 2 PDZs. Similar measures of specificity are used to describe binding of a PDZ to a plurality of PLs.

It will be recognized that high specificity PDZ-PL interactions represent potentially more valuable targets for achieving a desired biological effect. The ability of an inhibitor or enhancer to act with high specificity is often desirable. In particular, the most specific PDZ-ligand interactions are also the therapeutic targets, allowing specific disruption of an interaction.

Thus, in one embodiment, the invention provides a method of identifying a high specificity interaction between a particular PDZ domain and a ligand known or suspected of binding at least one PDZ domain, by providing a plurality of different immobilized polypeptides, each of said polypeptides comprising a PDZ domain and a non-PDZ domain; determining the affinity of the ligand for each of said polypeptides, and comparing the affinity of binding of the ligand to each of said polypeptides, wherein an interaction between the ligand and a particular PDZ domain is deemed to have high specificity when the ligand binds an immobilized polypeptide comprising the particular PDZ domain with at least 2-fold higher affinity than to immobilized polypeptides not comprising the particular PDZ domain.

In a related aspect, the affinity of binding of a specific PDZ domain to a plurality of ligands (or suspected ligands) is determined. For example, in one embodiment, the invention provides a method of identifying a high specificity interaction between a PDZ domain and a particular ligand known or suspected of binding at least one PDZ domain, by providing an immobilized polypeptide comprising the PDZ domain and a non-PDZ domain; determining the affinity of each of a plurality of ligands for the polypeptide, and comparing the affinity of binding of each of the ligands to the polypeptide, wherein an interaction between a particular ligand and the PDZ domain is deemed to have high specificity when the ligand binds an immobilized polypeptide comprising the PDZ domain with at least 2-fold higher affinity than other ligands tested. Thus, the binding may be designated as “specific” if the affinity of the PDZ to the particular PL is at least 2-fold that of the binding to other PLs in the plurality (e.g., present in that cell type). The binding is deemed “very specific” if the affinity is at least 10-fold higher than to any other PL in the plurality or, alternatively, at least 10-fold higher than to at least 90%, more often 95% of the other PLs in a defined plurality. Similarly, the binding is deemed “exceedingly specific” if it is at least 100-fold higher. Typically the plurality is at least 5 different ligands, more often at least 10.

VII. Assays for Detecting Oncogenic E6 Proteins

Oncogenic E6 proteins can be detected by their ability to bind to PDZ domains. This could be a developed into a single detection stage approach or more favorably as a two-stage or ‘sandwich’ approach for increased sensitivity and specificity.

For single stage approaches, a ‘tagged’ version of a PDZ domain that specifically recognizes oncogenic E6 proteins, such as those disclosed in TABLE 2, can be used to directly probe for the presence of oncogenic E6 protein in a sample. As noted supra, an example of this would be to attach the test sample to a solid support (for example, cervical cells or tissue could be coated on a slide and ‘fixed’ to permeablize the cell membranes), incubate the sample with a tagged ‘PL detector’ protein (a PDZ domain fusion) under appropriate conditions, wash away unbound PL detector, and assay for the presence of the ‘tag’ in the sample. One should note, however, that PDZ domains may also bind endogenous cellular proteins. Thus, frequency of binding must be compared to control cells that do not contain E6 oncoproteins or the ‘PL detector’ should be modified such that it is significantly more specific for the oncogenic E6 proteins (see section X).

For two-stage or sandwich approaches, use of the PL detector is coupled with a second method of either capturing or detecting captured proteins. The second method could be using an antibody that binds to the E6 oncoprotein or a second compound or protein that can bind to E6 oncoproteins at a location on the E6 protein that does not reduce the availability of the E6 PL. Such proteins may include, but not be limited to, p53, E6-AP, E6-BP or engineered compounds that bind E6 oncoproteins.

A. Antibodies

Many biological assays are designed as a ‘sandwich’, where an antibody constitutes one side of the sandwich. This method can improve the signal to noise ratio for a diagnostic by reducing background signal and amplifying appropriate signals. Antibodies can be generated that specifically recognize the diagnostic protein. Since this invention discloses the method of using PDZ or PL proteins to diagnose pathogen infections, antibodies should be generated that do not conflict with the PDZ-PL interaction.

For the production of antibodies, various host animals, including but not limited to rabbits, mice, rats, etc., may be immunized by injection with a peptide. The peptide may be attached to a suitable carrier, such as BSA or KLH, by means of a side chain functional group or linkers attached to a side chain functional group. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacilli Calmette-Guerin) and Corynebacterium parvum.

Monoclonal antibodies to a peptide may be prepared using any technique that provides for the production of antibody molecules by continuous cell lines in culture. These include but are not limited to the hybridoma technique originally described by Koehler and Milstein, 1975, Nature 256:495-497, the human B-cell hybridoma technique, Kosbor et al., 1983, Immunology Today 4:72; Cote et al., 1983, Proc. Natl. Acad. Sci. U.S.A. 80:2026-2030 and the EBV-hybridoma technique (Cole et al., 1985, Monoclonal Antibodies and Cancer Therapy, Alan R. Liss, Inc., pp. 77-96 (1985)). In addition, techniques developed for the production of “chimeric antibodies” (Morrison et al., 1984, Proc. Natl. Acad. Sci. U.S.A. 81:6851-6855; Neuberger et al., 1984, Nature 312:604-608; Takeda et al., 1985, Nature 314:452-454) by splicing the genes from a mouse antibody molecule of appropriate antigen specificity together with genes from a human antibody molecule of appropriate biological activity can be used. Alternatively, techniques described for the production of single chain antibodies (U.S. Pat. No. 4,946,778) can be adapted to produce peptide-specific single chain antibodies.

Antibody fragments containing deletions of specific binding sites may be generated by known techniques. For example, such fragments include but are not limited to F(ab′)₂ fragments, which can be produced by pepsin digestion of the antibody molecule and Fab fragments, which can be generated by reducing the disulfide bridges of the F(ab′)₂ fragments. Alternatively, Fab expression libraries may be constructed (Huse et al., 1989, Science 246:1275-1281) to allow rapid and easy identification of monoclonal Fab fragments with the desired specificity for the peptide of interest.

The antibody or antibody fragment specific for the desired peptide can be attached, for example, to agarose, and the antibody-agarose complex is used in immunochromatography to purify peptides of the invention. See, Scopes, 1984, Protein Purification: Principles and Practice, Springer-Verlag New York, Inc., NY, Livingstone, 1974, Methods Enzymology: Imnmunoaffinity Chromatography of Proteins 34:723-731. Antibodies can also be linked to other solid supports for diagnostic applications, or alternatively labeled with a means of detection such an enzyme that can cleave a colorimetric substrate, a fluorophore, a magnetic particle, or other measurable compositions of matter.

Specific antibodies against E6 proteins have historically been difficult to produce. In conjunction with the methods describe supra, one could employ a number of techniques to increase the likelihood of producing or selecting high affinity antibodies. An example is to prepare the E6 antigen (to raise antibodies against) in the same manner that one would prepare tissue or cell samples for testing. Alternatively, one could immunize with E6 fusion protein prepared in one manner, and screen for specific E6 antibodies using a second E6 protein prepared in a different manner. This should select for antibodies that recognize E6 epitopes that are conserved under different sample collection and preparation procedures. In another example, one could immunize animals with E6 antigen that has been rapidly denatured and renatured, such that epitopes that are insensitive to preparation conditions are selected for. Another method that could be employed is to use peptides corresponding to antigenic regions of the E6 proteins as predicted by Major Histocompatibility Complex (MHC) and T Cell Receptor (TCR) consensus binding.

These methods can be used for the detection of HPV strains in a sample, facilitating the treatment of HPV infection. Ongoing detection coupled with treatment programs can act as an effective prophylactic to prevent the development of diseases associated with HPV infection. In certain embodiments of the invention, detection of a particular PL motif(as shown in Table 2) in a patient allows for the use of treatments specific for strains containing that PL motif. Certain antagonists may disrupt interactions of one PL more effectively than another different PL motif. Treatments can be designed to target a certain HPV strain with a maximum specificity by using an antagonist that disrupts an interaction of a particular HPV PL with the highest possible efficiency.

In one embodiment of the invention, antibodies specific for the HPV C-terminal PL motif may be used for both detection and treatment of HPV infection. Antibodies against the PL of a HPV strain can not only detect the presence of a particular HPV strain in a sample, they can effectively block the PDZ binding motif of a HPV protein in vivo, preventing interaction with intracellular PDZ proteins and thus blocking the development or progression of HPV-associated diseases. Similarly, antibodies that block the binding pocket of a particular PDZ protein also prevent interactions between that PDZ protein and a PL protein. Antibodies can also be used to deliver peptide mimetics or small molecules to a specific cell type. Methods for generating human antibodies are well known in the art.

VIII. Use of Array for Global Predictions

One discovery of the present inventors relates to the important and extensive roles played by interactions between PDZ proteins and PL proteins, particularly in the biological function of cervical cells and other cells involved in the reproductive system. Further, it has been discovered that valuable information can be ascertained by analysis (e.g., simultaneous analysis) of a large number of PDZ-PL interactions. In a preferred embodiment, the analysis encompasses all of the PDZ proteins expressed in a particular tissue (e.g., reproductive tissue) or type or class of cell (e.g., cervical cell, muscle cell, epithelial cell and the like). Alternatively, the analysis encompasses at least about 5, or at least about 10, or at least about 12, or at least about 15 and often at least 50 different polypeptides, up to about 60, about 80, about 100, about 150, about 200, or even more different polypeptides; or a substantial fraction (e.g., typically a majority, more often at least 80%) of all of the PDZ proteins known to be, or suspected of being, expressed in the tissue or cell(s), e.g., all of the PDZ proteins known to be present in cervical cells.

It will be recognized that the arrays and methods of the invention are directed to the analysis of PDZ and PL interactions, and involve selection of such proteins for analysis. While the devices and methods of the invention may include or involve a small number of control polypeptides, they typically do not include significant numbers of proteins or fusion proteins that do not include either PDZ or PL domains (e.g., typically, at least about 90% of the arrayed or immobilized polypeptides in a method or device of the invention is a PDZ or PL sequence protein, more often at least about 95%, or at least about 99%).

It will be apparent from this disclosure that analysis of the relatively large number of different interactions preferably takes place simultaneously. In this context, “simultaneously” means that the analysis of several different PDZ-PL interactions (or the effect of a test agent on such interactions) is assessed at the same time. Typically the analysis is carried out in a high throughput (e.g., robotic) fashion. One advantage of this method of simultaneous analysis is that it permits rigorous comparison of multiple different PDZ-PL interactions. For example, as explained in detail elsewhere herein, simultaneous analysis (and use of the arrays described infra) facilitates, for example, the direct comparison of the effect of an agent (e.g., an potential interaction inhibitor) on the interactions between a substantial portion of PDZs and/or PLs in a tissue or cell.

Accordingly, in one aspect, the invention provides an array of immobilized polypeptide comprising the PDZ domain and a non-PDZ domain on a surface. Typically, the array comprises at least about 5, or at least about 10, or at least about 12, or at least about 15 and often at least 50 different polypeptides. In one preferred embodiment, the different PDZ proteins are from a particular tissue (e.g., reproductive tissue) or a particular class or type of cell, (e.g., a cervical cell, muscle cell, or epithelial cell) and the like. In a most preferred embodiment, the plurality of different PDZ proteins represents a substantial fraction (e.g., typically a majority, more often at least 60%, 70% or 80%) of all of the PDZ proteins known to be, or suspected of being, expressed in the tissue or cell(s), e.g., all of the PDZ proteins known to be present in cervical cells.

Certain embodiments are arrays that include a plurality, usually at least 5, 10, 25, 50 PDZ proteins present in a particular cell of interest. In this context, “array” refers to an ordered series of immobilized polypeptides in which the identity of each polypeptide is associated with its location. In some embodiments the plurality of polypeptides are arrayed in a “common” area such that they can be simultaneously exposed to a solution (e.g., containing a ligand or test agent). For example, the plurality of polypeptides can be on a slide, plate or similar surface, which may be plastic, glass, metal, silica, beads or other surface to which proteins can be immobilized. In a different embodiment, the different immobilized polypeptides are situated in separate areas, such as different wells of multi-well plate (e.g., a 24-well plate, a 96-well plate, a 384 well plate, and the like). It will be recognized that a similar advantage can be obtained by using multiple arrays in tandem.

IX. Assays to Identify Novel PDZ Domain Binding Moieties and Modulator of PDZ Protein-PL Protein Binding

Although described supra primarily in terms of identifying interactions between PDZ-domain polypeptides and PL proteins, the assays described supra and other assays can also be used to identify the binding of other molecules (e.g., peptide mimetics, small molecules, and the like) to PDZ domain sequences. For example, using the assays disclosed herein, combinatorial and other libraries of compounds can be screened, e.g., for molecules that specifically bind to PDZ domains. Screening of libraries can be accomplished by any of a variety of commonly known methods. See, e.g., the following references, which disclose screening of peptide libraries: Parmley and Smith, 1989, Adv. Exp. Med. Biol. 251:215-218; Scott and Smith, 1990, Science 249:386-390; Fowlkes et al., 1992; BioTechniques 13:422-427; Oldenburg et al., 1992, Proc. Natl. Acad. Sci. USA 89:5393-5397; Yu et al., 1994, Cell 76:933-945; Staudt et al., 1988, Science 241:577-580; Bock et al., 1992, Nature 355:564-566; Tuerk et al., 1992, Proc. Natl. Acad. Sci. USA 89:6988-6992; Ellington et al., 1992, Nature 355:850-852; U.S. Pat. No. 5,096,815, U.S. Pat. No. 5,223,409, and U.S. Pat. No. 5,198,346, all to Ladner et al.; Rebar and Pabo, 1993, Science 263:671-673; and PCT Publication No. WO 94/18318.

In a specific embodiment, screening can be carried out by contacting the library members with a PDZ-domain polypeptide immobilized on a solid support (e.g. as described supra in the “G” assay) and harvesting those library members that bind to the protein. Examples of such screening methods, termed “panning” techniques are described by way of example in Parmley and Smith, 1988, Gene 73:305-318; Fowlkes et al., 1992, BioTechniques 13:422-427; PCT Publication No. WO 94/18318; and in references cited-hereinabove.

In another embodiment, the two-hybrid system for selecting interacting proteins in yeast (Fields and Song, 1989, Nature 340:245-246; Chien et al., 1991, Proc. Natl. Acad. Sci. USA 88:9578-9582) can be used to identify molecules that specifically bind to a PDZ domain-containing protein. Furthermore, the identified molecules are further tested for their ability to inhibit transmembrane receptor interactions with a PDZ domain.

In one aspect of the invention, antagonists of an interaction between a PDZ protein and a PL protein are identified. In one embodiment, a modification of the “A” assay described supra is used to identify antagonists. In one embodiment, a modification of the “G” assay described supra is used to identify antagonists.

In one embodiment, screening assays are used to detect molecules that specifically bind to PDZ domains. Such molecules are useful as agonists or antagonists of PDZ-protein-mediated cell function (e.g., cell activation, e.g., T cell activation, vesicle transport, cytokine release, growth factors, transcriptional changes, cytoskeleton rearrangement, cell movement, chemotaxis, and the like). In one embodiment, such assays are performed to screen for leukocyte activation inhibitors for drug development. The invention thus provides assays to detect molecules that specifically bind to PDZ domain-containing proteins. For example, recombinant cells expressing PDZ domain-encoding nucleic acids can be used to produce PDZ domains in these assays and to screen for molecules that bind to the domains. Molecules are contacted with the PDZ domain (or fragment-thereof) under conditions conducive to binding, and then molecules that specifically bind to such domains are identified. Methods that can be used to carry out the foregoing are commonly known in the art.

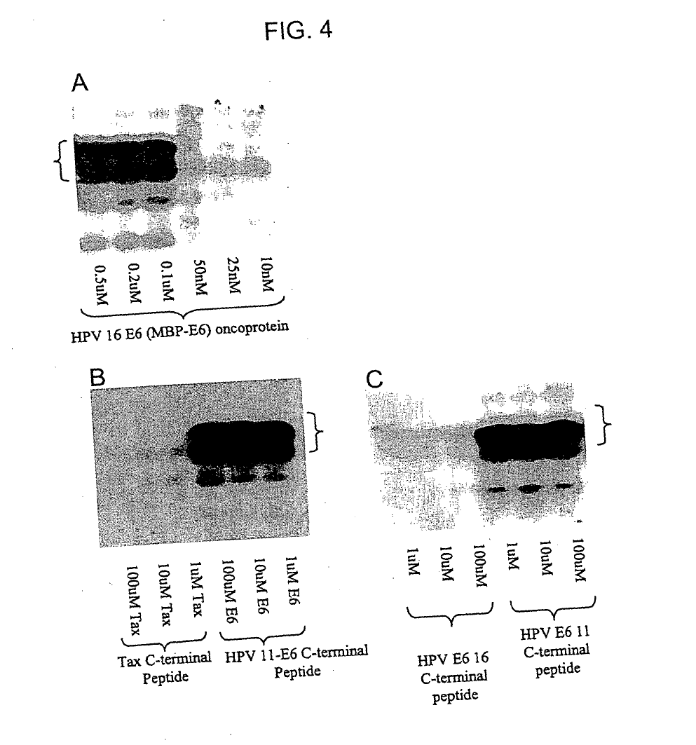

In one aspect of the invention, a biological test is used to identify agonists or antagonists of PDZ:PL binding. Examples of this are give in FIGS. 4, 5, and 6 and their corresponding examples. These assays are demonstrated to be effected by modulation of PDZ:PL interactions. In another aspect, biological assays such as those included herein can be used to examine the biological effect of modulators identified through biochemical assays or other assays described in this disclosure.

It will be appreciated by the ordinarily skilled practitioner that, in one embodiment, antagonists are identified by conducting the A or G assays in the presence and absence of a known or candidate antagonist. When decreased binding is observed in the presence of a compound, that compound is identified as an antagonist. Increased binding in the presence of a compound signifies that the compound is an agonist.

For example, in one assay, a test compound can be identified as an inhibitor (antagonist) of binding between a PDZ protein and a PL protein by contacting a PDZ domain polypeptide and a PL peptide in the presence and absence of the test compound, under conditions in which they would (but for the presence of the test compound) form a complex, and detecting the formation of the complex in the presence and absence of the test compound. It will be appreciated that less complex formation in the presence of the test compound than in the absence of the compound indicates that the test compound is an inhibitor of a PDZ protein-PL protein binding.

In one embodiment, the “G” assay is used in the presence or absence of a candidate inhibitor. In one embodiment, the “A” assay is used in the presence or absence of a candidate inhibitor.

In one embodiment (in which a G assay is used), one or more PDZ domain-containing GST-fusion proteins are bound to the surface of wells of a 96-well plate as described supra (with appropriate controls including nonfusion GST protein). All fusion proteins are bound in multiple wells so that appropriate controls and statistical analysis can be done. A test compound in BSA/PBS (typically at multiple different concentrations) is added to wells. Immediately thereafter, 30 uL of a detectably labeled (e.g., biotinylated) peptide known to bind to the relevant PDZ domain (see, e.g., TABLE 3) is added in each of the wells at a final concentration of, e.g., between about 2 uM and about 40 uM, typically 5 uM, 15 uM, or 25 uM. This mixture is then allowed to react with the PDZ fusion protein bound to the surface for 10 minutes at 4° C. followed by 20 minutes at 25° C. The surface is washed free of unbound peptide three times with ice cold PBS and the amount of binding of the peptide in the presence and absence of the test compound is determined. Usually, the level of binding is measured for each set of replica wells (e.g. duplicates) by subtracting the mean GST alone background from the mean of the raw measurement of peptide binding in these wells.

In an alternative embodiment, the A assay is carried out in the presence or absence of a test candidate to identify inhibitors of PL-PDZ interactions.

In one embodiment, a test compound is determined to be a specific inhibitor of the binding of the PDZ domain (P) and a PL (L) sequence when, at a test compound concentration of less than or equal to 1 mM (e.g., less than or equal to: 500 uM, 100 uM, 10 uM, 1 uM, 100 nM or 1 nM) the binding of P to L in the presence of the test compound less than about 50% of the binding in the absence of the test compound. (in various embodiments, less than about 25%, less than about 10%, or less than about 1%). Preferably, the net signal of binding of P to L in the presence of the test compound plus six (6) times the standard error of the signal in the presence of the test compound is less than the binding signal in the absence of the test compound.

In one embodiment, assays for an inhibitor are carried out using a single PDZ protein-PL protein pair (e.g., a PDZ domain fusion protein and a PL peptide). In a related embodiment, the assays are carried out using a plurality of pairs, such as a plurality of different pairs listed in TABLE 3.

In some embodiments, it is desirable to identify compounds that, at a given concentration, inhibit the binding of one PL-PDZ pair, but do not inhibit (or inhibit to a lesser degree) the binding of a specified second PL-PDZ pair. These antagonists can be identified by carrying out a series of assays using a candidate inhibitor and different PL-PDZ pairs (e.g., as shown in the matrix of TABLE 3) and comparing the results of the assays. All such pairwise combinations are contemplated by the invention (e.g., test compound inhibits binding of PL₁ to PDZ₁ to a greater degree than it inhibits binding of PL₁ to PDZ₂ or PL₂ to PDZ₂). Importantly, it will be appreciated that, based on the data provided in TABLE 3 and disclosed herein (and additional data that can be generated using the methods described herein) inhibitors with different specificities can readily be designed.

For example, according to the invention, the Ki (“potency”) of an inhibitor of a PDZ-PL interaction can be determined. Ki is a measure of the concentration of an inhibitor required to have a biological effect. For example, administration of an inhibitor of a PDZ-PL interaction in an amount sufficient to result in an intracellular inhibitor concentration of at least between about 1 and about 100 Ki is expected to inhibit the biological response mediated by the target PDZ-PL interaction. In one aspect of the invention, the Kd measurement of PDZ-PL binding as determined using the methods supra is used in determining Ki.

Thus, in one aspect, the invention provides a method of determining the potency (Ki) of an inhibitor or suspected inhibitor of binding between a PDZ domain and a ligand by immobilizing a polypeptide comprising the PDZ domain and a non-PDZ domain on a surface, contacting the immobilized polypeptide with a plurality of different mixtures of the ligand and inhibitor, wherein the different mixtures comprise a fixed amount of ligand and different concentrations of the inhibitor, determining the amount of ligand bound at the different concentrations of inhibitor, and calculating the Ki of the binding based on the amount of ligand bound in the presence of different concentrations of the inhibitor. In an embodiment, the polypeptide is immobilized by binding the polypeptide to an immobilized immunoglobulin that binds the non-PDZ domain. This method, which is based on the “G” assay described supra, is particularly suited for high-throughput analysis of the Ki for inhibitors of PDZ-ligand interactions. Further, using this method, the inhibition of the PDZ-ligand interaction itself is measured, without distortion of measurements by avidity effects.

Typically, at least a portion of the ligand is detectably labeled to permit easy quantitation of ligand binding.

It will be appreciated that the concentration of ligand and concentrations of inhibitor are selected to allow meaningful detection of inhibition. Thus, the concentration of the ligand whose binding is to be blocked is close to or less than its binding affinity (e.g., preferably less than the 5×Kd of the interaction, more preferably less than 2×Kd, most preferably less than 1×Kd). Thus, the ligand is typically present at a concentration of less than 2 Kd (e.g., between about 0.01 Kd and about 2 Kd) and the concentrations of the test inhibitor typically range from 1 nM to 100 uM (e.g. a 4-fold dilution series with highest concentration 10 uM or 1 mM). In a preferred embodiment, the Kd is determined using the assay disclosed supra.

The Ki of the binding can be calculated by any of a variety of methods routinely used in the art, based on the amount of ligand bound in the presence of different concentrations of the inhibitor. In an illustrative embodiment, for example, a plot of labeled ligand binding versus inhibitor concentration is fit to the equation:
S_{inhibitor}=S₀*Ki/([I]+Ki)
where S_{inhibitor} is the signal of labeled ligand binding to immobilized PDZ domain in the presence of inhibitor at concentration [I] and S₀ is the signal in the absence of inhibitor (i.e., [I]=0). Typically [I] is expressed as a molar concentration.

In another aspect of the invention, an enhancer (sometimes referred to as, augmentor or agonist) of binding between a PDZ domain and a ligand is identified by immobilizing a polypeptide comprising the PDZ domain and a non-PDZ domain on a surface, contacting the immobilized polypeptide with the ligand in the presence of a test agent and determining the amount of ligand bound, and comparing the amount of ligand bound in the presence of the test agent with the amount of ligand bound by the polypeptide in the absence of the test agent. At least two-fold (often at least 5-fold) greater binding in the presence of the test agent compared to the absence of the test agent indicates that the test agent is an agent that enhances the binding of the PDZ domain to the ligand. As noted supra, agents that enhance PDZ-ligand interactions are useful for disruption (dysregulation) of biological events requiring normal PDZ-ligand function (e.g., cancer cell division and metastasis).

The invention also provides methods for determining the “potency” or “K_{enhancer}” of an enhancer of a PDZ-ligand interaction. For example, according to the invention, the K_{enhancer} of an enhancer of a PDZ-PL interaction can be determined, e.g., using the Kd of PDZ-PL binding as determined using the methods described supra. K_{enhancer} is a measure of the concentration of an enhancer expected to have a biological effect. For example, administration of an enhancer of a PDZ-PL interaction in an amount sufficient to result in an intracellular inhibitor concentration of at least between about 0.1 and about 100 K_{enhancer} (e.g., between about 0.5 and about 50 K_{enhancer}) is expected to disrupt the biological response mediated by the target PDZ-PL interaction.

Thus, in one aspect the invention provides a method of determining the potency (K_{enhancer}) of an enhancer or suspected enhancer of binding between a PDZ domain and a ligand by immobilizing a polypeptide comprising the PDZ domain and a non-PDZ domain on a surface, contacting the immobilized polypeptide with a plurality of different mixtures of the ligand and enhancer, wherein the different mixtures comprise a fixed amount of ligand, at least a portion of which is detectably labeled, and different concentrations of the enhancer, determining the amount of ligand bound at the different concentrations of enhancer, and calculating the potency (K_{enhancer}) of the enhancer from the binding based on the amount of ligand bound in the presence of different concentrations of the enhancer. Typically, at least a portion of the ligand is detectably labeled to permit easy quantitation of ligand binding. This method, which is based on the “G” assay described supra, is particularly suited for high-throughput analysis of the K_{enhancer} for enhancers of PDZ-ligand interactions.

It will be appreciated that the concentration of ligand and concentrations of enhancer are selected to allow meaningful detection of enhanced binding. Thus, the ligand is typically present at a concentration of between about 0.01 Kd and about 0.5 Kd and the concentrations of the test agent/enhancer typically range from 1 nM to 1 mM (e.g. a 4-fold dilution series with highest concentration 10 uM or 1 mM). In a preferred embodiment, the Kd is determined using the assay disclosed supra.

The potency of the binding can be determined by a variety of standard methods based on the amount of ligand bound in the presence of different concentrations of the enhancer or augmentor. For example, a plot of labeled ligand binding versus enhancer concentration can be fit to the equation:
S([E])=S(0)+(S(0)*(D_{enhancer}−1)*[E]/([E]+K_{enhancer})
where “K_{enhancer}” is the potency of the augmenting compound, and “D_{enhancer}” is the fold-increase in binding of the labeled ligand obtained with addition of saturating amounts of the enhancing compound, [E] is the concentration of the enhancer. It will be understood that saturating amounts are the amount of enhancer such that further addition does not significantly increase the binding signal. Knowledge of “K_{enhancer}” is useful because it describes a concentration of the augmenting compound in a target cell that will result in a biological effect due to dysregulation of the PDZ-PL interaction. Typical therapeutic concentrations are between about 0.1 and about 100 K_{enhancer}.

A. Identification of Pharmaceutical Compounds that Inhibit PDZ-PL Proteins

For certain of the PDZ proteins and PL proteins shown to bind together and for which Kd values had been obtained, additional testing was conducted to determine whether certain pharmaceutical compounds would act to antagonize or agonize the interactions. Assays were conducted as for the G′ assay described supra both in the presence and absence of test compound, except that 50 ul of a 10 uM solution of the biotinylated PL peptide is allowed to react with the surface bearing the PDZ-domain polypeptide instead of a 20 uM solution as specified in step (2) of the assay.

B. Analysis of PDZ-PL Inhibition Profile

In one aspect, the invention provides a method for determining if a test compound inhibits any PDZ-ligand interaction in large set of PDZ-ligand interactions (e.g., a plurality of the PDZ-ligands interactions described in U.S. patent application Ser. No. 09/724553; a majority of the PDZ-ligands identified in a particular cell or tissue as described supra (e.g., cervical tissue) and the like. In one embodiment, the PDZ domains of interest are expressed as GST-PDZ fusion proteins and immobilized as described herein. For each PDZ domain, a labeled ligand that binds to the domain with a known affinity is identified as described herein.

For any known or suspected modulator (e.g., inhibitor) of a PDZ-PL interaction(s), it is useful to know which interactions are inhibited (or augmented). This information could be used to develop a highly specific treatment for a pathogen (e.g., an oncogenic HPV strain). The profile of PDZ interactions inhibited by a particular agent is referred to as the “inhibition profile”for the agent, and is described in detail below. The profile of PDZ interactions enhanced by a particular agent is referred to as the “enhancement profile” for the agent. It will be readily apparent to one of skill guided by the description of the inhibition profile how to determine the enhancement profile for an agent. The present invention provides methods for determining the PDZ interaction (inhibition/enhancement) profile of an agent in a single assay.

In one aspect, the invention provides a method for determining the PDZ-PL inhibition profile of a compound by providing (i) a plurality of different immobilized polypeptides, each of said polypeptides comprising a PDZ domain and a non-PDZ domain and (ii) a plurality of corresponding ligands, wherein each ligand binds at least one PDZ domain in (i), then contacting each of said immobilized polypeptides in (i) with a corresponding ligand in (ii) in the presence and absence of a test compound, and determining for each polypeptide-ligand pair whether the test compound inhibits binding between the immobilized polypeptide and the corresponding ligand.

Typically the plurality is at least 5, and often at least 25, or at least 40 different PDZ proteins. In a preferred embodiment, the plurality of different ligands and the plurality of different PDZ proteins are from the same tissue or a particular class or type of cell, e.g., a cervical cell, an endothelial cell and the like. In a most preferred embodiment, the plurality of different PDZs represents a substantial fraction (e.g., at least 80%) of all of the PDZs known to be, or suspected of being, expressed in the tissue or cell(s), e.g., all of the PDZs known to be present in cervical cells (for example, at least 80%, at least 90% or all of the PDZs disclosed herein as being expressed in cervical cells).

In one embodiment, the inhibition profile is determined as follows: A plurality (e.g., all known) PDZ domains expressed in a cell (e.g., cervical cells) are expressed as GST-fusion proteins and immobilized without altering their ligand binding properties as described supra. For each PDZ domain, a labeled ligand that binds to this domain with a known affinity is identified. If the set of PDZ domains expressed in cervical cells is denoted by {P1 . . . Pn}, any given PDZ domain Pi binds a (labeled) ligand Li with affinity K_{d}i. To determine the inhibition profile for a test agent “compound X” the “G” assay (supra) can be performed as follows in 96-well plates with rows A-H and columns 1-12. Column 1 is coated with P1 and washed. The corresponding ligand L1 is added to each washed coated well of column 1 at a concentration 0.5 K_{d}1 with (rows B, D, F, H) or without (rows A, C, E, F) between about 1 and about 1000 uM) of test compound X. Column 2 is coated with P2, and L2 (at a concentration 0.5.K_{d}2) is added with or without inhibitor X. Additional PDZ domains and ligands are similarly tested.

Compound X is considered to inhibit the binding of Li to Pi if the average signal in the wells of column i containing X is less than half the signal in the equivalent wells of the column lacking X. Thus, in this single assay one determines the full set of cervical cell PDZs that are inhibited by compound X.

In some embodiments, the test compound X is a mixture of compounds, such as the product of a combinatorial chemistry synthesis as described supra. In some embodiments, the test compound is known to have a desired biological effect, and the assay is used to determine the mechanism of action (i.e., if the biological effect is due to modulating a PDZ-PL interaction).

It will be apparent that an agent that modulates only one, or a few PDZ-PL interactions, in a panel (e.g., a panel of all known PDZs in cervical cells, a panel of at least 10, at least 20 or at least 50 PDZ domains) is a more specific modulator than an agent that modulate many or most interactions. Typically, an agent that modulates less than 20% of PDZ domains in a panel is deemed a “specific” inhibitor, less than 6% a “very specific” inhibitor, and a single PDZ domain a “maximally specific” inhibitor.

It will also be appreciated that “compound X” may be a composition containing mixture of compounds (e.g., generated using combinatorial chemistry methods) rather than a single compound.

Several variations of this assay are contemplated:

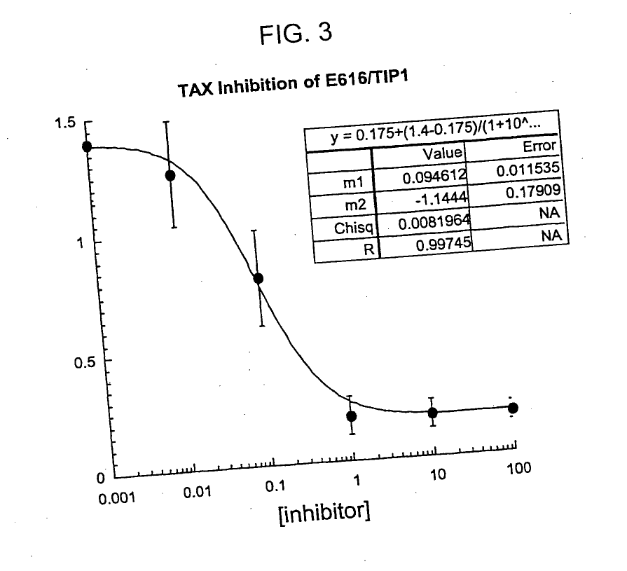

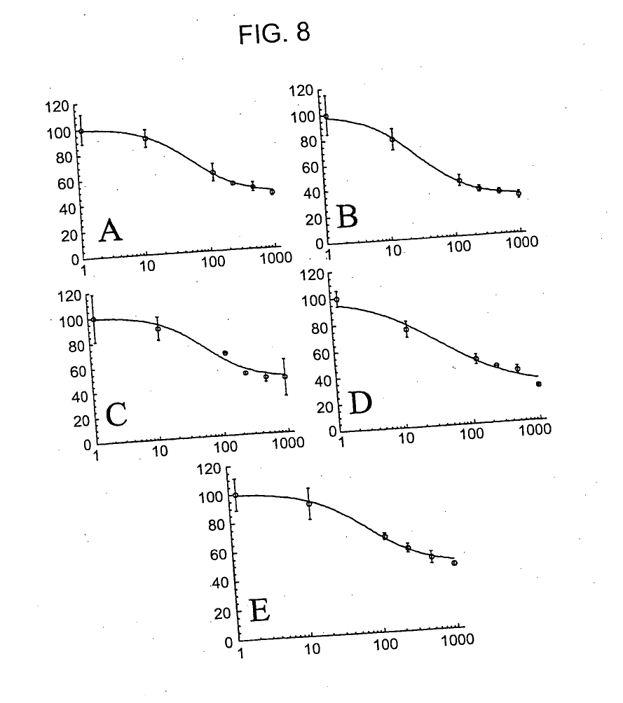

In some alternative embodiments, the assay above is performed using varying concentrations of the test compound X, rather than fixed concentration. This allows determination of the Ki of the X for each PDZ as described above. Examples of this is shown in FIG. 8 for small molecules, and in FIG. 3 for peptide inhibition.

In an alternative embodiment, instead of pairing each PDZ-PL with a specific labeled ligand Li, a mixture of different labeled ligands is created that such that for every PDZ at least one of the ligands in the mixture binds to this PDZ sufficiently to detect the binding in the “G” assay. This mixture is then used for every PDZ domain.

In one embodiment, compound X is known to have a desired biological effect, but the chemical mechanism by which it has that effect is unknown. The assays of the invention can then be used to determine if compound X has its effect by binding to a PDZ domain.

In one embodiment, PDZ-domain containing proteins are classified in to groups based on their biological function, e.g. into those that regulate chemotaxis versus those that regulate transcription. An optimal inhibitor of a particular function (e.g., including but not limited to an anti-chemotactic agent, an anti-T cell activation agent, cell-cycle control, vesicle transport, apoptosis, etc.) will inhibit multiple PDZ-ligand interactions involved in the function (e.g., chemotaxis, activation) but few other interactions. Thus, the assay is used in one embodiment in screening and design of a drug that specifically blocks a particular function. For example, an agent designed to block chemotaxis might be identified because, at a given concentration, the agent inhibits 2 or more PDZs involved in chemotaxis but fewer than 3 other PDZs, or that inhibits PDZs involved in chemotaxis with a Ki>10-fold better than for other PDZs. Thus, the invention provides a method for identifying an agent that inhibits a first selected PDZ-PL interaction or plurality of interactions-but does not inhibit a second selected PDZ-PL interaction or plurality of interactions. The two (or more) sets of interactions can be selected on the basis of the known biological function of the PDZ proteins, the tissue specificity of the PDZ proteins, or any other criteria. Moreover, the assay can be used to determine effective doses (i.e., drug concentrations) that result in desired biological effects while avoiding undesirable effects.

C. Agonists and Antagonists of PDZ-PL Interactions As described herein, interactions between PDZ proteins and PL proteins in cells (e.g., cervical cells) may be disrupted or inhibited by the presence of pathogens. Pathogens can be identified using screening assays described herein. Agonists and antagonists of PDZ-Pathogen PL interactions or PDZ-Cellular PL interactions can be useful in discerning or confirming specific interactions. In some embodiments, an agonist will increase the sensitivity of a PDZ-pathogen PL interaction. In other embodiments, an antagonist of a PDZ-pathogen PL interaction can be used to verify the specificity of an interaction. In one embodiment, the motifs disclosed herein are used to design modulators. In some embodiments, the antagonists of the invention have a structure (e.g., peptide sequence) based on the C-terminal residues of PL-domain proteins listed in TABLE 2. In some embodiments, the antagonists of the invention have a structure (e.g., peptide sequence) based on a PL motif disclosed herein or in U.S. patent application Ser. No. 09/724553.

The PDZ/PL antagonists and antagonists of the invention may be any of a large variety of compounds, both naturally occurring and synthetic, organic and inorganic, and including polymers (e.g., oligopeptides, polypeptides, oligonucleotides, and polynucleotides), small molecules, antibodies, sugars, fatty acids, nucleotides and nucleotide analogs, analogs of naturally occurring structures (e.g., peptide mimetics, nucleic acid analogs, and the like), and numerous other compounds. Although, for convenience, the present discussion primarily refers antagonists of PDZ-PL interactions, it will be recognized that PDZ-PL interaction agonists can also be use in the methods disclosed herein.

In one aspect, the peptides and peptide mimetics or analogues of the invention contain an amino acid sequence that binds a PDZ domain in a cell of interest. In one embodiment, the antagonists comprise a peptide that has a sequence corresponding to the carboxy-terminal sequence of a PL protein listed in TABLES 2 or 3, e.g., a peptide listed TABLES 2 or 3. Typically, the peptide comprises at least the C-terminal two (3), three (3) or four (4) residues of the PL protein, and often the inhibitory peptide comprises more than three residues (e.g., at least four, five, six, seven, eight, nine, ten, twelve or fifteen residues) from the PL protein C-terminus.

In some embodiments, the inhibitor is a peptide, e.g., having a sequence of a PL C-terminal protein sequence. An example of this is shown in FIG. 3.

In some embodiments, the antagonist is a fusion protein comprising such a sequence. Fusion proteins containing a transmembrane transporter amino acid sequence are particularly useful.

In some embodiments, the inhibitor is conserved variant of the PL C-terminal protein sequence having inhibitory activity.

In some embodiments, the antagonist is a peptide mimetic of a PL C-terminal sequence.

In some embodiments, the inhibitor is a small molecule (i.e., having a molecular weight less than 1 kD).

D. Peptide Antagonists

In one embodiment, the antagonists comprise a peptide that has a sequence of a PL protein carboxy-terminus listed in TABLE 2. The peptide comprises at least the C-terminal two (2) residues of the PL protein, and typically, the inhibitory peptide comprises more than two residues (e.g, at least three, four, five, six, seven, eight, nine, ten, twelve or fifteen residues) from the PL protein C-terminus. The peptide may be any of a variety of lengths (e.g., at least 2, at least 3, at least 4, at least 5, at least 6, at least 8, at least 10, or at least 20 residues) and ma contain additional residues not from the PL protein. It will be recognized that short PL peptides are sometime used in the rational design of other small molecules with similar properties.

Although most often, the residues shared by the inhibitory peptide with the PL protein are found at the C-terminus of the peptide. However, in some embodiments, the sequence is internal. Similarly, in some cases, the inhibitory peptide comprises residues from a PL sequence that is near, but not at the c-terminus of a PL protein (see, Gee et al., 1998, J Biological Chem. 273:21980-87).

Sometime the PL protein carboxy-terminus sequence is referred to as the “core PDZ motif sequence” referring to the ability of the short sequence to interact with the PDZ domain. For example, in an embodiment, the “core PDZ motif sequence” contains the last four C-terminus amino acids. As described above, the four amino acid core of a PDZ motif sequence may contain additional amino acids at its amino terminus to further increase its binding affinity and/or stability. Thus, in one embodiment, the PDZ motif sequence peptide can be from four amino acids up to 15 amino acids. It is preferred that the length of the sequence to be 6-10 amino acids. More preferably, the PDZ motif sequence contains 8 amino acids. Additional amino acids at the amino terminal end of the core sequence may be derived from the natural sequence in each HPV protein or a synthetic linker. The additional amino acids may also be conservatively substituted. When the third residue from the C-terminus is S, T or Y, this residue may be phosphorylated prior to the use of the peptide.

In some embodiments, the peptide and nonpeptide inhibitors of the are small, e.g., fewer than ten amino acid residues in length if a peptide. Further, it is reported that a limited number of ligand amino acids directly contact the PDZ domain (generally less than eight) (Kozlov et al., 2000, Biochemistry 39, 2572; Doyle et al., 1996, Cell 85, 1067) and that peptides as short as the C-terminal three amino acids often retain similar binding properties to longer (>15) amino acids peptides (Yanagisawa et al., 1997, J. Biol. Chem. 272, 8539).

E. Peptide Variants Having identified PDZ binding peptides and PDZ-PL interaction inhibitory sequences, variations of these sequences can be made and the resulting peptide variants can be tested for PDZ domain binding or PDZ-PL inhibitory activity. In embodiments, the variants have the same or a different ability to bind a PDZ domain as the parent peptide. Typically, such amino acid substitutions are conservative, i.e., the amino acid residues are replaced with other amino acid residues having physical and/or chemical properties similar to the residues they are replacing. Preferably, conservative amino acid substitutions are those wherein an amino acid is replaced with another amino acid encompassed within the same designated class, as shown in Table 1.

F. Peptide Mimetics

Having identified PDZ binding peptides and PDZ-PL interaction inhibitory sequences, peptide mimetics can be prepared using routine methods, and the inhibitory activity of the mimetics can be confirmed using the assays of the invention. Thus, in some embodiments, the agonist or antagonist is a peptide mimetic of a PL C-terminal sequence. The skilled artisan will recognize that individual synthetic residues and polypeptides incorporating mimetics can be synthesized using a variety of procedures and methodologies, which are well described in the scientific and patent literature, e.g., Organic Syntheses Collective Volumes, Gilman et al. (Eds) John Wiley & Sons, Inc., NY. Polypeptides incorporating mimetics can also be made using solid phase synthetic procedures, as described, e.g., by Di Marchi, et al., U.S. Pat. No. 5,422,426. Mimetics of the invention can also be synthesized using combinatorial methodologies. Various techniques for generation of peptide and peptidomimetic libraries are well known, and include, e.g., multipin, tea bag, and split-couple-mix techniques; see, e.g., al-Obeidi (1998) Mol. Biotechnol. 9:205-223; Hruby (1997) Curr. Opin. Chem. Biol. 1:114-119; Ostergaard (1997) Mol. Divers. 3:17-27; Ostresh (1996) Methods Enzymol. 267:220-234.

G. Small Molecules

In some embodiments, the agonist or antagonist is a small molecule (i.e., having a molecular weight less than 5 kD or 2 kD). Methods for screening small molecules are well known in the art and include those described supra. Small molecules agonists or antagonists can be identified using any of the biochemical PDZ:PL interaction assays disclosed herein. Following identification of small molecule antagonists/agonists, the effects of these compounds can be tested in the biological assays provided herein. An example of the identification of small molecule antagonists of binding between an oncogenic E6 protein and a PDZ protein is shown in FIG. 8.

In certain embodiments, the small molecules may be isolated peptide molecules, particularly peptides of no more that 5 amino acids in length and containing two, three or four amino acids corresponding to the amino acids at the C-terminus of an oncogenic E6 protein, may contain certain chemical moieties covalently bonded to the N- and/or C-terminus of the peptide.

Without wishing to limit these modified peptides to those having a particular amino acid sequence, 15 types of N-terminal addition, and three C-terminal additions are described below. Any subject polypeptide may be modified at the C-terminus, the N-terminus, or both the C- or N- terminus. In cases where both the C- and N-termini of a peptide are modified, any of the three C-terminal moieties may be combined with any of the 15 N-terminal moieties.

Solely to exemplify this aspect of the invention, the structures of four different peptides having at least two contiguous amino acids from the C-terminus of an oncogenic E6 protein, are shown below. The peptides are named “EV peptide”, “QL peptide”, “TEV peptide” and “TQL peptide”, corresponding to the E6 proteins of HPV strains 16 and 18, and others.

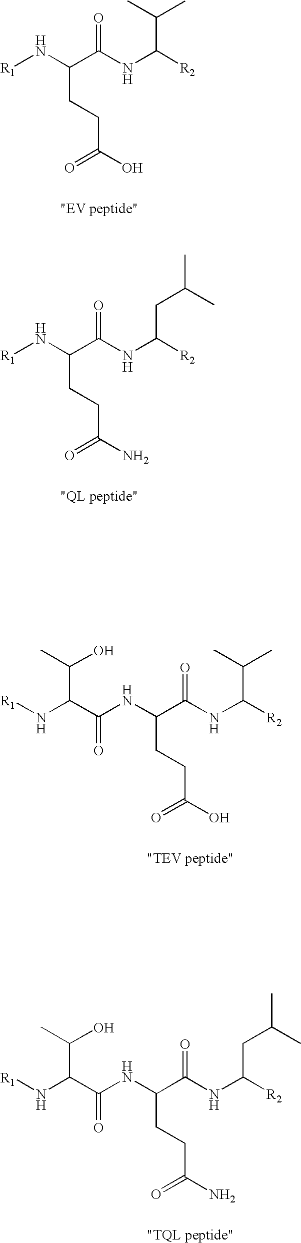

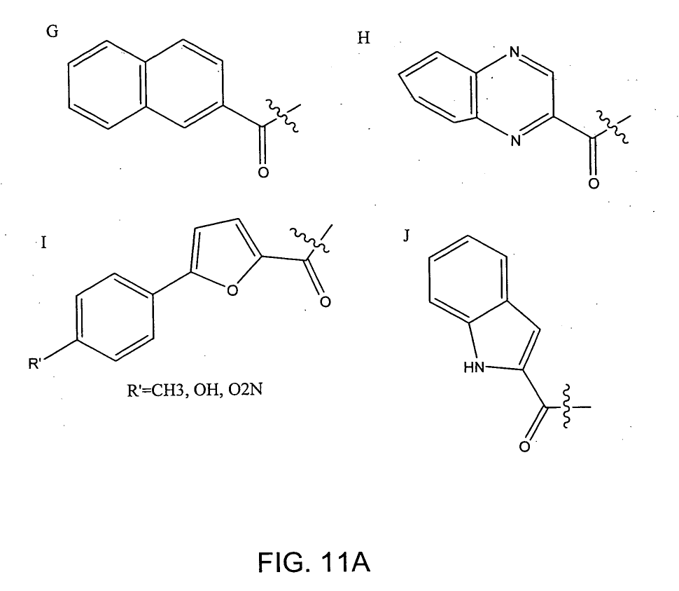

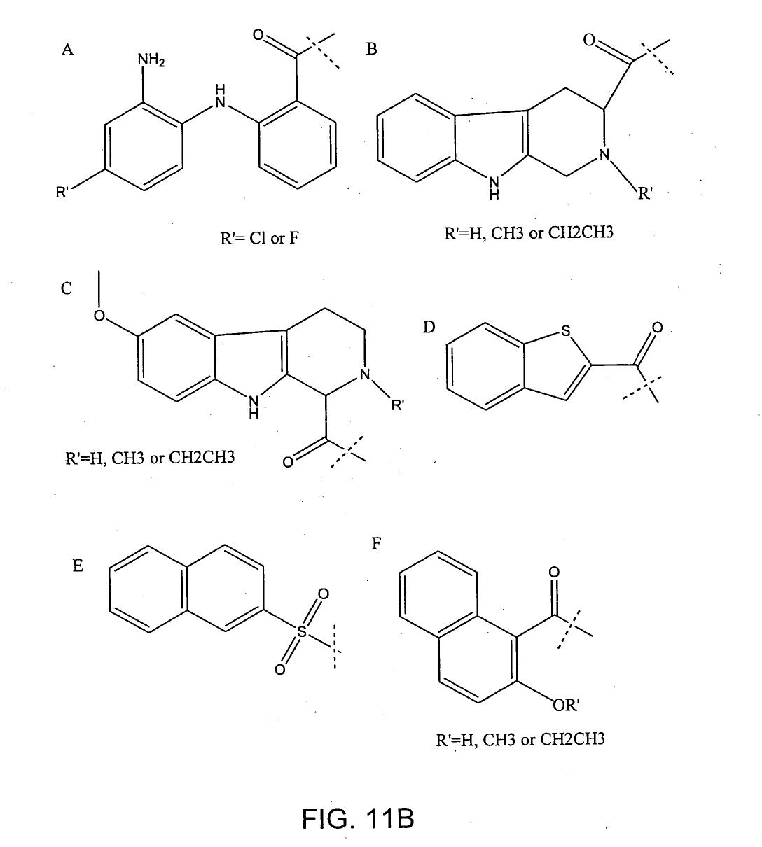

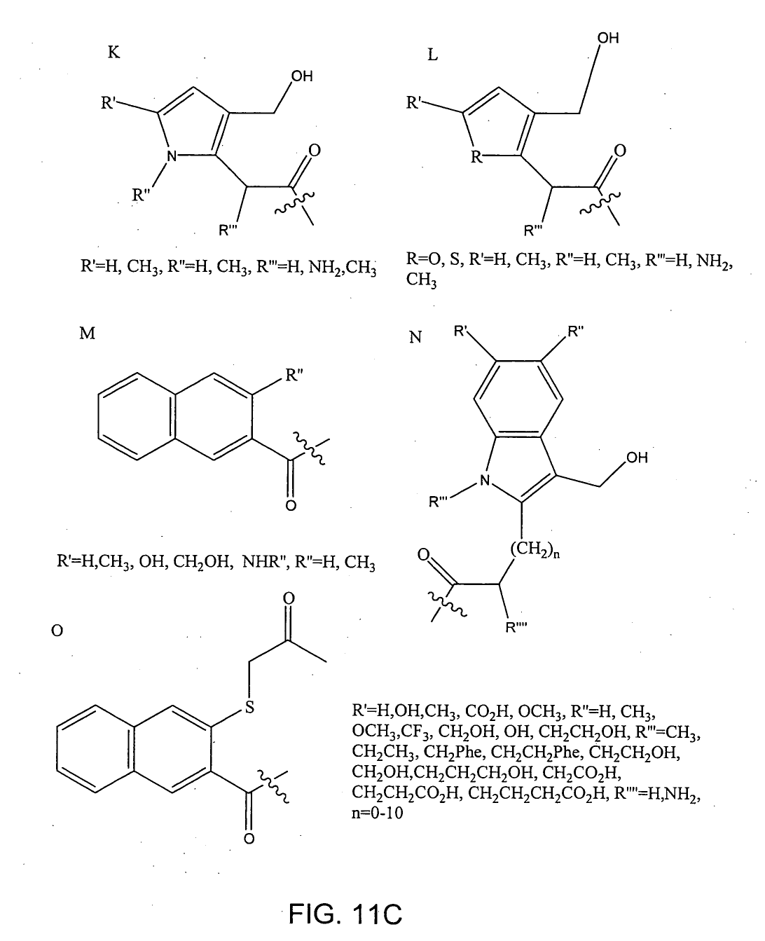

The R₂ groups of any of these peptides may be carboxyl, hydroxyl or tetrazole moieties. The R₁ groups of any of these peptides may be may be any of the moieties shown in FIG. 11, panels A-O.

For example, as shown in FIG. 11: R1 may be a substituted N-Phenyl-benzene-1,2-diamine (panel A), a substituted 2,3,4,9-Tetrahydro-1H-b-carboline group (panel B), a substituted 6-Methoxy-2,3,4,9-tetrahydro-1H-b-carboline group (panel C), a Benzo[b]thiophene group (panel D), a linked naphthalene group (panel E), a substituted Naphthalen-2-ol group (panel F), a Naphthalene group (panel G), a Quinoxaline group (panel H), a substituted 2-Phenyl-furan group (panel I), a 1H-Indole group (panel J) a substituted 2-methyl-1H-pyrrol-3-yl)-methanol group (panel K) a substituted (2-Methyl-furan-3-yl)-methanol or (2-Methyl-thiophen-3-yl)-methanol group (panel L), a substituted Naphthalene group (panel M), a substituted (1H-Indol-3-yl)-methanol group (panel N) or a 1-(Naphthalen-2-ylsulfanyl)-propan-2-one group (panel O).

X. Alternative Methods for Treatment of Cervical Cancer

As demonstrated in the examples included with this application, E6 oncoproteins activate cJUN N-terminal Kinase (JNK) in transformed cells. JNK has been demonstrated to be involved in a number of apoptotic signaling pathways. Inhibition of JNK activation using small molecules could be used injunction with PDZ:PL directed therapy or as an alternative to block oncogenic transformation in HPV transformed cells. Such an inhibitor could be effective in treating any of the forms of Cancer resulting from oncogenic HPV infection.

XI. Recombinant Modulator Synthesis

As indicated in the Background section, PDZ domain-containing proteins are involved in a number of biological functions, including, but not limited to, vesicular trafficking, tumor suppression, protein sorting, establishment of membrane polarity, apoptosis, regulation of immune response and organization of synapse formation. In general, this family of proteins has a common function of facilitating the assembly of multi-protein complexes, often serving as a bridge between several proteins, or regulating the function of other proteins. Additionally, as also noted supra, these proteins are found in essentially all cell types. Consequently, inappropriate PDZ-PL interactions or abnormal interactions can be targeted for the treatment of a wide variety of biological and physiological conditions. In particular, PL proteins from pathogenic organisms can be targeted using PDZ domains as therapeutics. Examples are given below.

A. Chemical Synthesis

The peptides of the invention or analogues thereof, may be prepared using virtually any art-known technique for the preparation of peptides and peptide analogues. For example, the peptides may be prepared in linear form using conventional solution or solid phase peptide syntheses and cleaved from the resin followed by purification procedures (Creighton, 1983, Protein Structures And Molecular Principles, W.H. Freeman and Co., N.Y.). Suitable procedures for synthesizing the peptides described herein are well known in the art. The composition of the synthetic peptides may be confirmed by amino acid analysis or sequencing (e.g., the Edman degradation procedure and mass spectroscopy).

In addition, analogues and derivatives of the peptides can be chemically synthesized. The linkage between each amino acid of the peptides of the invention may be an amide, a substituted amide or an isostere of amide. Nonclassical amino acids or chemical amino acid analogues can be introduced as a substitution or addition into the sequence. Non-classical amino acids include, but are not limited to, the D-isomers of the common amino acids, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosine, citrulline, cysteic acid, t-butylglycine, t-butylalanine, phenylglycine, cyclohexylalanine, β-alanine, fluoro-amino acids, designer amino acids such as β-methyl amino acids, Cα-methyl amino acids, Nα,-methyl amino acids, and amino acid analogues in general. Furthermore, the amino acid can be D (dextrorotary) or L (levorotary).

B. Recombinant Synthesis

If the peptide is composed entirely of gene-encoded amino acids, or a portion of it is so composed, the peptide or the relevant portion may also be synthesized using conventional recombinant genetic engineering techniques. For recombinant production, a polynucleotide sequence encoding a linear form of the peptide is inserted into an appropriate expression vehicle, i.e., a vector that contains the necessary elements for the transcription and translation of the inserted coding sequence, or in the case of an RNA viral vector, the necessary elements for replication and translation. The expression vehicle is then transfected into a suitable target cell that will express the peptide. Depending on the expression system used, the expressed peptide is then isolated by procedures well-established in the art. Methods for recombinant protein and peptide production are well known in the art (see, e.g., Maniatis et al., 1989, Molecular Cloning A Laboratory Manual, Cold Spring Harbor Laboratory, N.Y.; and Ausubel et al., 1989, Current Protocols in Molecular Biology, Greene Publishing Associates and Wiley Interscience, N.Y.).

A variety of host-expression vector systems may be utilized to express the peptides described herein. These include, but are not limited to, microorganisms such as bacteria transformed with recombinant bacteriophage DNA or plasmid DNA expression vectors containing an appropriate coding sequence; yeast or filamentous fungi transformed with recombinant yeast or fungi expression vectors containing an appropriate coding sequence; insect cell systems infected with recombinant virus expression vectors (e.g., baculovirus) containing an appropriate coding sequence; plant cell systems infected with recombinant virus expression vectors (e.g., cauliflower mosaic virus or tobacco mosaic virus) or transformed with recombinant plasmid expression vectors (e.g., Ti plasmid) containing an appropriate coding sequence; or animal cell systems.

In some embodiments, increasing the number of copies of a PL therapeutic may be used to increase the specificity or sensitivity of treatment. An example of this is presented in EXAMPLES 5. The TIP-TIP-IgG vector produces a fusion protein that has duplicated copies of the PDZ domain from TIP-1 and the protein itself should dimerize on the basis of the IgG constant region backbone. Hence, a single protein contains 2-4 copies of the TIP-1 PDZ domain. In a similar manner, addition tandem repeats of PL capturdetectors could be fashioned. In some embodiments, different PDZ domains from different proteins could be engineered to express as a single protein (e.g., the PDZ domains of TIP-1 and MAGI-1 could be engineered to detect or block oncogenic HPV E6 proteins). In a similar manner, a different Ig backbone could be used to increase the avidity of a construct. For example, the IgG constant regions will dimerize with itself, but the IgM constant regions will form a complex of ten monomers.

The expression elements of the expression systems vary in their strength and specificities. Depending on the host/vector system utilized, any of a number of suitable transcription and translation elements, including constitutive and inducible promoters, may be used in the expression vector. For example, when cloning in bacterial systems, inducible promoters such as pL of bacteriophage λ, plac, ptrp, ptac (ptrp-lac hybrid promoter) and the like may be used; when cloning in insect cell systems, promoters such as the baculovirus polyhedron promoter may be used; when cloning in plant cell systems, promoters derived from the genome of plant cells (e.g., heat shock promoters; the promoter for the small subunit of RUBISCO; the promoter for the chlorophyll a/b binding protein) or from plant viruses (e.g. the 35S RNA promoter of CaMV; the coat protein promoter of TMV) may be used; when cloning in mammalian cell systems, promoters derived from the genome of mammalian cells (e.g., metallothionein promoter) or from mammalian viruses (e.g., the adenovirus late promoter; the vaccinia virus 7.5 K promoter) may be used; when generating cell lines that contain multiple copies of expression product, SV40-, BPV- and EBV-based vectors may be used with an appropriate selectable marker.

In cases where plant expression vectors are used, the expression of sequences encoding the peptides of the invention may be driven by any of a number of promoters. For example, viral promoters such as the 35S RNA and 19S RNA promoters of CaMV (Brisson et al., 1984, Nature 310:511-514), or the coat protein promoter of TMV (Takamatsu et al., 1987, EMBO J. 6:307-311) may be used; alternatively, plant promoters such as the small subunit of RUBISCO (Corizi et al., 1984, EMBO J. 3:1671-1680; Broglie et al., 1984, Science 224:838-843) or heat shock promoters, e.g., soybean hspl7.5-E or hspl7.3-B (Gurley et al., 1986, Mol. Cell. Biol. 6:559-565) may be used. These constructs can be introduced into planleukocytes using Ti plasmids, Ri plasmids, plant virus vectors, direct DNA transformation, microinjection, electroporation, etc. For reviews of such techniques see, e.g., Weissbach & Weissbach, 1988, Methods for Plant Molecular Biology, Academic Press, NY, Section VIII, pp. 421-463; and Grierson & Corey, 1988, Plant Molecular Biology, 2d Ed., Blackie, London, Ch. 7-9.

In one insect expression system that may be used to produce the peptides of the invention, Autographa californica nuclear polyhidrosis virus (AcNPV) is used as a vector to express the foreign genes. The virus grows in Spodoptera frugiperda cells. A coding sequence may be cloned into non-essential regions (for example the polyhedron gene) of the virus and placed under control of an AcNPV promoter (for example, the polyhedron promoter). Successful insertion of a coding sequence will result in inactivation of the polyhedron gene and production of non-occluded recombinant virus (i.e., virus lacking the proteinaceous coat coded for by the polyhedron gene). These recombinant viruses are then used to infect Spodoptera frugiperda cells in which the inserted gene is expressed. (e.g., see Smith et al., 1983, J. Virol. 46:584; Smith, U.S. Pat. No. 4,215,051). Further examples of this expression system may be found in Current Protocols in Molecular Biology, Vol. 2, Ausubel et al., eds., Greene Publish. Assoc. & Wiley Interscience.

In mammalian host cells, a number of viral based expression systems may be utilized. In cases where an adenovirus is used as an expression vector, a coding sequence may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing peptide in infected hosts. (e.g., See Logan & Shenk, 1984, Proc. Natl. Acad. Sci. USA 81:3655-3659). Alternatively, the vaccinia 7.5 K promoter may be used, (see, e.g., Mackett et al., 1982, Proc. Natl. Acad. Sci. USA 79:7415-7419; Mackett et al., 1984, J. Virol. 49:857-864; Panicali et al., 1982, Proc. Natl. Acad. Sci. USA 79:4927-4931).

Other expression systems for producing linear peptides of the invention will be apparent to those having skill in the art.

C. Tags or Markers

Tags and markers are frequently used to aid in purification of components or delivery of treatments to cells or tissues. Examples of biological tags include, but are not limited to, glutathione-S-transferase, maltose binding protein, Immunoglobulin domains, Intein, Hemagglutinin epitopes, myc epitopes, etc. Examples of chemical tags include, but are not limited to, biotin, gold, paramagnetic particles or fluorophores. These examples can be used to deliver therapeutic agents to specific tissues or cells or can be used by those skilled in the art to purify proteins or compounds from complex mixtures.

D. Purification of the Peptides and Peptide Analogues

The peptides and peptide analogues of the invention can be purified by art-known techniques such as high performance liquid chromatography, ion exchange chromatography, gel electrophoresis, affinity chromatography and the like. The actual conditions used to purify a particular peptide or analogue will depend, in part, on factors such as net charge, hydrophobicity, hydrophilicity, etc., and will be apparent to those having skill in the art. The purified peptides can be identified by assays based on their physical or functional properties, including radioactive labeling followed by gel electrophoresis, radioimmuno-assays, ELISA, bioassays, and the like.

XII. Formulation and Route of Administration

A. Introduction of Agonists or Antagonists (e.g., Peptides and Fusion Proteins) into Cells

In one aspect, the PDZ-PL antagonists of the invention are introduced into a cell to modulate (i.e., increase or decrease) a biological function or activity of the cell. Many small organic molecules readily cross the cell membranes (or can be modified by one of skill using routine methods to increase the ability of compounds to enter cells, e.g., by reducing or eliminating charge, increasing lipophilicity, conjugating the molecule to a moiety targeting a cell surface receptor such that after interacting with the receptor). Methods for introducing larger molecules, e.g., peptides and fusion proteins are also well known, including, e.g., injection, liposome-mediated fusion, application of a hydrogel, conjugation to a targeting moiety conjugate endocytozed by the cell, electroporation, and the like).

In one embodiment, the antagonist or agent is a fusion polypeptide or derivatized polypeptide. A fusion or derivatized protein may include a targeting moiety that increases the ability of the polypeptide to traverse a cell membrane or causes the polypeptide to be delivered to a specified cell type (e.g., cancer cells) preferentially or cell compartment (e.g., nuclear compartment) preferentially. Examples of targeting moieties include lipid tails, amino acid sequences such as antennapoedia peptide or a nuclear localization signal (NLS; e.g., Xenopus nucleoplasmin Robbins et al., 1991, Cell 64:615).

In one embodiment of the invention, a peptide sequence or peptide analog determined to inhibit a PDZ domain-PL protein binding, in an assay of the invention is introduced into a cell by linking the sequence to an amino acid sequence that facilitates its transport through the plasma membrane (a “transmembrane transporter sequence”). The peptides of the invention may be used directly or fused to a transmembrane transporter sequence to facilitate their entry into cells. In the case of such a fusion peptide, each peptide may be fused with a heterologous peptide at its amino terminus directly or by using a flexible polylinker such as the pentamer G-G-G-G-S (SEQ ID NO:1) repeated 1 to 3 times. Such linker has been used in constructing single chain antibodies (scFv) by being inserted between V_{H} and V_{L} (Bird et al., 1988, Science 242:423-426; Huston et al., 1988, Proc. Natl. Acad. Sci. U.S.A. 85:5979-5883). The linker is designed to enable the correct interaction between two beta-sheets forming the variable region of the single chain antibody. Other linkers that may be used include Glu-Gly-Lys-Ser-Ser-Gly-Ser-Gly-Ser-Glu-Ser-Lys-Val-Asp (SEQ ID NO:2) (Chaudhary et al., 1990, Proc. Natl. Acad. Sci. U.S.A. 87:1066-1070) and Lys-Glu-Ser-Gly-Ser-Val-Ser-Ser-Glu-Gln-Leu-Ala-Gln-Phe-Arg-Ser-Leu-Asp (SEQ ID NO:3) (Bird et al., 1988, Science 242:423-426).

A number of peptide sequences have been described in the art as capable of facilitating the entry of a peptide linked to these sequences into a cell through the plasma membrane (Derossi et al., 1998, Trends in Cell Biol. 8:84). For the purpose of this invention, such peptides are collectively referred to as transmembrane transporter peptides. Examples of these peptide include, but are not limited to, tat derived from HIV (Vives et al., 1997, J. Biol. Chem. 272:16010; Nagahara et al., 1998, Nat. Med. 4:1449), antennapedia from Drosophila (Derossi et al., 1994, J. Biol. Chem. 261:10444), VP22 from herpes simplex virus (Elliot and D'Hare, 1997, Cell 88:223-233), complementarity-determining regions (CDR) 2 and 3 of anti-DNA antibodies (Avrameas et al., 1998, Proc. Natl Acad. Sci. U.S.A., 95:5601-5606), 70 KDa heat shock protein (Fujihara, 1999, EMBO J. 18:411-419) and transportan (Pooga et al., 1998, FASEB J. 12:67-77). In a preferred embodiment of the invention, a truncated HIV tat peptide having the sequence of GYGRKKRRQRRRG (SEQ ID NO:4) is used.

It is preferred that a transmembrane transporter sequence is fused to a HPV protein carboxyl terminal sequence at its amino-terminus with or without a linker. Generally, the C-terminus of a PDZ motif sequence (PL sequence) must be free in order to interact with a PDZ domain. The transmembrane transporter sequence may be used in whole or in part as long as it is capable of facilitating entry of the peptide into a cell.

In an alternate embodiment of the invention, a HPV protein C-terminal sequence may be used alone when it is delivered in a manner that allows its entry into cells in the absence of a transmembrane transporter sequence. For example, the peptide may be delivered in a liposome formulation or using a gene therapy approach by delivering a coding sequence for the PDZ motif alone or as a fusion molecule into a target cell.

The compounds of the of the invention may also be administered via liposomes, which serve to target the conjugates to a particular tissue, such as cervical tissue, or targeted selectively to infected cells, as well as increase the half-life of the peptide composition. Liposomes include emulsions, foams, micelles, insoluble monolayers, liquid crystals, phospholipid dispersions, lamellar layers and the like. In these preparations the peptide to be delivered is incorporated as part of a liposome, alone or in conjunction with a molecule that binds tooncogenic HPV protein or with other therapeutic or immunogenic compositions. Thus, liposomes filled with a desired peptide or conjugate of the invention can be directed to the site of transformed cervical cells, where the liposomes then deliver the selected inhibitor compositions. Liposomes for use in the invention are formed from standard vesicle-forming lipids, which generally include neutral and negatively charged phospholipids and a sterol, such as cholesterol. The selection of lipids is generally guided by consideration of, e.g., liposome size, acid lability and stability of the liposomes in the blood stream. A variety of methods are available for preparing liposomes, as described in, e.g., Szoka et al., Ann. Rev. Biophys. Bioeng. 9:467 (1980), U.S. Pat. Nos. 4,235,871, 4,501,728 and 4,837,028.

The targeting of liposomes using a variety of targeting agents is well known in the art (see, e.g., U.S. Pat. Nos. 4,957,773 and 4,603,044). For targeting to the cervical cells, a ligand to be incorporated into the liposome can include, e.g., antibodies or fragments thereof specific for cell surface determinants of the desired HPV-transformed cervical cells. A liposome suspension containing a peptide or conjugate may be administered intravenously, locally, topically, etc. in a dose which varies according to, the manner of administration, the conjugate being delivered, and the stage of the disease being treated.

In order to specifically deliver a PDZ ligand sequence (PL sequence) peptide into a specific cell type, the peptide may be linked to a cell-specific targeting moiety, which include but are not limited to, ligands for surface molecules that are preferentially presented on the surface of HPV-infected or cancerous cells, such as growth factors, hormones and cytokine receptors, as well as antibodies or antigen-binding fragments thereof. Proteins expressed on the surface of appropriate infected cells should be selected as the homing signal for increasing the concentration of therapeutic at the infected site.

Antibodies are the most versatile cell-specific targeting moieties because they can be generated against any cell surface antigen. Monoclonal antibodies have been generated against many cell-surface markers such as CD antigens, ion channels, and signal transduction molecules. Antibody variable region genes can be readily isolated from hybridoma cells by methods well known in the art. However, since antibodies are assembled between two heavy chains and two light chains, it is preferred that a scFv be used as a cell-specific targeting moiety in the present invention. Such scFv are comprised of V_{H} and V_{L} domains linked into a single polypeptide chain by a flexible linker peptide.

The PDZ motif sequence (PL sequence) may be linked to a transmembrane transporter sequence and a cell-specific targeting moiety to produce a tri-fusion molecule. This molecule can bind to a cervical cell surface molecule, passes through the membrane and targets PDZ domains. Alternatively, a PDZ motif sequence (PL sequence) may be linked to a cell-specific targeting moiety that binds to a surface molecule that internalizes the fusion peptide.

In another approach, microspheres of artificial polymers of mixed amino acids (proteinoids) have been used to deliver pharmaceuticals. For example, U.S. Pat. No. 4,925,673 describes drug-containing proteinoid microsphere carriers as well as methods for their preparation and use. These proteinoid microspheres are useful for the delivery of a number of active agents. Also see, U.S. Pat. Nos. 5,907,030 and 6,033,884, which are incorporated herein by reference.

B. Introduction of Polynucleotides into Cells

By introducing gene sequences into cells, gene therapy can be used to treat conditions in which cervical cells are activated to result in deleterious consequences. In one embodiment, a polynucleotide that encodes a PL sequence peptide of the invention is introduced into a cell where it is expressed. In another embodiment, a polynucleotide encoding a PDZ domain is introduced into a cell where it is expressed. The expressed peptide then inhibits the interaction of PDZ proteins and PL proteins in the cell.

Thus, in one embodiment, the polypeptides of the invention are expressed in a cell by introducing a nucleic acid (e.g., a DNA expression vector or mRNA) encoding the desired protein or peptide into the cell. Expression may be either constitutive or inducible depending on the vector and choice of promoter. Methods for introduction and expression of nucleic acids into a cell are well known in the art and described herein.

In a specific embodiment, nucleic acids comprising a sequence encoding a peptide disclosed herein, are administered to a human subject. In this embodiment of the invention, the nucleic acid produces its encoded product that mediates a therapeutic effect. Any of the methods for gene therapy available in the art can be used according to the present invention. Exemplary methods are described below.

For general reviews of the methods of gene therapy, see Goldspiel et al., 1993, Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May, 1993, TIBTECH 11(5):155-215. Methods commonly known in the art of recombinant DNA technology which can be used are described in Ausubel et al. (eds.), 1993, Current Protocols in Molecular Biology, John Wiley & Sons, NY; and Kriegler, 1990, Gene Transfer and Expression, A Laboratory Manual, Stockton Press, NY.

In a preferred embodiment of the invention, the therapeutic composition comprises a coding sequence that is part of an expression vector. In particular, such a nucleic acid has a promoter operably linked to the coding sequence, said promoter being inducible or constitutive, and, optionally, tissue-specific. In another specific embodiment, a nucleic acid molecule is used in which the coding sequence and any other desired sequences are flanked by regions that promote homologous recombination at a desired site in the genome, thus providing for intrachromosomal expression of the nucleic acid (Koller and Smithies, 1989, Proc. Natl. Acad. Sci. USA 86:8932-8935; Zijlstra et al., 1989, Nature 342:435-438).

Delivery of the nucleic acid into a patient may be either direct, in which case the patient is directly exposed to the nucleic acid or nucleic acid-carrying vector, or indirect, in which case, cells are first transformed with the nucleic acid in vitro, then transplanted into the patient. These two approaches are known, respectively, as in vivo or ex vivo gene therapy.

In a specific embodiment, the nucleic acid is directly administered in vivo, where it is expressed to produce the encoded product. This can be accomplished by any methods known in the art, e.g., by constructing it as part of an appropriate nucleic acid expression vector and administering it so that it becomes intracellular, e.g., by infection using a defective or attenuated retroviral or other viral vector (see U.S. Pat. No. 4,980,286), by direct injection of naked DNA, by use of microparticle bombardment (e.g., a gene gun; Biolistic, Dupont), by coating with lipids or cell-surface receptors or transfecting agents, by encapsulation in liposomes, microparticles, or microcapsules, by administering it in linkage to a peptide which is known to enter the nucleus, or by administering it in linkage to a ligand subject to receptor-mediated endocytosis (see e.g., Wu and Wu, 1987, J. Biol. Chem. 262:4429-4432) which can be used to target cell types specifically expressing the receptors. In another embodiment, a nucleic acid-ligand complex can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180 dated Apr. 16, 1992; WO 92/22635 dated Dec. 23, 1992; W092/20316 dated Nov. 26, 1992; W093/14188 dated Jul. 22, 1993; WO 93/20221 dated Oct. 14, 1993). Alternatively, the nucleic acid can be introduced intracellularly and incorporated within host cell DNA for expression, by homologous recombination (Koller and Smithies, 1989, Proc. Natl. Acad. Sci. USA 86:8932-8935; Zijlstra et al., 1989, Nature 342:435-438).

In a preferred embodiment of the invention, adenoviruses as viral vectors can be used in gene therapy. Adenoviruses have the advantage of being capable of infecting non-dividing cells (Kozarsky and Wilson, 1993, Current Opinion in Genetics and Development 3:499-503). Other instances of the use of adenoviruses in gene therapy can be found in Rosenfeld et al., 1991, Science 252:431-434; Rosenfeld et al., 1992, Cell 68:143-155; and Mastrangeli et al., 1993, J. Clin. Invest. 91:225-234. Furthermore, adenoviral vectors with modified tropism may be used for cell specific targeting (WO98/40508). Adeno-associated virus (AAV) has also been proposed for use in gene therapy (Walsh et al., 1993, Proc. Soc. Exp. Biol. Med. 204:289-300).

In addition, retroviral vectors (see Miller et al., 1993, Meth. Enzymol. 217:581-599) have been modified to delete retroviral sequences that are not necessary for packaging of the viral genome and integration into host cell DNA. The coding sequence to be used in gene therapy is cloned into the vector, which facilitates delivery of the gene into a patient. More detail about retroviral vectors can be found in Boesen et al., 1994, Biotherapy 6:291-302, which describes the use of a retroviral vector to deliver the mdr1 gene to hematopoietic stem cells in order to make the stem cells more resistant to chemotherapy. Other references illustrating the use of retroviral vectors in gene therapy are: Clowes et al., 1994, J. Clin. Invest. 93:644-651; Kiem et al., 1994, Blood 83:1467-1473; Salmons and Gunzberg, 1993, Human Gene Therapy 4:129-141; and Grossman and Wilson, 1993, Curr. Opin. in Genetics and Devel. 3:110-114.

Another approach to gene therapy involves transferring a gene to cells in tissue culture. Usually, the method of transfer includes the transfer of a selectable marker to the cells. The cells are then placed under selection to isolate those cells that have taken up and are expressing the transferred gene. Those cells are then delivered to a patient.

In this embodiment, the nucleic acid is introduced into a cell prior to administration in vivo of the resulting recombinant cell. Such introduction can be carried out by any method known in the art, including but not limited to transfection, electroporation, lipofection, microinjection, infection with a viral or bacteriophage vector containing the nucleic acid sequences, cell fusion, chromosome-mediated gene transfer, microcell-mediated gene transfer, spheroplast fusion, etc. Numerous techniques are known in the art for the introduction of foreign genes into cells (see e.g., Loeffler and Behr, 1993, Meth. Enzymol. 217:599-618; Cohen et al., 1993, Meth. Enzymol. 217:618-644; Cline, 1985, Pharmac. Ther. 29:69-92) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny. In a preferred embodiment, the cell used for gene therapy is autologous to the patient.

In a specific embodiment, the nucleic acid to be introduced for purposes of gene therapy comprises an inducible promoter operably linked to the coding sequence, such that expression of the nucleic acid is controllable by controlling the presence or absence of the appropriate inducer of transcription.

Oligonucleotides such as anti-sense RNA and DNA molecules, and ribozymes that function to inhibit the translation of a targeted mRNA, especially its C-terminus, are also within the scope of the invention. Anti-sense RNA and DNA molecules act to directly block the translation of mRNA by binding to targeted niRNA and preventing protein translation. In regard to antisense DNA, oligodeoxyribonucleotides derived from the translation initiation site, e.g., between −10 and +10 regions of a nucleotide sequence, are preferred.

The antisense oligonucleotide may comprise at least one modified base moiety which is selected from the group including, but not limited to, 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Within the scope of the invention are engineered hammerhead motif ribozyme molecules that specifically and efficiently catalyze endonucleolytic cleavage of target RNA sequences.

Specific ribozyme cleavage sites within any potential RNA target are initially identified by scanning the target molecule for ribozyme cleavage sites that include the following sequences, GUA, GUU and GUC. Once identified, short RNA sequences of between 15 and 20 ribonucleotides corresponding to the region of the target gene containing the cleavage site may be evaluated for predicted structural features such as secondary structure that may render the oligonucleotide sequence unsuitable. The suitability of candidate targets may also be evaluated by testing their accessibility to hybridization with complementary oligonucleotides, using ribonuclease protection assays.

The anti-sense RNA and DNA molecules and ribozymes of the invention may be prepared by any method known in the art for the synthesis of nucleic acid molecules. These include techniques for chemically synthesizing oligodeoxyribonucleotides well known in the art such as for example solid phase phosphoramidite chemical synthesis. Alternatively, RNA molecules may be generated by in vitro and in vivo transcription of DNA sequences encoding the RNA molecule. Such DNA sequences may be incorporated into a wide variety of vectors that contain suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

Various modifications to the DNA molecules may be introduced as a means of increasing intracellular stability and half-life. Possible modifications include, but are not limited to, the addition of flanking sequences of ribo- or deoxy- nucleotides to the 5′ and/or 3′ ends of the molecule or the use of phosphorothioate or 2′ O-methyl rather than phosphodiesterase linkages within the oligodeoxyribonucleotide backbone.

C. Other Pharmaceutical Compositions

The compounds of the invention may be administered to a subject per se or in the form of a sterile composition or a pharmaceutical composition. Pharmaceutical compositions comprising the compounds of the invention may be manufactured by means of conventional mixing, dissolving, granulating, dragee-making, levigating, emulsifying, encapsulating, entrapping or lyophilizing processes. Pharmaceutical compositions may be formulated in conventional manner using one or more physiologically acceptable carriers, diluents, excipients or auxiliaries that facilitate processing of the active peptides or peptide analogues into preparations which can be used pharmaceutically. Proper formulation is dependent upon the route of administration chosen.

For topical administration the compounds of the invention may be formulated as solutions, gels, ointments, creams, suspensions, etc. as are well-known in the art.

Systemic formulations include those designed for administration by injection, e.g. subcutaneous, intravenous, intramuscular, intrathecal or intraperitoneal injection, as well as those designed for transdermal, transmucosal, oral or pulmonary administration.

For injection, the compounds of the invention may be formulated in aqueous solutions, preferably in physiologically compatible buffers such as Hanks's solution, Ringer's solution, or physiological saline buffer. The solution may contain formulatory agents such as suspending, stabilizing and/or dispersing agents.

Alternatively, the compounds may be in powder form for constitution with a suitable vehicle, e.g., sterile pyrogen-free water, before use.

For transmucosal administration, penetrants appropriate to the barrier to be permeated are used in the formulation. Such penetrants are generally known in the art. This route of administration may be used to deliver the compounds to the nasal cavity.

For oral administration, the compounds can be readily formulated by combining the active peptides or peptide analogues with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. For oral solid formulations such as, for example, powders, capsules and tablets, suitable excipients include fillers such as sugars, such as lactose, sucrose, mannitol and sorbitol; cellulose preparations such as maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP); granulating agents; and binding agents. If desired, disintegrating agents may be added, such as the cross-linked polyvinylpyrrolidone, agar, or alginic acid or a salt thereof such as sodium alginate.

If desired, solid dosage forms may be sugar-coated or enteric-coated using standard techniques.

For oral liquid preparations such as, for example, suspensions, elixirs and solutions, suitable carriers, excipients or diluents include water, glycols, oils, alcohols, etc. Additionally, flavoring agents, preservatives, coloring agents and the like may be added.

For buccal administration, the compounds may take the form of tablets, lozenges, etc. formulated in conventional manner.

For administration by inhalation, the compounds for use according to the present invention are conveniently delivered in the form of an aerosol spray from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The compounds may also be formulated in rectal or vaginal compositions such as suppositories or retention enemas, e.g., containing conventional suppository bases such as cocoa butter or other glycerides. Topical compositions and medicated carriers (e.g., medicated “tampon”) may also be used for such routes of administration.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

Alternatively, other pharmaceutical delivery systems may be employed. Liposomes and emulsions are well known examples of delivery vehicles that may be used to deliver peptides and peptide analogues of the invention. Certain organic solvents such as dimethylsulfoxide also may be employed, although usually at the cost of greater toxicity. Additionally, the compounds may be delivered using a sustained-release system, such as semipermeable matrices of solid polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

As the compounds of the invention may contain charged side chains or termini, they may be included in any of the above-described formulations as the free acids or bases or as pharmaceutically acceptable salts. Pharmaceutically acceptable salts are those salts which substantially retain the biologic activity of the free bases and which are prepared by reaction with inorganic acids. Pharmaceutical salts tend to be more soluble in aqueous and other protic solvents than are the corresponding free base forms.

D. Effective Dosages

The compounds of the invention will generally be used in an amount effective to achieve the intended purpose. The compounds of the invention or pharmaceutical compositions thereof, are administered or applied in a therapeutically effective amount. By therapeutically effective amount is meant an amount effective ameliorate or prevent the symptoms, or prolong the survival of, the patient being treated. Determination of a therapeutically effective amount is well within the capabilities of those skilled in the art, especially in light of the detailed disclosure provided herein. An “inhibitory amount” or “inhibitory concentration” of a PL-PDZ binding inhibitor is an amount that reduces binding by at least about 40%, preferably at least about 50%, often at least about 70%, and even as much as at least about 90%. Binding can be measured in vitro (e.g., in an A assay or G assay) or in situ.

For systemic administration, a therapeutically effective dose can be estimated initially from in vitro assays. For example, a dose can be formulated in animal models to achieve a circulating concentration range that includes the IC₅₀ as determined in cell culture. Such information can be used to more accurately determine useful doses in humans.

Initial dosages can also be estimated from in vivo data, e.g., animal models, using techniques that are well known in the art. One having ordinary skill in the art could readily optimize administration to humans based on animal data.

Dosage amount and interval may be adjusted individually to provide plasma levels of the compounds that are sufficient to maintain therapeutic effect. Usual patient dosages for administration by injection range from about 0.1 to 5 mg/kg/day, preferably from about 0.5 to 1 mg/kg/day. Therapeutically effective serum levels may be achieved by administering multiple doses each day.

In cases of local administration or selective uptake, the effective local concentration of the compounds may not be related to plasma concentration. One having skill in the art will be able to optimize therapeutically effective local dosages without undue experimentation.

The amount of compound administered will, of course, be dependent on the subject being treated, on the subject's weight, the severity of the affliction, the manner of administration and the judgment of the prescribing physician.

The therapy may be repeated intermittently while symptoms detectable or even when they are not detectable. The therapy may be provided alone or in combination with other drugs. In the case of conditions associated with leukocyte activation such as transplantation rejection and autoimmunity, the drugs that may be used in combination with the compounds of the invention include, but are not limited to, steroid and non-steroid anti-inflammatory agents.

E. Toxicity

Preferably, a therapeutically effective dose of the compounds described herein ill provide therapeutic benefit without causing substantial toxicity.

Toxicity of the compounds described herein can be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. Compounds which exhibit high therapeutic indices are preferred. The data obtained from these cell culture assays and animal studies can be used in formulating a dosage range that is not toxic for use in human. The dosage of the compounds described herein lies preferably within a range of circulating concentrations that include the effective dose with little or no toxicity. The dosage may vary within this range depending upon the dosage form employed and the route of administration utilized. The exact formulation, route of administration and dosage can be chosen by the individual physician in view of the patient's condition. (See, e.g., Fingl et al., 1975, In: The Pharmacological Basis of Therapeutics, Ch. 1, p. 1).

Kits

Also provided are reagents and kits thereof for practicing one or more of the above-described methods. The subject reagents and kits thereof may vary greatly. Typically, the kits at least include a subject peptide that may or may not contain a cell permeable peptide carrier. The subject kits may also include one or more additional reagents, e.g., reagents employed in administering the peptides, such as diluents, syringes, etc.

In addition to the above components, the subject kits can further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

### EXAMPLE 1

### Sequence Analysis of HPV E6 Proteins to Determine Oncogenic Potential

PDZ proteins are known to bind certain carboxyl-terminal sequences of proteins (PLs). PL sequences that bind PDZ domains are predictable, and have been described in greater detail in U.S. patent application Ser. Nos. 09/710,059, 09/724,553 and 09/688,017. One of the major classes of PL motifs is the set of proteins terminating in the sequences -X-(S/T)-X-(V/I/L). We have examined the C-terminal sequences of E6 proteins from a number of HPV strains. All of the strains determined to be oncogenic by the National Cancer Institute exhibit a consensus PDZ binding sequence. Those E6 proteins from papillomavirus strains that are not cancerous lack a sequence that would be predicted to bind to PDZ domains, thus suggesting that interaction with PDZ proteins is a prerequisite for causing cancer in humans.

This correlation between presence of a PL and ability to cause cancer is 100% in the sequences examined. In theory, with the disclosed PL consensus sequences from the patents listed supra, new variants of HPVs can be assessed for their ability to bind PDZ proteins and oncogenicity can be predicted on the basis of whether a PL is present.

TABLE 2
Correlation of E6 PDZ-ligands and oncogenicity

| | | PL | |
|---|---|---|---|
| HPV | | yes/ | |
| strain | E6 C-terminal sequence | no | oncogenic |
| HPV 4 | GYCRNCIRKQ (SEQ ID NO:5) | No | No |
| HPV 11 | WTTCMEDLLP (SEQ ID NO:6) | No | No |
| HPV 20 | GICRLCKHFQ (SEQ ID NO:7) | No | No |
| HPV 24 | KGLCRQCKQI (SEQ ID NO:8) | No | No |
| HPV 28 | WLRCTVRIPQ (SEQ ID NO:9) | No | No |
| HPV 36 | RQCKHFYNDW (SEQ ID NO:10) | No | No |
| HPV 48 | CRNCISHEGR (SEQ ID NO:11) | No | No |
| HPV 50 | CCRNCYEHEG (SEQ ID NO:12) | No | No |
| HPV 16 | SSRTRRETQL (SEQ ID NO:13) | Yes | Yes |
| HPV 18 | RLQRRRETQV (SEQ ID NO:14) | Yes | Yes |
| HPV 26 | RPRRQTETQV (SEQ ID NO:15) | Yes | Yes |
| HPV 30 | RRTLRRETQV (SEQ ID NO:16) | Yes | Yes |
| HPV 31 | WRRPRTETQV (SEQ ID NO:17) | Yes | Yes |
| HPV 33 | RLQRRRETAL (SEQ ID NO:18) | Yes | Yes |
| HPV 35 | WKPTRRETEV (SEQ ID NO:19) | Yes | Yes |
| HPV 39 | RRLTRRETQV (SEQ ID NO:20) | Yes | Yes |
| HPV 45 | RLRRRRETQV (SEQ ID NO:21) | Yes | Yes |
| HPV 51 | RLQRRNETQV (SEQ ID NO:22) | Yes | Yes |
| HPV 52 | RLQRRRVTQV (SEQ ID NO:23) | Yes | Yes |
| HPV 53 | RHTTATESAV (SEQ ID NO:24) | Yes | Yes |
| HPV 56 | TSREPRESTV (SEQ ID NO:25) | Yes | Yes |
| HPV 58 | RLQRRRQTQV (SEQ ID NO:26) | Yes | Yes |
| HPV 59 | QRQARSETLV (SEQ ID NO:27) | Yes | Yes |
| HPV 66 | TSRQATESTV (SEQ ID NO:28) | Yes | Yes* |
| HPV 68 | RRRTRQETQV (SEQ ID NO:29) | Yes | Yes |
| HPV 69 | RRREATETQV (SEQ ID NO:30) | Yes | Yes |
| HPV 73 | RCWRPSATVV (SEQ ID NO:31) | Yes | Yes |
| HPV 82 | PPRQRSETQV (SEQ ID NO:32) | Yes | Yes |

Table 2 legend: E6 C-terminal sequences and oncogenicity. HPV variants are listed at the left. Sequences were identified from Genbank sequence records. PL Yes/No was defined by a match or non-match to the consenses determined at Arbor Vita and by Songyang et al. -X-(S/T)-X-(V/I/L) PLs are indicated in bold. Oncogenicity data collected from National Cancer Institute. # No sequence found in NCBI database. *Only found in oncogenic strains co-transfected with other oncogenic proteins.

### EXAMPLE 2

### Identification of PDZ Domains that Interact with the C-Termini of Oncogenic E6 Proteins

In order to determine the PDZ domains that can be used with oncogenic E6 proteins as targets for the treatment of HPV, the ‘G assay’ (described supra) was used to identify interactions between E6 PLs and PDZ domains. Peptides were synthesized as described supra, corresponding to the C-terminal amino acid sequences of E6 proteins from oncogenic strains of human papillomavirus. These peptides were assessed for the ability to bind PDZ domains using the G-assay described above and PDZ proteins synthesized from the expression constructs described in greater detail in Example 5, Table 5, and U.S. patent application Ser. Nos. 09/710,059, 09/724,553 and 09/688,017. Results of these assays that show a high binding affinity are listed in Table 3A below.

As we can see below, there a large number of PDZ domains that bind some of the oncogenic E6 proteins. However, only the second PDZ domain from MAGI-1 seems to bind all of the oncogenic E6 PLs tested at high affinity. The PDZ domains of TIP-1 and DLG1 (domain2) bind all but one of the oncogenic E6 PLs tested, and may be useful in conjunction with MAGI-1 domain 2 interactions as targets for the treatment of HPV.

In a similar manner, peptides corresponding to the C-terminal ends of several non-oncogenic E6 proteins were tested with the G-assay. None of the peptides showed any affinity for binding PDZ domains (data not shown).

Table 3B shows the results of the G assay looking at interactions between the E6 PDZ ligand and PDZ domains. Listed are interactions that gave a signal to noise of around 2 or higher. This demonstrates the extent of PDZ binding and the non-obvious nature of this ligands interaction with cellular PDZ proteins. However, we see a number of interactions that are common to most all PLs from oncogenic E6 proteins that can be specifically targeted to treat HPV induced cancers.

TABLE 3A
higher affinity interactions between HPV E6 PLs and PDZ domains

| | PDZ binding partner | | PDZ binding partner |
|---|---|---|---|
| HPV strain | (signal 4 and 5 of 0-5) | HPV strain | (signal 4 and 5 of 0-5) |
| HPV 35 | Atrophin-1 interact. prot. | HPV 33 | Magi1 (PDZ #2) |
| (E T E V) | (PDZ # 1, 3, 5) | (E T A L) | TIP1 |
| | Magi1 (PDZ # 2, 3, 4, 5) | | DLG1 |
| | Lim-Ril | | Vartul (PDZ #1) |
| | FLJ 11215 | | KIAA 0807 |
| | MUPP-1 (PDZ #10) | | KIAA 1095 (Semcap3) (PDZ #1) |
| | KIAA 1095 (PDZ #1) | | KIAA 1934 (PDZ #1) |
| | PTN-4 | | NeDLG (PDZ #1, 2) |
| | INADL (PDZ #8) | | Rat outer membrane (PDZ #1) |
| | Vartul (PDZ # 1, 2, 3) | | PSD 95 (PDZ #3 and 1-3) |
| | Syntrophin-1 alpha | | |
| | Syntrophin gamma-1 | | |
| | TAX IP2 | | |
| | KIAA 0807 | | |
| | KIAA 1634 (PDZ #1) | | |
| | DLG1 (POZ1, 2) | | |
| | NeDLG (1, 2, 3,) | | |
| | Sim. Rat outer membrane (PDZ | | |
| | #1) | | |
| | MUPP-1 (PDZ #13) | | |
| | PSD 95 (1, 2, 3) | | |
| HPV 58 | Atrophin-1 interact. prot. (PDZ # | HPV 66 | DLG1 (PDZ #1, 2) |
| (Q T Q V) | 1) | (E S T V) | NeDLG (PDZ #2) |
| | Magi1 (PDZ #2) | | PSD 95 (PDZ #1, 2, 3) |
| | DLG1 (PDZ1, 2) | | Magi1 (PDZ #2) |
| | DLG2 (PDZ #2) | | KIAA 0807 |
| | KIAA 0807 | | KIAA 1634 (PDZ #1) |
| | KIAA 1634 (PDZ #1) | | DLG2 (PDZ #2) |
| | NeDLG (1, 2) | | Rat outer membrane (PDZ #1) |
| | Sim. Rat outer membrane (PDZ | | NeDLG (1, 2) |
| | #1) | | TIP-1 |
| | PSD 95 (1, 2, 3) | | |
| | INADL (PDZ #8) | | |
| | TIP-1 | | |
| HPV 16 | TIP-1 | HPV 52 | Magi1 (PDZ #2) |
| (E T Q L) | Magi1 (PDZ #2) | (V T Q V) | |
| | | HPV 18* | TIP1 |
| | | (E T Q V) | Magi 1 (PDZ #2) |
| Table 3: Interactions between the E6 C-termini of several HPV variants and human PDZ domains. HPV strain denotes the strain from which the E6 C-terminal peptide sequence information was taken. Peptides used in the assay varied from 18 to 20 amino acids in length, and the terminal four residues are listed in parenthesis. Names to the right of each HPV E6 ariant denote the human PDZ domain(s) (with domain number in parenthesis for proteins | | | |
| # with multiple PDZ domains) that saturated binding with the E6 peptide in the G assay (See Description of the Invention). | | | |

*denotes that the PDZ domains of hDlg1 were not tested against these proteins yet due to limited material, although both have been shown to bind hDlg1 in the literature.

TABLE 3B
PDZ Domain interactions with HPV16 E6 PDZ Ligand

| PL | Gene name | Domain | [Peptide] | [Protein] | Ave OD | StDev | OD S/N |
|---|---|---|---|---|---|---|---|
| HPV E6 #16 | MAGI 1 | 2 | 10 | 5 | 4.1125 | 0.039 | 19.54 |
| HPV E6 #16 | KIAA0807 | 1 | 10 | 5 | 3.9105 | 0.074 | 23.21 |
| HPV E6 #16 | PSD95 | 1, 2, 3 | 10 | 5 | 3.866 | 0.010 | 19.67 |
| HPV E6 #16 | KIAA0973 #288.2 | 1 | 10 | 5 | 3.85 | 0.180 | 16.42 |
| HPV E6 #16 | KIAA0147 | 1 | 10 | 5 | 3.764 | 0.018 | 21.33 |
| HPV E6 #16 | PSD95 | 1, 2, 3 | 10 | 5 | 3.523 | 0.013 | 22.58 |
| HPV E6 #16 | KIAA1634 | 1 | 10 | 5 | 3.465 | 0.011 | 19.04 |
| HPV E6 #16 | DLG2 | 3 | 10 | 5 | 3.43 | 0.091 | 20.36 |
| HPV E6 #16 | NeDLG | 1, 2 | 10 | 5 | 3.401 | 0.346 | 19.83 |
| HPV E6 #16 | KIAA1634 | 1 | 10 | 5 | 3.336 | 0.034 | 19.45 |
| HPV E6 #16 | NeDLG | 1, 2 | 10 | 5 | 3.118 | 0.124 | 17.13 |
| HPV E6 #16 | SIP 1 | 1 | 10 | 5 | 3.0715 | 0.771 | 42.96 |
| HPV E6 #16 | KIAA0973 #288.2 | 1 | 10 | 5 | 2.8295 | 0.148 | 10.11 |
| HPV E6 #16 | KIAA1095 | 1 | 10 | 5 | 2.7045 | 0.069 | 13.13 |
| HPV E6 #16 | PSD95 | 2 | 10 | 5 | 2.703 | 1.114 | 12.23 |
| HPV E6 #16 | Outer Membrane | 1 | 10 | 5 | 2.392 | 0.223 | 11.39 |
| HPV E6 #16 | Magi2 | 1 | 10 | 5 | 2.387 | 0.110 | 11.59 |
| HPV E6 #16 | KIAA0147 | 1 | 10 | 5 | 2.1465 | 0.056 | 8.71 |
| HPV E6 #16 | KIAA0147 | 3 | 10 | 5 | 2.0785 | 0.484 | 11.78 |
| HPV E6 #16 | TIP 43 #264.1 | 1 | 10 | 5 | 1.8775 | 0.013 | 8.50 |
| HPV E6 #16 | DLG1 | 2 | 10 | 5 | 1.8605 | 0.441 | 6.63 |
| HPV E6 #16 | DLG2 #290.1 | 2 | 10 | 5 | 1.784 | 0.280 | 9.17 |
| HPV E6 #16 | KIAA1095 | 1 | 10 | 5 | 1.711 | 0.058 | 7.96 |
| HPV E6 #16 | KIAA0807 | 1 | 10 | 5 | 1.6885 | 0.152 | 11.37 |
| HPV E6 #16 | TIP1 | 1 | 10 | 5 | 1.6155 | 0.069 | 8.24 |
| HPV E6 #16 | DLG2 #290.1 | 2 | 10 | 5 | 1.439 | 0.593 | 13.39 |
| HPV E6 #16 | DLG1 | 1, 2 | 10 | 5 | 1.431 | 0.259 | 4.57 |
| HPV E6 #16 | KIAA1526 #125.1 | 1 | 10 | 5 | 1.379 | 0.436 | 6.69 |
| HPV E6 #16 | Outer Membrane | 1 | 10 | 5 | 1.3595 | 0.036 | 6.25 |
| HPV E6 #16 | Syntrophin 1 alpha | 1 | 10 | 5 | 1.3325 | 0.442 | 8.03 |
| HPV E6 #16 | PSD95 | 2 | 10 | 5 | 1.3095 | 0.476 | 12.53 |
| HPV E6 #16 | DLG2 | 3 | 10 | 5 | 1.2665 | 0.118 | 8.53 |
| HPV E6 #16 | Syntrophin beta 2 | 1 | 10 | 5 | 1.1585 | 0.060 | 5.81 |
| HPV E6 #16 | NeDLG | 2 | 10 | 5 | 1.1185 | 0.278 | 3.99 |
| HPV E6 #16 | DLG1 | 1 | 10 | 5 | 1.09 | 0.025 | 4.18 |
| HPV E6 #16 | KIAA0147 | 3 | 10 | 5 | 1.072 | 0.103 | 4.35 |
| HPV E6 #16 | Magi2 | 1 | 10 | 5 | 1.056 | 0.100 | 7.23 |
| HPV E6 #16 | Syntrophin beta 2 | 1 | 10 | 5 | 1.0325 | 0.210 | 5.35 |
| HPV E6 #16 | KIAA0973 #148.4 | 1 | 10 | 5 | 1.0105 | 0.052 | 6.22 |
| HPV E6 #16 | PSD95 | 1 | 10 | 5 | 0.942 | 0.025 | 4.81 |
| HPV E6 #16 | TIP 43 #264.1 | 1 | 10 | 5 | 0.915 | 0.102 | 8.76 |
| HPV E6 #16 | DLG2 #162.1 | 2 | 10 | 5 | 0.908 | 0.069 | 3.87 |
| HPV E6 #16 | KIAA0380 #25.6 | 1 | 10 | 5 | 0.857 | 0.147 | 3.06 |
| HPV E6 #16 | DLG2 #162.1 | 2 | 10 | 5 | 0.853 | 0.103 | 3.05 |
| HPV E6 #16 | FLJ00011 | 1 | 10 | 5 | 0.8185 | 0.004 | 3.97 |
| HPV E6 #16 | PTN-4 | 1 | 10 | 5 | 0.8085 | 0.019 | 4.03 |
| HPV E6 #16 | DLG2 | 1 | 10 | 5 | 0.793 | 0.170 | 3.59 |
| HPV E6 #16 | Syntrophin gamma 2 | 1 | 10 | 5 | 0.7725 | 0.074 | 3.13 |
| HPV E6 #16 | KIAA1526 #125.1 | 1 | 10 | 5 | 0.7665 | 0.090 | 3.57 |
| HPV E6 #16 | NSP #268.2 | 1 | 10 | 5 | 0.724 | 0.106 | 2.58 |
| HPV E6 #16 | KIAA0382 | 1 | 10 | 5 | 0.712 | 0.066 | 3.61 |
| HPV E6 #16 | MAGI 1 | 6 | 10 | 5 | 0.71 | 0.134 | 2.47 |
| HPV E6 #16 | APXL1 | 1 | 10 | 5 | 0.708 | 0.051 | 2.96 |
| HPV E6 #16 | KIAA0382 | 1 | 10 | 5 | 0.6995 | 0.070 | 2.66 |
| HPV E6 #16 | KIAA0973 #148.4 | 1 | 10 | 5 | 0.6885 | 0.042 | 5.12 |
| HPV E6 #16 | FLJ11215 | 1 | 10 | 5 | 0.666 | 0.066 | 2.74 |
| HPV E6 #16 | SIP 1 | 1 | 10 | 5 | 0.6615 | 0.129 | 3.14 |
| HPV E6 #16 | RGS12 | 1 | 10 | 5 | 0.661 | 0.023 | 4.64 |
| HPV E6 #16 | ELFIN 1 | 1 | 10 | 5 | 0.66 | 0.042 | 3.39 |
| HPV E6 #16 | Magi2 | 5 | 10 | 5 | 0.651 | 0.096 | 3.93 |
| HPV E6 #16 | NeDLG | 3 | 10 | 5 | 0.632 | 0.040 | 3.69 |
| HPV E6 #16 | ZO-2 | 1 | 10 | 5 | 0.622 | 0.061 | 2.56 |
| HPV E6 #16 | Syntrophin gamma 1 | 1 | 10 | 5 | 0.618 | 0.014 | 3.08 |
| HPV E6 #16 | KIAA0316 | 1 | 10 | 5 | 0.6125 | 0.004 | 2.56 |
| HPV E6 #16 | MINT1 | 1, 2 | 10 | 5 | 0.6075 | 0.005 | 2.11 |
| HPV E6 #16 | KIAA0380 #25.8 | 1 | 10 | 5 | 0.603 | 0.008 | 3.58 |
| HPV E6 #16 | LIM Mystique | 1 | 10 | 5 | 0.5955 | 0.037 | 2.84 |
| HPV E6 #16 | MINT1 | 2 | 10 | 5 | 0.5925 | 0.047 | 2.76 |
| HPV E6 #16 | MINT1 | 2 | 10 | 5 | 0.5925 | 0.013 | 2.24 |
| HPV E6 #16 | TIP1 | 1 | 10 | 5 | 0.576 | 0.115 | 5.67 |
| HPV E6 #16 | Syntrophin 1 alpha | 1 | 10 | 5 | 0.5635 | 0.002 | 3.70 |
| HPV E6 #16 | PDZ-73 | 2 | 10 | 5 | 0.5615 | 0.045 | 1.95 |
| HPV E6 #16 | AIPC | 1 | 10 | 5 | 0.5595 | 0.084 | 2.30 |
| HPV E6 #16 | DLG1 | 1, 2 | 10 | 5 | 0.5495 | 0.033 | 1.67 |
| HPV E6 #16 | novel PDZ gene | 1 | 10 | 5 | 0.5455 | 0.260 | 1.89 |
| HPV E6 #16 | MAGI 1 | 5 | 10 | 5 | 0.541 | 0.041 | 3.12 |
| HPV E6 #16 | DLG1 | 2 | 10 | 5 | 0.5325 | 0.088 | 2.76 |
| HPV E6 #16 | NeDLG | 3 | 10 | 5 | 0.5225 | 0.001 | 2.49 |
| HPV E6 #16 | ZO-1 | 1 | 10 | 5 | 0.5215 | 0.008 | 2.83 |
| HPV E6 #16 | INADL | 6 | 10 | 5 | 0.518 | 0.006 | 3.07 |
| HPV E6 #16 | PDZK1 | 2, 3, 4 | 10 | 5 | 0.518 | 0.093 | 2.64 |
| HPV E6 #16 | ZO-1 | 2 | 10 | 5 | 0.518 | 0.035 | 3.02 |
| HPV E6 #16 | HEMBA 1003117 #193.3 | 1 | 10 | 5 | 0.517 | 0.020 | 2.20 |
| HPV E6 #16 | NeDLG | 1 | 10 | 5 | 0.5165 | 0.049 | 1.98 |
| HPV E6 #16 | TAX IP 2 | 1 | 10 | 5 | 0.5085 | 0.004 | 2.37 |
| HPV E6 #16 | PDZK1 | 3 | 10 | 5 | 0.5 | 0.040 | 1.92 |
| HPV E6 #16 | EBP50 #287.1 | 2 | 10 | 5 | 0.499 | 0.031 | 2.57 |
| HPV E6 #16 | KIAA0973 #148.5 | 1 | 10 | 5 | 0.4985 | 0.037 | 2.71 |
| HPV E6 #16 | MUPP1 | 7 | 10 | 5 | 0.498 | 0.144 | 1.61 |
| HPV E6 #16 | MAGI 1 | 4 | 10 | 5 | 0.496 | 0.034 | 1.58 |
| HPV E6 #16 | INADL | 3 | 10 | 5 | 0.485 | 0.150 | 1.86 |
| HPV E6 #16 | SITAC-18 | 2 | 10 | 5 | 0.484 | 0.006 | 2.46 |
| HPV E6 #16 | TAX IP 2 | 1 | 10 | 5 | 0.478 | 0.133 | 2.48 |
| HPV E6 #16 | NOS1 | 1 | 10 | 5 | 0.4775 | 0.152 | 2.88 |
| HPV E6 #16 | HEMBA 1003117 #226.2 | 1 | 10 | 5 | 0.477 | 0.031 | 2.27 |
| HPV E6 #16 | KIAA1284 | 1 | 10 | 5 | 0.473 | 0.190 | 2.72 |
| HPV E6 #16 | Syntrophin gamma 2 | 1 | 10 | 5 | 0.469 | 0.028 | 2.76 |
| HPV E6 #16 | SSTRIP | 1 | 10 | 5 | 0.467 | 0.033 | 3.28 |
| HPV E6 #16 | Shank 1 | 1 | 10 | 5 | 0.466 | 0.008 | 2.53 |
| HPV E6 #16 | KIAA0147 | 4 | 10 | 5 | 0.464 | 0.017 | 2.17 |
| HPV E6 #16 | KIAA1526 #126.1 | 2 | 10 | 5 | 0.4625 | 0.026 | 1.87 |
| HPV E6 #16 | APXL1 | 1 | 10 | 5 | 0.4605 | 0.025 | 3.14 |
| HPV E6 #16 | FLJ12615 | 1 | 10 | 5 | 0.4605 | 0.035 | 2.24 |
| HPV E6 #16 | KIAA0751 | 1 | 10 | 5 | 0.4505 | 0.026 | 2.10 |
| HPV E6 #16 | FLJ11215 | 1 | 10 | 5 | 0.449 | 0.071 | 2.77 |
| HPV E6 #16 | LIM-RIL | 1 | 10 | 5 | 0.449 | 0.045 | 1.60 |
| HPV E6 #16 | PAR3 #182.1 | 3 | 10 | 5 | 0.445 | 0.033 | 2.07 |
| HPV E6 #16 | AF6 | 1 | 10 | 5 | 0.444 | 0.008 | 1.80 |
| HPV E6 #16 | KIAA0545 | 1 | 10 | 5 | 0.4395 | 0.009 | 3.96 |
| HPV E6 #16 | MUPP1 | 13 | 10 | 5 | 0.4355 | 0.002 | 1.56 |
| HPV E6 #16 | EBP50 #311.1 | 1 | 10 | 5 | 0.4325 | 0.011 | 2.05 |
| HPV E6 #16 | MAGI 1 | 5 | 10 | 5 | 0.4325 | 0.054 | 1.77 |
| HPV E6 #16 | X-11 beta | 2 | 10 | 5 | 0.4315 | 0.141 | 2.95 |
| HPV E6 #16 | EBP50 #341.1 | 1 | 10 | 5 | 0.43 | 0.037 | 1.65 |
| HPV E6 #16 | RGS12 | 1 | 10 | 5 | 0.4295 | 0.018 | 3.96 |
| HPV E6 #16 | X-11 beta | 2 | 10 | 5 | 0.4265 | 0.016 | 1.78 |
| HPV E6 #16 | NeDLG | 2 | 10 | 5 | 0.4255 | 0.129 | 2.20 |
| HPV E6 #16 | KIAA1526 #119.1 | 1 | 10 | 5 | 0.425 | 0.025 | 1.38 |
| HPV E6 #16 | FLJ00011 | 1 | 10 | 5 | 0.423 | 0.061 | 2.90 |
| HPV E6 #16 | Densin | 1 | 10 | 5 | 0.4205 | 0.033 | 1.96 |
| HPV E6 #16 | Magi2 | 3 | 10 | 5 | 0.4165 | 0.026 | 1.98 |
| HPV E6 #16 | NSP #42.5 | 1 | 10 | 5 | 0.4165 | 0.011 | 1.95 |
| HPV E6 #16 | HTRA 3 | 1 | 10 | 5 | 0.4135 | 0.156 | 1.34 |
| HPV E6 #16 | ZO-1 | 1 | 10 | 5 | 0.4125 | 0.053 | 1.83 |
| HPV E6 #16 | MUPP1 | 13 | 10 | 5 | 0.4115 | 0.004 | 1.75 |
| HPV E6 #16 | KIAA1634 | 5 | 10 | 5 | 0.4025 | 0.015 | 2.07 |
| HPV E6 #16 | DLG1 | 3 | 10 | 5 | 0.3975 | 0.054 | 2.45 |
| HPV E6 #16 | SITAC-18 | 1 | 10 | 5 | 0.3975 | 0.016 | 1.63 |
| HPV E6 #16 | Shank 3 | 1 | 10 | 5 | 0.396 | 0.028 | 2.61 |
| HPV E6 #16 | MAGI 1 | 4 | 10 | 5 | 0.395 | 0.160 | 1.20 |
| HPV E6 #16 | MUPP1 | 10 | 10 | 5 | 0.3925 | 0.011 | 2.03 |
| HPV E6 #16 | MUPP1 | 10 | 10 | 5 | 0.3915 | 0.053 | 1.96 |
| HPV E6 #16 | DLG1 | 1 | 10 | 5 | 0.391 | 0.041 | 2.98 |
| HPV E6 #16 | KIAA1719 | 5 | 10 | 5 | 0.3895 | 0.012 | 1.81 |
| HPV E6 #16 | INADL | 8 | 10 | 5 | 0.3875 | 0.019 | 1.58 |
| HPV E6 #16 | PIST | 1 | 10 | 5 | 0.3855 | 0.033 | 1.96 |
| HPV E6 #16 | Shank 1 | 1 | 10 | 5 | 0.385 | 0.018 | 1.71 |
| HPV E6 #16 | EBP50 #167.2 | 1 | 10 | 5 | 0.3805 | 0.054 | 1.94 |
| HPV E6 #16 | KIAA0147 | 4 | 10 | 5 | 0.3795 | 0.009 | 2.34 |
| HPV E6 #16 | KIAA0973 #148.5 | 1 | 10 | 5 | 0.378 | 0.020 | 1.68 |
| HPV E6 #16 | KIAA0380 #25.6 | 1 | 10 | 5 | 0.376 | 0.055 | 1.95 |
| HPV E6 #16 | KIAA0147 | 2 | 10 | 5 | 0.3745 | 0.073 | 2.46 |
| HPV E6 #16 | MINT1 | 1, 2 | 10 | 5 | 0.3655 | 0.018 | 1.42 |
| HPV E6 #16 | Shroom | 1 | 10 | 5 | 0.361 | 0.219 | 3.25 |
| HPV E6 #16 | CASK | 1 | 10 | 5 | 0.36 | 0.151 | 1.15 |
| HPV E6 #16 | ERBIN | 1 | 10 | 5 | 0.36 | 0.038 | 1.82 |
| HPV E6 #16 | HEMBA 1003117 #226.2 | 1 | 10 | 5 | 0.36 | 0.059 | 1.66 |
| HPV E6 #16 | Magi2 | 3 | 10 | 5 | 0.359 | 0.004 | 1.65 |
| HPV E6 #16 | Syntrophin gamma 1 | 1 | 10 | 5 | 0.3585 | 0.005 | 2.00 |
| HPV E6 #16 | INADL | 4 | 10 | 5 | 0.357 | 0.037 | 2.15 |
| HPV E6 #16 | ZO-2 | 2 | 10 | 5 | 0.3565 | 0.018 | 2.15 |
| HPV E6 #16 | TIAM 2 | 1 | 10 | 5 | 0.356 | 0.006 | 2.05 |
| HPV E6 #16 | EBP50 #167.2 | 1 | 10 | 5 | 0.3555 | 0.018 | 2.28 |
| HPV E6 #16 | MAGI 1 | 3 | 10 | 5 | 0.354 | 0.027 | 1.46 |
| HPV E6 #16 | Numb BP | 1 | 10 | 5 | 0.352 | 0.119 | 3.17 |
| HPV E6 #16 | PAR3 #278.1 | 3 | 10 | 5 | 0.352 | 0.055 | 1.80 |
| HPV E6 #16 | X-11 beta | 1 | 10 | 5 | 0.352 | 0.100 | 2.02 |
| HPV E6 #16 | Densin | 1 | 10 | 5 | 0.3505 | 0.043 | 1.82 |
| HPV E6 #16 | GTPase | 1 | 10 | 5 | 0.3505 | 0.101 | 1.12 |
| HPV E6 #16 | DLG-6 #333.1 | 1 | 10 | 5 | 0.3495 | 0.016 | 2.04 |
| HPV E6 #16 | PTPL1 | 2 | 10 | 5 | 0.348 | 0.045 | 1.80 |
| HPV E6 #16 | KIAA0561 | 1 | 10 | 5 | 0.3475 | 0.011 | 2.44 |
| HPV E6 #16 | KIAA1719 | 3 | 10 | 5 | 0.346 | 0.018 | 1.65 |
| HPV E6 #16 | EBP50 #168.2 | 2 | 10 | 5 | 0.3455 | 0.019 | 1.99 |
| HPV E6 #16 | KIAA0316 | 1 | 10 | 5 | 0.343 | 0.021 | 2.34 |
| HPV E6 #16 | Serine Protease | 1 | 10 | 5 | 0.3425 | 0.165 | 1.55 |
| HPV E6 #16 | CARD14 | 1 | 10 | 5 | 0.342 | 0.000 | 1.52 |
| HPV E6 #16 | ERBIN | 1 | 10 | 5 | 0.342 | 0.004 | 1.30 |
| HPV E6 #16 | ZO-2 | 1 | 10 | 5 | 0.3415 | 0.012 | 2.11 |

Table 3B legend:
PL - indicates the PDZ ligand from the E6 protein of HPV 16.
Gene Name - the name of the gene containing a PDZ domain.
Domain - the PDZ domain number as assigned from amino terminus, also listed in Table 8.
[Peptide] - concentration of peptide in micromolar used for the assay.
[protein] - concentration of PDZ domain fusion used for this assay in micromolar.
Ave OD - Average A450 nm reading from two independent reactions for that day; duplicate PDZs may be present from different days.
StDev - Standard deviation of the two points used to generate the average.
OD S/N - the Absorbance signal to noise ratio versus the GST only well for that specific G Assay plate.

### EXAMPLE 3

### Detection of E6 and PDZ Domain Transcripts in Cervical Cell Lines

Purpose: To determine whether the PDZ domains with the highest affinity and widest breadth of binding to oncogenic E6 PL proteins are expressed in the same cell types as E6 proteins.

Summary: Total RNA was isolated from various cervical cell lines, some of which expressed the E6 protein from HPV 16 or HPV 18 (both oncogenic strains of human papillomavirus) by Trizol extraction (GibcoBRL). Briefly, ˜20 mg Trizol-extracted total RNA was loaded per lane onto a 1.2% formaldehyde gel for electrophoresis and transfer to nitrocellulose membrane by standard methods (Sambrook, Fritsch and Maniatis; Molecular Cloning. Cold Spring Harbor Press, second edition). Probes corresponding to HPV E6 from strains 16 or 18 were generated using PCR with the oligos listed in Example 4. Probes for TIP-1 and MAGI-1 were generated using PCR with primers listed in Example 5. All probes were radioactively labeled with ³²P using the Ready-To-Go labeling kit (Amersham Pharmacia). Blots were crosslinked, blocked with CHURCH solution (7% SDS, 1% BSA and phosphate buffered), and hybridized with the appropriate probe for several hours at 42° C. in Church solution.

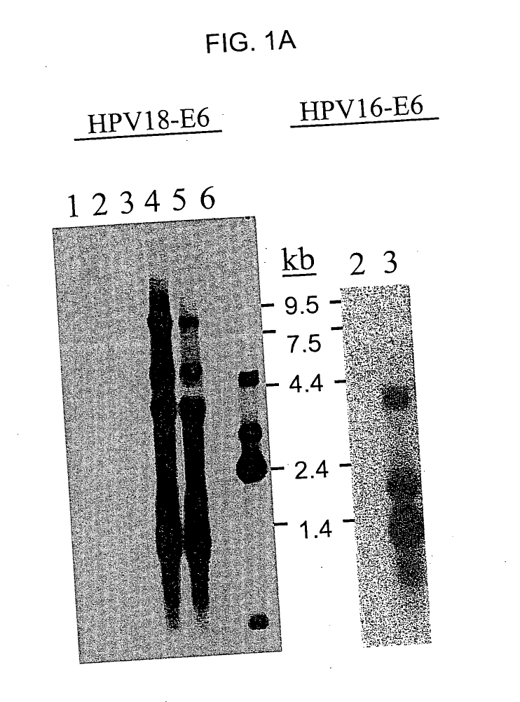

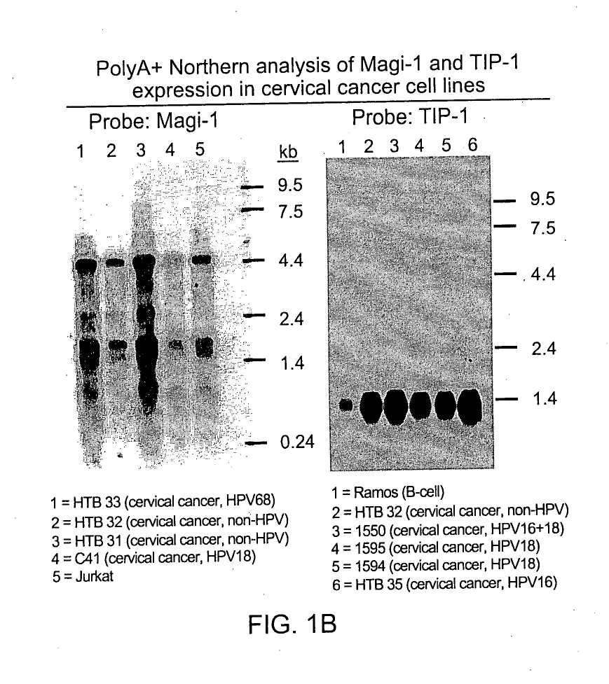

Hybridized blots were washed several times with 1×SSC, 0.2% SDS at 65° C. followed by 2-3 stringent washes of 0.2×SSC, 0.1% SDS at 65° C. Washed blots were exposed to film overnight and are shown in FIGS. 1A and 1B.

Results: FIG. 1A shows the expression of E6 from HPV16 or HPV18 in various cell lines used in these studies. Lanes: 1 B-cell (Ramos); 2 No HPV (HTB32); 3 1550 HPV 16+18; 4 1595 HPV18; 5 1594 HPV 18; 6 HTB 35 (HPV 16); 7 RNA marker. HPV18 E6 and HPV16 E6 refer to the radiolabeled probe used to detect expression in each of the cell lines. FIG. 1B shows the expression of TIP1 and MAGI1 in various cervical cell lines used in this study. Both genes are expressed in cervical cancers indicating that they could be involved in the mechanism of E6 oncogenicity.

### EXAMPLE 4

### Generations of Eukaryotic Expression Constructs Bearing DNA Fragments that Encode HPV E6 Genes or Portions of HPV E6 Genes

This example describes the cloning of HPV E6 genes or portions of HPV E6 genes into eukaryotic expression vectors in fusion with a number of protein tags, including but not limited to Glutathione S-Transferase (GST), Enhanced Green Fluorescent Protein (EGFP), or Hemagglutinin (HA).

A. Strategy

cDNA fragments were generated by RT-PCR from HPV cell line (cervical epidermoid carcinoma, ATCC# CRL-1550 and CRL-1595 for HPV E6 16 and 18, respectively) derived RNA, using random (oligo-nucleotide) primers (Invitrogen Cat. # 4819001 1). DNA fragments corresponding to HPV E6 were generated by standard PCR, using above purified cDNA fragments and specific primers (see Table 4). Primers used were designed to create restriction nuclease recognition sites at the PCR fragment's ends, to allow cloning of those fragments into appropriate expression vectors. Subsequent to PCR, DNA samples were submitted to agarose gel electrophoresis. Bands corresponding to the expected size were excised. DNA was extracted by Sephaglas Band Prep Kit (Amersham Pharmacia Cat # 27-9285-01) and digested with appropriate restriction endonuclease. Digested DNA samples were purified once more by gel electrophoresis, according to the same protocol used above. Purified DNA fragments were coprecipitated and ligated with the appropriate linearized vector. After transformation into E. coli, bacterial colonies were screened by colony PCR and restriction digest for the presence and correct orientation of insert. Positive clones were innoculated in liquid culture for large scale DNA purification. The insert and flanking vector sites from the purified plasmid DNA were sequenced to ensure correct sequence of fragments and junctions between the vectors and fusion proteins.

B. Vectors:

Cloning vectors were pGEX-3X (Amersham Pharmacia #27-4803-01), MIE (a derivative of MSCV, containing IRES and EGFP, generated by recombinant DNA technology), pmKit, pcDNA3.1 (Invitrogen, modified to include a HA tag upstream of the cloning site) and pMAL (New England Biolabs Cat # N8076S, polylinker modified in house to include BamH1 and EcoR1 sites).

DNA fragments containing the ATG-start codon and the TAG-stop codon of HPV E6 were cloned into pGEX3×. HPV E6 genes, and 3′ truncated (ΔPL) versions, were subsequently cloned into MIE (MSCV-IRES-EGFP) vector, pcDNA-HA vector, and pmKit vector, using the purified HPV E6-pGEX3× fusion plasmid as the PCR template, and using the same purification protocols as listed above. Truncated versions of HPV E6 have a stop codon inserted after the −3 position amino acid, so as to delete the last three amino acids from the coding region of the gene.

C. Constructs:

Primers used to generate DNA fragments by PCR are listed in Table 4. PCR primer combinations and restriction sites for insert and vector are listed below.

TABLE 4

Primers used in cloning of HPV E6 into
representative expression vectors.

| ID# | | |
|---|---|---|
| (Primer | | |
| Name) | Primer Sequence | Description |
| 2548 | AAAAGATCTACAATA | Forward (5′ to 3′) |
| (1054EF) | CTATGGCGC | primer corresponding |
| | (SEQ ID NO:33) | to HPV E6 18, gene- |
| | | rates a BG1 II site. |
| | | Used for cloning into |
| | | pGEX3x. |
| 2549 | AGGGAATTCCAGACT | Reverse (3′ to 5′) |
| (1058ER) | TAATATTATAC | primer corresponding |
| | (SEQ ID NO:34) | to HPV E6 18, gene- |
| | | rates an EcoR1 site. |
| | | Used for cloning into |
| | | pGEX3x. |
| 2542 | AAAGGATCCATTTTA | Forward (5′ to 3′) |
| (1050EF) | TGCACCAAAAG | primer corresponding |
| | (SEQ ID NO:35) | to HPV E6 16, gene- |
| | | rates a BamH1 site. |
| | | Used for cloning into |
| | | pGEX3x. |
| 2543 | ATGGAATTCTATCTC | Reverse (3′ to 5′) |
| (1051ER) | CATGCATGATTAC | primer corresponding |
| | (SEQ ID NO:36) | to HPV E6 16, gene- |
| | | rates an EcoR1 site. |
| | | Used for cloning into |
| | | pGEX3x. |
| 2563 | GAGGAATTCACCACA | Forward (5′ to 3′) |
| (1071EF) | ATACTATGGCG | primer corresponding |
| | (SEQ ID NO:37) | to HPV E6 18, gene- |
| | | rates an EcoR1 site. |
| | | Used for cloning into |
| | | MIE. |
| 2564 | AGGAGATCTCATACT | Reverse (3′ to 5′) |
| (1072ER) | TAATATTATAC | primer corresponding |
| | (SEQ ID NO:38) | to HPV E6 18, gene- |
| | | rates a Bg1 II site. |
| | | Used for cloning into |
| | | MIE. |
| 2565 | TTGAGATCTTCAGCG | Reverse (3′ to 5′) |
| (1073ERPL) | TCGTTGGAGTCG | primer corresponding |
| | (SEQ ID NO:39) | to HPV E6 18 ΔPL, |
| | | generates a Bg1 II |
| | | site. Used for |
| | | cloning into MIE. |
| 2560 | AAAGAATTCATTTTA | Forward (5′ to 3′) |
| (1074EF) | TGCACCAAAAG | primer corresponding |
| | (SEQ ID NO:40) | to HPV E6 16, gene- |
| | | rates an EcoR1 site. |
| | | Used for cloning into |
| | | MIE. |
| 2561 | ATGGGATCCTATCTC | Reverse (3′ to 5′) |
| (1075ER) | CATGCATGATTAC | primer corresponding |
| | (SEQ ID NO:41) | to HPV E6 16, gene- |
| | | rates a BamH1 site. |
| | | Used for cloning into |
| | | MIE. |
| 2562 | CTGGGATCCTCATCA | Reverse (3′ to 5′) |
| (1076ERPL) | ACGTGTTCTTGATGA | primer corresponding |
| | TC | to HPV E6 16 ΔPL, |
| | (SEQ ID NO:42) | generates a BamH1 |
| | | site. Used for |
| | | cloning into MIE. |
| 2603 | AAGAAAGCTTTTTAT | Forward (5′ to 3′) |
| (1080EF) | GCACCAAAAGAG | primer corresponding |
| | (SEQ ID NO:43) | to HPV E6 16, gene- |
| | | rates A Hind III site. |
| | | Used for cloning into |
| | | pcDNA-HA. |
| 2604 | AATCAAGCTTTATCT | Reverse (3′ to 5′) |
| (1081ER) | CCATGCATGATTAC | primer corresponding |
| | (SEQ ID NO:44) | to HPV E6 16, gene- |
| | | rates a Hind III site. |
| | | Used for cloning into |
| | | pcDNA-HA. |
| 2605 | GCTGAAGCTTTCAAC | Reverse (3′ to 5′) |
| (1082ERPL) | GTGTTCTTGATGATC | primer corresponding |
| | (SEQ ID NO:45) | to HPV E6 16 ΔPL, |
| | | generates a Hind III |
| | | site. Used for cloning |
| | | into pcDNA-HA. |
| 2606 | AAGCGTCGACTTTAT | Forward (5′ to 3′) |
| (1083EF) | GCACCAAAAGAG | primer corresponding |
| | (SEQ ID NO:46) | to HPV E6 16, gene- |
| | | rates a Sal I site. |
| | | Used for cloning into |
| | | pmKit. |
| 2607 | AATGCTCGAGTATCT | Reverse (3′ to 5′) |
| (1084ER) | CCATGCATGATTAC | primer corresponding |
| | (SEQ ID NO:47) | to HPV E6 16, gene- |
| | | rates a Xho I site. |
| | | Used for cloning into |
| | | pmKit. |
| 2608 | GCTGCTCGAGTCAAC | Reverse (3′ to 5′) |
| (1085ERPL) | GTGTTCTTGATGATC | primer corresponding |
| | (SEQ ID NO:48) | to HPV E6 16 ΔPL, |
| | | generates a Xho I |
| | | site. Used for cloning |
| | | into pmKit. |
| 2612 | AGAAGTCGACCACAA | Forward (5′ to 3′) |
| (1086EF) | TACTATGGCGC | primer corresponding |
| | (SEQ ID NO:49) | to HPV E6 18, gene- |
| | | rates a Sal I site. |
| | | Used for cloning into |
| | | pmKit. |
| 2613 | TAGGCTCGAGCATAC | Reverse (3′ to 5′) |
| (1087ER) | TTAATATTATAC | primer corresponding |
| | (SEQ ID NO:50) | to HPV E6 18, gene- |
| | | rates a Xho I site. |
| | | Used for cloning into |
| | | pmKit. |
| 2614 | CTTGCTCGAGTCAGC | Reverse (3′ to 5′) |
| (1088ERPL) | GTCGTTGGAGTCG | primer corresponding |
| | (SEQ ID NO:51) | to HPV E6 18 ΔPL, |
| | | generates a Xho I |
| | | site. Used for cloning |
| | | into pmKit. |
| 2615 | AGAAAAGCTTCACAA | Forward (5′ to 3′) |
| (1089EF) | TACTATGGCGC | primer corresponding |
| | (SEQ ID NO:52) | to HPV E6 18, gene- |
| | | rates A Hind III site. |
| | | Used for cloning into |
| | | pcDNA-HA. |
| 2616 | TAGAAGCTTGCATAC | Reverse (3′ to 5′) |
| (1090ER) | TTAATATTATAC | primer corresponding |
| | (SEQ ID NO:53) | to HPV E6 18, gene- |
| | | rates a Hind III site. |
| | | Used for cloning into |
| | | pcDNA-HA. |
| 2617 | CTTGAAGCTTTCAGC | Reverse (3′ to 5′) |
| (1091ERPL) | GTCGTTGAGGTCG | primer corresponding |
| | (SEQ ID NO:54) | to HPV E6 18 ΔPL, |
| | | generates a Hind III |
| | | site. Used for cloning |
| | | into pcDNA-HA. |

Table 4 legend: Table 4 discloses a list of oligonucleotides used to amplify and clone specific regions E6 proteins from various HPV strains into expression vectors. The first designation is an internal name for the primer. The second column represents the sequence presented 5′ to 3′. The third column is a description of directions of the primer, intended construct, and restriction site for cloning.

1. Human Papillomavirus (HPV) E6 16
Acc#:------------
GI#: 4927719
   Construct: HPV E6 16WT-pGEX-3×
Primers: 2542 & 2543
Vector Cloning Sites(5′/3′): Bam H1/EcoR1
Insert Cloning Sites(5′/3′): BamH1/EcoR1
pGEX-3× contains GST to the 5′end (upstream) of the cloning site
   Construct: HPV E6 16WT-MIE
Primers: 2560 & 2561
Vector Cloning Sites(5′/3′): EcoR1/BamH1
Insert Cloning Sites(5′/3′): EcoR1/BamH1
MIE contains IRES and EGFP to the 3′ end (downstream) of the cloning site
   Construct: HPV E6 16ΔPL-MIE
Primers: 2560 & 2562
Vector Cloning Sites(5′/3′): EcoR1/BamH1
Insert Cloning Sites(5′/3′): EcoR1/BamH1
MIE contains IRES and EGFP to the 3′ end (downstream) of the cloning site
   Construct: HPV E6 16WT-pcDNA3.1-HA
Primers: 2603 & 2604
Vector Cloning Sites(5′/3′): Hind III/Hind III
Insert Cloning Sites(5′/3′): Hind III/Hind III
pcDNA3.1 (modified) contains HA to the 5′ end (upstream) of the cloning site
   Construct: HPV E6 16ΔPL - pcDNA3.1-HA
Primers: 2603 & 2605
Vector Cloning Sites(5′/3′): Hind III/Hind III
Insert Cloning Sites(5′/3′): Hind III/Hind III
pcDNA3.1 (modified) contains HA to the 5′ end (upstream) of the cloning site
   Construct: HPV E6 16WT-pmKit
Primers: 2606 & 2607
Vector Cloning Sites(5′/3′): Sal I/Xho I
Insert Cloning Sites(5′/3′): Sal I/Xho I
   Construct: HPV E6 16ΔPL -pmKit
Primers: 2606 & 2608
Vector Cloning Sites(5′/3′): Sal I/Xho I
Insert Cloning Sites(5′/3′): Sal I/Xho I
   2. Human Papillomavirus (IPV) E6 18
Acc#: ------------
GI#: 413673
   Construct: HPV E6 18WT-pGEX-3×
Primers: 2548 & 2549
Vector Cloning Sites(5′/3′): Bam H1/EcoR1
Insert Cloning Sites(5′/3′): Bgl II/EcoR1
pGEX-3× contains GST to the 5′ end (upstream) of the cloning site
   Construct: HPV E6 18WT-MIE
Primers: 2563 & 2564
Vector Cloning Sites(5′/3′): EcoR1/BamH1
Insert Cloning Sites(5′/3′): EcoR1/Bgl II
MIE contains IRES and EGFP to the 3′ end (downstream) of the cloning site
   Construct: HPV E6 18ΔPL-MIE
Primers: 2563 & 2565
Vector Cloning Sites(5′/3′): EcoR1/BamH1
Insert Cloning Sites(5′/3′): EcoR1/Bgl II
MIE contains IRES and EGFP to the 3′ end (downstream) of the cloning site
   Construct: HPV E6 18WT-pcDNA3.1-HA
Primers: 2615 & 2616
Vector Cloning Sites(5′/3′): Hind III/Hind III
Insert Cloning Sites(5′/3′): Hind III/Hind III
pcDNA3.1 (modified) contains HA to the 5′ end (upstream) of the cloning site
   Construct: HPV E6 18ΔPL-pcDNA3.1-HA
Primers: 2615 & 2617
Vector Cloning Sites(5′/3′): Hind III/Hind III
Insert Cloning Sites(5′/3′): Hind III/Hind III
pcDNA3.1 (modified) contains HA to the 5′ end (upstream) of the cloning site
   Construct: HPV E6 18WT-pmKit
Primers: 2612 & 2613
Vector Cloning Sites(5′/3′): Sal I/Xho I
Insert Cloning Sites(5′/3′): Sal I/Xho I
   Construct: HPV E6 18ΔPL-pmKit
Primers: 2612 & 2614
Vector Cloning Sites(5′/3′): Sal I/Xho I
Insert Cloning Sites(5′/3′): Sal I/Xho I

D. GST Fusion Protein Production and Purification

The constructs using pGEX-3× expression vector were used to make fusion proteins according to the protocol outlined in the GST Fusion System, Second Edition, Revision 2, Pharmacia Biotech. Method II and was optimized for a 1 L LgPP.

Purified DNA was transformed into E. coli and allowed to grow to an OD of 0.4-0.8 (600λ). Protein expression was induced for 1-2 hours by addition of IPTG to cell culture. Cells were harvested and lysed. Lysate was collected and GS4B beads (Pharmacia Cat# 17-0756-01) were added to bind GST fusion proteins. Beads were isolated and GST fusion proteins were eluted with GEB II. Purified proteins were stored in GEB II at −80° C.

Purified proteins were used for ELISA-based assays, functional assays and antibody production.

Other vectors encoding portions of HPV proteins or cellular proteins were transfected directly into mammalian cells by various means for testing. E6 and E7 expression constructs from a variety of HPV strains (both oncogenic and non-oncogenic) were constructed in a similar manner to those described above.

### EXAMPLE 5

### Generation of Eukaryotic Expression Constructs Bearing DNA Fragments that Encode PDZ Domain Containing Genes or Portions of PDZ Domain Genes

This example describes the cloning of PDZ domain containing genes or portions of PDZ domain containing genes were into eukaryotic expression vectors in fusion with a number of protein tags, including but not limited to Glutathione S-Transferase (GST), Enhanced Green Fluorescent Protein (EGFP), or Hemagglutinin (HA).

A. Strategy

DNA fragments corresponding to PDZ domain containing genes were generated by RT-PCR from RNA from a library of individual cell lines (CLONTECH Cat# K4000-1) derived RNA, using random (oligo-nucleotide) primers (Invitrogen Cat.# 48190011). DNA fragments corresponding to PDZ domain containing genes or portions of PDZ domain containing genes were generated by standard PCR, using above purified cDNA fragments and specific primers (see Table 5 for example). Primers used were designed to create restriction nuclease recognition sites at the PCR fragment's ends, to allow cloning of those fragments into appropriate expression vectors. Subsequent to PCR, DNA samples were submitted to agarose gel electrophoresis. Bands corresponding to the expected size were excised. DNA was extracted by Sephaglas Band Prep Kit (Amersham Pharmacia Cat# 27-9285-01) and digested with appropriate restriction endonuclease. Digested DNA samples were purified once more by gel electrophoresis, according to the same protocol used above. Purified DNA fragments were coprecipitated and ligated with the appropriate linearized vector. After transformation into E. coli, bacterial colonies were screened by colony PCR and restriction digest for the presence and correct orientation of insert. Positive clones were innoculated in liquid culture for large scale DNA purification. The insert and flanking vector sites from the purified plasmid DNA were sequenced to ensure correct sequence of fragments and junctions between the vectors and fusion proteins.

B. Vectors:

All PDZ domain-containing genes were cloned into the vector pGEX-3× (Amersham Pharmacia #27-4803-01, Genemed Acc#U13852, GI#595717), containing a tac promoter, GST, Factor Xa, β-lactamase, and lac repressor.

The amino acid sequence of the pGEX-3× coding region including GST, Factor Xa, and the multiple cloning site is listed below. Note that linker sequences between the cloned inserts and GST-Factor Xa vary depending on the restriction endonuclease used for cloning. Amino acids in the translated region below that may change depending on the insertion used are indicated in small caps, and are included as changed in the construct sequence listed in (C).

aa 1 - aa 232:

(SEQ ID NO:55)

MSPILGYWKIKGLVQPTRLLLEYLEEKYEEHLYERDEGDKWRNKKFELGL
EFPNLPYYIDGDVKLTQSMAIIRYIADKHNMLGGCPKERAEISMLEGAVL
DIRYGVSRIAYSKDFETLKVDFLSKLPEMLKMFEDRLCHKTYLNGDHVTH
PDFMLYDALDVVLYMDPMCLDAFPKLVCFKKRIEAIPQIDKYLKSSKYIA
WPLQGWQATFGGGDHPPKSDLIEGRgipgnss

In addition, TAX Interacting Protein 1 (TIP 1), in whole or part, was cloned into many other expression vectors, including but not limited to CD5γ, PEAK10 (both provided by the laboratory of Dr. Brian Seed at Harvard University and generated by recombinant DNA technology, containing an IgG region), and MIN (a derivative of MSCV, containing IRES and NGFR, generated by recombinant DNA technology).

C. Constructs:

Primers used to generate DNA fragments by PCR are listed in Table 5. PCR primer combinations and restriction sites for insert and vector are listed below, along with amino acid translation for insert and restriction sites. Non-native amino acid sequences are shown in lower case. A comprehensive list of all PDZ domain constructs tested and their amino acid sequences are shown in Table 8.

TABLE 5

Primers used in cloning of DLG 1 (domain 2
of 3), MAGI 1 (domain 2 of 6), and TIP1
into representative expression vectors.

| ID# | | |
|---|---|---|
| (Primer | | |
| Name) | Primer Sequence | Description |
| 1928 | AATGGGGATCCAGCT | Forward (5′ to 3′) |
| (654DL1 | CATTAAAGG | primer corresponding to |
| 2F) | (SEQ ID NO:56) | DLG 1, domain 2 of 3. |
| | | Generates a Bam H1 site |
| | | upstream (5′) of the PDZ |
| | | boundary. Used for |
| | | cloning into pGEX-3X. |
| 1929 | ATACATACTTGTGGA | Reverse (3′ to 5′) |
| (655DL1 | ATTCGCCAC | primer corresponding to |
| 2R) | (SEQ ID NO:57) | DLG 1, domain 2 of 3. |
| | | Generates an EcoR1 site |
| | | downstream (3′) of the |
| | | PDZ boundary. Used for |
| | | cloning into pGEX-3X. |
| 1453 | CACGGATCCCTTCTG | Forward (5′ to 3′) |
| (435BAF) | AGTTGAAAGGC | primer corresponding to |
| | (SEQ ID NO:58) | MAGI 1, domain 2 of 6. |
| | | Generates a BamH1 site |
| | | upstream (5′) of the PDZ |
| | | boundary. Used for |
| cloning into pGEX-3X. | | |
| 1454 | TATGAATTCCATCTG | Reverse (3′ to 5′) |
| (436BAR) | GATCAAAAGGCAATG | primer corresponding to |
| | (SEQ ID NO:59) | MAGI 1, domain 2 of 6. |
| | | Generates an EcoR1 site |
| | | downstream (3′) of the |
| | | PDZ boundary. Used for |
| | | cloning into pGEX-3X. |
| 399 | CAGGGATCCAAAGAG | Forward (5′ to 3′) |
| (86TAF) | TTGAAATTCACAAGC | primer corresponding to |
| | (SEQ ID NO:60) | TIP1. Generates a Bam H1 |
| | | site upstream (5′) of |
| | | the PDZ boundary. Used |
| | | for cloning into |
| | | pGEX-3X. |
| 400 | ACGGAATTCTGCAGC | Reverse (3′ to 5′) |
| (87TAR) | GACTGCCGCGTC | primer corresponding to |
| | (SEQ ID NO:61) | TIP1. Generates an EcoR1 |
| | | site downstream (3′) of |
| | | the PDZ boundary. Used |
| | | for cloning into |
| | | pGEX-3X. |
| 1319 | AGGATCCAGATGTCC | Forward (5′ to 3′) |
| (TIP | TACATCCC | primer corresponding to |
| G5-1) | (SEQ ID NO:62) | TIP1. Generates a Bam H1 |
| | | site upstream (5′) of |
| | | the start codon. Used |
| | | for cloning into |
| | | pGEX-3X. |
| 1320 | GGAATTCATGGACTG | Reverse (3′ to 5′) |
| (TIP | CTGCACGG | primer corresponding to |
| G3-1) | (SEQ ID NO:63) | TIP1. Generates an EcoR1 |
| | | site downstream (3′) of |
| | | the stop codon. Used for |
| | | cloning into pGEX-3X. |
| 2753 | AGAGAATTCTCGAGA | Forward (5′ to 3′) |
| (1109TIF) | TGTCCTACATCCC | primer corresponding to |
| | (SEQ ID NO:64) | TIP1. Generates an EcoR1 |
| | | site upstream (5′) of |
| | | the start codon. Used for |
| | | cloning into MIN. |
| 2762 | TGGGAATTCCTAGGA | Reverse (3′ to 5′) |
| (1117TIR) | CAGCATGGACTG | primer corresponding to |
| | (SEQ ID NO:65) | TIP1. Generates an EcoR1 |
| | | site downstream (3′) of |
| | | the stop codon. Used for |
| | | cloning into MIN. |
| 2584 | CTAGGATCCGGGCCA | Forward (5′ to 3′) |
| (1080TIF) | GCCGGTCACC | primer corresponding to |
| | (SEQ ID NO:66) | TIP1. Generates a Bam H1 |
| | | site upstream (5′) of |
| | | the PDZ boundary. Used |
| | | for cloning into PEAK10 |
| | | or CD5γ. |
| 2585 | GACGGATCCCCCTGC | Reverse (3′ to 5′) |
| (1081TIR) | TGCACGGCCTTCTG | primer corresponding to |
| | (SEQ ID NO:67) | TIP1. Generates a Bam H1 |
| | | site downstream (3′) of |
| | | the PDZ boundary. Used |
| | | for cloning into PEAK10 |
| | | or CD5γ. |
| 2586 | GAGGAATTCCCCTGC | Reverse (3′ to 5′) |
| (1082TIR) | TGCACGGCCTTCTG | primer corresponding to |
| | (SEQ ID NO:68) | TIP1. Generates an EcoR1 |
| | | site downstream (3′) of |
| | | the PDZ boundary. Used |
| | | for cloning into PEAK10 |
| | | or CD5γ. |
| 2587 | CTAGAATTCGGGCCA | Forward (5′ to 3′) |
| (1083TIF) | GCCGGTCACC | primer corresponding to |
| | (SEQ ID NO:69) | TIP1. Generates an EcoR1 |
| | | site upstream (5′) of |
| | | the PDZ boundary. Used |
| | | for cloning into PEAK10 |
| | | or CD5γ. |

1. DLG 1, PDZ domain 2 of 3:
Acc#: U13897
GI#: 558437
   Construct: DLG 1, PDZ domain 2 of 3-pGEX-3×
Primers: 1928 & 1929
Vector Cloning Sites(5′/3′): Bam H1/EcoR1

Insert Cloning Sites(5′/3′): BamH1/EcoR1

aa 1 - aa 88

(SEQ ID NO:70)

giqLIKGPKGLGFSIAGGVGNQHIPGDNSIYVTKIIEGGAAHKDGKLQIG
DKLLAVNNVCLEEVTHEEAVTALKNTSDFVYLKVAnss

2. MAGI 1, PDZ domain 2 of 6:
Acc#: AB010894
GI#: 3370997
   Construct: MAGI 1, PDZ domain 2 of 6-pGEX-3×
Primers: 1453 & 1454
Vector Cloning Sites(5′/3′): Bam H1/EcoR1

Insert Cloning Sites(5′/3′): BamH1/EcoR1

aa 1 - aa 108

(SEQ ID NO:71)

giPSELKGKFIHTKLRKSSRGFGFTVVGGDEPDEFLQIKSLVLDGPAALD
GKMETGDVIVSVNDTCVLGHTHAQVVKIFQSIPIGASVDLELCRGYPLPF
DPDgihrd

3. TAX Interacting Protein 1 (TIP 1):
Acc#: AF028823.2
GI#: 11908159
   Construct: TIP 1, PDZ domain 1 of 1-pGEX-3×
Primers: 399& 400
Vector Cloning Sites(5′/3′): Bam H1/EcoR1

Insert Cloning Sites(5′/3′): BamH1/EcoR1

aa 1 - aa 107

(SEQ ID NO:72)

giQRVEIHKLRQGENLILGFSIGGGIDQDPSQNPFSEDKTDKGIYVTRVS
EGGPAEIAGLQIGDKIMQVNGWDMTMVTHDQARKRLTKRSEEVVRLLVTR
QSLQnss

Construct: TIP1-pGEX-3×
Primers: 1319& 1320
Vector Cloning Sites(5′/3′): Bam H1/EcoR1

Insert Cloning Sites(5′/3′): BamH1/EcoR1

aa 1 - aa 128

(SEQ ID NO:73)

giqMSYIPGQPVTAVVQRVEIHKLRQGENLILGFSIGGGIDQDPSQNPFS
EDKTDKGIYVTRVSEGGPAEIAGLQIGDKIMQVNGWDMTMVTHDQARKRL
TKRSEEVVRLLVTRQSLQKAVQQSMnss

Construct: TIP1-MIN
Primers: 2753& 2762
Vector Cloning Sites(5′/3′): EcoR1/EcoR1

Insert Cloning Sites(5′/3′): EcoR1/EcoR1

aa 1 - aa 129

(SEQ ID NO:74)

agilEMSYIPGQPVTAVVQRVEIHKLRQGENLILGFSIGGGIDQDPSQNP
FSEDKTDKGIYVTRVSEGGPAEIAGLQIGDKIMQVNGWDMTMVTHDQARK
IRLTKRSEEXWRLLVTRQSLQKAVQQSMLS

Construct: TIP1-CD5γ
Primers: 2584& 2585
Vector Cloning Sites(5′/3′): Bam H1/Bam H1

Insert Cloning Sites(5′/3′): BamH1/ Bam H1

aa 1 - aa 122

(SEQ ID NO:75)

adPGQPVTAVVQRVEIHKLRQGENLILGFSIGGGIDQDPSQNPFSEDKTD
KGIYVTRVSEGGPAEIAGLQIGDMMQVNGWDMTMVTHDQARKRLTKRSEE
VVRLLVTRQSLQKAVQQSdpe

D. GST Fusion Protein Production and Purification

The constructs using pGEX-3× expression vector were used to make fusion proteins according to the protocol outlined in the GST Fusion System, Second Edition, Revision 2, Pharmacia Biotech. Method II and was optimized for a 1L LGPP.

Purified DNA was transformed into E. coli and allowed to grow to an OD of 0.4-0.8 (600λ). Protein expression was induced for 1-2 hours by addition of IPTG to cell culture. Cells were harvested and lysed. Lysate was collected and GS4B beads (Pharmacia Cat# 17-0756-01) were added to bind GST fusion proteins. Beads were isolated and GST fusion proteins were eluted with GEB II. Purified proteins were stored in GEB II at −80° C.

Purified proteins were used for ELISA-based assays and antibody production. A list of PDZ domains fused to GST with amino acid sequences of the inserts is presented in Table 8.

E. IgG Fusion Protein Production and Purification

The constructs using the CD5gamma or Peak10IgG expression vectors were used to make fusion protein. Purified DNA vectors were transfected into 293 EBNA T cells under standard growth conditions (DMEM +10% FCS) using standard calcium phosphate precipitation methods (Sambrook, Fritsch and Maniatis, Cold Spring Harbor Press) at a ratio of ˜1 ug vector DNA for 1 million cells. This vector results in a fusion protein that is secreted into the growth medium. Transiently transfected cells are tested for peak expression, and growth media containing fusion protein is collected at that maxima (usually 1-2 days). Fusion proteins are either purified using Protein A chromatography or frozen directly in the growth media without addition.

### EXAMPLE 6

### TIP-1 and MAGI-1(D2) PDZs Specifically Bind to Oncogenic E6 Proteins

A. Abstract

An experiment was conducted to demonstrate and confirm that PDZ domains would only recognize the C-termini of recombinant oncogenic HPV E6 proteins and not non-oncogenic E6 variants. This validates the method of using peptides representing the PL sequences of E6 proteins by asking if the PDZ binding can be reproduced using full length E6 fusion proteins.

Briefly, GST-HPV E6 fusion proteins were constructed as described in Example 4 corresponding to the full length protein sequence of E6 from HPV18 (oncogenic) and HPV11 (non-oncogenic). Using a modified ELISA assay, binding of a TIP-TIP-IgG fusion protein (two copies of the TIP-1 PDZ domain fused to the hIgG constant region, purification of fusion protein partially described in Example 5) to these two E6 variants was assessed using the ELISA listed below.

B. Modified ELISA Method

Reagents and Materials
Nunc Polysorp 96 well Immuno-plate (Nunc cat#62409-005) (Maxisorp plates have been shown to have higher background signal)
PBS pH 7.4 (Gibco BRL cat#16777-148) or AVC phosphate buffered saline, 8 gm NaCl, 0.29 gm KCl, 1.44 gm Na₂HPO4, 0.24 gm KH₂PO4, add H2O to 1 L and pH 7.4; 0.2 micron filter
2% BSA/PBS (10 g of bovine serum albumin, fraction V (ICN Biomedicals cat#IC15142983) into 500 ml PBS
Goat anti-GST mAb stock @ 5 mg/ml, store at 4° C., (Amersham Pharmacia cat#27-4577-01), dilute 1:1000 in PBS, final concentration 5 ug/ml
Wash Buffer, 0.2% Tween 20 in 50 mM Tris pH 8.0
TMB ready to use (Dako cat#S1600)
1M H₂SO₄
12 w multichannel pipettor,
50 ml reagent reservoirs,
15 ml polypropylene conical tubes
anti E6HPV18 antibody(OEM Sciences)
Anti-hIgG-HRP (Biomeda)
   Protocol
1) Coat plate with 5 ug/ml GST-E6 fusion protein, O/N @ 4° C.
2) Dump proteins out and tap dry
3) Blocking—Add 200 ul per well 2% BSA/PBS, 2 hrs at 4° C.
4) Prepare PDZ proteins (50:50 mixture of supernatant from TIP-TIP-IgG transfection and 2% BSA/PBS)
5) 3× wash with cold PBS
6) Add PDZ protein prepared in step 7 or anti-E6 Ab at 1 ug/ml in 2% BSA/PBS (or anti-GST Ab as control).
7) 3× wash with cold PBS
8) Add appropriate concentration of enzyme-conjugated detection Ab (anti-hIgG-HRP, anti-goat-HRP, or anti-mouse-HRP) 100 ul per well on ice, 20 minutes at 4° C.
9) Turn on plate reader and prepare files
10) 5× wash with Tween wash buffer, avoiding bubbles
11) Using gloves, add TMB substrate at 100 ul per well
   incubate in dark at room temp
   check plate periodically (5, 10, & 20 minutes)
   take early readings, if necessary, at 650 nm (blue)
   at 30 minutes, stop reaction with 100 ul of 1M H₂SO₄
   take final reading at 450 nm (yellow)

C. Results of Binding Experiments

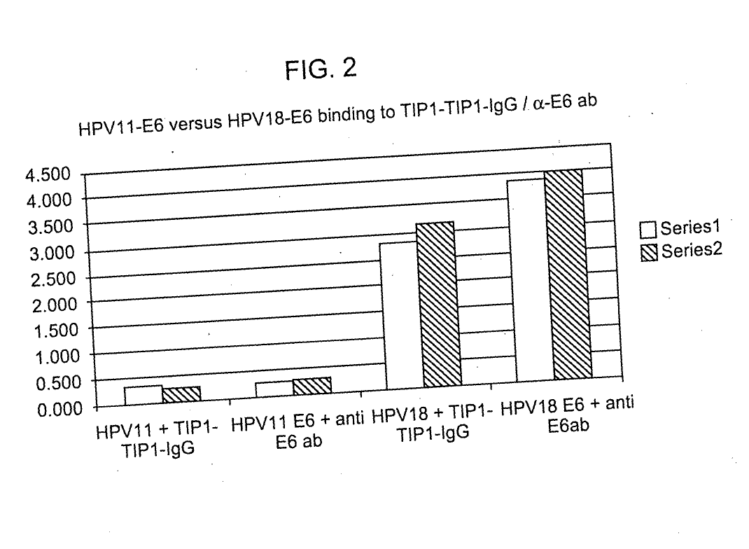

TIP-1, a representative PDZ domain that binds most oncogenic E6 PLs (EXAMPLE 2, TABLES 3A, 3B), is able to specifically recognize PLs from full length oncogenic E6 variants (HPV18-E6) without binding to non-oncogenic variants (HPV11-E6; FIG. 2). Furthermore, even unpurified TIP-TIP-IgG fusion protein is able to recognize GST-HPV18E6 fusion protein at levels comparable to an antibody generated against HPV18-E6. Antibodies against GST were used to confirm that the GST-HPV18E6 and GST-HPV11E6 were uniformly plated (data not shown). This confirms that the results from the assay using E6 PL peptides to define interactions between oncogenic E6 proteins and PDZ domains is representative of full length protein interactions, and that the PDZ domain of TIP-1 can recognize full length recombinant E6 from oncogenic E6 proteins but does not bind to Non-oncogenic E6 variants. MAGI-1 was demonstrated to bind oncogenic E6 proteins in a similar manner (data not shown).

### EXAMPLE 7

### Inhibition of TIP1-HPV E6 16 Binding by PL Peptides

Purpose: To demonstrate that specific peptides can disrupt the interaction between an oncogenic E6 protein and the PDZ domain of TIP-1.

Materials and Methods: A. The modified G assay was performed as described below, adding putative inhibitors concurrent with the addition of E6 PDZ Ligand peptide to the plated PDZ protein.

B. Modified ELISA Method

Reagents and Materials
Nunc Polysorp 96 well Immuno-plate (Nunc cat#62409-005) (Maxisorp plates have been shown to have higher background signal)
PBS pH 7.4 (Gibco BRL cat#16777-148) or AVC phosphate buffered saline, 8 gm NaCl, 0.29 gm KCl, 1.44 gm Na₂HPO4, 0.24 gm KH₂PO4, add H2O to 1 L and pH 7.4; 0.2 micron filter
2% BSA/PBS (10 g of bovine serum albumin, fraction V (ICN Biomedicals cat#IC15142983) into 500 ml PBS
Goat anti-GST mAb stock @ 5 mg/ml, store at 4° C., (Amersham Pharmacia cat#27-4577-01), dilute 1:1000 in PBS, final concentration 5 ug/ml
GST-TIP1 fusion protein (stock stored at −80° C. in 35% glycerol), diluted to 5 ug/ml in 2% BSA/PBS
Peptide mix: 10 uM HPV E6 16 biotin labeled peptide+titrating amounts (0.001 uM, 0.01 uM, 0.1 uM, 1 uM, 10 uM, or 100 uM) of Tax unlabeled peptide in 2% BSA/PBS or small molecule compounds at described concentrations
Wash Buffer, 0.2% Tween 20 in 50 mM Tris pH 8.0
TMB ready to use (Dako cat#S1600)
0.18M H₂SO₄
12 w multichannel pipettor,
50 ml reagent reservoirs,
15 ml polypropylene conical tubes
Anti-hIgG-HRP (Biomeda)
   Protocol
1. Coat plate with 100 ul of 5 ug/ml anti-GST Ab, O/N @ 4° C.
2. Dump excess antibody and tap dry
3. Blocking—Add 200 ul per well 2% BSA/PBS
4. Incubate for 2 hrs at 4° C.
5. Rinse off blocking by washing 3 times with 200 ul per well cold PBS, then tap dry
6. Add 50 ul 5 ug/ml GST-TIP1 fusion protein in 2% BSA/PBS (or GST alone as control).
7. Incubate at 4° C. for 1-2 hours
8. Rinse off excess protein by washing 3 times with 200 ul per well cold PBS, then tap dry.
9. Add 50 ul of the peptide mixture reagent (HPV E6 16+Tax peptides).
10. Incubate on ice for 10 minutes, then RT for 20 minutes
11. Rinse off excess peptide by washing 3 times with 200 ul per well cold PBS, then tap dry.
12. Add 100 ul per well 0.5 ug/ml of HRP-Streptavidin on ice, 20 minutes at 4° C.
13. Rinse by washing 5 times with Tween wash buffer, then tap dry
14. Add 100 ul per well TMB substrate
15. Incubate in dark at room temp, checking plate periodically (5, 10, & 20 minutes)
16. Take early readings, if necessary, at 650 nm
17. At 30 minutes, stop reaction with 100 ul of 0.18M H₂SO₄, and take final reading at 450 nm

C. Results of binding experiments

FIG. 3 shows the results of inhibition assay with Tax PL peptide. Inhibition was measured by depression of A₄₅₀ reading compared to positive control (HPV E6 16+TIP1 without Tax PL). As shown in the figure, increasing concentrations of Tax PL peptide decrease binding between TIP1 and HPV E6 16 in vitro. These results suggest that peptides, peptide mimetics, or other inhibitory molecules may effectively block HPV PL-PDZ interactions in vivo.

### EXAMPLE 8

### Pathogen PL Proteins

Many other diseases can potentially be treated via manipulation of interactions between intracellular PDZ proteins and disease-associated PL proteins. Table 6 contains examples of some pathogens that are known to involve proteins containing a PL motif. These PL proteins may provide valuable therapeutic targets for the treatment of diseases resulting from pathogen infections. As for HPV E6, the C-terminal PL domains of these proteins may be used as an anti-viral therapy.

TABLE 6
Example Pathogens amenable to PDZ:PL directed therapeutics

| Pathogen | Protein | Gi or ACC number | PL/PDZ |
|---|---|---|---|
| Adenovirus | E4 | 19263371 | PL |
| Hepatitus B virus | Protein X | 1175046 | PL |
| Human T Cell | TAX | 6983836 | PL |
| Leukemia Virus | | | |
| Herpesvirus | DNA polymerase | 18307584 | PL |
| Herpesvirus | US2 | 9629443 | PL |

### EXAMPLE 9

### Migration and Proliferation of Cells Bearing Oncogenic HPV Protein or Mutations thereof

The following example shows the results of assays to determine the rate of migration and proliferation of cells bearing oncogenic HPV E6 16 proteins or fragments thereof.

A. Constructs:

Plasmid constructs of HPV E6 16 wild type and HPV E6 16 ΔPL were generated using the vector pmKit, containing an HA tag. Recombinant plasmids were generated by recombinant DNA cloning methods known in the art and outlined in Examples 4 and 5. Primers used to generate HPV DNA fragments are shown in Table 7.

TABLE 7

Primers used for generation of HPV E6 16
protein and fragments thereof

| ID# | | |
|---|---|---|
| (Primer | | |
| Name) | Primer Sequence | Description |
| 2606 | AAGCGTCGACTTTAT | Forward (5′ to 3′) |
| (1083EF) | GCACCAAAAGAG | primer corresponding |
| | (SEQ ID NO:76) | to HPV E6 16, gene- |
| | | rates a Sal I site. |
| | | Used for cloning into |
| | | pmKit. |
| 2607 | AATGCTCGAGTATCT | Reverse (3′ to 5′) |
| (1084ER) | CCATGCATGATTAC | primer corresponding |
| | (SEQ ID NO:77) | to HPV E6 16, gene- |
| | | rates a Xho I site. |
| | | Used for cloning into |
| | | pmKit. |
| 2608 | GCTGCTCGAGTCAAC | Reverse (3′ to 5′) |
| (1085ERPL) | GTGTTCTTGATGATC | primer corresponding |
| | (SEQ ID NO:78) | to HPV E6 16 ΔPL, |
| | | generates a Xho I |
| | | site. Used for |
| | | cloning into pmKit. |

pmKit-HPV E6 16 wild-type
Primers: 2606, 2607
GI#: 4927719
Vector Cloning Sites(5′/3′): SalI/Xhol
Insert Cloning Sites(5′/3′): SalI/Xhol
   pmKit-HPV E6 16 ΔPL
Primers: 2606, 2608
GI#: 4927719
Vector Cloning Sites(5′/3′): SalI/Xhol
Insert Cloning Sites(5′/3′): SalI/Xhol

B. Transfection

The above-mentioned constructs were transfected into HELF69 primary cells using the LipofectAMINE™ 2000 Reagent (Invitrogen Cat# 11668-027) and accompanying protocol. PmKit-HA without insert was transfected as a negative control. Cells were incubated at 37° in RPMI media with non-essential amino acids, 10% FBS, and 1 ug/mL G418 until confluent (about 4 days).

Each of the three transfected cell groups (pmKit-HA-HPV E6 16 wt, pmKit-HA-HPV E6 16 ΔPL, pmKit-HA control) were seeded onto a 12-well plate, and allowed to adhere and grow to confluent (about 24 hours) in RPMI media with 4% FBS and non-essential amino acids. A sterile pipet tip (about 1 mm diameter) was dragged through the cells, creating a gap in the lawn. Cells were monitored and photographed at 48-hour intervals.

C. Results:

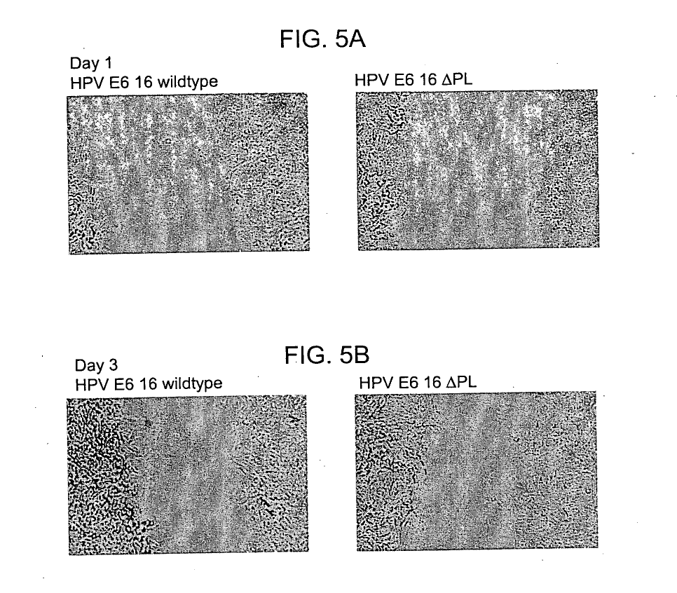

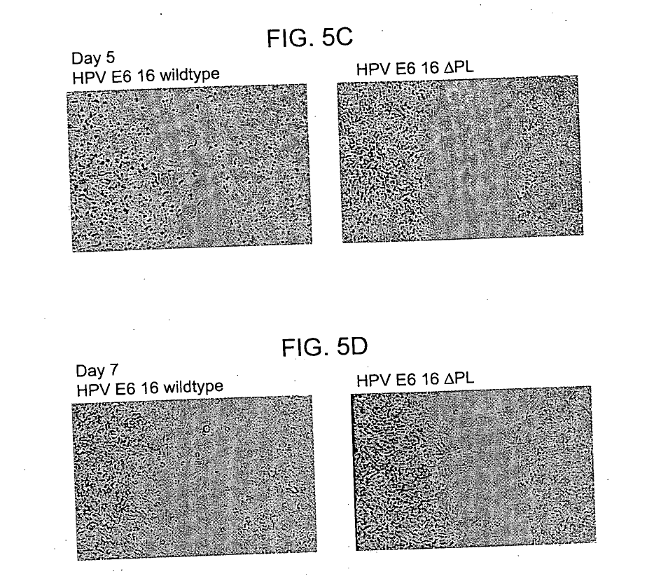

Results of migration assays are shown in FIG. 5. FIG. 5A shows HPV E6 16 wildtype and ΔPL transfections 1 day after scratching. FIG. 5B shows HPV E6 16 wildtype and ΔPL transfections 3 days after scratching. FIG. 5C shows HPV E6 16 wildtype and ΔPL transfections 5 days after scratching. FIG. 5D shows HPV E6 16 wildtype and ΔPL transfections 7 days after scratching.

D. Conclusions:

Cells transfected with HPV E6 16 wild type fill the gap faster than those transfected with HPV E6 16 ΔPL. These results suggest that the PL motif on E6 proteins from oncogenic strains of HPV is essential for the development of cancerous characteristics in cells. This assay could be used to demonstrate the effect of E6 directed therapeutics in a biological system.

### EXAMPLE 10

### Exogenus Oncogenic E6 Protein Activities JNK Activity in Xenopus Oocytes that can be Blocked by Peptide Inhibitors

Experimental Design: This experiment was divided into two phases. In the first phase, an MBP-E6 fusion protein (HPV16; see example 4) was microinjected into Xenopus oocytes at different concentrations and then the oocytes were assayed for JNK activity. In the second phase, peptides corresponding to the C-termini of non-oncogenic E6 protein (HPV 11), HPV16 E6 (oncogenic) or Tax (shown to block oncogenic E6 binding to PDZ domains, FIG. 3) were co-injected with an activating amount of MBP-E6 fusion protein to assess there abilities to block JNK activation.

Isolation and Microinjection of Oocytes—Xenopus ovarian tissue was surgically removed, and oocytes were defolliculated for 1-1.5 h at room temperature with 2 mg/ml collagenase and 0.5 mg/ml polyvinylpyrrolidone in Ca21-free modified Barth's solution (88 mM NaCl, 1 mM KCl, 0.82 mM MgSO4, 2.4 mM NaHCO3, 10 mM HEPES, pH 7.5). The oocytes were then washed four times with modified Barth's solution. Stage VI oocytes were sorted manually and incubated at 16° C. for at least 10 h in OR2 solution (82.5 mM NaCl, 2.5 mM KCl, 1 mM CaCl2, 1 mM Na2HPO4, 5 mM HEPES, pH 7.5) supplemented with 1 mg/ml bovine serum albumin and 50 mg/ml gentamicin. Immature oocytes were microinjected with purified MBP-E6 protein or E6 protein and peptide and transferred to fresh OR2 for the duration of the time course. Five oocytes were collected per time point, frozen on dry ice, and stored at −80° C.

Lysis of Oocytes, Eggs, and Embryos—Frozen oocytes, eggs, and embryos were thawed rapidly and lysed by pipetting up and down in 60 ml of ice-cold extraction buffer (EB) (0.25 M sucrose, 0.1 M NaCl, 2.5 mM MgCl2, 20 mM HEPES, pH 7.2) containing 10 mM EDTA, protease inhibitors (10 mg/ml leupeptin, 10 mg/ml pepstatin, 10 mg/ml aprotinin, 1 mM phenylmethylsulfonyl fluoride), and phosphatase inhibitors (50 mM 2-glycerophosphate, 1 mM sodium orthovanadate, 2 mM microcystin). Samples were clarified by centrifugation for 2.5 min in a Beckman E microcentrifuge with a right angle rotor. Crude cytoplasm was collected and processed for immunoblotting or kinase assays, as described below.

Immunoblotting—Aliquots of oocyte, egg, or embryo lysates were added to 0.2 volumes of 63 Laemmli sample buffer. Samples were separated on 10% SDS-polyacrylamide gels (bisacrylamide:acrylamide, 100:1) and the proteins transferred to PVDF blotting membrane (Amersham Pharmacia Biotech). The membrane was blocked with 3% nonfat milk in Tris-buffered saline (150 mM NaCl, 20 mM Tris, pH 7.6) and incubated with primary antibodies. Blots were washed five times with TBS, 0.5% Tween 20 and probed with an peroxidase-conjugated secondary antibody for detection by enhanced chemiluminescence (ECL-Plus, Amersham Pharmacia Biotech). For reprobing, blots were stripped by incubation with 100 mM Tris-HCl, pH 7.4, 100 mM 2-mercaptoethanol, and 2% SDS at 70° C. for 40 min.

Jun Kinase Assay—Jun kinase assays were performed as described. Crude oocyte, egg, or embryo cytoplasm was diluted 1:1 in EB and pre-cleared with 20 ml of glutathione-Sepharose beads (Amersham Pharmacia Biotech) for 1 h at 4° C. with moderate shaking. Lysates were incubated with glutathione S-transferase GST-c-Jun-(1-79) fusion protein (hereafter denoted GST-Jun) immobilized on glutathione-Sepharose beads. After 3 h at 4° C., the beads were washed three times with 50 mM HEPES, pH 7.5, 150 mM NaCl, 1% Triton X-100, 1 mM EDTA, 10% glycerol,10 mM sodium pyrophosphate, 2 mM sodium orthovanadate, 10 mM sodium fluoride, 1 mM phenylmethylsulfonyl fluoride, and 10 mg/ml aprotinin (24) and once with 0.4 ml of kinase buffer (20 mM HEPES, pH 7.5, 10 mM MgCl2, 1 mM dithiothreitol, 200 mM sodium orthovanadate). The bound JNK activity was detected by the addition of 1 mCi of [g-32P]ATP. The reaction was terminated after 20 min at 30° C., and the products were resolved by SDS-PAGE. The gels were transferred to PVDF membranes (Hybond; Amersham Pharmacia Biotech) and the incorporation of [32P]phosphate into GST-Jun was visualized by autoradiography.

Results

FIG. 4A shows that oncogenic HPV16 E6, but not non-oncogenic HPV11 E6, activates c-JUN N-terminal kinase (JNK), a kinase known to be involved in numerous oncogenic pathways. FIG. 4B demonstrates that HPV16 E6-dependent activation of JNK can be inhibited by co-injection of peptide corresponding to the C-terminus of Tax (an independent PDZ ligand that binds similar PDZ domains), but not with peptide representing the C-terminus of non-oncogenic HPV E6 11. FIG. 4C demonstrates that HPV 16 E6 dependent activation of JNK can be inhibited by peptide representing the c-terminus of the HPV16 E6 oncoprotein, but not by peptide representing the C-terminus of nononcogenic HPV11 E6 protein.

### CONCLUSION/DISCUSSION

This assay clearly demonstrates that oncogenic E6 proteins can activate JNK activity whereas non-oncogenic E6 proteins cannot. In addition, this activation can be blocked using peptides that mimic the PL sequences of proteins that bind these specific PDZ domains, demonstrating complete blocked of oncogenic transformation as assayed by JNK activity. These data demonstrate not only that blocking this PDZ:PL is a potent method of preventing oncogenic transformation, but that this assay is suitable for testing the effect of other oncogenic E6 inhibitors on biological function.

### EXAMPLE 11

### Oncogenic HPV E6 16 Activation of Cancer-Associated Kinase is Dependent on PDZ Binding

This example demonstrates that oncogenic E6 proteins will activate JNK in mammalian cells and that this activation is dependent on the. C-terminal PDZ Ligand (PL) sequence.

Methods: Mammalian 293 cells were transfected by standard Calcim Phosphate methods with pmKIT vectors carrying inserts from the group: A (no insert), HPV16 E6, HPV16 E6 ΔPL (C-terminal 3 amino acids deleted), or HPV16 E7. Transfected cells were collected after 2 days and assayed of JNK activity through the lysates ability to phophorylate GST-cJUN (see Example 10). JNK activity positive controls were treated with EGF or Sorbitol prior to lysis to activate JNK.

Results

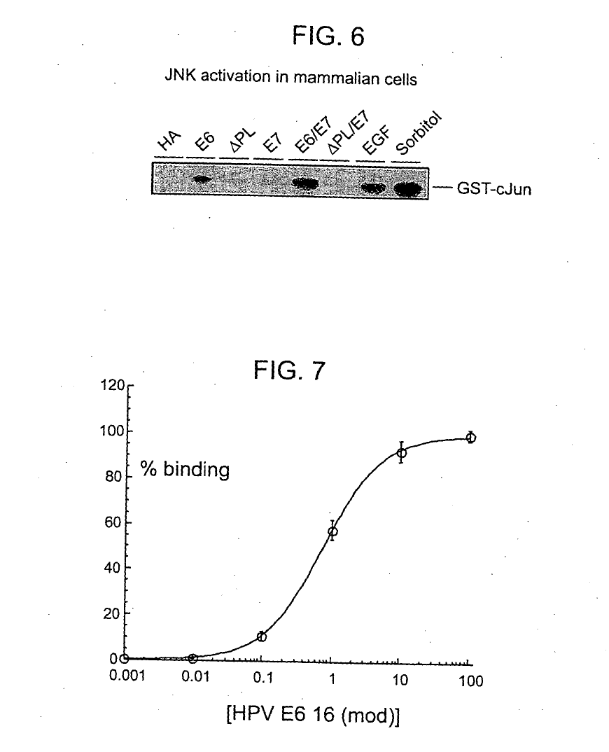

FIG. 6 shows the results of these experiments. HPV16 E6 protein alone can activate JNK activity in mammalian 293 cells. This activity is dependent on the PDZ Ligand (PL), as the ΔPL construct that is identical to HPV16 E6 construct except for a deletion of the c-terminal 3 amino acids fails to activate JNK. This activation is not dependent upon E7 co-transfection.

Discussion

This experiment demonstrates that the E6 protein from oncogenic HPV strain 16 is able to activate JNK, but that this activation is dependent on it's ability to bind PDZ proteins. Hence, therapeutics directed at disrupting the ability of oncogenic E6 proteins to interact with cellular PDZ proteins should be effective at preventing oncogenic transformation of cells. In addition, this provides another assay in a mammalian system that can be used to test the biological effects of inhibitors of E6 PL:PDZ interactions, whether they are peptides, peptidomimetic or small molecules.

### EXAMPLE 12

### Small Molecule Drugs can Block the Interaction of Oncogenic E6 Protein with the PDZ Domain of TIP-1

The C-terminal motif of HPV E6 16 is required for cellular transformation in rodent cells. Further cellular assays have demonstrated that cell migration of HPV E6 16 transfected cells is PL dependent, where E6 wt cells migrate faster than ΔPL cells.

In this example, a library of FDA approved drugs was tested for potential small molecule inhibitors of the HPV16 E6/TIP 1 interaction (shown in FIG. 7). From this drug screen, five potential drug inhibitors were selected (drugs 43 (benztropine mesylate), 102 (clomipramine hydrochloride), 264 (methotrimeprazine), 276 (mitoxantrone hydrochloride) and 410 (verapamil hydrochloride)) and titrated against the TIP 1/HPV E6 16 interaction as shown in FIG. 8 (FIGS. 8A-8E respectively). The IC50 for these reactions was on the order of 100-200 μM. The inhibition reactions were performed using the G assay protocol described supra at a HPV16 E6 concentration of 2 μM for the drug screen experiments.

From these results, we have demonstrated the potential of inhibiting HPV16 E6 /PDZ domain interactions with small molecule compounds. Further work may be done for optimizing the potency of these inhibitors through chemical modifications of these compounds.

### EXAMPLE 12

### The PDZ-Ligand Motif of Oncogenic E6 is Necessary to Regulate MAGI 1 and Activate the JNK Pathway

Due a change in nomenclature, in the following example, what is referred to as MAGI-1 PDZ domain 1, or the like, may the same as MAGI PDZ domain 2, as referenced in the rest of this patent application.

Material and Methods

Antibodies, Cell Lines, Reagents Recombinant Proteins and Plasmids

Antibodies—Anti-JNK antibodies used were mouse monoclonal anti-phospho-JNK (P-Thr 183/P-Tyr 185) (Cell Signaling), rabbit polyclonal anti-JNK1 (SC571; Santa Cruz Biotechnology), and anti-JNK2 (SC572; Santa Cruz Biotechnology). Mouse IgG-2a R-phycoerythrin and the isotype control Ab were from Caltag Laboratories. Phycoerythrin-labeled mouse monoclonal Ab to human CD69 were purchased from Caltag laboratories. Anti-CD69 Microbeads were obtained from Miltenyi Biotec.

Cell lines—Human cervical cancer cell lines HeLa, SiHa, Caski and C 33A, and human embryonic kidney cells (HEK 293) were obtained from the American Type Culture Collection (ATCC). All cell lines were maintained in Dulbecco's modified Eagle's medium (DMEM) supplemented with 10% fetal calf serum (FCS) (Sigma) at 37° C. and 5% CO₂. Culture media was purchased from Gibco-BRL.

Reagents—Gluthathione Sepharose 4B and Protein A Sepharose were obtained from Pharmacia/Amersham Biotech Inc. all other reagents were from Sigma. Recombinant Proteins and Plasmids—GST-Jun was expressed and purified from BL21 E. coli cells.

Lysis, Transfection and Microinjection of Mammalian Cells and Xenopus oocytes

Cell lysis—Cells grown to 80-90% confluence were treated as indicated, washed once with phosphate-buffered saline and then lysed for 10 min on ice in buffer containing 50 mM HEPES pH 7.5, 150 mM NaCl, 1% Triton X-100, 1 mM EDTA, 10% glycerol, 10 mM sodium pyrophosphate, 2 mM sodium orthovanadate, 10 mM sodium fluoride, 1 mM phenylmethylsulfonyl fluoride and 10 μg/ml aprotinin. Lysates were cleared by centrifugation at 13 000 r.p.m. for 10 min at 4° C. and either used in kinase assays (described below) or directly separated by SDS-Page an immunoblotted.

Calcium phosphate transfections—Human embryonic kidney cells (HEK 293) cells were maintained in culture under Dulbecco's modified Eagle medium containing 10% fetal bovine serum. They were sustained in a 37° C. incubator with a 5% CO₂ atmosphere. Immediately before transfection cholorquine at a final concentration of 25 uM was added to the cell culture media. Plasmids were transfected into HEK 293 ET cells via calcium phosphate DNA precipitation, using 30 ug DNA per 95% confluent 10 cm diameter plate. Cells were incubated at 37° C. for 8 hours, after which the media was changed. Harvesting of the cells took place at 24- and 48-hour post-transfection intervals. Transfection efficiency was checked by analyzing cells that had been transfected in parallel with an eGFP plasmid, transfection efficancies were 85-95%.

Lysis of Xenopus Oocytes—Frozen oocytes, eggs, and embryos were thawed rapidly and lysed by pipetting up and down in 60 μl of ice-cold extraction buffer (EB) (0.25 M sucrose, 0.1 M NaCl, 2.5 mM MgCl2, 20 mM HEPES, pH 7.2) containing 10 mM EDTA, protease inhibitors (10 μg/ml leupeptin, 10 μg/ml pepstatin, 10 μg/ml aprotinin, 1 mM phenylmethylsulfonyl fluoride), and phosphatase inhibitors (50 mM 2-glycerophosphate, 1 mM sodium orthovanadate, 2 μM microcystin). Samples were clarified by centrifugation for 2.5 min in a Beckman E microcentrifuge with a right angle rotor. Crude oocyte cytoplasm. was diluted 1:1 in EB and pre-cleared with 20 μl of glutathione-Sepharose beads (Amersham Pharmacia Biotech) for 1 h at 4° C. with moderate shaking. These oocyte lysates were then used for kinase assays, as described below.

JNK kinase assay—Lysates were obtained as described above, incubated with glutathione S-transferase GST-c-Jun-(1-79) fusion protein (hereafter denoted GST-Jun) immobilized on glutathione-Sepharose beads. After 3 h at 4° C., the beads were washed three times with 50 mM HEPES, pH 7.5, 150mM NaCl, 1% Triton X-100, 1 mM EDTA, 10% glycerol, 10 mM sodium pyrophosphate, 2 mM sodium orthovanadate, 10 mM sodium fluoride, 1 mM phenylmethylsulfonyl fluoride, and 10 μg/ml aprotinin (24) and once with 0.4 ml of kinase buffer (20 mM HEPES, pH 7.5, 10 MM MgCl₂, 1 mM dithiothreitol, 200 μM sodium orthovanadate). The bound JNK activity was detected by the addition of 1 μCi of [γ-³²P]ATP. The reaction was terminated after 20 min at 30° C., and the products were resolved by SDS-PAGE. The gels were transferred to PVDF membranes (Hybond; Amersham Pharmacia Biotech) and the incorporation of ³²P into GST-Jun was visualized by autoradiography and quantified by PhosphorImaging.

Matrix Assays

Footnote: HPV16-E6-PL was usually considered to bind a particular PDZ when (1) the OD signal was greater or equal to 0.5, the relative standard deviation of the measurement was less than 0.25, and the signal to noise ratio was greater than 2 ([OD measurement/OD background (GST alone)]>2) These criteria together with high OD measurement values were used to determine the strongest interactions.

In this section, we describe the development of a small molecule-and peptide based drug screen that we plan to use for the identification of compounds targeting E6-PL:PDZ interactions.

To determine whether HPV-E6:PDZ interactions can be disrupted, we developed a modified G-assay for an in vitro screen of small molecule or peptide inhibitors. (see C3.1) A biotinylated peptide is premixed with an inhibitor (at a 100 μM concentration). The inhibitor concentration is relatively high compared to “traditional” drug screens that use 5-10 μM concentrations. This will permit identification of weak inhibitors whose potency may be potentially improved by structural modifications. The mixture is applied to an antibody-bound GST-PDZ on the surface of an ELISA well and allowed to react. The extent of reaction is compared to a control with no inhibitor present, and is based on a colorimetric test. Small molecules that inhibit the peptide/PDZ binding by at least 25% are further tested in titrations to determine the EC50 of inhibition by the drug (see D.1 for general design).

We decided to use 100 selected FDA approved drugs to determine, whether the G-assay is compatible with a small molecule drug screen, and to gain an initial insight regarding the question whether PL-PDZ interactions can be targeted by small molecule drugs. The criteria for drug selection included water solubility and presence of polar functional groups that could potentially interact with the PDZ.

Results

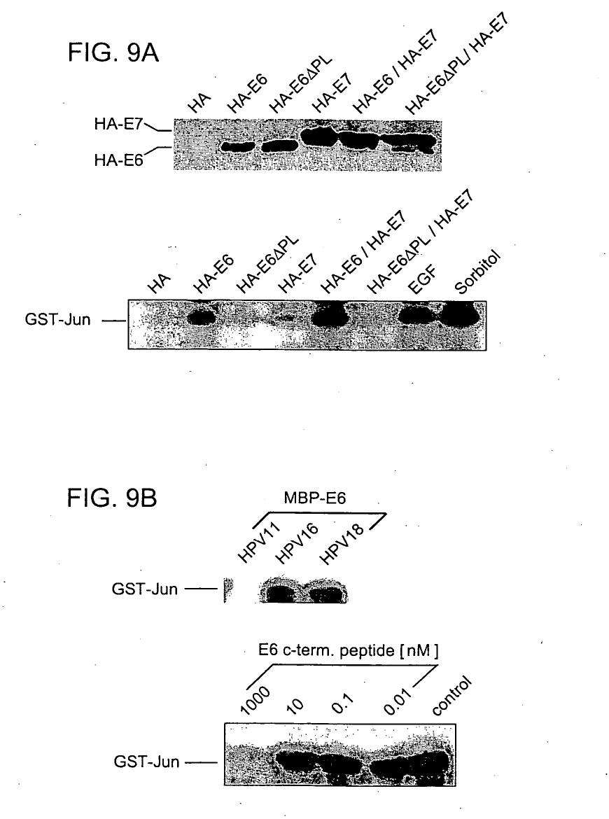

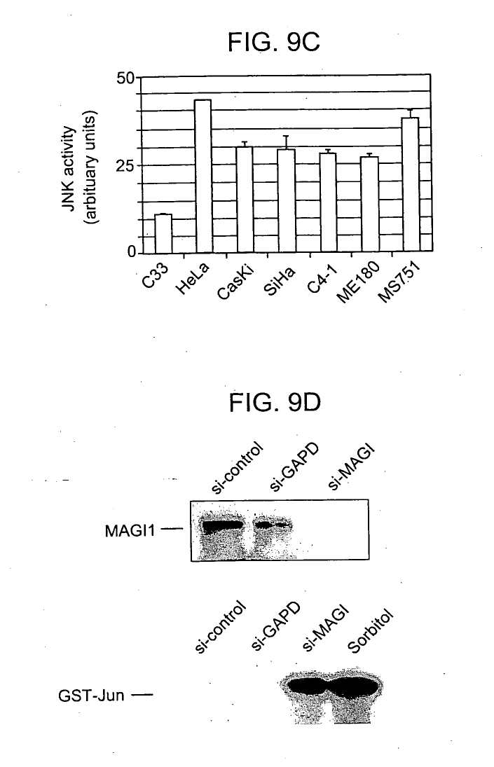

First, we analyzed basal JNK activity levels in cervical cancer cell lines and we found that in six HPV-positive cell lines SiHa, HeLa, C4-1, ME180, MS751 and Caski, JNK activity was significantly higher than in the HPV-negative cervical cancer cell line, C33A (FIG. 9C). We then examined whether high-risk HPV E6 protein can activate JNK. We transiently transfected 293 cells with plasmids encoding HPV16-E6 and HPV16-E6 □PL. As shown in FIG. 9A, the JNK pathway was activated in cells expressing HPV16-E6 but not E6 □PL, thus demonstrating that JNK activation is E6-PL dependent. JNK activation was not affected by expression of E7 (FIG. 9A), and under similar conditions the ERK MAPK pathway was not activated (data not shown). To test whether interruption of the E6/PDZ interaction interferes with JNK activation, we used Xenopus oocytes, which have been shown to have an inducible robust JNK pathway (19) while at the same time can be microinjected with proteins, peptides and drugs. Recombinant GST-E6 fusion proteins (HPV16-E6, HPV18-E6 and HPV11-E6) were microinjected in Xenopus oocytes and JNK activity was measured. Only HPV16 E6 and HPV18 E6 proteins activated the JNK MAPK pathway in the oocyte (FIG. 1B). In contrast, low-risk HPV11E6 was unable to activate JNK (FIG. 9B). We tested a 20 mer peptide corresponding to the C-terminus of HPV 16 E6 for its ability to inhibit the E6 PDZ/PL interaction in vitro (data not shown). When coinjected with GST-HPV16E6 into the oocytes, the peptide blocked E6-dependent JNK activation (FIG. 9B). A control peptide corresponding to the C-terminus of low-risk HPV11 E6 which lacks the PL had no effect (FIG. 9B).

In summary, we have demonstrated that inhibiting the PL interaction of the high-risk HPVE6 PL with an unknown PDZ protein in intact cells interfered with E6-induced JNK activation.

The C-termini of high-risk HPV E6 proteins have recently been identified as ligands for cellular PDZ domain-containing proteins, including the human homologue of the Drosophila tumor suppressor discs large (Dlg) (3). Removal of a single C-terminal amino acid from the PDZ ligand (PL) sequence of a high-risk HPV E6 protein abolished its ability to transform cell lines or form tumors in nude mice (4). Moreover, K14-HPV16 E6 transgenic mice developed skin tumors and cervical carcinomas dependent on the presence of the PL (29). We examined the C-terminal sequences of the E6 proteins encoded by all high-risk and low-risk HPVs. We found a 100% correlation between the presence of a PL consensus sequence with the classification as high-risk HPV (Table 9).

TABLE 9

Correlation of E6 PDZ-ligands
and oncogenicity

| HPV | E6 C-terminal | | onco- | PL-motif |
|---|---|---|---|---|
| strain | sequence | PL | genic | representation |
| HPV 4 | GYCRNCIRKQ | No | No | n.a. |
| | (SEQ ID NO:79) | | | |
| HPV 11 | WTTCMEDLLP | No | No | n.a. |
| | (SEQ ID NO:80) | | | |
| HPV 20 | GICRLCKHFQ | No | No | n.a. |
| | (SEQ ID NO:81) | | | |
| HPV 24 | KGLCRQCKQI | No | No | n.a. |
| | (SEQ ID NO:82) | | | |
| HPV 28 | WLRCTVRIPQ | No | No | n.a. |
| | (SEQ ID NO:83) | | | |
| HPV 36 | RQCKHFYNDW | No | No | n.a. |
| | (SEQ ID NO:84) | | | |
| HPV 48 | CRNCISHEGR | No | No | n.a. |
| | (SEQ ID NO:85) | | | |
| HPV 50 | CCRNCYEHEG | No | No | n.a. |
| | (SEQ ID NO:86) | | | |
| HPV 16 | SSRTRRETQL | Yes | Yes | 33 |
| | (SEQ ID NO:87) | | | |
| HPV 18 | RLQRRRETQV | Yes | Yes | 68 |
| | (SEQ ID NO:88) | | | |
| HPV 30 | RRTLRRETQV | Yes | Yes | |
| | (SEQ ID NO:89) | | | |
| HPV 35 | WKPTRRETEV | Yes | Yes | 18,30, |
| | (SEQ ID NO:90) | | | 39,45, |
| | | | | 51,68, |
| | | | | 59 |
| HPV 39 | RRLTRRETQV | Yes | Yes | |
| | (SEQ ID NO:91) | | | |
| HPV 45 | RLRRRRETQV | Yes | Yes | |
| | (SEQ ID NO:92) | | | |
| HPV 51 | RLQRRNETQV | Yes | Yes | |
| | (SEQ ID NO:93) | | | |
| HPV 52 | RLQRRRVTQV | Yes | Yes | 18,39, |
| | (SEQ ID NO:94) | | | 45,51, |
| | | | | 59, |
| HPV 56 | TSREPRESTV | Yes | Yes | |
| | (SEQ ID NO:95) | | | |
| HPV 59 | QRQARSETLV | Yes | Yes | |
| | (SEQ ID NO:96) | | | |
| HPV 58 | RLQRRRQTQV | Yes | Yes | 18,68, |
| | (SEQ ID NO:97) | 52,68 | | |
| HPV 33 | RLQRRRETAL | Yes | Yes | 16 |
| | (SEQ ID NO:98) | | | |
| HPV 66 | TSRQATESTV | Yes | Yes | 56 |
| | (SEQ ID NO:99) | | | |
| HPV 68 | RRRTRQETQV | Yes | Yes | |
| | (SEQ ID NO:100) | | | |
| HPV 69 | n.d. | n.d. | Yes | |

Table 9: E6 C-terminal sequences and oncogenicity. HPV variants are listed at the left. Sequences were identified from. Genbank sequence records. PL Yes/No was defined by a match or non-match to the consensus X-(S/T)-X-(V/I/L). Oncogenicity data collected from National Cancer Institute.

In contrast, no PL motifs were found in any of the E6 proteins encoded by low-risk HPVs (Table 9), suggesting a role for a PDZ/PL interaction in cervical cancer development. Upon examination of the C-terminal 4 residues of other HPVs not yet classified as high-risk by epidemiological studies, we noted that HPV26 E6 (ETQV), HPV34 E6 (ATWV) and HPV53 E6 (ESAV) contain C-terminal amino acid sequences consistent with PL motifs. Interestingly, these HPVs were recently classified as high-risk (5). In addition the E6 proteins encoded by two additional newly identified high-risk viruses, HPV73 (ATVV) and HPV82 (ETQV), also contain PL consensus motives (5). HPV E6 proteins bind to a number of cellular proteins, including E6AP (6), PAXILLIN (7), IRF-3 (8), BAK (9), and to the PDZ containing proteins DLG 1 (3), MUPP 1 (10), VARTUL (11), MAGI 1 (12), MAGI 2 (13) and MAGI 3 (13). Currently, however, no systematic studies of HPV E6 binding to all PDZ proteins have been done. We have identified 255 human PDZ domains, the PDZ domain complement of the human genome (the “PDZome”; see Suppl. 1). Since most of the high-risk HPVs cause the same clinical condition (premalignant cervical intraepithelial neoplasia), we hypothesized that high-risk HPV E6 proteins might target a common PDZ-dependent cellular signal transduction process in cervical epithelial cells. In order to identify the relevant human PDZ domain-containing proteins targeted by high-risk HPV E6 proteins we applied our Matrix TM platform to screen for the PbZ/PL interactions of HPV E6. We have cloned 215 individual human PDZ domains, representing the entire set of human PDZ domains identified at the time when these experiments were performed, and expressed them as GST-fusion proteins. The fusion proteins were used in the ELISA-based Matrix TM assay to determine binding of the 215 PDZ domains to a 20 mer C-terminal peptide of HPV 16 E6 (Complete binding data see Suppl. 1). In addition, we examined the binding of 6 high-risk and 3 low-risk HPV E6 C-termini to a subset (approx. 130) of human PDZ domains. The seven high-risk HPV E6 PL peptides tested were chosen because they represent all PL sequence variations (positions 0 and −2 of consensus motif) present in the 15 E6 proteins encoded by known high-risk HPVs (see Table 9). The three low-risk HPV E6 PL (HPV57, HPV63, and HPV77) failed to interact with any PDZ domains tested. Besides confirming the 6 interactions previously described in the literature, we discovered eight novel PDZ-interactions for HPV16 E6 (Table 10). Relative binding affinities for the 14 most significant interactions with different PDZ domains were determined by E6 peptide titrations. A compilation of relative EC50 values for these interactions with HPV 16 E6 is shown in Table 10.

TABLE 10
Qualitative hierarchy of EC50 values for interactions of HPV
E6 16 C-terminal peptide with different PDZs.

| | | RNA expression |
|---|---|---|
| | | (Cervical Cancer |
| PDZ gene name | EC50^{a} [uM] | cell lines) |
| Magi1C (1) | 0.056 | ++ |
| Magi3 (1) | 0.31 | neg. |
| SAST1 | 0.58 | neg. |
| TIP1 | | +++ |
| VARTUL | 0.94 | + |
| PSD95 (1-3) | 1.0 | n.d. |
| SAST2 (1) | 1.2 | n.d. |
| DLG1 (2) | To be determined | ++++ |
| DLG2 (3) | 1.6 | n.d. |
| DLG3 (1-2) | 3.8 | n.d. |
| PSD95 (2) | 6.8 | n.d. |
| SIP1 (1) | 7.5 | n.d. |
| SynBP1 | To be determined | ++ |

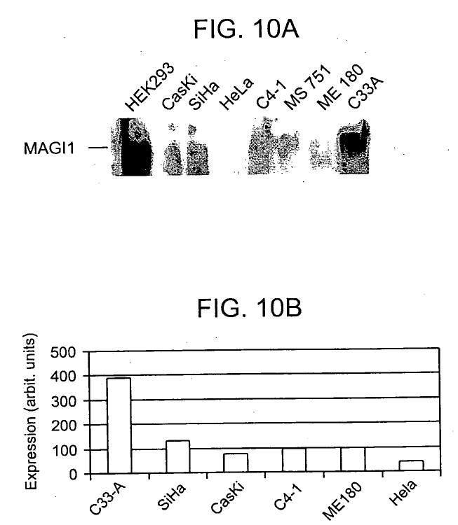

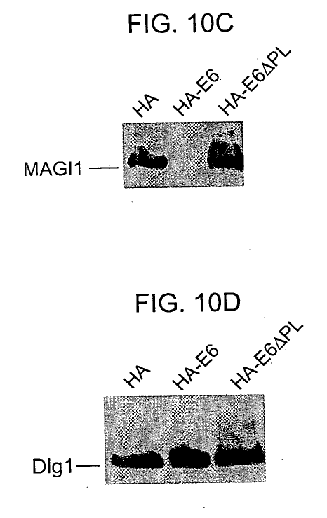

MAGI 1 domain 1 bound with the highest relative affinity and only domain 1 of its 5 PDZ domains bound to HPV 16 E6 in the Matrix™ assay, consistent with data previously described in the literature (14). MAGI 1 domain 1 was the only PDZ domain tested that bound to each of the 7 high-risk HPVE6 PLs tested. TIP 1, a small protein with a single PDZ domain, bound each of the high-risk HPV E6 PLs except HPV52 E6 (data not shown). Our data demonstrate that all high-risk HPV E6 proteins tested bound PDZ domains with a rather conserved binding pattern. To narrow down the number of potentially physiologically relevant PDZ protein targets of HPV E6 protein, we-tested expression of these PDZ-proteins in cervical cancer cell lines. We performed gene expression profiling of selected candidate PDZ proteins by real time RT-PCR. Table 10 shows the mRNA expression in cervical cancer cell lines of selected PDZ genes: Tip1, SAST1, Vartul (hScrib), MAGI 1, MAGI 3, Synaptojanin 2 binding protein (Syn2bp) and DLG 1. Two of the PDZ genes, Sast 2 and Magi 3, showed no mRNA expression in any of the cell lines tested, and consequently were therefore ruled out as physiological targets of E6. A comparison of MAGI 1 mRNA expression levels of HPV-negative, C33A cells and the HPV-positive cervical carcinoma cell lines: HeLa (HPV18), SiHa (HPV16), Caski (HPV16), C4-1 (HPV18), ME1180 (HPV68), and MS751 (HPV45) is shown in FIG. 10B. MAGI 1 mRNA expression levels were markedly lower in all HPV positive cell lines compared to the HPV-negative cells (FIG. 10B). All six HPV-positive lines expressed significantly lower levels of MAGI 1 protein compared to the HPV-negative C33A cells or the HEK293 cells (FIG. 10A). It has been reported that PDZ-domain containing proteins including DLG-1(15), MUPP-1 (9), Vartul (10), MAGI 1 and MAGI 3 (12) are targets of E6-dependent degradation through the proteasome. We investigated MAGI 1 levels in human embryonic kidney (293) cells that were transiently expressing either HPV16 E6 protein or a deletion mutant missing the last 3 C-terminal amino acids (HPV16 E6 ΔPL). Protein levels of endogenous MAGI 1 were significantly reduced in the presence of full-length E6 but not in the presence of the mutant protein (FIG. 10C). In the same experiment, binding to MAGI 1 domain 1 was only observed for the full-length HPV 16 E6 protein but not for the C-terminal ΔPL mutant (data not shown). Interestingly, protein levels of another MAGUK family protein reported to bind E6, DLG-1 , were not similarly affected by the HPV 16 E6 protein (FIG. 10C).

Our data show that basal JNK activity and MAGI 1 levels are negatively correlated and dependent on the presence of the PL motif of high-risk HPV E6. We then used RNA interference to investigate whether decreasing MAGI 1 levels in HEK293 cells had an effect on JNK activity. A small interfering RNA for MAGI 1 significantly reduced Magi1 protein expression levels and led to JNK activation when transfected in HEK293 cells (FIG. 9D). These findings show that the cellular PDZ-protein negatively involved in E6-mediated JNK activation may be MAGI 1. MAGI 1 is a membrane-associated protein of the MAGUK family that localizes to tight junctions in epithelial cells (20), where it may function as a scaffold protein. Scaffold proteins are very important for the JNK cascade (reviewed in 21). For example, the JNK interacting proteins JIP1 and JIP2 can either enhance or inhibit JNK activation dependent on their cellular abundance (21). Overexpression of the MAPK scaffold protein POSH (Plenty of SH-3) also causes JNK activation without external stimuli (22). Recently, it was demonstrated that scaffold recruitment interaction in the yeast MAPK pathway can be replaced by PDZ domain-mediated interactions (23). If MAGI 1 functions as a scaffold protein in the JNK pathway and by sequestering components of the signaling cascade, inhibits JNK activation, then E6 may abrogate this blockade by downregulating MAGI 1 levels. E6 also activates the JNK pathway through interfering with the tumor suppressor PTEN. Like E6, PTEN contains a C-terminal PL and has been shown to bind to MAGI 1, MAGI 2 and MAGI 3 (24). PTEN dephosphorylates and thereby inhibits Focal Adhesion Kinase activation (FAK) (25). Importantly, activation of FAK can lead to JNK activation (26). Our hypothesis is that E6, by disrupting the MAGI/PTEN interaction, prevents PTEN from dephosphorylating and deactivating FAK, thus leading to higher JNK activity. Supportive evidence comes from recent studies on transgenic mice showing that a conditional null mutant for PTEN and an E6 transgene give rise to a similar phenotype. Keratinocyte-specific PTEN deficiency in mice resulted in epidermal hyperplasia and tumor formation (27) and keratinocyte-specific E6 expression resulted in epidermal hyperplasia and caused skin tumors (28). In addition, it was shown that this E6 phenotype is dependent on the presence of the E6 PL (29). Our results show that both the JNK pathway and MAGI 1 constitute potential targets for therapeutic treatments of cervical cancer. The JNK pathway has previously been implicated in cellular transformation and shown to mediate proliferation and tumor growth of human prostate carcinomas (30). We are currently investigating the role of MAGI 1 in epithelial cell transformation.

### REFERENCES

1. K. Munger, P. M. Howley, Virus Res 89, 213 (2002)
2. N. Takebe N, Y. Tsunokawa, S. Nozawa, M. Terada, T. Sugimura Biochem Biophys Res Commun 143, 837 (1987)
3. S. Lee, R. Weiss, R. Javier Proc. Natl. Acad. Sci 94, 6670 (1996)
4. T. Kiyono et al., Proc Natl. Acad. Sci 94, 6670 (1996)
5. N. Munoz et al., N Engl J Med 348, 518 (2003)
6. M. Scheffner et al., Cell 75, 495 (1993)
7. X. Tong, P. M. Howley Proc. Natl. Acad. Sci 94, 4412 (1997)
8. L. V. Ronco et al., A. Y. Karpova, M. Vidal, P. M. Howley Genes Dev 12, 2061 (1998)
9. M. Thomas, L. Banks, Oncogene 17, 2943 (1998)
10. S. S. Lee et al., J. Virol. 74, 9680 (2000)
11. S. Nakagawa, J. M. Huibrgtse, Mol Cell Biol 20, 8244 (2000)
12. B. A. Glaunsinger, S. S. Lee, M. Thomas, L. Banks, R. Javier, Oncogene 19, 5270 (2000)
13. M. Thomas et al., Oncogene 21, 5088 (2002)
14. T. M. Glaunsinger, D. Pim, R. Javier, L. Banks, Oncogene 20, 5431 (2001)
15. D. Gardiol et al., Oncogene 18, 5487 (1999)
16. F. Rosl, B. C. Das, M. Lengert, K. Geleneky, H. zurHausen, J. Virol. 71, 362 (1997)
17. J. J. Li, N. H. Colburn Oncogene 16 2711 (1998)
18. A. J. Whitmarsh, R. J. Davis J. Mol. Med 74, 589 (1996)
19. C. P. Bagowski, W. Xiong, J. E. Ferrell J Biol. Chem. 276, 1459 (2001)
20. R. P. Laura, S. Ross, H. Koeppen, L. A. Laskey Exp Cell Res 275, 155 (2002)
21. J. Yasuda, A. J. Whitmarsh, J. Cavanagh, M. Sharma, R. J. Davis, Mol Cell Biol 19, 7245 (1999)
22. Z. Xu et al., EMBO 22 252 (2003)
23. S. Park, A. Zarrinpar, W. A. Lim, Science 299 1061 (2003)
24. F. Vazquez et al., J Biol Chem 276, 48627 (2001)
25. M. Tamura, K et al., Science 280, 1614 (1998)
26. M. Oktay, K. K. Wary, M. Dans, R. Birge, F. G. Giancotti, J Cell Biol 145, 1461 (1999)
27. A Suzuki et al., Cancer Res 63, 674 (2003)
28. S. Song, Pitot H., P. F. Lambert, J. Virol 73, 5887 (1999)
29. M. L. Nguyen et al., J. Virol 77, 6957 (2003)
30. Yang Y M et al., Clinical Cancer Res 9, 391 (2003)

### EXAMPLE 13

### EC50 Determination for PDZ Domain Interactions with HPV16 E6

Using the G-assay described above, several GST-PDZ domain fusion proteins were tested to determine their relative binding strength to the PL of the HPV16 E6 protein. Peptide corresponding to the PL of HPV16 E6 was titrated against a constant amount of GST-PDZ domain fusion and the results are shown below. These results demonstrate that although a number of PDZ domains can bind the E6 protein from HPV16, the first functional domain of MAGI1 (domain 2 in this specification) binds the most tightly, making it the most suitable for. diagnostic purposes. This is unexpected, especially in conjunction with MAGI1 being the only PDZ domain containing protein demonstrated to bind to all classes of oncogenic E6 proteins identified. Together, disruption of this interaction represents a useful therapy for oncogenic HPV infections.

TABLE 11
EC50 values for HPV16 E6 protein with various PDZ domains

| | | RNA |
|---|---|---|
| | | expression (Cervical |
| PDZ gene | EC50^{a} [uM] | cell lines) |
| Magi1C (PDZ2) | 0.056 | ++ |
| Magi3 (PDZ1) | 0.31 | neg. |
| SAST1 KIAA | 0.58 | neg. |
| TIP1 | 0.75 | +++ |
| VARTUL | 0.94 | + |
| DLG1 (PDZ2) | ND | ++++ |
| PSD95 (PDZ1-3) | 1.0 | ND |
| SAST2 | 1.2 | ND |
| DLG2 (PDZ3) | 1.6 | ND |
| DLG3 (PDZ1-2) | 3.8 | ND |
| PSD95 (PDZ2) | 6.8 | ND |
| SIP1 (PDZ1) | 7.5 | ND |

Table 7 legend:
ND = not done.

The present invention is not to be limited in scope by the exemplified embodiments which are intended as illustrations of single aspects of the invention and any sequences which are functionally equivalent are within the scope of the invention. Indeed, various modifications of the invention in addition to those shown and described herein will become apparent to those skilled in the art from the foregoing description and accompanying drawings. Such modifications are intended to fall within the scope of the appended claims.

All publications cited herein are incorporated by reference in their entirety and for all purposes.

TABLE 8
PDZ Domains Used in Assays of the Invention

| | | | |
|---|---|---|---|
| Gene | GI or | PDZ | |
| Name | Acc# | # | Sequence fused to GST Construct |

| | | | |
|---|---|---|---|
| 26s sub- | 9184389 | 1 | RDMAEAHKEAMSRKLGQSESQGPPRAF |
| unit p27 | | | AKVNSISPGSPSIAGLQVDDEIVEFGS |
| | | | VNTQNFQSLHNIGSVVQHSEGALAPTI |
| | | | LLSVSM |
| | | | (SEQ ID NO:100) |
| AF6 | 430993 | 1 | LRKEPEIITVTLKKQNGMGLSIVAAKG |
| | | | AGQDKLGIYVKSVVKGGAADVDGRLAA |
| | | | GDQLLSVDGRSLVGLSQERAAELMTRT |
| | | | SSVVTLEVAKQG |
| | | | (SEQ ID NO:101) |
| AIPC | 12751451 | 1 | LIRPSVISIIGLYKEKGKGLGFSIAGG |
| | | | RDCIRGQMGIFVKTIFPNGSAAEDGRL |
| | | | KEGDEILDVNGIPIKGLTFQEAIHTFK |
| | | | QIRSGLFVLTVRTKLVSPSLTNSS |
| | | | (SEQ ID NO:102) |
| AIPC | 12751451 | 2 | GISSLGRKTPGPKDRIVMEVTLNKEPR |
| | | | VGLGIGACCLALENSPPGIYIHSLAPG |
| | | | SVAKMESNLSRGDQILEVNSVNVRHAA |
| | | | LSKVHAILSKCPPGPVRLVIGRHPNPK |
| | | | VSEQEMDEVIARSTYQESKEANSS |
| | | | (SEQ ID NO:103) |
| AIPC | 12751451 | 3 | QSENEEDVCFIVLNRKEGSGLGFSVAG |
| | | | GTDVEPKSITVHRVFSQGAASQEGTMN |
| | | | RGDFLLSVNGASLAGLAHGNVLKVLHQ |
| | | | AQLHKDALVIKKGMDQPRPSNSS |
| | | | (SEQ ID NO:104) |
| AIPC | 12751451 | 4 | LGRSVAVHDALCVEVLKTSAGLGLSLD |
| | | | GGKSSVTGDGPLVIKRVYKGGAAEQAG |
| | | | IIEAGDEILAINGKPLVGLMHFDAWNI |
| | | | MKSVPEGPVQLLIRKHRNSS |
| | | | (SEQ ID NO:105) |
| alpha | 2773059 | 1 | QTVILPGPAAWGFRLSGGIDFNQPLVI |
| actinin-2 | | | TRITPGSKAAAANLCPGDVILAIDGFG |
| associated | | | TESMTHADGQDRIKAAEFIV |
| LIM protein | | | (SEQ ID NO:106) |
| APXL-1 | 13651263 | 1 | ILVEVQLSGGAPWGFTLKGGREHGEPL |
| | | | VITKIEEGSKAAAVDKLLAGDEIVGIN |
| | | | DIGLSGFRQEAICLVKGSHKTLKLVVK |
| | | | RNSS |
| | | | (SEQ ID NO:107) |
| Atrophin-1 | 2947231 | 1 | REKPLFTRDASQLKGTFLSTTLKKSNM |
| Interacting | | | GFGFTIIGGDEPDEFLQVKSVIPDGPA |
| Protein | | | AQDGKMETGDVIVYINEVCVLGHTHAD |
| | | | VVKLFQSVPIGQSVNLVLCRGYP |
| | | | (SEQ ID NO:108) |
| Atrophin-1 | 2947231 | 2 | LSGATQAELMTLTIVKGAQGFGFTIAD |
| Interacting | | | SPTGQRVKQILDIQGCPGLCEGDLIVE |
| Protein | | | INQQNVQNLSHTEVVDILKDCPIGSET |
| | | | SLIIHRGGFF |
| | | | (SEQ ID NO:109) |
| Atrophin-1 | 2947231 | 3 | HYKELDVHLRRMESGFGFRILGGDEPG |
| Interacting | | | QPILIGAVIAMGSADRDGRLHPGDELV |
| Protein | | | YVDGIPVAGKTHRYVIDLMHHAARNGQ |
| | | | VNLTVRRKVLCG |
| | | | (SEQ ID NO:110) |
| Atrophin-1 | 2947231 | 4 | EGRGISSHSLQTSDAVIHRKENEGFGF |
| Interacting | | | VIISSLNRPESGSTITVPHKIGRIIDG |
| Protein | | | SPADRCAKLKVGDRILAVNGQSIINMP |
| | | | HADIVKLIKDAGLSVTLRIIPQEEL |
| | | | (SEQ ID NO:111) |
| Atrophin-1 | 2947231 | 5 | LSDYRQPQDFDYFTVDMEKGAKGFGFS |
| Interacting | | | IRGGREYKMDLYVLRLAEDGPAIRNGR |
| Protein | | | MRVGDQIIEINGESTRDMTHARAIELI |
| | | | KSGGRRVRLLLKRGTGQ |
| | | | (SEQ ID NO:112) |
| Atrophin-1 | 2947231 | 6 | HESVIGRNPEGQLGFELKGGAENGQFP |
| Interacting | | | YLGEVKPGKVAYESGSKLVSEELLLEV |
| Protein | | | NETPVAGLTIRDVLAVIKHCKDPLRLK |
| | | | CVKQGGIHR |
| | | | (SEQ ID NO:113) |
| CARD11 | 12382772 | 1 | NLMFRKFSLERPFRPSVTSVGHVRGPG |
| | | | PSVQHTTLNGDSLTSQLTLLGGNARGS |
| | | | FVHSVKPGSLAEKAGLREGHQLLLLEG |
| | | | CIRGERQSVPLDTCTKEEAHWTIQRCS |
| | | | GPVTLHYKVNHEGYRKLV |
| | | | (SEQ ID NO:114) |
| CARD14 | 13129123 | 1 | ILSQVTMLAFQGDALLEQISVIGGNLT |
| | | | GIFIHRVTPGSAADQMALRPGTQIVMV |
| | | | DYEASEPLFKAVLEDTTLEEAVGLLRR |
| | | | VDGFCCLSVKVNTDGYKRL |
| | | | (SEQ ID NO:115) |
| CASK | 3087815 | 1 | TRVRLVQFQKNTDEPMGITLKMNELNH |
| | | | CIVARIMHGGMIHRQGTLHVGDEIREI |
| | | | NGISVANQTVEQLQKMLREMRGSITFK |
| | | | IVPSYRTQS |
| | | | (SEQ ID NO:116) |
| Connector | 3930780 | 1 | LEQKAVLEQVQLDSPLGLEIHTTSNCQ |
| Enhancer | | | HFVSQVDTQVPTDSRLQIQPGDEVVQI |
| | | | NEQVVVGWPRKNMVRELLREPAGLSLV |
| | | | LKKIPIP |
| | | | (SEQ ID NO:117) |
| Cytohesin | 3192908 | 1 | QRKLVTVEKQDNETFGFEIQSYRPQNQ |
| Binding | | | NACSSEMFTLIGKIQEDSPAHCAGLQA |
| Protein | | | GDVLANINGVSTEGFTYKQVVDLIRSS |
| | | | GNLLTIETLNG |
| | | | (SEQ ID NO:118) |
| Densin | 16755892 | 1 | RCLIQTKGQRSMDGYPEQFCVRIEKNP |
| 180 | | | GLGFSISGGISGQGNPFKPSDKGIFVT |
| | | | RVQPDGPASNLLQPGDKILQANGHSFV |
| | | | HMEHEKAVLLLKSFQNTVDLVIQRELT |
| | | | V |
| | | | (SEQ ID NO:119) |
| DLG1 | 475816 | 1 | IQVNGTDADYEYEEITLERGNSGLGFS |
| | | | IAGGTDNPHIGDDSSIFITKIITGGAA |
| | | | AQDGRLRVNDCILQVNEVDVRDVTHSK |
| | | | AVEALKEAGSIVRLYVKRRN |
| | | | (SEQ ID NO:120) |
| DLG1 | 475816 | 2 | IQLIKGPKGLGFSIAGGVGNQHIPGDN |
| | | | SIYVTKIIEGGAAHKDGKLQIGDKLLA |
| | | | VNNVCLEEVTHEEAVTALKNTSDFVYL |
| | | | KVAKPTSMYMNDGN |
| | | | (SEQ ID NO:121) |
| DLG1 | 475816 | 3 | ILHRGSTGLGFNIVGGEDGEGIFISFI |
| | | | LAGGPADLSGELRKGDRIISVNSVDLR |
| | | | AASHEQAAAALKNAGQAVTIVAQYRPE |
| | | | EYSR |
| | | | (SEQ ID NO:122) |
| DLG2 | 12736552 | 1 | ISYVNGTEIEYEFEEITLERGNSGLGF |
| | | | SIAGGTDNPHIGDDPGIFITKIIPGGA |
| | | | AAEDGRLRVNDCILRVNEVDVSEVSHS |
| | | | KAVEALKEAGSIVRLYVRRR |
| | | | (SEQ ID NO:123) |
| DLG2 | 12736552 | 2 | ISVVEIKLFKGPKGLGFSIAGGVGNQH |
| | | | IPGDNSIYVTKIIDGGAAQKDGRLQVG |
| | | | DRLLMVNNYSLEEVTHEEAVAILKNTS |
| | | | EVVYLKVGNPTTI |
| | | | (SEQ ID NO:124) |
| DLG2 | 12736552 | 3 | IWAVSLEGEPRKVVLHKGSTGLGFNIV |
| | | | GGEDGEGIFVSFILAGGPADLSGELQR |
| | | | GDQILSVNGIDLRGASHEQAAAALKGA |
| | | | GQTVTIIAQYQPED |
| | | | (SEQ ID NO:125) |
| DLG5 | 3650451 | 1 | GIPYVEEPRHVKVQKGSEPLGISIVSG |
| | | | EKGGIYVSKVTVGSIAHQAGLEYGDQL |
| | | | LEFNGINLRSATEQQARLIIGQQCDTI |
| | | | TILAQYNPHVHQLRNSSZLTD |
| | | | (SEQ ID NO:126) |
| DLG5 | 3650451 | 2 | GILAGDANKKTLEPRVVFIKKSQLELG |
| | | | VHLCGGNLHGVFVAEVEDDSPAKGPDG |
| | | | LVPGDLILEYGSLDVRNKTVEEVYVEM |
| | | | LKPRDGVRLKVQYRPEEFIVTD |
| | | | (SEQ ID NO:127) |
| DLG6, | 14647140 | 1 | PTSPEIQELRQMLQAPHFKALLSAHDT |
| splice | | | IAQKDFEPLLPPLPDNIPESEEAMRIV |
| variant 1 | | | CLVKNQQPLGATIKRHEMTGDILVARI |
| | | | IHGGLAERSGLLYAGDKLVEVNGVSVE |
| | | | GLDPEQVIHILAMSRGTIMFKWPVSDP |
| | | | PVNSS |
| | | | (SEQ ID NO:128) |
| DLG6, | AB053303 | 1 | PTSPEIQELRQMLQAPHFKGATIKRHE |
| splice | | | MTGDILVARIIHGGLAERSGLLYAGDK |
| variant 2 | | | LVEVNGVSVEGLDPEQVIHILAMSRGT |
| | | | IMFKWPVSDPPVNSS |
| | | | (SEQ ID NO:129) |
| DVL1 | 2291005 | 1 | LNIVTVTLNMERHHFLGISIVGQSNDR |
| | | | GDGGIYIGSIMKGGAVAADGRIEPGDM |
| | | | LLQVNDVNFENMSNDDAVRVLREIVSQ |
| | | | TGPISLTVAKCW |
| | | | (SEQ ID NO:130) |
| DVL2 | 2291007 | 1 | LNIITVTLNMEKYNFLGISIVGQSNER |
| | | | GDGGIYIGSIMKGGAVAADGRIEPGDM |
| | | | LLQVNDMNFENMSNDDAVRVLRDIVHK |
| | | | PGPIVLTVAKCWDPSPQNS |
| | | | (SEQ ID NO:131) |
| DVL3 | 6806886 | 1 | IITVTLNMEKYNFLGISIVGQSNERGD |
| | | | GGIYIGSIMKGGAVAADGRIEPGDMLL |
| | | | QVNEINFENMSNDDAVRVLREIVHKPG |
| | | | PITLTVAKCWDPSP |
| | | | (SEQ ID NO:132) |
| ELFIN 1 | 2957144 | 1 | TTQQIDLQGPGPWGFRLVGRKDFEQPL |
| | | | AISRVTPGSKAALANLCIGDVITAIDG |
| | | | ENTSNMTHLEAQNRIKGCTDNLTLTVA |
| | | | RSEHKVWSPLV |
| | | | (SEQ ID NO:133) |
| ENIGMA | 561636 | 1 | IFMDSFKVVLEGPAPWGFRLQGGKDFN |
| | | | VPLSISRLTPGGKAAQAGVAVGDWVLS |
| | | | IDGENAGSLTHIEAQNKIRACGERLSL |
| | | | GLSRAQPV |
| | | | (SEQ ID NO:134) |
| ERBIN | 8923908 | 1 | QGHELAKQEIRVRVEKDPELGFSISGG |
| | | | VGGRGNPFRPDDDGIFVTRVQPEGPAS |
| | | | KLLQPGDKIIQANGYSFINIEHGQAVS |
| | | | LLKTFQNTVELIIVREVSS |
| | | | (SEQ ID NO:135) |
| EZRIN | 3220018 | 1 | ILCCLEKGPNGYGFHLHGEKGKLGQYI |
| Binding | | | RLVEPGSPAEKAGLLAGDRLVEVNGEN |
| Protein 50 | | | VEKETHQQVVSRIRAALNAVRLLVVDP |
| | | | EFIVTD |
| | | | (SEQ ID NO:136) |
| EZRIN | 3220018 | 2 | IRLCTMKKGPSGYGFNLHSDKSKPGQF |
| Binding | | | IRSVDPDSPAEASGLRAQDRIVEVNGV |
| Protein 50 | | | CMEGKQHGDWSAIRAGGDETKLLVVDR |
| | | | ETDEFFMNSS |
| | | | (SEQ ID NO:137) |
| FLJ00011 | 10440352 | 1 | KNPSGELKTVTLSKMKQSLGISISGGI |
| | | | ESKVQPMVKIEKIFPGGAAFLSGALQA |
| | | | GFELVAVDGENLEQVTHQRAVDTIRRA |
| | | | YRNKAREPMELVVRVPGPSPRPSPSD |
| | | | (SEQ ID NO:138) |
| FLJ11215 | 11436365 | 1 | EGHSHPRVVELPKTEEGLGFNIMGGKE |
| | | | QNSPIYISRIIPGGIADRHGGLKRGDQ |
| | | | LLSVNGVSVEGEHHEKAVELLKAAQGK |
| | | | VKLWRYTPKVLEEME |
| | | | (SEQ ID NO:139) |
| FLJ12428 | BC012040 | 1 | PGAPYARKTFTIVGDAVGWGFVVRGSK |
| | | | PCHIQAVDPSGPAAAAGMKVCQFVVSV |
| | | | NGLNVLHVDYRTVSNLILTGPRTIVME |
| | | | VMEELEC |
| | | | (SEQ ID NO:140) |
| FLJ12615 | 10434209 | 1 | GQYGGETVKIVRIEKARDIPLGATVRN |
| | | | EMDSVIISRIVKGGAAEKSGLLHEGDE |
| | | | VLEINGIEIRGKDVNEVFDLLSDMHGT |
| | | | LTFVLIPSQQIKPPPA |
| | | | (SEQ ID NO:141) |
| FLJ20075 | 7019938 | 1 | ILAHVKGIEKEVNVYKSEDSLGLTITD |
| | | | NGVGYAFIKRIKDGGVIDSVKTICVGD |
| | | | HIESINGENIVGWRHYDVAKKLKELKK |
| | | | EELFTMKLIEPKKAFEI |
| | | | (SEQ ID NO:142) |
| FLJ21687 | 10437836 | 1 | KPSQASGHFSVELVRGYAGFGLTLGGG |
| | | | RDVAGDTPLAVRGLLKDGPAQRCGRLE |
| | | | VGDLVLHINGESTQGLTHAQAVERIRA |
| | | | GGPQLHLVIRRPLETHPGKPRGV |
| | | | (SEQ ID NO:143) |
| FLJ31349 | AK055911 | 1 | PVMSQCACLEEVHLPNIKPGEGLGMYI |
| | | | KSTYDGLHVITGTTENSPADRSQKIHA |
| | | | GDEVIQVNQQTVVGWQLKNLVKKLREN |
| | | | PTGVVLLLKKRPTGSFNFTPEFIVTD |
| | | | (SEQ ID NO:144) |
| FLJ32798 | AK057360 | 1 | LDDEEDSVKIIRLVKNREPLGATIKKD |
| | | | EQTGAIIVARIMRGGAADRSGLIHVGD |
| | | | ELREVNGIPVEDKRPEEIIQILAQSQG |
| | | | AITFKIIPGSKEETPSNSS |
| | | | (SEQ ID NO:145) |
| GRIP 1 | 4539083 | 1 | VVELMKKEGTTLGLTVSGGIDKDGKPR |
| | | | VSNLRQGGIAARSDQLDVGDYIKAVNG |
| | | | INLAKFRHDEIISLLKNVGERVVLEVE |
| | | | YE |
| | | | (SEQ ID NO:146) |
| GRIP 1 | 4539083 | 2 | RSSVIFRTVEVTLHKEGNTFGFVIRGG |
| | | | AHDDRNKSRPVVITCVRPGGPADREGT |
| | | | IKPGDRLLSVDGIRLLGTTHAEAMSIL |
| | | | KQCGQEAALLIEYDVSVMDSVATASGN |
| | | | SS |
| | | | (SEQ ID NO:147) |
| GRIP 1 | 4539083 | 3 | HVATASGPLLVEVAKTPGASLGVALTT |
| | | | SMCCNKQVIVIDKIKSASIADRCGALH |
| | | | VGDHILSIDGTSMEYCTLAEATQFLAN |
| | | | TTDQVKLEILPHHQTRLALKGPNSS |
| | | | (SEQ ID NO:148) |
| GRIP 1 | 4539083 | 4 | TETTEVVLTADPVTGFGIQLQGSVFAT |
| | | | ETLSSPPLISYIEADSPAERCGVLQIG |
| | | | DRVMAINGIPTEDSTFEEASQLLRDSS |
| | | | ITSKVTLEIEFDVAES |
| | | | (SEQ ID NO:149) |
| GRIP 1 | 4539083 | 5 | AESVIPSSGTFHVKLPKKHNVELGITI |
| | | | SSPSSRKPGDPLVISDIKKGSVAHRTG |
| | | | TLELGDKLLAIDNIRLDNCSMEDAVQI |
| | | | LQQCEDLVKLKIRKDEDNSD |
| | | | (SEQ ID NO:150) |
| GRIP 1 | 4539083 | 6 | IYTVELKRYGGPLGITISGTEEPFDPI |
| | | | IISSLTKGGLAERTGAIHIGDRILAIN |
| | | | SSSLKGKPLSEAIHLLQMAGETVTLKI |
| | | | KKQTDAQSA |
| | | | (SEQ ID NO:151) |
| GRIP 1 | 4539083 | 7 | IMSPTPVELHKVTLYKDSDMEDFGFSV |
| | | | ADGLLEKGVYVKNIRPAGPGDLGGLKP |
| | | | YDRLLQVNHVRTRDFDCCLVVPLIAES |
| | | | GNKLDLVISRNPLA |
| | | | (SEQ ID NO:152) |
| GTPase | 2389008 | 1 | SRGCETRELALPRDGQGRLGFEVDAEG |
| Activating | | | FVTHVERFTFAETAGLRPGARLLRVCG |
| Enzyme | | | QTLPSLRPEAAAQLLRSAPKVCVTVLP |
| | | | PDESGRP |
| | | | (SEQ ID NO:153) |
| Guanine | 6650765 | 1 | AKAKWRQVVLQKASRESPLQFSLNGGS |
| Exchange | | | EKGFGIFVEGVEPGSKAADSGLKRGDQ |
| Factor | | | IMEVNGQNFENITFMKAVEILRNNTHL |
| | | | ALTVKTNIFVFKEL |
| | | | (SEQ ID NO:154) |
| HEMBA | 10436367 | 1 | LENVIAKSLLIKSNEGSYGFGLEDKNK |
| 1000505 | | | VPIIKLVEKGSNAEMAGMEVGKKIFAI |
| | | | NGDLVFMRPFNEVDCFLKSCLNSRKPL |
| | | | RVLVSTKP |
| | | | (SEQ ID NO:155) |
| HEMBA | 10436367 | 2 | PRETVKIPDSADGLGFQIRGFGPSVVH |
| 1000505 | | | AVGRGTVAAAAGLHPGQCIIKVNGINV |
| | | | SKETHASVIAHVTACRKYRRPTKQDSI |
| | | | Q |
| | | | (SEQ ID NO:156) |
| HEMBA | 7022001 | 1 | EDFCYVFTVELERGPSGLGMGLIDGMH |
| 1003117 | | | THLGAPGLYIQTLLPGSPAAADGRLSL |
| | | | GDRILEVNGSSLLGLGYLRAVDLIRHG |
| | | | GKKMRFLVAKSDVETAKKI |
| | | | (SEQ ID NO:157) |
| HTRA3 | AY040094 | 1 | LTEFQDKQIKDWKKRFIGIRMRTITPS |
| | | | LVDELKASNPDFPEVSSGIYVQEVAPN |
| | | | SPSQRGGIQDGDIIVKVNGRPLVDSSE |
| | | | LQEAVLTESPLLLEVRRGNDDLLFSNS |
| | | | S |
| | | | (SEQ ID NO:158) |
| HTRA4 | AL576444 | 1 | HKKYLGLQMLSLTVPLSEELKMHYPDF |
| | | | PDVSSGVYVCKWEGTAAQSSGLRDHDV |
| | | | IVNINGKPITTTTDWKALDSDSLSMAV |
| | | | LRGKDNLLLTVNSS |
| | | | (SEQ ID NO:159) |
| INADL | 2370148 | 1 | IWQIEYIDIERPSTGGLGFSVVALRSQ |
| | | | NLGKVDIFVKDVQPGSVADRDQRLKEN |
| | | | DQILAINHTPLDQNISHQQAIALLQQT |
| | | | TGSLRLIVAREPVHTKSSTSSSE |
| | | | (SEQ ID NO:160) |
| INADL | 2370148 | 2 | PGHVEEVELINDGSGLGFGIVGGKTSG |
| | | | VVVRTIVPGGLADRDGRLQTGDHILKI |
| | | | GGTNVQGMTSEQVAQVLRNCGNSS |
| | | | (SEQ ID NO:161) |
| INADL | 2370148 | 3 | PGSDSSLFETYNVELVRKDGQSLGIRI |
| | | | VGYVGTSHTGEASGIYVKSIIPGSAAY |
| | | | HNGHIQVNDKIVAVDGVNIQGFANHDV |
| | | | VEVLRNAGQVVHLTLVRRKTSSSTSRI |
| | | | HRD |
| | | | (SEQ ID NO:162) |
| INADL | 2370148 | 4 | NSDDAELQKYSKLLPIHTLRLGVEVDS |
| | | | FDGHHYISSIVSGGPVDTLGLLQPEDE |
| | | | LLEVNGMQLYGKSRREAVSFLKEVPPP |
| | | | FTLVCCRRLFDDEAS |
| | | | (SEQ ID NO:163) |
| INADL | 2370148 | 5 | LSSPEVKIVELVKDCKGLGFSILDYQD |
| | | | PLDPTRSVIVIRSLVADGVAERSGGLL |
| | | | PGDRLVSVNEYCLDNTSLAEAVEILKA |
| | | | VPPGLVHLGICKPLVEFIVTD |
| | | | (SEQ ID NO:164) |
| INADL | 2370148 | 6 | PNFSHWGPPRIVEIFREPNVSLGISIV |
| | | | VGQTVIKRLKNGEELKGIFIKQVLEDS |
| | | | PAGKTNALKTGDKILEVSGVDLQNASH |
| | | | SEAVEAIKNAGNPVVFIVQSLSSTPRV |
| | | | IPNVHNKANSS |
| | | | (SEQ ID NO:165) |
| INADL | 2370148 | 7 | PGELHIIELEKDKNGLGLSLAGNKDRS |
| | | | RMSIFVVGINPEGPAAADGRMRIGDEL |
| | | | LEINNQILYGRSHQNASAIIKTAPSKV |
| | | | KLVFIRNEDAVNQMANSS |
| | | | (SEQ ID NO:166) |
| INADL | 2370148 | 8 | PATCPIVPGQEMIIEISKGRSGLGLSI |
| | | | VGGKDTPLNAIVIHEVYEEGAAARDGR |
| | | | LWAGDQILEVNGVDLRNSSHEEAITAL |
| | | | RQTPQKVRLVVY |
| | | | (SEQ ID NO:167) |
| KIAA0147 | 1469875 | 1 | ILTLTILRQTGGLGISIAGGKGSTPYK |
| | | | GDDEGIFISRVSEEGPAARAGVRVGDK |
| | | | LLEVNGVALQGAEHHEAVEALRGAGTA |
| | | | VQMRVWRERMVEPENAEFIVTD |
| | | | (SEQ ID NO:168) |
| KIAA0147 | 1469875 | 2 | PLRQRHVACLARSERGLGFSIAGGKGS |
| | | | TPYRAGDAGIFVSRIAEGGAAHRAGTL |
| | | | QVGDRVLSINGVDVTEARHDHAVSLLT |
| | | | MSPTIALLLEREAGG |
| | | | (SEQ ID NO:169) |
| KIAA0147 | 1469875 | 3 | ILEGPYPVEEIRLPRAGGPLGLSIVGG |
| | | | SDHSSHPFGVQEPGVFISKVLPRGLAA |
| | | | RSGLRVGDRILAVNGQDVRDATHQEAV |
| | | | SALLRPCLELSLLVRRDPAEFIVTD |
| | | | (SEQ ID NO:170) |
| KIAA0147 | 1469875 | 4 | RELCIQKAPGERLGISIRGGARGHAGN |
| | | | PRDPTDEGIFISKVSPTGAAGRDGRLR |
| | | | VGLRLLEVNQQSLLGLTHGEAVQLLRS |
| | | | VGDTLTVLVCDGFEASTDAALEVS |
| | | | (SEQ ID NO:171) |
| KIAA0303 | 2224546 | 1 | PHQPIVIHSSGKNYGFTIRAIRVYVGD |
| | | | SDIYTVHHIVWNVEEGSPACQAGLKAG |
| | | | DLITHINGEPVHGLVHTEVIELLLKSG |
| | | | NKVSITTTPF |
| | | | (SEQ ID NO:172) |
| KIAA0313 | 7657260 | 1 | ILACAAKAKRRLMTLTKPSREAPLPFI |
| | | | LLGGSEKGFGIFVDSVDSGSKATEAGL |
| | | | KRGDQILEVNGQNFENIQLSKAMEILR |
| | | | NNTHLSITVKTNLFVFKELLTNSS |
| | | | (SEQ ID NO:173) |
| KIAA0316 | 6683123 | 1 | IPPAPRKVEMRRDPVLGFGFVAGSEKP |
| | | | VVVRSVTPGGPSEGKLIPGDQIVMIND |
| | | | EPVSAAPRERVIDLVRSCKESILLTVI |
| | | | QPYPSPK |
| | | | (SEQ ID NO:174) |
| KIAA0340 | 2224620 | 1 | LNKRTTMPKDSGALLGLKVVGGKMTDL |
| | | | GRLGAFITKVKKGSLADVVGHLRAGDE |
| | | | VLEWNGKPLPGATNEEVYNIILESKSE |
| | | | PQVEIIVSRPIGDIPRIHRD |
| | | | (SEQ ID NO:175) |
| KIAA0380 | 2224700 | 1 | QRCVIIQKDQHGFGFTVSGDRIVLVQS |
| | | | VRPGGAAMKAGVKEGDRIIKVNGTMVT |
| | | | NSSHLEWKLIKSGAYVALTLLGSS |
| | | | (SEQ ID NO:176) |
| KIAA0382 | 7662087 | 1 | ILVQRCVIIQKDDNGFGLTVSGDNPVF |
| | | | VQSVKEDGAAMRAGVQTGDRIIKVNGT |
| | | | LVTHSNHLEVVKLIKSGSYVALTVQGR |
| | | | PPGNSS |
| | | | (SEQ ID NO:177) |
| KIAA0440 | 2662160 | 1 | SVEMTLRRNGLGQLGFHVNYEGIVADV |
| | | | EPYGYAWQAGLRQGSRLVEICKVAVAT |
| | | | LSHEQMIDLLRTSVTVKVVIIPPHD |
| | | | (SEQ ID NO:178) |
| KIAA0545 | 14762850 | 1 | LKVMTSGWETVDMTLRRNGLGQLGFHV |
| | | | KYDGTVAEVEDYGFAWQAGLRQGSRLV |
| | | | EICKVAVVTLTHDQMIDLLRTSVTVKV |
| | | | VIIPPFEDGTPRRGW |
| | | | (SEQ ID NO:179) |
| KIAA0559 | 3043641 | 1 | HYIFPHARIKITRDSKDHTVSGNGLGI |
| | | | RIVGGKEIPGHSGEIGAYIAKILPGGS |
| | | | AEQTGKLMEGMQVLEWNGIPLTSKTYE |
| | | | EVQSIISQQSGEAEICVRLDLNML |
| | | | (SEQ ID NO:180) |
| KIAA0561 | 3043645 | 1 | LCGSLRPPIVIHSSGKKYGFSLRAIRV |
| | | | YMGDSDVYTVHHVVWSVEDGSPAQEAG |
| | | | LRAGDLITHINGESVLGLVHMDVVELL |
| | | | LKSGNKISLRTTALENTSIKVG |
| | | | (SEQ ID NO:181) |
| KIAA0613 | 3327039 | 1 | SYSVTLTGPGPWGFRLQGGKDFNMPLT |
| | | | ISRITPGSKAAQSQLSQGDLVVAIDGV |
| | | | NTDTMTHLEAQNKIKSASYNLSLTLQK |
| | | | SKNSS |
| | | | (SEQ ID NO:182) |
| KIAA0751 | 12734165 | 1 | ISRDSGAMLGLKVVGGKMTESGRLCAF |
| | | | ITKVKKGSLADTVGHLRPGDEVLEWNG |
| | | | RLLQGATFEEVYNIILESKPEPQVELV |
| | | | VSRPIAIHRD |
| | | | (SEQ ID NO:183) |
| KIAA0807 | 3882334 | 1 | ISALGSMRPPIIIHRAGKKYGFTLRAI |
| | | | RVYMGDSDVYTVHHMVWHVEDGGPASE |
| | | | AGLRQGDLITHVNGEPVHGLVHTEVVE |
| | | | LILKSGNKVAISTTPLENSS |
| | | | (SEQ ID NO:184) |
| KIAA0858 | 4240204 | 1 | FSDMRISINQTPGKSLDFGFTIKWDIP |
| | | | GIFVASVEAGSPAEFSQLQVDDEIIAI |
| | | | NNTKFSYNDSKEWEEAMAKAQETGHLV |
| | | | MDVRRYGKAGSPE |
| | | | (SEQ ID NO:185) |
| KIAA0902 | 4240292 | 1 | QSAHLEVIQLANIKPSEGLGMYIKSTY |
| | | | DGLHVITGTTENSPADRCKKIHAGDEV |
| | | | IQVNHQTVVGWQLKNLVNALREDPSGV |
| | | | ILTLKKRPQSMLTSAPA |
| | | | (SEQ ID NO:186) |
| KIAA0967 | 4589577 | 1 | ILTQTLIPVRHTVKIDKDTLLQDYGFH |
| | | | ISESLPLTVVAVTAGGSAHGKLFPGDQ |
| | | | ILQMNNEPAEDLSWERAVDILREAEDS |
| | | | LSITVVRCTSGVPKSSNSS |
| | | | (SEQ ID NO:187) |
| KIAA0973 | 4589589 | 1 | GLRSPITIQRSGKKYGFTLRAIRVYMG |
| | | | DTDVYSVHHIVWHVEEGGPAQEAGLCA |
| | | | GDLITHVNGEPVHGMVHPEVVELILKS |
| | | | GNKVAVTTTPFE |
| | | | (SEQ ID NO:188) |
| KIAA1095 | 5889526 | 1 | QGEETKSLTLVLHRDSGSLGFNIIGGR |
| | | | PSVDNHDGSSSEGIFVSKIVDSGPAAK |
| | | | EGGLQIHDRIIEVNGRDLSRATHDQAV |
| | | | EAFKTAKEPIVVQVLRRTPRTKMFTP |
| | | | (SEQ ID NO:189) |
| KIAA1095 | 5889526 | 2 | QEMDREELELEEVDLYRMNSQDKLGLT |
| | | | VCYRTDDEDDIGIYISEIDPNSIAAKD |
| | | | GRIREGDRIIQINGIEVQNREEAVALL |
| | | | TSEENKNFSLLIARPELQLD |
| | | | (SEQ ID NO:190) |
| KIAA1202 | 6330421 | 1 | RSFQYVPVQLQGGAPWGFTLKGGLEHC |
| | | | EPLTVSKIEDGGKAALSQKMRTGDELV |
| | | | NINGTPLYGSRQEALILIKGSFRILKL |
| | | | IVRRRNAPVS |
| | | | (SEQ ID NO:191) |
| KIAA1222 | 6330610 | 1 | ILEKLELFPVELEKDEDGLGISIIGMG |
| | | | VGADAGLEKLGIFVKTVTEGGAAQRDG |
| | | | RIQVNDQIVEVDGISLVGVTQNFAATV |
| | | | LRNTKGNVRFVIGREKPGQVS |
| | | | (SEQ ID NO:192) |
| KIAA1284 | 6331369 | 1 | KDVNVYVNPKKLTVIKAKEQLKLLEVL |
| | | | VGIIHQTKWSWRRTGKQGDGERLVVHG |
| | | | LLPGGSAMKSGQVLIGDVLVAVNDVDV |
| | | | TTENIERVLSCIPGPMQVKLTFENAYD |
| | | | VKRET |
| | | | (SEQ ID NO:193) |
| KIAA1389 | 7243158 | 1 | TRGCETVEMTLRRNGLGQLGFHVNFEG |
| | | | IVADVEPFGFAWKAGLRQGSRLVEICK |
| | | | VAVATLTHEQMIDLLRTSVTVKVVIIQ |
| | | | PHDDGSPRR |
| | | | (SEQ ID NO:194) |
| KIAA1415 | 7243210 | 1 | VENILAKRLLILPQEEDYGFDIEEKNK |
| | | | AVVVKSVQRGSLAEVAGLQVGRKIYSI |
| | | | NEDLVFLRPFSEVESILNQSFCSRRPL |
| | | | RLLVATKAKEIIKIP |
| | | | (SEQ ID NO:195) |
| KIAA1526 | 5817166 | 1 | PDSAGPGEVRLVSLRRAKAHEGLGFSI |
| | | | RGGSEHGVGIYVSLVEPGSLAEKEGLR |
| | | | VGDQILRVNDKSLARVTHAEAVKALKG |
| | | | SKKLVLSVYSAGRIPGGYVTNH |
| | | | (SEQ ID NO:196) |
| KIAA1526 | 5817166 | 2 | LQGGDEKKVNLVLGDGRSLGLTIRGGA |
| | | | EYGLGIYITGVDPGSEAEGSGLKVGDQ |
| | | | ILEVNWRSFLNILHDEAVRLLKSSRHL |
| | | | ILTVKDVGRLPHARTTVDE |
| | | | (SEQ ID NO:197) |
| KIAA1526 | 5817166 | 3 | WTSGAHVHSGPCEEKCGHPGHRQPLPR |
| | | | IVTIQRGGSAHNCGQLKVGHVILEVNG |
| | | | LTLRGKEHREAARIIAEAFKTKDRDYI |
| | | | DFLDSL |
| | | | (SEQ ID NO:198) |
| KIAA1620 | 10047316 | 1 | ELRRAELVEIIVETEAQTGVSGINVAG |
| | | | GGKEGIFVRELREDSPAARSLSLQEGD |
| | | | QLLSARVFFENFKYEDALRLLQCAEPY |
| | | | KVSFCLKRTVPTGDLALRP |
| | | | (SEQ ID NO:199) |
| KIAA1634 | 10047344 | 1 | PSQLKGVLVRASLKKSTMGFGFTIIGG |
| | | | DRPDEFLQVKNVLKDGPAAQDGKIAPG |
| | | | DVIVDINGNCVLGHTHADVVQMFQLVP |
| | | | VNQYVNLTLCRGYPLPDDSED |
| | | | (SEQ ID NO:200) |
| KIAA1634 | 10047344 | 2 | ASSGSSQPELVTIPLIKGPKGFGFAIA |
| | | | DSPTGQKVKMILDSQWCQGLQKGDIIK |
| | | | EIYHQNVQNLTHLQVVEVLKQFPVGAD |
| | | | VPLLILRGGPPSPTKTAKM |
| | | | (SEQ ID NO:201) |
| KIAA1634 | 10047344 | 3 | LYEDKPPLTNTFLISNPRTTADPRILY |
| | | | EDKPPNTKDLDVFLRKQESGFGFRVLG |
| | | | GDGPDQSIYIGAIIPLGAAEKDGRLRA |
| | | | ADELMCIDGIPVKGKSHKQVLDLMTTA |
| | | | ARNGHVLLTVRRKIFYGEKQPEDDSGS |
| | | | PGIHRELT |
| | | | (SEQ ID NO:202) |
| KIAA1634 | 10047344 | 4 | PAPQEPYDVVLQRKENEGFGFVILTSK |
| | | | NKPPPGVIPHKIGRVIEGSPADRCGKL |
| | | | KVGDHISAVNGQSIVELSHDNIVQLIK |
| | | | DAGVTVTLTVIAEEEHHGPPS |
| | | | (SEQ ID NO:203) |
| KIAA1634 | 10047344 | 5 | QNLGCYPVELERGPRGFGFSLRGGKEY |
| | | | NMGLFILRLAEDGPAIKDGRIHVGDQI |
| | | | VEINGEPTQGITHTRAIELIQAGGNKV |
| | | | LLLLRPGTGLIPDHGLA |
| | | | (SEQ ID NO:204) |
| KIAA1719 | 1267982 | 0 | ITVVELIKKEGSTLGLTISGGTDKDGK |
| | | | PRVSNLRPGGLAARSDLLNIGDYIRSV |
| | | | NGIHLTRLRHDEIITLLKNVGERVVLE |
| | | | VEY |
| | | | (SEQ ID NO:205) |
| KIAA1719 | 1267982 | 1 | ILDVSLYKEGNSFGFVLRGGAHEDGHK |
| | | | SRPLVLTYVRPGGPADREGSLKVGDRL |
| | | | LSVDGIPLHGASHATALATLRQCSHEA |
| | | | LFQVEYDVATP |
| | | | (SEQ ID NO:206) |
| KIAA1719 | 1267982 | 2 | IHTVANASGPLMVEIVKTPGSALGISL |
| | | | TTTSLRNKSVITIDRIKPASVVDRSGA |
| | | | LHPGDHILSIDGTSMEHCSLLEATKLL |
| | | | ASISEKVRLEILPVPQSQRPL |
| | | | (SEQ ID NO:207) |
| KIAA1719 | 1267982 | 3 | IQIVHTETTEVVLCGDPLSGFGLQLQG |
| | | | GIFATETLSSPPLVCFIEPDSPAERCG |
| | | | LLQVGDRVLSINGIATEDGTMEEANQL |
| | | | LRDAALAHKVVLEVEFDVAESV |
| | | | (SEQ ID NO:208) |
| KIAA1719 | 1267982 | 4 | IQFDVAESVIPSSGTFHVKLPKKRSVE |
| | | | LGITISSASRKRGEPLIISDIKKGSVA |
| | | | HRTGTLEPGDKLLAIDNIRLDNCPMED |
| | | | AVQILRQCEDLVKLKIRKDEDN |
| | | | (SEQ ID NO:209) |
| KIAA1719 | 1267982 | 5 | IQTTGAVSYTVELKRYGGPLGITISGT |
| | | | EEPFDPIVISGLTKRGLAERTGAIHVG |
| | | | DRILAINNVSLKGRPLSEAIHLLQVAG |
| | | | ETVTLKIKKQLDR |
| | | | (SEQ ID NO:210) |
| KIAA1719 | 1267982 | 6 | ILEMEELLLPTPLEMHKVTLHKDPMRH |
| | | | DFGFSVSDGLLEKGVYVHTVRPDGPAH |
| | | | RGGLQPFDRVLQVNHVRTRDFDCCLAV |
| | | | PLLAEAGDVLELIISRKPHTAHSS |
| | | | (SEQ ID NO:211) |
| LIM | 12734250 | 1 | MALTVDVAGPAPWGFRITGGRDFHTPI |
| Mystique | | | MVTKVAERGKAKDADLRPGDIIVAING |
| | | | ESAEGMLHAEAQSKIRQSPSPLRLQLD |
| | | | RSQATSPGQT |
| | | | (SEQ ID NO:212) |
| LIM | 3108092 | 1 | SNYSVSLVGPAPWGFRLQGGKDFNMPL |
| Protein | | | TISSLKDGGKAAQANVRIGDVVLSIDG |
| | | | INAQGMTHLEAQNKIKGCTGSLNMTLQ |
| | | | RAS |
| | | | (SEQ ID NO:213) |
| LIMK1 | 4587498 | 1 | TLVEHSKLYCGHCYYQTVVTPVIEQIL |
| | | | PDSPGSHLPHTVTLVSIPASSHGKRGL |
| | | | SVSIDPPHGPPGCGTEHSHTVRVQGVD |
| | | | PGCMSPDVKNSIHVGDRILEINGTPIR |
| | | | NVPLDEIDLLIQETSRLLQLTLEHD |
| | | | (SEQ ID NO:214) |
| LIMK2 | 1805593 | 1 | PYSVTLISMPATTEGRRGESVSVESAC |
| | | | SNYATTVQVKEVNRMHISPNNRNAIHP |
| | | | GDRILEINGTPVRTLRVEEVEDAISQT |
| | | | SQTLQLLIEHD |
| | | | (SEQ ID NO:215) |
| LIM-RIL | 1085021 | 1 | IHSVTLRGPSPWGFRLVGRDFSAPLTI |
| | | | SRVHAGSKASLAALCPGDLIQAINGES |
| | | | TELMTHLEAQNRIKGCHDHLTLSVSRP |
| | | | E |
| | | | (SEQ ID NO:216) |
| LU-1 | U52111 | 1 | VCYRTDDEEDLGIYVGEVNPNSIAAKD |
| | | | GRIREGDRIIQINGVDVQNREEAVAIL |
| | | | SQEENTNISLLVARPESQLA |
| | | | (SEQ ID NO:217) |
| MAGI1 | 3370997 | 1 | IQKKNHWTSRVHECTVKRGPQGELGVT |
| | | | VLGGAEHGEFPYVGAVAAVEAAGLPGG |
| | | | GEGPRLGEGELLLEVQGVRVSGLPRYD |
| | | | VLGVIDSCKEAVTFKAVRQGGR |
| | | | (SEQ ID NO:218) |
| MAGI1 | 3370997 | 2 | PSELKGKFIHTKLRKSSRGFGFTVVGG |
| | | | DEPDEFLQIKSLVLDGPAALDGKMETG |
| | | | DVIVSVNDTCVLGHTHAQVVKIFQSIP |
| | | | IGASVDLELCRGYPLPFDPDDPN |
| | | | (SEQ ID NO:219) |
| MAGI1 | 3370997 | 3 | PATQPELITVHIVKGPMGFGFTIADSP |
| | | | GGGGQRVKQIVDSPRCRGLKEGDLIVE |
| | | | VNKKNVQALTHNQVVDMLVECPKGSEV |
| | | | TLLVQRGGNLS |
| | | | (SEQ ID NO:220) |
| MAGI1 | 3370997 | 4 | PDYQEQDIFLWRKETGFGFRILGGNEP |
| | | | GEPIYIGHIVPLGAADTDGRLRSGDEL |
| | | | ICVDGTPVIGKSHQLVVQLMQQAAKQG |
| | | | HVNLTVRRKVVFAVPKTENSS |
| | | | (SEQ ID NO:221) |
| MAGI1 | 3370997 | 5 | GVVSTVVQPYDVEIRRGENEGFGFVIV |
| | | | SSVSRPEAGTTFAGNACVAMPHKIGRI |
| | | | IEGSPADRCGKLKVGDRILAVNGCSIT |
| | | | NKSHSDIVNLIKEAGNTVTLRIIPGDE |
| | | | SSNA |
| | | | (SEQ ID NO:222) |
| MAGI1 | 3370997 | 6 | QATQEQDFYTVELERGAKGFGFSLRGG |
| | | | REYNMDLYVLRLAEDGPAERCGKMRIG |
| | | | DEILEINGETTKNMKHSRAIELIKNGG |
| | | | RRVRLFLKRG |
| | | | (SEQ ID NO:223) |
| MGC5395 | BC012477 | 1 | PAKMEKEETTRELLLPNWQGSGSHGLT |
| | | | IAQRDDGVFVQEVTQNSPAARTGVVKE |
| | | | GDQIVGATIYFDNLQSGEVTQLLNTMG |
| | | | HHTVGLKLHRKGDRSPNSS |
| | | | (SEQ ID NO:224) |
| MINT1 | 2625024 | 1 | SENCKdVFIEKQKGEILGVVIVESGWG |
| | | | SILPTVIIANMMHGGPAEKSGKLNIGD |
| | | | QIMSINGTSLVGLPLSTCQSIIKGLKN |
| | | | QSRVKLNIVRCPPVNSS |
| | | | (SEQ ID NO:225) |
| MINT1 | 2625024 | 2 | LRCPPVTTVLIRRPDLRYQLGFSVQNG |
| | | | IICSLMRGGIAERGGVRVGHRIIEING |
| | | | QSVVATPHEKIVHILSNAVGEIHMKTM |
| | | | PAAMYRLLNSS |
| | | | (SEQ ID NO:226) |
| MINT3 | 3169808 | 1 | LSNSONCREVHLEKRRGEGLGVALVES |
| | | | GWGSLLPTAVIANLLHGGPAERSGALS |
| | | | IGDRLTAINGTSLVGLPLAACQAAVRE |
| | | | TKSQTSVTLSIVHCPPVTTAIM |
| | | | (SEQ ID NO:227) |
| MINT3 | 3169808 | 2 | LVHCPPVTTAIIHRPHAREQLGFCVED |
| | | | GIICSLLRGGIAERGGIRVGHRIIEIN |
| | | | GQSVVATPHARIIELLTEAYGEVHIKT |
| | | | MPAATYRLLTG |
| | | | (SEQ ID NO:228) |
| MPP1 | 189785 | 1 | RKVRLIQFEKVTEEPMGITLKLNEKQS |
| | | | CTVARILHGGMIHRQGSLHVGDEILEI |
| | | | NGTNVTNHSVDQLQKAMKETKGMISLK |
| | | | VIPNQ |
| | | | (SEQ ID NO:229) |
| MPP2 | 939884 | 1 | PVPPDAVRMVGIRKTAGEHLGVTFRVE |
| | | | GGELVIARILHGGMVAQQGLLHVGDII |
| | | | KEVNGQPVGSDPRALQELLRNASGSVI |
| | | | LKILPNYQ |
| | | | (SEQ ID NO:230) |
| MUPP1 | 2104784 | 1 | QGRHVEVFELLKPPSGGLGFSVVGLRS |
| | | | ENRGELGIFVQEIQEGSVAHRDGRLKE |
| | | | TDQILAINGQALDQTITHQQAISILQK |
| | | | AKDTVQLVIARGSLPQLV |
| | | | (SEQ ID NO:231) |
| MUPP1 | 2104784 | 2 | PVHWQHMETIELVNDGSGLGFGIIGGK |
| | | | ATGVIVKTILPGGVADQHGRLCSGDHI |
| | | | LKIGDTDLAGMSSEQVAQVLRQCGNRV |
| | | | KLMIARGAIEERTAPT |
| | | | (SEQ ID NO:232) |
| MUPP1 | 2104784 | 3 | QESETFDVELTKNVQGLGITIAGYIGD |
| | | | KKLEPSGIFVKSITKSSAVEHDGRIQI |
| | | | GDQIIAVDGTNLQGFTNQQAVEVLRHT |
| | | | GQTVLLTLMRRGMKQEA |
| | | | (SEQ ID NO:233) |
| MUPP1 | 2104784 | 4 | LNYEIVVAHVSKFSENSGLGISLEATV |
| | | | GHHFIRSVLPEGPVGHSGKLFSGDELL |
| | | | EVNGITLLGENHQDVVNILKELPIEVT |
| | | | MVCCRRTVPPT |
| | | | (SEQ ID NO:234) |
| MUPP1 | 2104784 | 5 | WEAGIQHIELEKGSKGLGFSILDYQDP |
| | | | IDPASTVIIIRSLVPGGIAEKDGRLLP |
| | | | GDRLMFVNDVNLENSSLEEAVEALKGA |
| | | | PSGTVRIGVAKPLPLSPEE |
| | | | (SEQ ID NO:235) |
| MUPP1 | 2104784 | 6 | RNVSKESFERTINIAKGNSSLGMTVSA |
| | | | NKDGLGMIVRSIIHGGAISRDGRIAIG |
| | | | DCILSINEESTISVTNAQARAMLRRHS |
| | | | LIGPDIKITYVPAEHLEE |
| | | | (SEQ ID NO:236) |
| MUPP1 | 2104784 | 7 | LNWNQPRRVELWREPSKSLGISIVGGR |
| | | | GMGSRLSNGEVMRGIFIKHVLEDSPAG |
| | | | KNGTLKPGDRIVEVDGMDLRDASHEQA |
| | | | VEAIRKAGNPVVFMVQSIINRPRKSPL |
| | | | PSLL |
| | | | (SEQ ID NO:237) |
| MUPP1 | 2104784 | 8 | LTGELHMIELEKGHSGLGLSLAGNKDR |
| | | | SRMSVFIVGIDPNGAAGKDGRLQIADE |
| | | | LLEINGQILYGRSHQNASSIIKCAPSK |
| | | | VKIIFIRNKDAVNQ |
| | | | (SEQ ID NO:238) |
| MUPP1 | 2104784 | 9 | LSSFKNVQHLELPKDQGGLGIAISEED |
| | | | TLSGVIIKSLTEHGVAATDGRLKVGDQ |
| | | | ILAVDDEIVVGYPIEKFISLLKTAKMT |
| | | | VKLTIHAENPDSQ |
| | | | (SEQ ID NO:239) |
| MUPP1 | 2104784 | 10 | LPGCETTIEISKGRTGLGLSIVGGSDT |
| | | | LLGAIIIHEVYEEGAACKDGRLWAGDQ |
| | | | ILEVNGIDLRKATHDEAINVLRQTPQR |
| | | | VRLTLYRDEAPYKE |
| | | | (SEQ ID NO:240) |
| MUPP1 | 2104784 | 11 | KEEEVCDTLTIELQKKPGKGLGLSIVG |
| | | | KRNDTGVFVSDIVKGGIADADGRLMQG |
| | | | DQILMVNGEDVRNATQEAVAALLKCSL |
| | | | GTVTLEVGRIKAGPFHS |
| | | | (SEQ ID NO:241) |
| MUPP1 | 2104784 | 12 | LQGLRTVEMKKGPTDSLGISIAGGVGS |
| | | | PLGDVPIFIAMMHPTGVAAQTQKLRVG |
| | | | DRIVTICGTSTEGMTHTQAVNLLKNAS |
| | | | GSIEMQVVAGGDVSV |
| | | | (SEQ ID NO:242) |
| MUPP1 | 2104784 | 13 | LGPPQCKSITLERGPDGLGFSIVGGYG |
| | | | SPHGDLPIYVKTVFAKGAASEDGRLKR |
| | | | GDQIIAVNGQSLEGVTHEEAVAILKRT |
| | | | KGTVTLMVLS |
| | | | (SEQ ID NO:243) |
| NeDLG | 10863920 | 1 | IQYEEIVLERGNSGLGFSIAGGIDNPH |
| | | | VPDDPGIFITKIIPGGAAAMDGRLGVN |
| | | | DCVLRVNEVEVSEVVHSRAVEALKEAG |
| | | | PVVRLVVRRRQN |
| | | | SEQ ID NO:244 |
| NeDLG | 10863920 | 2 | ITLLKGPKGLGFSIAGGIGNQHIPGDN |
| | | | SIYITKIIEGGAAQKDGRLQIGDRLLA |
| | | | VNNTNLQDVRHEEAVASLKNTSDMVYL |
| | | | KVAKPGSLE |
| | | | (SEQ ID NO:245) |
| NeDLG | 10863920 | 3 | ILLHKGSTGLGFNIVGGEDGEGIFVSF |
| | | | ILAGGPADLSGELRRGDRILSVNGVNL |
| | | | RNATHEQAAAALKRAGQSVTIVAQYRP |
| | | | EEYSRFESKIHDLREQMMNSSMSSGSG |
| | | | SLRTSEKRSLE |
| | | | (SEQ ID NO:246) |
| Neurabin II | AJ401189 | 1 | CVERLELFPVELEKDSEGLGISIIGMG |
| | | | AGADMGLEKLGIFVKTVTEGGAAHRDG |
| | | | RIQVNDLLVEVDGTSLVGVTQSFPASV |
| | | | LRNTKGRVRFMIGRERPGEQSEVAQRI |
| | | | HRD |
| | | | (SEQ ID NO:247) |
| NOS1 | 642525 | 1 | IQPNVISVRLFKRKVGGLGFLVKERVS |
| | | | KPPVIISDLIRGGAAEQSGLIQAGDII |
| | | | LAVNGRPLVDLSYDSALEVLRGIASET |
| | | | HVVLILRGP |
| | | | (SEQ ID NO:248) |
| novel PDZ | 7228177 | 1 | QANSDESDIIHSVRVEKSPAGRLGFSV |
| gene | | | RGGSEHGLGIFVSKVEEGSAERAGLCV |
| | | | GDKITEVNGLSLESTTMGSAVKVLTSS |
| | | | SRLHMMVRRMGRVPGIKFSKEKNSS |
| | | | (SEQ ID NO:249) |
| novel PDZ | 7228177 | 2 | PSDTSSEDGVRRIVHLYTTSDDFCLGF |
| gene | | | NIRGGKEFGLGIYVSKVDHGGLAEENG |
| | | | IKVGDQVLAANGVRFDDISHSQAVEVL |
| | | | KGQTHIMLTIKETGRYPAYKEMNSS |
| | | | (SEQ ID NO:250) |
| Novel | 1621243 | 1 | KIKKFLTESHDRQAKGKAITKKKYIGI |
| Serine | | | RMMSLTSSKAKELKDRHRDFPDVISGA |
| Protease | | | YIIEVIPDTPAEAGGLKENDVIISING |
| | | | QSWSANDVSDVIKRESTLNMWRRGNED |
| | | | IMITV |
| | | | (SEQ ID NO:251) |
| Numb | AK056823 | 1 | PDGEITSIKINRVDPSESLSIRLVGGS |
| Binding | | | ETPLVHIIIQHIYRDGVIARDGRLLPG |
| Protein | | | DIILKVNGMDISNVPHNYAVRLLRQPC |
| | | | QVLWLTVMREQKFRSRNSS |
| | | | (SEQ ID NO:252) |
| Numb | AK056823 | 2 | HRPRDDSFHVILNKSSPEEQLGIKLVR |
| Binding | | | KVDEPGVFIFNVLDGGVAYRHGQLEEN |
| Protein | | | DRVLAINGHDLRYGSPESAAHLIQASE |
| | | | RRVHLVVSRQVRQRSPENSS |
| | | | (SEQ ID NO:253) |
| Numb | AK056823 | 3 | PTITCHEKVVNIQKDPGESLGMTVAGG |
| Binding | | | ASHREWDLPIYVISVEPGGVISRDGRI |
| Protein | | | KTGDILLNVDGVELTEVSRSEAVALLK |
| | | | RTSSSIVLKALEVKEYEPQEFIV |
| | | | (SEQ ID NO:254) |
| Numb | AK056823 | 4 | PRCLYNCKDIVLRRNTAGSLGFCIVGG |
| Binding | | | YEEYNGNKPFFIKSIVEGTPAYNDGRI |
| Protein | | | RCGDILLAVNGRSTSGMIHACLARLLK |
| | | | ELKGRITLTIVSWPGTFL |
| | | | (SEQ ID NO:255) |
| Outer | 7023825 | 1 | LLTEEEINLTRGPSGLGFNIVGGTDQQ |
| Membrane | | | YVSNDSGIYVSRIKENGAAALDGRLQE |
| | | | GDKILSVNGQDLKNLLHQDAVDLFRNA |
| | | | GYAVSLRVQHRLQVQNGIHS |
| | | | (SEQ ID NO:256) |
| p55T | 12733367 | 1 | PVDAIRILGIHKRAGEPLGVTFRVENN |
| | | | DLVIARILHGGMIDRQGLLHVGDIIKE |
| | | | VNGHEVGNNPKELQELLKNISGSVTLK |
| | | | ILPSYRDTITPQQ |
| | | | (SEQ ID NO:257) |
| PAR3 | 8037914 | 1 | DDMVKLVEVPNDGGPLGIHVVPFSARG |
| | | | GRTLGLLVKRLEKGGKAEHENLFREND |
| | | | CIVRINDGDLRNRRFEQAQHMFRQAMR |
| | | | TPIIWFHVVWPAA |
| | | | (SEQ ID NO:258) |
| PAR3 | 8037914 | 2 | GKRLNIQLKKGTEGLGFSITSRDVTIG |
| | | | GSAPIYVKNILPRGAAIQDGRLKAGDR |
| | | | LIEVNGVDLVGKSQEEVVSLLRSTKME |
| | | | GTVSLLVFRQEDA |
| | | | (SEQ ID NO:259) |
| PAR3 | 8037914 | 3 | TPDGTREFLTFEVPLNDSGSAGLGVSV |
| | | | KGNRSKENHADLGIFVKSIINGGAASK |
| | | | DGRLRVNDQLIAVNGESLLGKTNQDAM |
| | | | ETLRRSMSTEGNKRGMIQLIVA |
| | | | (SEQ ID NO:260) |
| PAR6 | 2613011 | 1 | LPETHRRVRLHKHGSDRPLGFYIRDGM |
| | | | SVRVAPQGLERVPGIFISRLVRGGLAE |
| | | | STGLLAVSDEILEVNGIEVAGKTLDQV |
| | | | TDMMVANSHNLIVTVKPANQR |
| | | | (SEQ ID NO:261) |
| PAR6 | 13537118 | 1 | IDVDLVPETHRRVRLHRHGCEKPLGFY |
| GAMMA | | | IRDGASVRVTPHGLEKVPGIFISRMVP |
| | | | GGLAESTGLLAVNDEVLEVNGIEVAGK |
| | | | TLDQVTDMMIANSHNLIVTVKPANQRN |
| | | | NVV |
| | | | (SEQ ID NO:262) |
| PDZ-73 | 5031978 | 1 | RSRKLKEVRLDRLHPEGLGLSVRGGLE |
| | | | FGCGLFISHLIKGGQADSVGLQVGDEI |
| | | | VRINGYSISSCTHEEVINLIRTKKTVS |
| | | | IKVRHIGLIPVKSSPDEFH |
| | | | (SEQ ID NO:263) |
| PDZ-73 | 5031978 | 2 | IPGNRENKEKKVFISLVGSRGLGCSIS |
| | | | SGPIQKPGIFISHVKPGSLSAEVGLEI |
| | | | GDQIVEVNGVDFSNLDHKEAVNVLKSS |
| | | | RSLTISIVAAAGRELFMTDEF |
| | | | (SEQ ID NO:264) |
| PDZ-73 | 50319738 | 3 | PEQIMGKDVRLLRIKKEGSLDLALEGG |
| | | | VDSPIGKVVVSAVYERGAAERHGGIVK |
| | | | GDEIMAINGKIVTDYTLAEADAALQKA |
| | | | WNQGGDWIDLVVAVCPPKEYDD |
| | | | (SEQ ID NO:265) |
| PDZK1 | 2944188 | 1 | LTSTFNPRECKLSKQEGQNYGFFLRIE |
| | | | KDTEGHLVRVVEKCSPAEKAGLQDGDR |
| | | | VLRINGVFVDKEEHMQVVDLVRKSGNS |
| | | | VTLLVLDGDSYEKAGSPGIHRD |
| | | | (SEQ ID NO:266) |
| PDZK1 | 2944188 | 2 | RLCYLVKEGGSYGFSLKTVQGKKGVYM |
| | | | TDITPQGVAMRAGVLADDHLIEVNGEN |
| | | | VEDASHEEVVEKVKKSGSRVMFLLVDK |
| | | | ETDKREFIVTD |
| | | | (SEQ ID NO:267) |
| PDZK1 | 2944188 | 3 | QFKRETASLKLLPHQPRIVEMKKGSNG |
| | | | YGFYLRAGSEQKGQIIKDIDSGSPAEE |
| | | | AGLKNNDLVVAVNGESVETLDHDSVVE |
| | | | MIRKGGDQTSLLVVDKETDNMYRLAEF |
| | | | IVTD |
| | | | (SEQ ID NO:268) |
| PDZK1 | 2944188 | 4 | PDTTEEVDHKPKLCRLAKGENGYGFHL |
| | | | NAIRGLPGSFIKEVQKGGPADLAGLED |
| | | | EDVIIEVNGVNVLDEPYEKVVDRIQSS |
| | | | GKNVTLLVZGKNSS |
| | | | (SEQ ID NO:269) |
| PICK1 | 4678411 | 1 | PTVPGKVTLQKDAQNLIGISIGGGAQY |
| | | | CPCLYIVQVFDNTPAALDGTVAAGDEI |
| | | | TGVNGRSIKGKTKVEVAKMIQEVKGEV |
| | | | TIHYNKLQ |
| | | | (SEQ ID NO:270) |
| PIST | 98374330 | 1 | SQGVGPIRKVLLLKEDHEGLGISITGG |
| | | | KEHGVPILISEIHPGQPADRCGGLHVG |
| | | | DAILAVNGVNLRDTKHKEAVTILSQQR |
| | | | GEIEFEVVYVAPEVDSD |
| | | | (SEQ ID NO:271) |
| prIL16 | 1478492 | 1 | IHVTILHKEEGAGLGFSLAGGADLENK |
| | | | VITVHRVFPNGLASQEGTIQKGNEVLS |
| | | | INGKSLKGTTHHDALAILRQAREPRQA |
| | | | VIVTRKLTPEEFIVTD |
| | | | (SEQ ID NO:272) |
| prIL16 | 1478492 | 2 | TAEATVCTVTLEKMSAGLGFSLEGGKG |
| | | | SLHGDKPLTINRIFKGAASEQSETVQP |
| | | | GDEILQLGGTAMQGLTRFEAWNIIKAL |
| | | | PDGPVTIVIRRKSLQSK |
| | | | (SEQ ID NO:273) |
| PSD95 | 3318652 | 1 | LEYEeITLERGNSGLGFSIAGGTDNPH |
| | | | IGDDPSIFITKIIPGGAAAQDGRLRVN |
| | | | DSILFVNEVDVREVTHSAAVEALKEAG |
| | | | SIVRLYVMRRKPPAENSS |
| | | | (SEQ ID NO:274) |
| PSD95 | 3318652 | 2 | HVMRRKPPAEKVMEIKLIKGPKGLGFS |
| | | | IAGGVGNQHIPGDNSIYVTKIIEGGAA |
| | | | HKDGRLQIGDKILAVNSVGLEDVMHED |
| | | | AVAALKNTYDVVYLKVAKPSNAYL |
| | | | (SEQ ID NO:275) |
| PSD95 | 3318652 | 3 | REDIPREPRRIVIHRGSTGLGFNIVGG |
| | | | EDGEGIFISFILAGGPADLSGELRKGD |
| | | | QILSVNGVDLRNASHEQAAIALKNAGQ |
| | | | TVTIIAQYKPEFIVTD |
| | | | (SEQ ID NO:276) |
| PTN-3 | 179912 | 1 | LIRITPDEDGKFGFNLKGGVDQKMPLV |
| | | | VSRINPESPADTCIPKLNEGDQIVLIN |
| | | | GRDISEHTHDQVVMFIKASRESHSREL |
| | | | ALVIRRR |
| | | | (SEQ ID NO:277) |
| PTN-4 | 190747 | 1 | IRMKPDENGRFGFNVKGGYDQKMPVIV |
| | | | SRVAPGTPADLCVPRLNEGDQVVLING |
| | | | RDIAEHTHDQVVLFIKASCERHSGELM |
| | | | LLVRPNA |
| | | | (SEQ ID NO:278) |
| PTPL1 | 515030 | 1 | PEREITLVNLKKDAKYGLGFQIIGGEK |
| | | | MGRLDLGIFISSVAPGGPADFHGCLKP |
| | | | GDRLISVNSVSLEGVSHHAAIEILQNA |
| | | | PEDVTLVISQPKEKISKVPSTPVHL |
| | | | (SEQ ID NO:279) |
| PTPL1 | 515030 | 2 | GDIFEVELAKNDNSLGISVTGGVNTSV |
| | | | RHGGIYVKAVIPQGAAESDGRIHKGDR |
| | | | VLAVNGVSLEGATHKQAVETLRNTGQV |
| | | | VHLLLEKGQSPTSK |
| | | | (SEQ ID NO:280) |
| PTPL1 | 515030 | 3 | TEENTFEVKLFKNSSGLGFSFSREDNL |
| | | | IPEQINASIVRVKKLFAGQPAAESGKI |
| | | | DVGDVILKVNGASLKGLSQQEVISALR |
| | | | GTAPEVFLLLCRPPPGVLPEIDT |
| | | | (SEQ ID NO:281) |
| PTPL1 | 515030 | 4 | ELEVELLITLIKSEKASLGFTVTKGNQ |
| | | | RIGCYVHDVIQDPAKSDGRLKPGDRLI |
| | | | KVNDTDVTNMTHTDAVNLLRAASKTVR |
| | | | LVIGRVLELPRIPMLPH |
| | | | (SEQ ID NO:282) |
| PTPL1 | 515030 | 5 | MLPHLLPDITLTCNKEELGFSLCGGHD |
| | | | SLYQVVYISDINPRSVAAIEGNLQLLD |
| | | | VIHYVNGVSTQGMTLEEVNRALDMSLP |
| | | | SLVLKATRNDLPV |
| | | | (SEQ ID NO:283) |
| RGS12 | 3290015 | 1 | RPSPPRVRSVEVARGRAGYGFTLSGQA |
| | | | PCVLSCVMRGSPADFVGLRAGDQILAV |
| | | | NEINVKKASHEDVVKLIGKCSGVLHMV |
| | | | IAEGVGRFESCS |
| | | | (SEQ ID NO:284) |
| RGS3 | 18644735 | 1 | LCSERRYRQITIPRGKDGFGFTICCDS |
| | | | PVRVQAVDSGGPAERAGLQQLDTVLQL |
| | | | NERPVEHWKCVELAHEIRSCPSEIILL |
| | | | VWRMVPQVKPGIHRD |
| | | | (SEQ ID NO:285) |
| Rhophilin- | 14279408 | 1 | ISFSANKRWTPPRSIRFTAEEGDLGFT |
| like | | | LRGNAPVQVHFLDPYCSASVAGAREGD |
| | | | YIVSIQLVDCKWLTLSEVMKLLKSFGE |
| | | | DEIEMKVVSLLDSTSSMHNKSAT |
| | | | (SEQ ID NO:286) |
| Serine | 2738914 | 1 | RGEKKNSSSGISGSQRRYIGVMMLTLS |
| Protease | | | PSILAELQLREPSFPDVQHGVLIHKVI |
| | | | LGSPAHRAGLRPGDVILAIGEQMVQNA |
| | | | EDVYEAVRTQSQLAVQIRRGRETLTLY |
| | | | V |
| | | | (SEQ ID NO:287) |
| Shank 1 | 6049185 | 1 | EEKTVVLQKKDNEGFGFVLRGAKADTP |
| | | | IEEFTPTPAFPALQYLESVDEGGVAWQ |
| | | | AGLRTGDFLIEVNNENWKVGHRQVVNM |
| | | | IRQGGNHLVLKVVTVTRNLDPDDTARK |
| | | | KA |
| | | | (SEQ ID NO:288) |
| Shank 3 | * | 1 | SDYVIDDKVAVLQKRDHEGFGFVLRGA |
| | | | KAETPIEEFTPTPAFPALQYLESVDVE |
| | | | GVAWRAGLRTGDFLIEVNGVNVVKVGH |
| | | | KQVVALIRQGGNRLVMKVVSVTRKPEE |
| | | | DG |
| | | | (SEQ ID NO:289) |
| Shroom | 18652858 | 1 | IYLEAFLEGGAPWGFTLKGGLEHGEPL |
| | | | IISKVEEGGKADTLSSKLQAGDEVVHI |
| | | | NEVTLSSSRKEAVSLVKGSYKTLRLVV |
| | | | RRDVCTDPGH |
| | | | (SEQ ID NO:290) |
| SIP1 | 2047327 | 1 | IRLCRLVRGEQGYGFHLHGEKGRRGQF |
| | | | IRRVEPGSPAEAAALRAGDRLVEVNGV |
| | | | NVEGETHHQVVQRIKAVEGQTRLLVVD |
| | | | QN |
| | | | (SEQ ID NO:291) |
| SIP1 | 2047327 | 2 | IRHLRKGPQGYGFNLHSDKSRPGQYIR |
| | | | SVDPGSPAARSGLRAQDRLIEVNGQNV |
| | | | EGLRHAEVVASIKAREDEARLLVVDPE |
| | | | TDE |
| | | | (SEQ ID NO:292) |
| SITAC-18 | 8886071 | 1 | PGVREIHLCKDERGKTGLRLRKVDQGL |
| | | | FVQLVQANTPASLVGLRFGDQLLQIDG |
| | | | RDCAGWSSHKAHQVVKKASGDKIVVVV |
| | | | RDRPFQRTVTM |
| | | | (SEQ ID NO:293) |
| SITAC-18 | 8886071 | 2 | PFQRTVTMHKDSMGHVGFVIKKGKIVS |
| | | | LVKGSSAARNGLLTNHYVCEVDGQNVI |
| | | | GLKDKKIMEILATAGNVVTLTIIPSVI |
| | | | YEHIVEFIV |
| | | | (SEQ ID NO:294) |
| SSTRIP | 7025450 | 1 | LKEKTVLLQKKDSEGFGFVLRGAKAQT |
| | | | PIEEFTPTPAFPALQYLESVDEGGVAW |
| | | | RAGLRMGDFLIEVNGQNWKVGHRQVVN |
| | | | MIRQGGNTLMVKVVMVTRHPDMDEAVQ |
| | | | (SEQ ID NO:295) |
| SYNTENIN | 2795862 | 1 | LEIKQGIREVILCKDQDGKIGLRLKSI |
| | | | DNGIFVQLVQANSPASLVGLRFGDQVL |
| | | | QINGENCAGWSSDKAHKVLKQAFGEKI |
| | | | TMRIHRD |
| | | | (SEQ ID NO:296) |
| SYNTENIN | 2795862 | 2 | RDRPFERTITMHKDSTGHVGFIFKNGK |
| | | | ITSIVKDSSAARNGLLTEHNICEINGQ |
| | | | NVIGLKDSQIADILSTSGNSS |
| | | | (SEQ ID NO:297) |
| Syntrophin | 1145727 | 1 | QRRRVTVRKADAGGLGISIKGGRENKM |
| 1 alpha | | | PILISKIFKGLAADQTEALFVGDAILS |
| | | | VNGEDLSSATHDEAVQVLKKTGKEVVL |
| | | | EVKYMKDVSPYFK |
| | | | (SEQ ID NO:298) |
| Syntrophin | 476700 | 1 | IRVVKQEAGGLGISIKGGRENRMPILI |
| beta 2 | | | SKIFPGLAADQSRALRLGDAILSVNGT |
| | | | DLRQATHDQAVQALKRAGKEVLLEVKF |
| | | | IREFIVTD |
| | | | (SEQ ID NO:299) |
| Syntrophin | 9507162 | 1 | EPFYSGERTVTIRRQTVGGFGLSIKGG |
| gamma 1 | | | AEHNIPVVVSKISKEQRAELSGLLFIG |
| | | | DAILQINGINVRKCRHEEVVQVLRNAG |
| | | | EEVTLTVSFLKRAPAFLKLP |
| | | | (SEQ ID NO:300) |
| Syntrophin | 9507162 | 1 | SHQGRNRRTVTLRRQPVGGLGLSIKGG |
| gamma 2 | | | SEHNVPVVISKIFEDQAADQTGMLFVG |
| | | | DAVLQVNGIHVENATHEEVVHLLRNAG |
| | | | DEVTITVEYLREAPAFLK |
| | | | (SEQ ID NO:301) |
| TAX2-like | 3253116 | 1 | RGETKEVEVTKTEDALGLTITDNGAGY |
| protein | | | AFIKRIKEGSIINRIEAVCVGDSIEAI |
| | | | NDHSIVGCRHYEVAKMLRELPKSQPFT |
| | | | LRLVQPKRAF |
| | | | (SEQ ID NO:302) |
| TIAM 1 | 4507500 | 1 | HSIHIEKSDTAADTYGFSLSSVEEDGI |
| | | | RRLYVNSVKETGLASKKGLKAGDEILE |
| | | | INNRAADALNSSMLKDFLSQPSLGLLV |
| | | | RTYPELE |
| | | | (SEQ ID NO:303) |
| TIAM 2 | 6912703 | 1 | PLNVYDVQLTKTGSVCDFGFAVTAQVD |
| | | | ERQHLSRIFISDVLPDGLAYGEGLRKG |
| | | | NEIMTLNGEAVSDLDLKQMEALFSEKS |
| | | | VGLTLIARPPDTKATL |
| | | | (SEQ ID NO:304) |
| TIP1 | 2613001 | 1 | QRVEIHKLRQGENLILGFSIGGGIDQD |
| | | | PSQNPFSEDKTDKGIYVTRVSEGGPAE |
| | | | IAGLQIGDKIMQVNGWDMTMVTHDQAR |
| | | | KRLTKRSEEVVRLLVTRQSLQK |
| | | | (SEQ ID NO:305) |
| TIP2 | 2613003 | 1 | RKEVEVFKSEDALGLTITDNGAGYAFI |
| | | | KRIKEGSVIDHIHLISVGDMIEAINGQ |
| | | | SLLGCRHYEVARLLKELPRGRTFTLKL |
| | | | TEPRK |
| | | | (SEQ ID NO:306) |
| TIP33 | 2613007 | 1 | HSHPRVVELPKTDEGLGFNVMGGKEQN |
| | | | SPIYISRIIPGGVAERHGGLKRGDQLL |
| | | | SVNGVSVEGEHHEKAVELLKAAKDSVK |
| | | | LVVRYTPKVL |
| | | | (SEQ ID NO:307) |
| TIP43 | 2613011 | 1 | ISNQKRGVKVLKQELGGLGISIKGGKE |
| | | | NKMPILISKIFKGLAADQTQALYVGDA |
| | | | ILSVNGADLRDATHDEAVQALKRAGKE |
| | | | VLLEVKYMREATPYV |
| | | | (SEQ ID NO:308) |
| X-11 beta | 3005559 | 1 | IHFSNSENCKELQLEKHKGEILGVVVV |
| | | | ESGWGSILPTVILANMMNGGPAARSGK |
| | | | LSIGDQIMSINGTSLVGLPLATCQGII |
| | | | KGLKNQTQVKLNIVSCPPVTTVLIKRN |
| | | | SS |
| | | | (SEQ ID NO:309) |
| X-11 beta | 3005559 | 2 | IPPVTTVLIKRPDLKYQLGFSVQNGII |
| | | | CSLMRGGIAERGGVRVGHRIIEINGQS |
| | | | VVATAHEKIVQALSNSVGEIHMKTMPA |
| | | | AMFRLLTGQENSS |
| | | | (SEQ ID NO:310) |
| ZO-1 | 292937 | 1 | IWEQHTVTLHRAPGFGFGIAISGGRDN |
| | | | PHFQSGETSIVISDVLKGGPAEGQLQE |
| | | | NDRVAMVNGVSMDNVEHAFAVQQLRKS |
| | | | GKNAKITIRRKKKVQIPNSS |
| | | | (SEQ ID NO:311) |
| ZO-1 | 292937 | 2 | ISSQPAKPTKVTLVKSRKNEEYGLRLA |
| | | | SHIFVKEISQDSLAARDGNIQEGDVVL |
| | | | KINGTVTENMSLTDAKTLIERSKGKLK |
| | | | MVVQRDRATLLNSS |
| | | | (SEQ ID NO:312) |
| ZO-1 | 292937 | 3 | IRMKLVKFRKGDSVGLRLAGGNDVGIF |
| | | | VAGVLEDSPAAKEGLEEGDQILRVNNV |
| | | | DFTNIIREEAVLFLLDLPKGEEVTILA |
| | | | QKKKDVFSN |
| | | | (SEQ ID NO:313) |
| ZO-2 | 12734763 | 1 | LIWEQYTVTLQKDSKRGFGIAVSGGRD |
| | | | NPHFENGETSIVISDVLPGGPADGLLQ |
| | | | ENDRVVMVNGTPMEDVLHSFAVQQLRK |
| | | | SGKVAAIVVKRPRKV |
| | | | (SEQ ID NO:314) |
| ZO-2 | 12734763 | 2 | RVLLMKSRANEEYGLRLGSQIFVKEMT |
| | | | RTGLATKDGNLHEGDIILKINGTVTEN |
| | | | MSLTDARKLIEKSRGKLQLVVLRDS |
| | | | (SEQ ID NO:315) |
| ZO-2 | 12734763 | 3 | HAPNTKMVRFKKGDSVGLRLAGGNDVG |
| | | | IFVAGIQEGTSAEQEGLQEGDQILKVN |
| | | | TQDFRGLVREDAVLYLLEIPKGEMVTI |
| | | | LAQSRADVY |
| | | | (SEQ ID NO:316) |
| ZO-3 | 10092690 | 1 | IPGNSTIWEQHTATLSKDPRRGFGIAI |
| | | | SGGRDRPGGSMVVSDVVPGGPAEGRLQ |
| | | | TGDHIVMVNGVSMENATSAFAIQILKT |
| | | | CTKMANITVKRPRRIHLPAEFIVTD |
| | | | (SEQ ID NO:317) |
| ZO-3 | 10092690 | 2 | QDVQMKPVKSVLVKRRDSEEFGVKLGS |
| | | | QIFIKHITDSGLAARHRGLQEGDLILQ |
| | | | INGVSSQNLSLNDTRRLIEKSEGKLSL |
| | | | LVLRDRGQFLVNIPNSS |
| | | | (SEQ ID NO:318) |
| ZO-3 | 10092690 | 3 | RGYSPDTRVVRFLKGKSIGLRLAGGND |
| | | | VGIFVSGVQAGSPADGQGIQEGDQILQ |
| | | | VNDVPFQNLTREEAVQFLLGLPPGEEM |
| | | | ELVTQRKQDIFWKMVQSEFIVTD |
| | | | (SEQ ID NO:319) |

*No GI number for this PDZ domain containing protein - it was computer cloned by J.S. using rat Shank3 seq against human genomic clone AC000036. In silico spliced together nt6400-6496, 6985-7109, 7211-7400 to create hypothetical human Shank3.

What is claimed is

1. A method of screening, comprising:
determining an effect of a candidate agent on binding of an oncogenic E6 protein to a polypeptide comprising the amino acid sequence of a second PDZ domain from MAGI-1.

2. The method of claim 1, wherein said binding is detected in both the absence and presence of said candidate agent.

3. The method of claim 1, further comprising determining an effect of a plurality of candidate agents and identifying a candidate agent that reduces said binding.

4. The method of claim 1, further comprising testing said agent in a cellular assay for HPV oncogenicity.

5. The method of claim 1, wherein said candidate agent is small molecule, antibody or peptide.

6. The method of claim 1, wherein said determining is a cellular assay.

7. The method of claim 1, wherein said oncogenic E6 protein and said polypeptide are isolated.

8. An isolated peptide comprising an amino acid sequence corresponding to two contiguous amino acids at the C-terminus of an oncogenic E6 protein.

9. The isolated peptide of claim 1, wherein said peptide no greater than 5 amino acids in length.

10. The isolated peptide of claim 1, wherein said peptide contains non-amino acid moieties bonded to its C- or N-terminus.

11. The isolated peptide of claim 10, wherein said peptide contains a carboxyl, hydroxyl or tetrazole group at its C-terminus and a moiety selected from those shown in FIG. 11 at its N-terminus.

12. The isolated peptide of claim 8, further comprising a cell permeable peptide carrier moiety.

13. The isolated peptide of claim 8, wherein said two contiguous amino acids are at the C-terminus of said isolated peptide.

14. A pharmaceutical composition comprising:
the isolated peptide of claim 8; and
a pharmaceutically acceptable carrier.

15. A method of modulating an interaction between a MAGI-1 protein and an oncogenic E6 protein, comprising:
contacting said MAGI-1 protein with an isolated peptide of claim 8.

16. A method of reducing the oncogenicity of an oncogenic strain of HPV in a cell, comprising:
reducing binding of an E6 protein of said HPV to a MAGI-1 protein of said cell.

17. The method of claim 16, wherein said cell is a cell in vitro.

18. The method of claim 16, wherein said cell is a cell in vivo.

19. The method of claim 16, wherein said reducing binding is done by contacting said E6 protein with a peptide of claim 8.

20. A method of treating a cancer associated with HPV infection, comprising,
administering to a subject in need thereof the pharmaceutical composition of claim 14.

21. The method of claim 20, wherein said subject has cervical cancer, uterine cancer, anal cancer, colorectal cancer, penile cancer, oral cancer, skin cancer or esophageal cancer.

22. A kit comprising,
the isolated peptide of claim 8; and
instructions for using said peptide to treat a cancer associated with HPV infection.


SEQUENCE LISTING (357 sequences)

SEQ ID NO: 1 - protein, 5 aa, Homo sapiens
GGGGS

SEQ ID NO: 2 - protein, 12 aa, Homo sapiens
KSSGSGSESKVD

SEQ ID NO: 3 - protein, 18 aa, Homo sapiens
KESGSVSSEQLAQFRSLD

SEQ ID NO: 4 - protein, 13 aa, Homo sapiens
GYGRKKRRQRRRG

SEQ ID NO: 5 - protein, 10 aa, Homo sapiens
GYCRNCIRKQ

SEQ ID NO: 6 - protein, 10 aa, Homo sapiens
WTTCMEDLLP

SEQ ID NO: 7 - protein, 10 aa, Homo sapiens
GICRLCKHFQ

SEQ ID NO: 8 - protein, 10 aa, Homo sapiens
KGLCRQCKQI

SEQ ID NO: 9 - protein, 10 aa, Homo sapiens
WLRCTVRIPQ

SEQ ID NO: 10 - protein, 10 aa, Homo sapiens
RQCKHFYNDW

SEQ ID NO: 11 - protein, 10 aa, Homo sapiens
CRNCISHEGR

SEQ ID NO: 12 - protein, 10 aa, Homo sapiens
CCRNCYEHEG

SEQ ID NO: 13 - protein, 10 aa, Homo sapiens
SSRTRRETQL

SEQ ID NO: 14 - protein, 10 aa, Homo sapiens
RLQRRRETQV

SEQ ID NO: 15 - protein, 10 aa, Homo sapiens
RPRRQTETQV

SEQ ID NO: 16 - protein, 10 aa, Homo sapiens
RRTLRRETQV

SEQ ID NO: 17 - protein, 10 aa, Homo sapiens
WRRPRTETQV

SEQ ID NO: 18 - protein, 10 aa, Homo sapiens
RLQRRRETAL

SEQ ID NO: 19 - protein, 10 aa, Homo sapiens
WKPTRRETEV

SEQ ID NO: 20 - protein, 10 aa, Homo sapiens
RRLTRRETQV

SEQ ID NO: 21 - protein, 10 aa, Homo sapiens
RLRRRRETQV

SEQ ID NO: 22 - protein, 10 aa, Homo sapiens
RLQRRNETQV

SEQ ID NO: 23 - protein, 10 aa, Homo sapiens
RLQRRRVTQV

SEQ ID NO: 24 - protein, 10 aa, Homo sapiens
RHTTATESAV

SEQ ID NO: 25 - protein, 10 aa, Homo sapiens
TSREPRESTV

SEQ ID NO: 26 - protein, 10 aa, Homo sapiens
RLQRRRQTQV

SEQ ID NO: 27 - protein, 10 aa, Homo sapiens
QRQARSETLV

SEQ ID NO: 28 - protein, 10 aa, Homo sapiens
TSRQATESTV

SEQ ID NO: 29 - protein, 10 aa, Homo sapiens
RRRTRQETQV

SEQ ID NO: 30 - protein, 10 aa, Homo sapiens
RRREATETQV

SEQ ID NO: 31 - protein, 10 aa, Homo sapiens
RCWRPSATVV

SEQ ID NO: 32 - protein, 10 aa, Homo sapiens
PPRQRSETQV

SEQ ID NO: 33 - DNA, 24 bp, Homo sapiens
aaaagatctacaatactatggcgc

SEQ ID NO: 34 - DNA, 26 bp, Homo sapiens
agggaattccagacttaatattatac

SEQ ID NO: 35 - DNA, 26 bp, Homo sapiens
aaaggatccattttatgcaccaaaag

SEQ ID NO: 36 - DNA, 28 bp, Homo sapiens
atggaattctatctccatgcatgattac

SEQ ID NO: 37 - DNA, 26 bp, Homo sapiens
gaggaattcaccacaatactatggcg

SEQ ID NO: 38 - DNA, 26 bp, Homo sapiens
aggagatctcatacttaatattatac

SEQ ID NO: 39 - DNA, 27 bp, Homo sapiens
ttgagatcttcagcgtcgttggagtcg

SEQ ID NO: 40 - DNA, 26 bp, Homo sapiens
aaagaattcattttatgcaccaaaag

SEQ ID NO: 41 - DNA, 28 bp, Homo sapiens
atgggatcctatctccatgcatgattac

SEQ ID NO: 42 - DNA, 32 bp, Homo sapiens
ctgggatcctcatcaacgtgttcttgatgatc

SEQ ID NO: 43 - DNA, 27 bp, Homo sapiens
aagaaagctttttatgcaccaaaagag

SEQ ID NO: 44 - DNA, 29 bp, Homo sapiens
aatcaagctttatctccatgcatgattac

SEQ ID NO: 45 - DNA, 30 bp, Homo sapiens
gctgaagctttcaacgtgttcttgatgatc

SEQ ID NO: 46 - DNA, 27 bp, Homo sapiens
aagcgtcgactttatgcaccaaaagag

SEQ ID NO: 47 - DNA, 29 bp, Homo sapiens
aatgctcgagtatctccatgcatgattac

SEQ ID NO: 48 - DNA, 30 bp, Homo sapiens
gctgctcgagtcaacgtgttcttgatgatc

SEQ ID NO: 49 - DNA, 26 bp, Homo sapiens
agaagtcgaccacaatactatggcgc

SEQ ID NO: 50 - DNA, 27 bp, Homo sapiens
taggctcgagcatacttaatattatac

SEQ ID NO: 51 - DNA, 28 bp, Homo sapiens
cttgctcgagtcagcgtcgttggagtcg

SEQ ID NO: 52 - DNA, 26 bp, Homo sapiens
agaaaagcttcacaatactatggcgc

SEQ ID NO: 53 - DNA, 27 bp, Homo sapiens
tagaagcttgcatacttaatattatac

SEQ ID NO: 54 - DNA, 28 bp, Homo sapiens
cttgaagctttcagcgtcgttgaggtcg

SEQ ID NO: 55 - protein, 225 aa, Homo sapiens
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLIEGR

SEQ ID NO: 56 - DNA, 24 bp, Homo sapiens
aatggggatccagctcattaaagg

SEQ ID NO: 57 - DNA, 24 bp, Homo sapiens
atacatacttgtggaattcgccac

SEQ ID NO: 58 - DNA, 26 bp, Homo sapiens
cacggatcccttctgagttgaaaggc

SEQ ID NO: 59 - DNA, 30 bp, Homo sapiens
tatgaattccatctggatcaaaaggcaatg

SEQ ID NO: 60 - DNA, 30 bp, Homo sapiens
cagggatccaaagagttgaaattcacaagc

SEQ ID NO: 61 - DNA, 27 bp, Homo sapiens
acggaattctgcagcgactgccgcgtc

SEQ ID NO: 62 - DNA, 23 bp, Homo sapiens
aggatccagatgtcctacatccc

SEQ ID NO: 63 - DNA, 23 bp, Homo sapiens
ggaattcatggactgctgcacgg

SEQ ID NO: 64 - DNA, 28 bp, Homo sapiens
agagaattctcgagatgtcctacatccc

SEQ ID NO: 65 - DNA, 27 bp, Homo sapiens
tgggaattcctaggacagcatggactg

SEQ ID NO: 66 - DNA, 25 bp, Homo sapiens
ctaggatccgggccagccggtcacc

SEQ ID NO: 67 - DNA, 29 bp, Homo sapiens
gacggatccccctgctgcacggccttctg

SEQ ID NO: 68 - DNA, 29 bp, Homo sapiens
gacgaattcccctgctgcacggccttctg

SEQ ID NO: 69 - DNA, 25 bp, Homo sapiens
ctagaattcgggccagccggtcacc

SEQ ID NO: 70 - protein, 101 aa, Homo sapiens
PSELKGKFIHTKLRKSSRGFGFTVVGGDEPDEFLQIKSLVLDGPAALDGKMETGDVIVSVNDTCVLGHTHAQVVKIFQSIPIGASVDLELCRGYPLPFDPD

SEQ ID NO: 71 - protein, 102 aa, Homo sapiens
QRVEIHKLRQGENLILGFSIGGGIDQDPSQNPFSEDKTDKGIYVTRVSEGGPAEIAGLQIGDKIMQVNGWDMTMVTHDQARKRLTKRSEEVVRLLVTRQSLQ

SEQ ID NO: 72 - protein, 122 aa, Homo sapiens
MSYIPGQPVTAVVQRVEIHKLRQGENLILGFSIGGGIDQDPSQNPFSEDKTDKGIYVTRVSEGGPAEIAGLQIGDKIMQVNGWDMTMVTHDQARKRLTKRSEEVVRLLVTRQSLQKAVQQSM

SEQ ID NO: 73 - protein, 125 aa, Homo sapiens
EMSYIPGQPVTAVVQRVEIHKLRQGENLILGFSIGGGIDQDPSQNPFSEDKTDKGIYVTRVSEGGPAEIAGLQIGDKIMQVNGWDMTMVTHDQARKRLTKRSEEVVRLLVTRQSLQKAVQQSMLS

SEQ ID NO: 74 - protein, 117 aa, Homo sapiens
PGQPVTAVVQRVEIHKLRQGENLILGFSIGGGIDQDPSQNPFSEDKTDKGIYVTRVSEGGPAEIAGLQIGDKIMQVNGWDMTMVTHDQARKRLTKRSEEVVRLLVTRQSLQKAVQQS

SEQ ID NO: 75 - protein, 27 aa, Homo sapiens
AAGCGTCGACTTTATGCACCAAAAGAG

SEQ ID NO: 76 - protein, 29 aa, Homo sapiens
AATGCTCGAGTATCTCCATGCATGATTAC

SEQ ID NO: 77 - protein, 30 aa, Homo sapiens
GCTGCTCGAGTCAACGTGTTCTTGATGATC

SEQ ID NO: 78 - protein, 10 aa, Homo sapiens
GYCRNCIRKQ

SEQ ID NO: 79 - protein, 10 aa, Homo sapiens
WTTCMEDLLP

SEQ ID NO: 80 - protein, 10 aa, Homo sapiens
GICRLCKHFQ

SEQ ID NO: 81 - protein, 10 aa, Homo sapiens
KGLCRQCKQI

SEQ ID NO: 82 - protein, 10 aa, Homo sapiens
WLRCTVRIPQ

SEQ ID NO: 83 - protein, 10 aa, Homo sapiens
RQCKHFYNDW

SEQ ID NO: 84 - protein, 10 aa, Homo sapiens
CRNCISHEGR

SEQ ID NO: 85 - protein, 10 aa, Homo sapiens
CCRNCYEHEG

SEQ ID NO: 86 - protein, 10 aa, Homo sapiens
SSRTRRETQL

SEQ ID NO: 87 - protein, 10 aa, Homo sapiens
RLQRRRETQV

SEQ ID NO: 88 - protein, 10 aa, Homo sapiens
RRTLRRETQV

SEQ ID NO: 89 - protein, 10 aa, Homo sapiens
WKPTRRETEV

SEQ ID NO: 90 - protein, 10 aa, Homo sapiens
RRLTRRETQV

SEQ ID NO: 91 - protein, 10 aa, Homo sapiens
RLRRRRETQV

SEQ ID NO: 92 - protein, 10 aa, Homo sapiens
RLQRRNETQV

SEQ ID NO: 93 - protein, 10 aa, Homo sapiens
RLQRRRVTQV

SEQ ID NO: 94 - protein, 10 aa, Homo sapiens
TSREPRESTV

SEQ ID NO: 95 - protein, 10 aa, Homo sapiens
QRQARSETLV

SEQ ID NO: 96 - protein, 10 aa, Homo sapiens
RLQRRRQTQV

SEQ ID NO: 97 - protein, 10 aa, Homo sapiens
RLQRRRETAL

SEQ ID NO: 98 - protein, 10 aa, Homo sapiens
TSRQATESTV

SEQ ID NO: 99 - protein, 10 aa, Homo sapiens
RRRTRQETQV

SEQ ID NO: 100 - protein, 87 aa, Homo sapiens
RDMAEAHKEAMSRKLGQSESQGPPRAFAKVNSISPGSPSIAGLQVDDEIVEFGSVNTQNFQSLHNIGSVVQHSEGALAPTILLSVSM

SEQ ID NO: 101 - protein, 93 aa, Homo sapiens
LRKEPEIITVTLKKQNGMGLSIVAAKGAGQDKLGIYVKSVVKGGAADVDGRLAAGDQLLSVDGRSLVGLSQERAAELMTRTSSVVTLEVAKQG

SEQ ID NO: 102 - protein, 105 aa, Homo sapiens
LIRPSVISIIGLYKEKGKGLGFSIAGGRDCIRGQMGIFVKTIFPNGSAAEDGRLKEGDEILDVNGIPIKGLTFQEAIHTFKQIRSGLFVLTVRTKLVSPSLTNSS

SEQ ID NO: 103 - protein, 132 aa, Homo sapiens
GISSLGRKTPGPKDRIVMEVTLNKEPRVGLGIGACCLALENSPPGIYIHSLAPGSVAKMESNLSRGDQILEVNSVNVRHAALSKVHAILSKCPPGPVRLVIGRHPNPKVSEQEMDEVIARSTYQESKEANSS

SEQ ID NO: 104 - protein, 105 aa, Homo sapiens
QSENEEDVCFIVLNRKEGSGLGFSVAGGTDVEPKSITVHRVFSQGAASQEGTMNRGDFLLSVNGASLAGLAHGNVLKVLHQAQLHKDALVVIKKGMDQPRPSNSS

SEQ ID NO: 105 - protein, 101 aa, Homo sapiens
LGRSVAVHDALCVEVLKTSAGLGLSLDGGKSSVTGDGPLVIKRVYKGGAAEQAGIIEAGDEILAINGKPLVGLMHFDAWNIMKSVPEGPVQLLIRKHRNSS

SEQ ID NO: 106 - protein, 74 aa, Homo sapiens
QTVILPGPAAWGFRLSGGIDFNQPLVITRITPGSKAAAANLCPGDVILAIDGFGTESMTHADGQDRIKAAEFIV

SEQ ID NO: 107 - protein, 85 aa, Homo sapiens
ILVEVQLSGGAPWGFTLKGGREHGEPLVITKIEEGSKAAAVDKLLAGDEIVGINDIGLSGFRQEAICLVKGSHKTLKLVVKRNSS

SEQ ID NO: 108 - protein, 104 aa, Homo sapiens
REKPLFTRDASQLKGTFLSTTLKKSNMGFGFTIIGGDEPDEFLQVKSVIPDGPAAQDGKMETGDVIVYINEVCVLGHTHADVVKLFQSVPIGQSVNLVLCRGYP

SEQ ID NO: 109 - protein, 91 aa, Homo sapiens
LSGATQAELMTLTIVKGAQGFGFTIADSPTGQRVKQILDIQGCPGLCEGDLIVEINQQNVQNLSHTEVVDILKDCPIGSETSLIIHRGGFF

SEQ ID NO: 110 - protein, 93 aa, Homo sapiens
HYKELDVHLRRMESGFGFRILGGDEPGQPILIGAVIAMGSADRDGRLHPGDELVYVDGIPVAGKTHRYVIDLMHHAARNGQVNLTVRRKVLCG

SEQ ID NO: 111 - protein, 106 aa, Homo sapiens
EGRGISSHSLQTSDAVIHRKENEGFGFVIISSLNRPESGSTITVPHKIGRIIDGSPADRCAKLKVGDRILAVNGQSIINMPHADIVKLIKDAGLSVTLRIIPQEEL

SEQ ID NO: 112 - protein, 98 aa, Homo sapiens
LSDYRQPQDFDYFTVDMEKGAKGFGFSIRGGREYKMDLYVLRLAEDGPAIRNGRMRVGDQIIEINGESTRDMTHARAIELIKSGGRRVRLLLKRGTGQ

SEQ ID NO: 113 - protein, 90 aa, Homo sapiens
HESVIGRNPEGQLGFELKGGAENGQFPYLGEVKPGKVAYESGSKLVSEELLLEVNETPVAGLTIRDVLAVIKHCKDPLRLKCVKQGGIHR

SEQ ID NO: 114 - protein, 126 aa, Homo sapiens
NLMFRKFSLERPFRPSVTSVGHVRGPGPSVQHTTLNGDSLTSQLTLLGGNARGSFVHSVKPGSLAEKAGLREGHQLLLLEGCIRGERQSVPLDTCTKEEAHWTIQRCSGPVTLHYKVNHEGYRKLV

SEQ ID NO: 115 - protein, 100 aa, Homo sapiens
ILSQVTMLAFQGDALLEQISVIGGNLTGIFIHRVTPGSAADQMALRPGTQIVMVDYEASEPLFKAVLEDTTLEEAVGLLRRVDGFCCLSVKVNTDGYKRL

SEQ ID NO: 116 - protein, 90 aa, Homo sapiens
TRVRLVQFQKNTDEPMGITLKMNELNHCIVARIMHGGMIHRQGTLHVGDEIREINGISVANQTVEQLQKMLREMRGSITFKIVPSYRTQS

SEQ ID NO: 117 - protein, 88 aa, Homo sapiens
LEQKAVLEQVQLDSPLGLEIHTTSNCQHFVSQVDTQVPTDSRLQIQPGDEVVQINEQVVVGWPRKNMVRELLREPAGLSLVLKKIPIP

SEQ ID NO: 118 - protein, 92 aa, Homo sapiens
QRKLVTVEKQDNETFGFEIQSYRPQNQNACSSEMFTLICKIQEDSPAHCAGLQAGDVLANINGVSTEGFTYKQVVDLIRSSGNLLTIETLNG

SEQ ID NO: 119 - protein, 109 aa, Homo sapiens
RCLIQTKGQRSMDGYPEQFCVRIEKNPGLGFSISGGISGQGNPFKPSDKGIFVTRVQPDGPASNLLQPGDKILQANGHSFVHMEHEKAVLLLKSFQNTVDLVIQRELTV

SEQ ID NO: 120 - protein, 101 aa, Homo sapiens
IQVNGTDADYEYEEITLERGNSGLGFSIAGGTDNPHIGDDSSIFITKIITGGAAAQDGRLRVNDCILQVNEVDVRDVTHSKAVEALKEAGSIVRLYVKRRN

SEQ ID NO: 121 - protein, 95 aa, Homo sapiens
IQLIKGPKGLGFSIAGGVGNQHIPGDNSIYVTKIIEGGAAHKDGKLQIGDKLLAVNNVCLEEVTHEEAVTALKNTSDFVYLKVAKPTSMYMNDGN

SEQ ID NO: 122 - protein, 85 aa, Homo sapiens
ILHRGSTGLGFNIVGGEDGEGIFISFILAGGPADLSGELRKGDRIISVNSVDLRAASHEQAAAALKNAGQAVTIVAQYRPEEYSR

SEQ ID NO: 123 - protein, 101 aa, Homo sapiens
ISYVNGTEIEYEFEEITLERGNSGLGFSIAGGTDNPHIGDDPGIFITKIIPGGAAAEDGRLRVNDCILRVNEVDVSEVSHSKAVEALKEAGSIVRLYVRRR

SEQ ID NO: 124 - protein, 94 aa, Homo sapiens
ISVVEIKLFKGPKGLGFSIAGGVGNQHIPGDNSIYVTKIIDGGAAQKDGRLQVGDRLLMVNNYSLEEVTHEEAVAILKNTSEVVYLKVGNPTTI

SEQ ID NO: 125 - protein, 95 aa, Homo sapiens
IWAVSLEGEPRKVVLHKGSTGLGFNIVGGEDGEGIFVSFILAGGPADLSGELQRGDQILSVNGIDLRGASHEQAAAALKGAGQTVTIIAQYQPED

SEQ ID NO: 126 - protein, 102 aa, Homo sapiens
GIPYVEEPRHVKVQKGSEPLGISIVSGEKGGIYVSKVTVGSIAHQAGLEYGDQLLEFNGINLRSATEQQARLIIGQQCDTITILAQYNPHVHQLRNSSZLTD

SEQ ID NO: 127 - protein, 103 aa, Homo sapiens
GILAGDANKKTLEPRVVFIKKSQLELGVHLCGGNLHGVFVAEVEDDSPAKGPDGLVPGDLILEYGSLDVRNKTVEEVYVEMLKPRDGVRLKVQYRPEEFIVTD

SEQ ID NO: 128 - protein, 141 aa, Homo sapiens
PTSPEIQELRQMLQAPHFKALLSAHDTIAQKDFEPLLPPLPDNIPESEEAMRIVCLVKNQQPLGATIKRHEMTGDILVARIIHGGLAERSGLLYAGDKLVEVNGVSVEGLDPEQVIHILAMSRGTIMFKVVPVSDPPVNSS

SEQ ID NO: 129 - protein, 97 aa, Homo sapiens
PTSPEIQELRQMLQAPHFKGATIKRHEMTGDILVARIIHGGLAERSGLLYAGDKLVEVNGVSVEGLDPEQVIHILAMSRGTIMFKVVPVSDPPVNSS

SEQ ID NO: 130 - protein, 93 aa, Homo sapiens
LNIVTVTLNMERHHFLGISIVGQSNDRGDGGIYIGSIMKGGAVAADGRIEPGDMLLQVNDVNFENMSNDDAVRVLREIVSQTGPISLTVAKCW

SEQ ID NO: 131 - protein, 100 aa, Homo sapiens
LNIITVTLNMEKYNFLGISIVGQSNERGDGGIYIGSIMKGGAVAADGRIEPGDMLLQVNDMNFENMSNDDAVRVLRDIVHKPGPIVLTVAKCWDPSPQNS

SEQ ID NO: 132 - protein, 95 aa, Homo sapiens
IITVTLNMEKYNFLGISIVGQSNERGDGGIYIGSIMKGGAVAADGRIEPGDMLLQVNEINFENMSNDDAVRVLREIVHKPGPITLTVAKCWDPSP

SEQ ID NO: 133 - protein, 92 aa, Homo sapiens
TTQQIDLQGPGPWGFRLVGRKDFEQPLAISRVTPGSKAALANLCIGDVITAIDGENTSNMTHLEAQNRIKGCTDNLTLTVARSEHKVWSPLV

SEQ ID NO: 134 - protein, 89 aa, Homo sapiens
IFMDSFKVVLEGPAPWGFRLQGGKDFNVPLSISRLTPGGKAAQAGVAVGDWVLSIDGENAGSLTHIEAQNKIRACGERLSLGLSRAQPV

SEQ ID NO: 135 - protein, 100 aa, Homo sapiens
QGHELAKQEIRVRVEKDPELGFSISGGVGGRGNPFRPDDDGIFVTRVQPEGPASKLLQPGDKIIQANGYSFINIEHGQAVSLLKTFQNTVELIIVREVSS

SEQ ID NO: 136 - protein, 87 aa, Homo sapiens
ILCCLEKGPNGYGFHLHGEKGKLGQYIRLVEPGSPAEKAGLLAGDRLVEVNGENVEKETHQQVVSRIRAALNAVRLLVVDPEFIVTD

SEQ ID NO: 137 - protein, 92 aa, Homo sapiens
IRLCTMKKGPSGYGFNLHSDKSKPGQFIRSVDPDSPAEASGLRAQDRIVEVNGVCMEGKQHGDVVSAIRAGGDETKLLVVDRETDEFFMNSS

SEQ ID NO: 138 - protein, 107 aa, Homo sapiens
KNPSGELKTVTLSKMKQSLGISISGGIESKVQPMVKIEKIFPGGAAFLSGALQAGFELVAVDGENLEQVTHQRAVDTIRRAYRNKAREPMELVVRVPGPSPRPSPSD

SEQ ID NO: 139 - protein, 97 aa, Homo sapiens
EGHSHPRVVELPKTEEGLGFNIMGGKEQNSPIYISRIIPGGIADRHGGLKRGDQLLSVNGVSVEGEHHEKAVELLKAAQGKVKLVVRYTPKVLEEME

SEQ ID NO: 140 - protein, 88 aa, Homo sapiens
PGAPYARKTFTIVGDAVGWGFVVRGSKPCHIQAVDPSGPAAAAGMKVCQFVVSVNGLNVLHVDYRTVSNLILTGPRTIVMEVMEELEC

SEQ ID NO: 141 - protein, 97 aa, Homo sapiens
GQYGGETVKIVRIEKARDIPLGATVRNEMDSVIISRIVKGGAAEKSGLLHEGDEVLEINGIEIRGKDVNEVFDLLSDMHGTLTFVLIPSQQIKPPPA

SEQ ID NO: 142 - protein, 98 aa, Homo sapiens
ILAHVKGIEKEVNVYKSEDSLGLTITDNGVGYAFIKRIKDGGVIDSVKTICVGDHIESINGENIVGWRHYDVAKKLKELKKEELFTMKLIEPKKAFEI

SEQ ID NO: 143 - protein, 104 aa, Homo sapiens
KPSQASGHFSVELVRGYAGFGLTLGGGRDVAGDTPLAVRGLLKDGPAQRCGRLEVGDLVLHINGESTQGLTHAQAVERIRAGGPQLHLVIRRPLETHPGKPRGV

SEQ ID NO: 144 - protein, 107 aa, Homo sapiens
PVMSQCACLEEVHLPNIKPGEGLGMYIKSTYDGLHVITGTTENSPADRSQKIHAGDEVIQVNQQTVVGWQLKNLVKKLRENPTGVVLLLKKRPTGSFNFTPEFIVTD

SEQ ID NO: 145 - protein, 100 aa, Homo sapiens
LDDEEDSVKIIRLVKNREPLGATIKKDEQTGAIIVARIMRGGAADRSGLIHVGDELREVNGIPVEDKRPEEIIQILAQSQGAITFKIIPGSKEETPSNSS

SEQ ID NO: 146 - protein, 83 aa, Homo sapiens
VVELMKKEGTTLGLTVSGGIDKDGKPRVSNLRQGGIAARSDQLDVGDYIKAVNGINLAKFRHDEIISLLKNVGERVVLEVEYE

SEQ ID NO: 147 - protein, 110 aa, Homo sapiens
RSSVIFRTVEVTLHKEGNTFGFVIRGGAHDDRNKSRPVVITCVRPGGPADREGTIKPGDRLLSVDGIRLLGTTHAEAMSILKQCGQEAALLIEYDVSVMDSVATASGNSS

SEQ ID NO: 148 - protein, 106 aa, Homo sapiens
HVATASGPLLVEVAKTPGASLGVALTTSMCCNKQVIVIDKIKSASIADRCGALHVGDHILSIDGTSMEYCTLAEATQFLANTTDQVKLEILPHHQTRLALKGPNSS

SEQ ID NO: 149 - protein, 97 aa, Homo sapiens
TETTEVVLTADPVTGFGIQLQGSVFATETLSSPPLISYIEADSPAERCGVLQIGDRVMAINGIPTEDSTFEEASQLLRDSSITSKVTLEIEFDVAES

SEQ ID NO: 150 - protein, 101 aa, Homo sapiens
AESVIPSSGTFHVKLPKKHNVELGITISSPSSRKPGDPLVISDIKKGSVAHRTGTLELGDKLLAIDNIRLDNCSMEDAVQILQQCEDLVKLKIRKDEDNSD

SEQ ID NO: 151 - protein, 90 aa, Homo sapiens
IYTVELKRYGGPLGITISGTEEPFDPIIISSLTKGGLAERTGAIHIGDRILAINSSSLKGKPLSEAIHLLQMAGETVTLKIKKQTDAQSA

SEQ ID NO: 152 - protein, 95 aa, Homo sapiens
IMSPTPVELHKVTLYKDSDMEDFGFSVADGLLEKGVYVKNIRPAGPGDLGGLKPYDRLLQVNHVRTRDFDCCLVVPLIAESGNKLDLVISRNPLA

SEQ ID NO: 153 - protein, 88 aa, Homo sapiens
SRGCETRELALPRDGQGRLGFEVDAEGFVTHVERFTFAETAGLRPGARLLRVCGQTLPSLRPEAAAQLLRSAPKVCVTVLPPDESGRP

SEQ ID NO: 154 - protein, 95 aa, Homo sapiens
AKAKWRQVVLQKASRESPLQFSLNGGSEKGFGIFVEGVEPGSKAADSGLKRGDQIMEVNGQNFENITFMKAVEILRNNTHLALTVKTNIFVFKEL

SEQ ID NO: 155 - protein, 89 aa, Homo sapiens
LENVIAKSLLIKSNEGSYGFGLEDKNKVPIIKLVEKGSNAEMAGMEVGKKIFAINGDLVFMRPFNEVDCFLKSCLNSRKPLRVLVSTKP

SEQ ID NO: 156 - protein, 82 aa, Homo sapiens
PRETVKIPDSADGLGFQIRGFGPSVVHAVGRGTVAAAAGLHPGQCIIKVNGINVSKETHASVIAHVTACRKYRRPTKQDSIQ

SEQ ID NO: 157 - protein, 100 aa, Homo sapiens
EDFCYVFTVELERGPSGLGMGLIDGMHTHLGAPGLYIQTLLPGSPAAADGRLSLGDRILEVNGSSLLGLGYLRAVDLIRHGGKKMRFLVAKSDVETAKKI

SEQ ID NO: 158 - protein, 109 aa, Homo sapiens
LTEFQDKQIKDWKKRFIGIRMRTITPSLVDELKASNPDFPEVSSGIYVQEVAPNSPSQRGGIQDGDIIVKVNGRPLVDSSELQEAVLTESPLLLEVRRGNDDLLFSNSS

SEQ ID NO: 159 - protein, 97 aa, Homo sapiens
HKKYLGLQMLSLTVPLSEELKMHYPDFPDVSSGVYVCKVVEGTAAQSSGLRDHDVIVNINGKPITTTTDVVKALDSDSLSMAVLRGKDNLLLTVNSS

SEQ ID NO: 160 - protein, 104 aa, Homo sapiens
IWQIEYIDIERPSTGGLGFSVVALRSQNLGKVDIFVKDVQPGSVADRDQRLKENDQILAINHTPLDQNISHQQAIALLQQTTGSLRLIVAREPVHTKSSTSSSE

SEQ ID NO: 161 - protein, 78 aa, Homo sapiens
PGHVEEVELINDGSGLGFGIVGGKTSGVVVRTIVPGGLADRDGRLQTGDHILKIGGTNVQGMTSEQVAQVLRNCGNSS

SEQ ID NO: 162 - protein, 111 aa, Homo sapiens
PGSDSSLFETYNVELVRKDGQSLGIRIVGYVGTSHTGEASGIYVKSIIPGSAAYHNGHIQVNDKIVAVDGVNIQGFANHDVVEVLRNAGQVVHLTLVRRKTSSSTSRIHRD

SEQ ID NO: 163 - protein, 96 aa, Homo sapiens
NSDDAELQKYSKLLPIHTLRLGVEVDSFDGHHYISSIVSGGPVDTLGLLQPEDELLEVNGMQLYGKSRREAVSFLKEVPPPFTLVCCRRLFDDEAS

SEQ ID NO: 164 - protein, 102 aa, Homo sapiens
LSSPEVKIVELVKDCKGLGFSILDYQDPLDPTRSVIVIRSLVADGVAERSGGLLPGDRLVSVNEYCLDNTSLAEAVEILKAVPPGLVHLGICKPLVEFIVTD

SEQ ID NO: 165 - protein, 119 aa, Homo sapiens
PNFSHWGPPRIVEIFREPNVSLGISIVVGQTVIKRLKNGEELKGIFIKQVLEDSPAGKTNALKTGDKILEVSGVDLQNASHSEAVEAIKNAGNPVVFIVQSLSSTPRVIPNVHNKANSS

SEQ ID NO: 166 - protein, 99 aa, Homo sapiens
PGELHIIELEKDKNGLGLSLAGNKDRSRMSIFVVGINPEGPAAADGRMRIGDELLEINNQILYGRSHQNASAIIKTAPSKVKLVFIRNEDAVNQMANSS

SEQ ID NO: 167 - protein, 93 aa, Homo sapiens
PATCPIVPGQEMIIEISKGRSGLGLSIVGGKDTPLNAIVIHEVYEEGAAARDGRLWAGDQILEVNGVDLRNSSHEEAITALRQTPQKVRLVVY

SEQ ID NO: 168 - protein, 103 aa, Homo sapiens
ILTLTILRQTGGLGISIAGGKGSTPYKGDDEGIFISRVSEEGPAARAGVRVGDKLLEVNGVALQGAEHHEAVEALRGAGTAVQMRVWRERMVEPENAEFIVTD

SEQ ID NO: 169 - protein, 97 aa, Homo sapiens
PLRQRHVACLARSERGLGFSIAGGKGSTPYRAGDAGIFVSRIAEGGAAHRAGTLQVGDRVLSINGVDVTEARHDHAVSLLTAASPTIALLLEREAGG

SEQ ID NO: 170 - protein, 106 aa, Homo sapiens
ILEGPYPVEEIRLPRAGGPLGLSIVGGSDHSSHPFGVQEPGVFISKVLPRGLAARSGLRVGDRILAVNGQDVRDATHQEAVSALLRPCLELSLLVRRDPAEFIVTD

SEQ ID NO: 171 - protein, 105 aa, Homo sapiens
RELCIQKAPGERLGISIRGGARGHAGNPRDPTDEGIFISKVSPTGAAGRDGRLRVGLRLLEVNQQSLLGLTHGEAVQLLRSVGDTLTVLVCDGFEASTDAALEVS

SEQ ID NO: 172 - protein, 91 aa, Homo sapiens
PHQPIVIHSSGKNYGFTIRAIRVYVGDSDIYTVHHIVWNVEEGSPACQAGLKAGDLITHINGEPVHGLVHTEVIELLLKSGNKVSITTTPF

SEQ ID NO: 173 - protein, 105 aa, Homo sapiens
ILACAAKAKRRLMTLTKPSREAPLPFILLGGSEKGFGIFVDSVDSGSKATEAGLKRGDQILEVNGQNFENIQLSKAMEILRNNTHLSITVKTNLFVFKELLTNSS

SEQ ID NO: 174 - protein, 88 aa, Homo sapiens
IPPAPRKVEMRRDPVLGFGFVAGSEKPVVVRSVTPGGPSEGKLIPGDQIVMINDEPVSAAPRERVIDLVRSCKESILLTVIQPYPSPK

SEQ ID NO: 175 - protein, 101 aa, Homo sapiens
LNKRTTMPKDSGALLGLKVVGGKMTDLGRLGAFITKVKKGSLADVVGHLRAGDEVLEWNGKPLPGATNEEVYNIILESKSEPQVEIIVSRPIGDIPRIHRD

SEQ ID NO: 176 - protein, 79 aa, Homo sapiens
QRCVIIQKDQHGFGFTVSGDRIVLVQSVRPGGAAMKAGVKEGDRIIKVNGTMVTNSSHLEVVKLIKSGAYVALTLLGSS

SEQ ID NO: 177 - protein, 87 aa, Homo sapiens
ILVQRCVIIQKDDNGFGLTVSGDNPVFVQSVKEDGAAMRAGVQTGDRIIKVNGTLVTHSNHLEVVKLIKSGSYVALTVQGRPPGNSS

SEQ ID NO: 178 - protein, 79 aa, Homo sapiens
SVEMTLRRNGLGQLGFHVNYEGIVADVEPYGYAWQAGLRQGSRLVEICKVAVATLSHEQMIDLLRTSVTVKVVIIPPHD

SEQ ID NO: 179 - protein, 96 aa, Homo sapiens
LKVMTSGWETVDMTLRRNGLGQLGFHVKYDGTVAEVEDYGFAWQAGLRQGSRLVEICKVAVVTLTHDQMIDLLRTSVTVKVVIIPPFEDGTPRRGW

SEQ ID NO: 180 - protein, 105 aa, Homo sapiens
HYIFPHARIKITRDSKDHTVSGNGLGIRIVGGKEIPGHSGEIGAYIAKILPGGSAEQTGKLMEGMQVLEWNGIPLTSKTYEEVQSIISQQSGEAEICVRLDLNML

SEQ ID NO: 181 - protein, 103 aa, Homo sapiens
LCGSLRPPIVIHSSGKKYGFSLRAIRVYMGDSDVYTVHHVVWSVEDGSPAQEAGLRAGDLITHINGESVLGLVHMDVVELLLKSGNKISLRTTALENTSIKVG

SEQ ID NO: 182 - protein, 86 aa, Homo sapiens
SYSVTLTGPGPWGFRLQGGKDFNMPLTISRITPGSKAAQSQLSQGDLVVAIDGVNTDTMTHLEAQNKIKSASYNLSLTLQKSKNSS

SEQ ID NO: 183 - protein, 91 aa, Homo sapiens
ISRDSGAMLGLKVVGGKMTESGRLCAFITKVKKGSLADTVGHLRPGDEVLEWNGRLLQGATFEEVYNIILESKPEPQVELVVSRPIAIHRD

SEQ ID NO: 184 - protein, 101 aa, Homo sapiens
ISALGSMRPPIIIHRAGKKYGFTLRAIRVYMGDSDVYTVHHMVWHVEDGGPASEAGLRQGDLITHVNGEPVHGLVHTEVVELILKSGNKVAISTTPLENSS

SEQ ID NO: 185 - protein, 94 aa, Homo sapiens
FSDMRISINQTPGKSLDFGFTIKWDIPGIFVASVEAGSPAEFSQLQVDDEIIAINNTKFSYNDSKEWEEAMAKAQETGHLVMDVRRYGKAGSPE

SEQ ID NO: 186 - protein, 98 aa, Homo sapiens
QSAHLEVIQLANIKPSEGLGMYIKSTYDGLHVITGTTENSPADRCKKIHAGDEVIQVNHQTVVGWQLKNLVNALREDPSGVILTLKKRPQSMLTSAPA

SEQ ID NO: 187 - protein, 100 aa, Homo sapiens
ILTQTLIPVRHTVKIDKDTLLQDYGFHISESLPLTVVAVTAGGSAHGKLFPGDQILQMNNEPAEDLSWERAVDILREAEDSLSITVVRCTSGVPKSSNSS

SEQ ID NO: 188 - protein, 93 aa, Homo sapiens
GLRSPITIQRSGKKYGFTLRAIRVYMGDTDVYSVHHIVWHVEEGGPAQEAGLCAGDLITHVNGEPVHGMVHPEVVELILKSGNKVAVTTTPFE

SEQ ID NO: 189 - protein, 107 aa, Homo sapiens
QGEETKSLTLVLHRDSGSLGFNIIGGRPSVDNHDGSSSEGIFVSKIVDSGPAAKEGGLQIHDRIIEVNGRDLSRATHDQAVEAFKTAKEPIVVQVLRRTPRTKMFTP

SEQ ID NO: 190 - protein, 101 aa, Homo sapiens
QEMDREELELEEVDLYRMNSQDKLGLTVCYRTDDEDDIGIYISEIDPNSIAAKDGRIREGDRIIQINGIEVQNREEAVALLTSEENKNFSLLIARPELQLD

SEQ ID NO: 191 - protein, 91 aa, Homo sapiens
RSFQYVPVQLQGGAPWGFTLKGGLEHCEPLTVSKIEDGGKAALSQKMRTGDELVNINGTPLYGSRQEALILIKGSFRILKLIVRRRNAPVS

SEQ ID NO: 192 - protein, 102 aa, Homo sapiens
ILEKLELFPVELEKDEDGLGISIIGMGVGADAGLEKLGIFVKTVTEGGAAQRDGRIQVNDQIVEVDGISLVGVTQNFAATVLRNTKGNVRFVIGREKPGQVS

SEQ ID NO: 193 - protein, 113 aa, Homo sapiens
KDVNVYVNPKKLTVIKAKEQLKLLEVLVGIIHQTKWSWRRTGKQGDGERLVVHGLLPGGSAMKSGQVLIGDVLVAVNDVDVTTENIERVLSCIPGPMQVKLTFENAYDVKRET

SEQ ID NO: 194 - protein, 90 aa, Homo sapiens
TRGCETVEMTLRRNGLGQLGFHVNFEGIVADVEPFGFAWKAGLRQGSRLVEICKVAVATLTHEQMIDLLRTSVTVKVVIIQPHDDGSPRR

SEQ ID NO: 195 - protein, 96 aa, Homo sapiens
VENILAKRLLILPQEEDYGFDIEEKNKAVVVKSVQRGSLAEVAGLQVGRKIYSINEDLVFLRPFSEVESILNQSFCSRRPLRLLVATKAKEIIKIP

SEQ ID NO: 196 - protein, 103 aa, Homo sapiens
PDSAGPGEVRLVSLRRAKAHEGLGFSIRGGSEHGVGIYVSLVEPGSLAEKEGLRVGDQILRVNDKSLARVTHAEAVKALKGSKKLVLSVYSAGRIPGGYVTNH

SEQ ID NO: 197 - protein, 100 aa, Homo sapiens
LQGGDEKKVNLVLGDGRSLGLTIRGGAEYGLGIYITGVDPGSEAEGSGLKVGDQILEVNWRSFLNILHDEAVRLLKSSRHLILTVKDVGRLPHARTTVDE

SEQ ID NO: 198 - protein, 87 aa, Homo sapiens
WTSGAHVHSGPCEEKCGHPGHRQPLPRIVTIQRGGSAHNCGQLKVGHVILEVNGLTLRGKEHREAARIIAEAFKTKDRDYIDFLDSL

SEQ ID NO: 199 - protein, 100 aa, Homo sapiens
ELRRAELVEIIVETEAQTGVSGINVAGGGKEGIFVRELREDSPAARSLSLQEGDQLLSARVFFENFKYEDALRLLQCAEPYKVSFCLKRTVPTGDLALRP

SEQ ID NO: 200 - protein, 102 aa, Homo sapiens
PSQLKGVLVRASLKKSTMGFGFTIIGGDRPDEFLQVKNVLKDGPAAQDGKIAPGDVIVDINGNCVLGHTHADVVQMFQLVPVNQYVNLTLCRGYPLPDDSED

SEQ ID NO: 201 - protein, 100 aa, Homo sapiens
ASSGSSQPELVTIPLIKGPKGFGFAIADSPTGQKVKMILDSQWCQGLQKGDIIKEIYHQNVQNLTHLQVVEVLKQFPVGADVPLLILRGGPPSPTKTAKM

SEQ ID NO: 202 - protein, 143 aa, Homo sapiens
LYEDKPPLTNTFLISNPRTTADPRILYEDKPPNTKDLDVFLRKQESGFGFRVLGGDGPDQSIYIGAIIPLGAAEKDGRLRAADELMCIDGIPVKGKSHKQVLDLMTTAARNGHVLLTVRRKIFYGEKQPEDDSGSPGIHRELT

SEQ ID NO: 203 - protein, 102 aa, Homo sapiens
PAPQEPYDVVLQRKENEGFGFVILTSKNKPPPGVIPHKIGRVIEGSPADRCGKLKVGDHISAVNGQSIVELSHDNIVQLIKDAGVTVTLTVIAEEEHHGPPS

SEQ ID NO: 204 - protein, 98 aa, Homo sapiens
QNLGCYPVELERGPRGFGFSLRGGKEYNMGLFILRLAEDGPAIKDGRIHVGDQIVEINGEPTQGITHTRAIELIQAGGNKVLLLLRPGTGLIPDHGLA

SEQ ID NO: 205 - protein, 84 aa, Homo sapiens
ITVVELIKKEGSTLGLTISGGTDKDGKPRVSNLRPGGLAARSDLLNIGDYIRSVNGIHLTRLRHDEIITLLKNVGERVVLEVEY

SEQ ID NO: 206 - protein, 92 aa, Homo sapiens
ILDVSLYKEGNSFGFVLRGGAHEDGHKSRPLVLTYVRPGGPADREGSLKVGDRLLSVDGIPLHGASHATALATLRQCSHEALFQVEYDVATP

SEQ ID NO: 207 - protein, 102 aa, Homo sapiens
IHTVANASGPLMVEIVKTPGSALGISLTTTSLRNKSVITIDRIKPASVVDRSGALHPGDHILSIDGTSMEHCSLLEATKLLASISEKVRLEILPVPQSQRPL

SEQ ID NO: 208 - protein, 103 aa, Homo sapiens
IQIVHTETTEVVLCGDPLSGFGLQLQGGIFATETLSSPPLVCFIEPDSPAERCGLLQVGDRVLSINGIATEDGTMEEANQLLRDAALAHKVVLEVEFDVAESV

SEQ ID NO: 209 - protein, 103 aa, Homo sapiens
IQFDVAESVIPSSGTFHVKLPKKRSVELGITISSASRKRGEPLIISDIKKGSVAHRTGTLEPGDKLLAIDNIRLDNCPMEDAVQILRQCEDLVKLKIRKDEDN

SEQ ID NO: 210 - protein, 94 aa, Homo sapiens
IQTTGAVSYTVELKRYGGPLGITISGTEEPFDPIVISGLTKRGLAERTGAIHVGDRILAINNVSLKGRPLSEAIHLLQVAGETVTLKIKKQLDR

SEQ ID NO: 211 - protein, 105 aa, Homo sapiens
ILEMEELLLPTPLEMHKVTLHKDPMRHDFGFSVSDGLLEKGVYVHTVRPDGPAHRGGLQPFDRVLQVNHVRTRDFDCCLAVPLLAEAGDVLELIISRKPHTAHSS

SEQ ID NO: 212 - protein, 91 aa, Homo sapiens
MALTVDVAGPAPWGFRITGGRDFHTPIMVTKVAERGKAKDADLRPGDIIVAINGESAEGMLHAEAQSKIRQSPSPLRLQLDRSQATSPGQT

SEQ ID NO: 213 - protein, 84 aa, Homo sapiens
SNYSVSLVGPAPWGFRLQGGKDFNMPLTISSLKDGGKAAQANVRIGDVVLSIDGINAQGMTHLEAQNKIKGCTGSLNMTLQRAS

SEQ ID NO: 214 - protein, 133 aa, Homo sapiens
TLVEHSKLYCGHCYYQTVVTPVIEQILPDSPGSHLPHTVTLVSIPASSHGKRGLSVSIDPPHGPPGCGTEHSHTVRVQGVDPGCMSPDVKNSIHVGDRILEINGTPIRNVPLDEIDLLIQETSRLLQLTLEHD

SEQ ID NO: 215 - protein, 92 aa, Homo sapiens
PYSVTLISMPATTEGRRGFSVSVESACSNYATTVQVKEVNRMHISPNNRNAIHPGDRILEINGTPVRTLRVEEVEDAISQTSQTLQLLIEHD

SEQ ID NO: 216 - protein, 82 aa, Homo sapiens
IHSVTLRGPSPWGFRLVGRDFSAPLTISRVHAGSKASLAALCPGDLIQAINGESTELMTHLEAQNRIKGCHDHLTLSVSRPE

SEQ ID NO: 217 - protein, 74 aa, Homo sapiens
VCYRTDDEEDLGIYVGEVNPNSIAAKDGRIREGDRIIQINGVDVQNREEAVAILSQEENTNISLLVARPESQLA

SEQ ID NO: 218 - protein, 103 aa, Homo sapiens
IQKKNHWTSRVHECTVKRGPQGELGVTVLGGAEHGEFPYVGAVAAVEAAGLPGGGEGPRLGEGELLLEVQGVRVSGLPRYDVLGVIDSCKEAVTFKAVRQGGR

SEQ ID NO: 219 - protein, 104 aa, Homo sapiens
PSELKGKFIHTKLRKSSRGFGFTVVGGDEPDEFLQIKSLVLDGPAALDGKMETGDVIVSVNDTCVLGHTHAQVVKIFQSIPIGASVDLELCRGYPLPFDPDDPN

SEQ ID NO: 220 - protein, 92 aa, Homo sapiens
PATQPELITVHIVKGPMGFGFTIADSPGGGGQRVKQIVDSPRCRGLKEGDLIVEVNKKNVQALTHNQVVDMLVECPKGSEVTLLVQRGGNLS

SEQ ID NO: 221 - protein, 102 aa, Homo sapiens
PDYQEQDIFLWRKETGFGFRILGGNEPGEPIYIGHIVPLGAADTDGRLRSGDELICVDGTPVIGKSHQLVVQLMQQAAKQGHVNLTVRRKVVFAVPKTENSS

SEQ ID NO: 222 - protein, 112 aa, Homo sapiens
GVVSTVVQPYDVEIRRGENEGFGFVIVSSVSRPEAGTTFAGNACVAMPHKIGRIIEGSPADRCGKLKVGDRILAVNGCSITNKSHSDIVNLIKEAGNTVTLRIIPGDESSNA

SEQ ID NO: 223 - protein, 91 aa, Homo sapiens
QATQEQDFYTVELERGAKGFGFSLRGGREYNMDLYVLRLAEDGPAERCGKMRIGDEILEINGETTKNMKHSRAIELIKNGGRRVRLFLKRG

SEQ ID NO: 224 - protein, 100 aa, Homo sapiens
PAKMEKEETTRELLLPNWQGSGSHGLTIAQRDDGVFVQEVTQNSPAARTGVVKEGDQIVGATIYFDNLQSGEVTQLLNTMGHHTVGLKLHRKGDRSPNSS

SEQ ID NO: 225 - protein, 97 aa, Homo sapiens
SENCKVFIEKQKGEILGVVIVESGWGSILPTVIIANMMHGGPAEKSGKLNIGDQIMSINGTSLVGLPLSTCQSIIKGLKNQSRVKLNIVRCPPVNSS

SEQ ID NO: 226 - protein, 92 aa, Homo sapiens
LRCPPVTTVLIRRPDLRYQLGFSVQNGIICSLMRGGIAERGGVRVGHRIIEINGQSVVATPHEKIVHILSNAVGEIHMKTMPAAMYRLLNSS

SEQ ID NO: 227 - protein, 103 aa, Homo sapiens
LSNSDNCREVHLEKRRGEGLGVALVESGWGSLLPTAVIANLLHGGPAERSGALSIGDRLTAINGTSLVGLPLAACQAAVRETKSQTSVTLSIVHCPPVTTAIM

SEQ ID NO: 228 - protein, 92 aa, Homo sapiens
LVHCPPVTTAIIHRPHAREQLGFCVEDGIICSLLRGGIAERGGIRVGHRIIEINGQSVVATPHARIIELLTEAYGEVHIKTMPAATYRLLTG

SEQ ID NO: 229 - protein, 86 aa, Homo sapiens
RKVRLIQFEKVTEEPMGITLKLNEKQSCTVARILHGGMIHRQGSLHVGDEILEINGTNVTNHSVDQLQKAMKETKGMISLKVIPNQ

SEQ ID NO: 230 - protein, 89 aa, Homo sapiens
PVPPDAVRMVGIRKTAGEHLGVTFRVEGGELVIARILHGGMVAQQGLLHVGDIIKEVNGQPVGSDPRALQELLRNASGSVILKILPNYQ

SEQ ID NO: 231 - protein, 99 aa, Homo sapiens
QGRHVEVFELLKPPSGGLGFSVVGLRSENRGELGIFVQEIQEGSVAHRDGRLKETDQILAINGQALDQTITHQQAISILQKAKDTVQLVIARGSLPQLV

SEQ ID NO: 232 - protein, 97 aa, Homo sapiens
PVHWQHMETIELVNDGSGLGFGIIGGKATGVIVKTILPGGVADQHGRLCSGDHILKIGDTDLAGMSSEQVAQVLRQCGNRVKLMIARGAIEERTAPT

SEQ ID NO: 233 - protein, 98 aa, Homo sapiens
QESETFDVELTKNVQGLGITIAGYIGDKKLEPSGIFVKSITKSSAVEHDGRIQIGDQIIAVDGTNLQGFTNQQAVEVLRHTGQTVLLTLMRRGMKQEA

SEQ ID NO: 234 - protein, 92 aa, Homo sapiens
LNYEIVVAHVSKFSENSGLGISLEATVGHHFIRSVLPEGPVGHSGKLFSGDELLEVNGITLLGENHQDVVNILKELPIEVTMVCCRRTVPPT

SEQ ID NO: 235 - protein, 100 aa, Homo sapiens
WEAGIQHIELEKGSKGLGFSILDYQDPIDPASTVIIIRSLVPGGIAEKDGRLLPGDRLMFVNDVNLENSSLEEAVEALKGAPSGTVRIGVAKPLPLSPEE

SEQ ID NO: 236 - protein, 99 aa, Homo sapiens
RNVSKESFERTINIAKGNSSLGMTVSANKDGLGMIVRSIIHGGAISRDGRIAIGDCILSINEESTISVTNAQARAMLRRHSLIGPDIKITYVPAEHLEE

SEQ ID NO: 237 - protein, 112 aa, Homo sapiens
LNWNQPRRVELWREPSKSLGISIVGGRGMGSRLSNGEVMRGIFIKHVLEDSPAGKNGTLKPGDRIVEVDGMDLRDASHEQAVEAIRKAGNPVVFMVQSIINRPRKSPLPSLL

SEQ ID NO: 238 - protein, 95 aa, Homo sapiens
LTGELHMIELEKGHSGLGLSLAGNKDRSRMSVFIVGIDPNGAAGKDGRLQIADELLEINGQILYGRSHQNASSIIKCAPSKVKIIFIRNKDAVNQ

SEQ ID NO: 239 - protein, 94 aa, Homo sapiens
LSSFKNVQHLELPKDQGGLGIAISEEDTLSGVIIKSLTEHGVAATDGRLKVGDQILAVDDEIVVGYPIEKFISLLKTAKMTVKLTIHAENPDSQ

SEQ ID NO: 240 - protein, 95 aa, Homo sapiens
LPGCETTIEISKGRTGLGLSIVGGSDTLLGAIIIHEVYEEGAACKDGRLWAGDQILEVNGIDLRKATHDEAINVLRQTPQRVRLTLYRDEAPYKE

SEQ ID NO: 241 - protein, 98 aa, Homo sapiens
KEEEVCDTLTIELQKKPGKGLGLSIVGKRNDTGVFVSDIVKGGIADADGRLMQGDQILMVNGEDVRNATQEAVAALLKCSLGTVTLEVGRIKAGPFHS

SEQ ID NO: 242 - protein, 96 aa, Homo sapiens
LQGLRTVEMKKGPTDSLGISIAGGVGSPLGDVPIFIAMMHPTGVAAQTQKLRVGDRIVTICGTSTEGMTHTQAVNLLKNASGSIEMQVVAGGDVSV

SEQ ID NO: 243 - protein, 91 aa, Homo sapiens
LGPPQCKSITLERGPDGLGFSIVGGYGSPHGDLPIYVKTVFAKGAASEDGRLKRGDQIIAVNGQSLEGVTHEEAVAILKRTKGTVTLMVLS

SEQ ID NO: 244 - protein, 93 aa, Homo sapiens
IQYEEIVLERGNSGLGFSIAGGIDNPHVPDDPGIFITKIIPGGAAAMDGRLGVNDCVLRVNEVEVSEVVHSRAVEALKEAGPVVRLVVRRRQN

SEQ ID NO: 245 - protein, 90 aa, Homo sapiens
ITLLKGPKGLGFSIAGGIGNQHIPGDNSIYITKIIEGGAAQKDGRLQIGDRLLAVNNTNLQDVRHEEAVASLKNTSDMVYLKVAKPGSLE

SEQ ID NO: 246 - protein, 119 aa, Homo sapiens
ILLHKGSTGLGFNIVGGEDGEGIFVSFILAGGPADLSGELRRGDRILSVNGVNLRNATHEQAAAALKRAGQSVTIVAQYRPEEYSRFESKIHDLREQMMNSSMSSGSGSLRTSEKRSLE

SEQ ID NO: 247 - protein, 111 aa, Homo sapiens
CVERLELFPVELEKDSEGLGISIIGMGAGADMGLEKLGIFVKTVTEGGAAHRDGRIQVNDLLVEVDGTSLVGVTQSFAASVLRNTKGRVRFMIGRERPGEQSEVAQRIHRD

SEQ ID NO: 248 - protein, 90 aa, Homo sapiens
IQPNVISVRLFKRKVGGLGFLVKERVSKPPVIISDLIRGGAAEQSGLIQAGDIILAVNGRPLVDLSYDSALEVLRGIASETHVVLILRGP

SEQ ID NO: 249 - protein, 107 aa, Homo sapiens
QANSDESDIIHSVRVEKSPAGRLGFSVRGGSEHGLGIFVSKVEEGSSAERAGLCVGDKITEVNGLSLESTTMGSAVKVLTSSSRLHMMVRRMGRVPGIKFSKEKNSS

SEQ ID NO: 250 - protein, 106 aa, Homo sapiens
PSDTSSEDGVRRIVHLYTTSDDFCLGFNIRGGKEFGLGIYVSKVDHGGLAEENGIKVGDQVLAANGVRFDDISHSQAVEVLKGQTHIMLTIKETGRYPAYKEMNSS

SEQ ID NO: 251 - protein, 115 aa, Homo sapiens
KIKKFLTESHDRQAKGKAITKKKYIGIRMMSLTSSKAKELKDRHRDFPDVISGAYIIEVIPDTPAEAGGLKENDVIISINGQSVVSANDVSDVIKRESTLNMVVRRGNEDIMITV

SEQ ID NO: 252 - protein, 100 aa, Homo sapiens
PDGEITSIKINRVDPSESLSIRLVGGSETPLVHIIIQHIYRDGVIARDGRLLPGDIILKVNGMDISNVPHNYAVRLLRQPCQVLWLTVMREQKFRSRNSS

SEQ ID NO: 253 - protein, 101 aa, Homo sapiens
HRPRDDSFHVILNKSSPEEQLGIKLVRKVDEPGVFIFNVLDGGVAYRHGQLEENDRVLAINGHDLRYGSPESAAHLIQASERRVHLVVSRQVRQRSPENSS

SEQ ID NO: 254 - protein, 104 aa, Homo sapiens
PTITCHEKVVNIQKDPGESLGMTVAGGASHREWDLPIYVISVEPGGVISRDGRIKTGDILLNVDGVELTEVSRSEAVALLKRTSSSIVLKALEVKEYEPQEFIV

SEQ ID NO: 255 - protein, 99 aa, Homo sapiens
PRCLYNCKDIVLRRNTAGSLGFCIVGGYEEYNGNKPFFIKSIVEGTPAYNDGRIRCGDILLAVNGRSTSGMIHACLARLLKELKGRITLTIVSWPGTFL

SEQ ID NO: 256 - protein, 101 aa, Homo sapiens
LLTEEEINLTRGPSGLGFNIVGGTDQQYVSNDSGIYVSRIKENGAAALDGRLQEGDKILSVNGQDLKNLLHQDAVDLFRNAGYAVSLRVQHRLQVQNGIHS

SEQ ID NO: 257 - protein, 94 aa, Homo sapiens
PVDAIRILGIHKRAGEPLGVTFRVENNDLVIARILHGGMIDRQGLLHVGDIIKEVNGHEVGNNPKELQELLKNISGSVTLKILPSYRDTITPQQ

SEQ ID NO: 258 - protein, 93 aa, Homo sapiens
DDMVKLVEVPNDGGPLGIHVVPFSARGGRTLGLLVKRLEKGGKAEHENLFRENDCIVRINDGDLRNRRFEQAQHMFRQAMRTPIIWFHVVPAA

SEQ ID NO: 259 - protein, 94 aa, Homo sapiens
GKRLNIQLKKGTEGLGFSITSRDVTIGGSAPIYVKNILPRGAAIQDGRLKAGDRLIEVNGVDLVGKSQEEVVSLLRSTKMEGTVSLLVFRQEDA

SEQ ID NO: 260 - protein, 103 aa, Homo sapiens
TPDGTREFLTFEVPLNDSGSAGLGVSVKGNRSKENHADLGIFVKSIINGGAASKDGRLRVNDQLIAVNGESLLGKTNQDAMETLRRSMSTEGNKRGMIQLIVA

SEQ ID NO: 261 - protein, 102 aa, Homo sapiens
LPETHRRVRLHKHGSDRPLGFYIRDGMSVRVAPQGLERVPGIFISRLVRGGLAESTGLLAVSDEILEVNGIEVAGKTLDQVTDMMVANSHNLIVTVKPANQR

SEQ ID NO: 262 - protein, 111 aa, Homo sapiens
IDVDLVPETHRRVRLHRHGCEKPLGFYIRDGASVRVTPHGLEKVPGIFISRMVPGGLAESTGLLAVNDEVLEVNGIEVAGKTLDQVTDMMIANSHNLIVTVKPANQRNNVV

SEQ ID NO: 263 - protein, 100 aa, Homo sapiens
RSRKLKEVRLDRLHPEGLGLSVRGGLEFGCGLFISHLIKGGQADSVGLQVGDEIVRINGYSISSCTHEEVINLIRTKKTVSIKVRHIGLIPVKSSPDEFH

SEQ ID NO: 264 - protein, 102 aa, Homo sapiens
IPGNRENKEKKVFISLVGSRGLGCSISSGPIQKPGIFISHVKPGSLSAEVGLEIGDQIVEVNGVDFSNLDHKEAVNVLKSSRSLTISIVAAAGRELFMTDEF

SEQ ID NO: 265 - protein, 103 aa, Homo sapiens
PEQIMGKDVRLLRIKKEGSLDLALEGGVDSPIGKVVVSAVYERGAAERHGGIVKGDEIMAINGKIVTDYTLAEADAALQKAWNQGGDWIDLVVAVCPPKEYDD

SEQ ID NO: 266 - protein, 103 aa, Homo sapiens
LTSTFNPRECKLSKQEGQNYGFFLRIEKDTEGHLVRVVEKCSPAEKAGLQDGDRVLRINGVFVDKEEHMQVVDLVRKSGNSVTLLVLDGDSYEKAGSPGIHRD

SEQ ID NO: 267 - protein, 92 aa, Homo sapiens
RLCYLVKEGGSYGFSLKTVQGKKGVYMTDITPQGVAMRAGVLADDHLIEVNGENVEDASHEEVVEKVKKSGSRVMFLLVDKETDKREFIVTD

SEQ ID NO: 268 - protein, 112 aa, Homo sapiens
QFKRETASLKLLPHQPRIVEMKKGSNGYGFYLRAGSEQKGQIIKDIDSGSPAEEAGLKNNDLVVAVNGESVETLDHDSVVEMIRKGGDQTSLLVVDKETDNMYRLAEFIVTD

SEQ ID NO: 269 - protein, 95 aa, Homo sapiens
PDTTEEVDHKPKLCRLAKGENGYGFHLNAIRGLPGSFIKEVQKGGPADLAGLEDEDVIIEVNGVNVLDEPYEKVVDRIQSSGKNVTLLVZGKNSS

SEQ ID NO: 270 - protein, 89 aa, Homo sapiens
PTVPGKVTLQKDAQNLIGISIGGGAQYCPCLYIVQVFDNTPAALDGTVAAGDEITGVNGRSIKGKTKVEVAKMIQEVKGEVTIHYNKLQ

SEQ ID NO: 271 - protein, 98 aa, Homo sapiens
SQGVGPIRKVLLLKEDHEGLGISITGGKEHGVPILISEIHPGQPADRCGGLHVGDAILAVNGVNLRDTKHKEAVTILSQQRGEIEFEVVYVAPEVDSD

SEQ ID NO: 272 - protein, 97 aa, Homo sapiens
IHVTILHKEEGAGLGFSLAGGADLENKVITVHRVFPNGLASQEGTIQKGNEVLSINGKSLKGTTHHDALAILRQAREPRQAVIVTRKLTPEEFIVTD

SEQ ID NO: 273 - protein, 98 aa, Homo sapiens
TAEATVCTVTLEKMSAGLGFSLEGGKGSLHGDKPLTINRIFKGAASEQSETVQPGDEILQLGGTAMQGLTRFEAWNIIKALPDGPVTIVIRRKSLQSK

SEQ ID NO: 274 - protein, 98 aa, Homo sapiens
LEYEITLERGNSGLGFSIAGGTDNPHIGDDPSIFITKIIPGGAAAQDGRLRVNDSILFVNEVDVREVTHSAAVEALKEAGSIVRLYVMRRKPPAENSS

SEQ ID NO: 275 - protein, 105 aa, Homo sapiens
HVMRRKPPAEKVMEIKLIKGPKGLGFSIAGGVGNQHIPGDNSIYVTKIIEGGAAHKDGRLQIGDKILAVNSVGLEDVMHEDAVAALKNTYDVVYLKVAKPSNAYL

SEQ ID NO: 276 - protein, 97 aa, Homo sapiens
REDIPREPRRIVIHRGSTGLGFNIVGGEDGEGIFISFILAGGPADLSGELRKGDQILSVNGVDLRNASHEQAAIALKNAGQTVTIIAQYKPEFIVTD

SEQ ID NO: 277 - protein, 88 aa, Homo sapiens
LIRITPDEDGKFGFNLKGGVDQKMPLVVSRINPESPADTCIPKLNEGDQIVLINGRDISEHTHDQVVMFIKASRESHSRELALVIRRR

SEQ ID NO: 278 - protein, 88 aa, Homo sapiens
IRMKPDENGRFGFNVKGGYDQKMPVIVSRVAPGTPADLCVPRLNEGDQVVLINGRDIAEHTHDQVVLFIKASCERHSGELMLLVRPNA

SEQ ID NO: 279 - protein, 106 aa, Homo sapiens
PEREITLVNLKKDAKYGLGFQIIGGEKMGRLDLGIFISSVAPGGPADFHGCLKPGDRLISVNSVSLEGVSHHAAIEILQNAPEDVTLVISQPKEKISKVPSTPVHL

SEQ ID NO: 280 - protein, 95 aa, Homo sapiens
GDIFEVELAKNDNSLGISVTGGVNTSVRHGGIYVKAVIPQGAAESDGRIHKGDRVLAVNGVSLEGATHKQAVETLRNTGQVVHLLLEKGQSPTSK

SEQ ID NO: 281 - protein, 104 aa, Homo sapiens
TEENTFEVKLFKNSSGLGFSFSREDNLIPEQINASIVRVKKLFAGQPAAESGKIDVGDVILKVNGASLKGLSQQEVISALRGTAPEVFLLLCRPPPGVLPEIDT

SEQ ID NO: 282 - protein, 98 aa, Homo sapiens
ELEVELLITLIKSEKASLGFTVTKGNQRIGCYVHDVIQDPAKSDGRLKPGDRLIKVNDTDVTNMTHTDAVNLLRAASKTVRLVIGRVLELPRIPMLPH

SEQ ID NO: 283 - protein, 94 aa, Homo sapiens
MLPHLLPDITLTCNKEELGFSLCGGHDSLYQVVYISDINPRSVAAIEGNLQLLDVIHYVNGVSTQGMTLEEVNRALDMSLPSLVLKATRNDLPV

SEQ ID NO: 284 - protein, 93 aa, Homo sapiens
RPSPPRVRSVEVARGRAGYGFTLSGQAPCVLSCVMRGSPADFVGLRAGDQILAVNEINVKKASHEDVVKLIGKCSGVLHMVIAEGVGRFESCS

SEQ ID NO: 285 - protein, 96 aa, Homo sapiens
LCSERRYRQITIPRGKDGFGFTICCDSPVRVQAVDSGGPAERAGLQQLDTVLQLNERPVEHWKCVELAHEIRSCPSEIILLVWRMVPQVKPGIHRD

SEQ ID NO: 286 - protein, 104 aa, Homo sapiens
ISFSANKRWTPPRSIRFTAEEGDLGFTLRGNAPVQVHFLDPYCSASVAGAREGDYIVSIQLVDCKWLTLSEVMKLLKSFGEDEIEMKVVSLLDSTSSMHNKSAT

SEQ ID NO: 287 - protein, 109 aa, Homo sapiens
RGEKKNSSSGISGSQRRYIGVMMLTLSPSILAELQLREPSFPDVQHGVLIHKVILGSPAHRAGLRPGDVILAIGEQMVQNAEDVYEAVRTQSQLAVQIRRGRETLTLYV

SEQ ID NO: 288 - protein, 111 aa, Homo sapiens
EEKTVVLQKKDNEGFGFVLRGAKADTPIEEFTPTPAFPALQYLESVDEGGVAWQAGLRTGDFLIEVNNENVVKVGHRQVVNMIRQGGNHLVLKVVTVTRNLDPDDTARKKA

SEQ ID NO: 289 - protein, 110 aa, Homo sapiens
SDYVIDDKVAVLQKRDHEGFGFVLRGAKAETPIEEFTPTPAFPALQYLESVDVEGVAWRAGLRTGDFLIEVNGVNVVKVGHKQVVALIRQGGNRLVMKVVSVTRKPEEDG

SEQ ID NO: 290 - protein, 91 aa, Homo sapiens
IYLEAFLEGGAPWGFTLKGGLEHGEPLIISKVEEGGKADTLSSKLQAGDEVVHINEVTLSSSRKEAVSLVKGSYKTLRLVVRRDVCTDPGH

SEQ ID NO: 291 - protein, 83 aa, Homo sapiens
IRLCRLVRGEQGYGFHLHGEKGRRGQFIRRVEPGSPAEAAALRAGDRLVEVNGVNVEGETHHQVVQRIKAVEGQTRLLVVDQN

SEQ ID NO: 292 - protein, 84 aa, Homo sapiens
IRHLRKGPQGYGFNLHSDKSRPGQYIRSVDPGSPAARSGLRAQDRLIEVNGQNVEGLRHAEVVASIKAREDEARLLVVDPETDE

SEQ ID NO: 293 - protein, 92 aa, Homo sapiens
PGVREIHLCKDERGKTGLRLRKVDQGLFVQLVQANTPASLVGLRFGDQLLQIDGRDCAGWSSHKAHQVVKKASGDKIVVVVRDRPFQRTVTM

SEQ ID NO: 294 - protein, 90 aa, Homo sapiens
PFQRTVTMHKDSMGHVGFVIKKGKIVSLVKGSSAARNGLLTNHYVCEVDGQNVIGLKDKKIMEILATAGNVVTLTIIPSVIYEHIVEFIV

SEQ ID NO: 295 - protein, 109 aa, Homo sapiens
LKEKTVLLQKKDSEGFGFVLRGAKAQTPIEEFTPTPAFPALQYLESVDEGGVAWRAGLRMGDFLIEVNGQNVVKVGHRQVVNMIRQGGNTLMVKVVMVTRHPDMDEAVQ

SEQ ID NO: 296 - protein, 88 aa, Homo sapiens
LEIKQGIREVILCKDQDGKIGLRLKSIDNGIFVQLVQANSPASLVGLRFGDQVLQINGENCAGWSSDKAHKVLKQAFGEKITMRIHRD

SEQ ID NO: 297 - protein, 75 aa, Homo sapiens
RDRPFERTITMHKDSTGHVGFIFKNGKITSIVKDSSAARNGLLTEHNICEINGQNVIGLKDSQIADILSTSGNSS

SEQ ID NO: 298 - protein, 94 aa, Homo sapiens
QRRRVTVRKADAGGLGISIKGGRENKMPILISKIFKGLAADQTEALFVGDAILSVNGEDLSSATHDEAVQVLKKTGKEVVLEVKYMKDVSPYFK

SEQ ID NO: 299 - protein, 89 aa, Homo sapiens
IRVVKQEAGGLGISIKGGRENRMPILISKIFPGLAADQSRALRLGDAILSVNGTDLRQATHDQAVQALKRAGKEVLLEVKFIREFIVTD

SEQ ID NO: 300 - protein, 101 aa, Homo sapiens
EPFYSGERTVTIRRQTVGGFGLSIKGGAEHNIPVVVSKISKEQRAELSGLLFIGDAILQINGINVRKCRHEEVVQVLRNAGEEVTLTVSFLKRAPAFLKLP

SEQ ID NO: 301 - protein, 99 aa, Homo sapiens
SHQGRNRRTVTLRRQPVGGLGLSIKGGSEHNVPVVISKIFEDQAADQTGMLFVGDAVLQVNGIHVENATHEEVVHLLRNAGDEVTITVEYLREAPAFLK

SEQ ID NO: 302 - protein, 91 aa, Homo sapiens
RGETKEVEVTKTEDALGLTITDNGAGYAFIKRIKEGSIINRIEAVCVGDSIEAINDHSIVGCRHYEVAKMLRELPKSQPFTLRLVQPKRAF

SEQ ID NO: 303 - protein, 88 aa, Homo sapiens
HSIHIEKSDTAADTYGFSLSSVEEDGIRRLYVNSVKETGLASKKGLKAGDEILEINNRAADALNSSMLKDFLSQPSLGLLVRTYPELE

SEQ ID NO: 304 - protein, 97 aa, Homo sapiens
PLNVYDVQLTKTGSVCDFGFAVTAQVDERQHLSRIFISDVLPDGLAYGEGLRKGNEIMTLNGEAVSDLDLKQMEALFSEKSVGLTLIARPPDTKATL

SEQ ID NO: 305 - protein, 103 aa, Homo sapiens
QRVEIHKLRQGENLILGFSIGGGIDQDPSQNPFSEDKTDKGIYVTRVSEGGPAEIAGLQIGDKIMQVNGWDMTMVTHDQARKRLTKRSEEVVRLLVTRQSLQK

SEQ ID NO: 306 - protein, 86 aa, Homo sapiens
RKEVEVFKSEDALGLTITDNGAGYAFIKRIKEGSVIDHIHLISVGDMIEAINGQSLLGCRHYEVARLLKELPRGRTFTLKLTEPRK

SEQ ID NO: 307 - protein, 91 aa, Homo sapiens
HSHPRVVELPKTDEGLGFNVMGGKEQNSPIYISRIIPGGVAERHGGLKRGDQLLSVNGVSVEGEHHEKAVELLKAAKDSVKLVVRYTPKVL

SEQ ID NO: 308 - protein, 96 aa, Homo sapiens
ISNQKRGVKVLKQELGGLGISIKGGKENKMPILISKIFKGLAADQTQALYVGDAILSVNGADLRDATHDEAVQALKRAGKEVLLEVKYMREATPYV

SEQ ID NO: 309 - protein, 110 aa, Homo sapiens
IHFSNSENCKELQLEKHKGEILGVVVVESGWGSILPTVILANMMNGGPAARSGKLSIGDQIMSINGTSLVGLPLATCQGIIKGLKNQTQVKLNIVSCPPVTTVLIKRNSS

SEQ ID NO: 310 - protein, 94 aa, Homo sapiens
IPPVTTVLIKRPDLKYQLGFSVQNGIICSLMRGGIAERGGVRVGHRIIEINGQSVVATAHEKIVQALSNSVGEIHMKTMPAAMFRLLTGQENSS

SEQ ID NO: 311 - protein, 101 aa, Homo sapiens
IWEQHTVTLHRAPGFGFGIAISGGRDNPHFQSGETSIVISDVLKGGPAEGQLQENDRVAMVNGVSMDNVEHAFAVQQLRKSGKNAKITIRRKKKVQIPNSS

SEQ ID NO: 312 - protein, 95 aa, Homo sapiens
ISSQPAKPTKVTLVKSRKNEEYGLRLASHIFVKEISQDSLAARDGNIQEGDVVLKINGTVTENMSLTDAKTLIERSKGKLKMVVQRDRATLLNSS

SEQ ID NO: 313 - protein, 90 aa, Homo sapiens
IRMKLVKFRKGDSVGLRLAGGNDVGIFVAGVLEDSPAAKEGLEEGDQILRVNNVDFTNIIREEAVLFLLDLPKGEEVTILAQKKKDVFSN

SEQ ID NO: 314 - protein, 96 aa, Homo sapiens
LIWEQYTVTLQKDSKRGFGIAVSGGRDNPHFENGETSIVISDVLPGGPADGLLQENDRVVMVNGTPMEDVLHSFAVQQLRKSGKVAAIVVKRPRKV

SEQ ID NO: 315 - protein, 79 aa, Homo sapiens
RVLLMKSRANEEYGLRLGSQIFVKEMTRTGLATKDGNLHEGDIILKINGTVTENMSLTDARKLIEKSRGKLQLVVLRDS

SEQ ID NO: 316 - protein, 90 aa, Homo sapiens
HAPNTKMVRFKKGDSVGLRLAGGNDVGIFVAGIQEGTSAEQEGLQEGDQILKVNTQDFRGLVREDAVLYLLEIPKGEMVTILAQSRADVY

SEQ ID NO: 317 - protein, 106 aa, Homo sapiens
IPGNSTIWEQHTATLSKDPRRGFGIAISGGRDRPGGSMVVSDVVPGGPAEGRLQTGDHIVMVNGVSMENATSAFAIQILKTCTKMANITVKRPRRIHLPAEFIVTD

SEQ ID NO: 318 - protein, 98 aa, Homo sapiens
QDVQMKPVKSVLVKRRDSEEFGVKLGSQIFIKHITDSGLAARHRGLQEGDLILQINGVSSQNLSLNDTRRLIEKSEGKLSLLVLRDRGQFLVNIPNSS

SEQ ID NO: 319 - protein, 104 aa, Homo sapiens
RGYSPDTRVVRFLKGKSIGLRLAGGNDVGIFVSGVQAGSPADGQGIQEGDQILQVNDVPFQNLTREEAVQFLLGLPPGEEMELVTQRKQDIFWKMVQSEFIVTD

SEQ ID NO: 320 - protein, 72 aa, Homo sapiens
RKSSRGFGFTVVGGDEPDEFLQIKSLVLDGPAALDGKMETGDVIVSVNDTCVLGHTHAQVVKIFQSIPIGAS

SEQ ID NO: 321 - protein, 76 aa, Homo sapiens
FIHTKLRKSSRGFGFTVVGGDEPDEFLQIKSLVLDGPAALDGKMETGDVIVSVNDTCVLGHTHAQVVKIFQSIPIG

SEQ ID NO: 322 - protein, 85 aa, Homo sapiens
FIHTKLRKSSRGFGFTVVGGDEPDEFLQIKSLVLDGPAALDGKMETGDVIVSVNDTCVLGHTHAQVVKIFQSIPIGASVDLELCR

SEQ ID NO: 323 - protein, 78 aa, Homo sapiens
KSSRGFGFTVVGGDEPDEFLQIKSLVLDGPAALDGKMETGDVIVSVNDTCVLGHTHAQVVKIFQSIPIGASVDLELCR

SEQ ID NO: 324 - protein, 88 aa, Homo sapiens
FIHTKLRKSSRGFGFTVVGGDEPDEFLQIKSLVLDGPAALDGKMETGDVIVSVNDTCVLGHTHAQVVKIFQSIPIGASVDLELCRGYP

SEQ ID NO: 325 - protein, 88 aa, Homo sapiens
KGKFIHTKLRKSSRGFGFTVVGGDEPDEFLQIKSLVLDGPAALDGKMETGDVIVSVNDTCVLGHTHAQVVKIFQSIPIGASVDLELCR

SEQ ID NO: 326 - protein, 81 aa, Homo sapiens
KGKFIHTKLRKSSRGFGFTVVGGDEPDEFLQIKSLVLDGPAALDGKMETGDVIVSVNDTCVLGHTHAQVVKIFQSIPIGAS

SEQ ID NO: 327 - protein, 94 aa, Homo sapiens
ELKGKFIHTKLRKSSRGFGFTVVGGDEPDEFLQIKSLVLDGPAALDGKMETGDVIVSVNDTCVLGHTHAQVVKIFQSIPIGASVDLELCRGYPL

SEQ ID NO: 328 - protein, 99 aa, Homo sapiens
SELKGKFIHTKLRKSSRGFGFTVVGGDEPDEFLQIKSLVLDGPAALDGKMETGDVIVSVNDTCVLGHTHAQVVKIFQSIPIGASVDLELCRGYPLPFDP

SEQ ID NO: 329 - protein, 72 aa, Homo sapiens
RKSARGFGFTVVGGDEPDEFLQIKSLVLDGPAALDGKMETGDVIVSVNDTCVLGHTHAQVVKIFQSIPIGAS

SEQ ID NO: 330 - protein, 72 aa, Homo sapiens
RKSSRGFGFTVVGGEEPDEFLQIKSLVLDGPAALDGKMETGDVIVSVNDTCVLGHTHAQVVKIFQSIPIGAS

SEQ ID NO: 331 - protein, 72 aa, Homo sapiens
RKSSRGFGFTVVGGDEPDEFLQLKSLVLDGPAALDGKMETGDVIVSVNDTCVLGHTHAQVVKIFQSIPIGAS

SEQ ID NO: 332 - protein, 72 aa, Homo sapiens
RKSSRGFGFTVVGGDEPDEFLQIKSLVLDGPASLDGKMETGDVIVSVNDTCVLGHTHAQVVKIFQSIPIGAS

SEQ ID NO: 333 - protein, 72 aa, Homo sapiens
RKSSRGFGFTVVGGDEPDEFLQIKSLVLDGPAALDGRMETGDVIVSVNDTCVLGHTHAQVVKIFQSIPIGAS

SEQ ID NO: 334 - protein, 72 aa, Homo sapiens
RKSSRGFGFTVVGGDEPDEFLQIKSLVLDGPAALDGKMETGDVIVAVNDTCVLGHTHAQVVKIFQSIPIGAS

SEQ ID NO: 335 - protein, 72 aa, Homo sapiens
RKSSRGFGFTVVGGDEPDEFLQIKSLVLDGPAALDGKMETGDVIVSVNETCVLGHTHAQVVKIFQSIPIGAS

SEQ ID NO: 336 - protein, 72 aa, Homo sapiens
RKSSRGFGFTVVGGDEPDEFLQIKSLVLDGPAALDGKMETGDVIVSVNDTCLLGHTHAQVVKIFQSIPIGAS

SEQ ID NO: 337 - protein, 72 aa, Homo sapiens
RKSSRGFGFTVVGGDEPDEFLQIKSLVLDGPAALDGKMETGDVIVSVNDTCVLGHTHSQVVKIFQSIPIGAS

SEQ ID NO: 338 - protein, 72 aa, Homo sapiens
RKSSRGFGFTVVGGDEPDEFLQIKSLVLDGPAALDGKMETGDVIVSVNDTCVLGHTHAQVVKLFQSIPIGAS

SEQ ID NO: 339 - protein, 72 aa, Homo sapiens
RKSSRGFGFTVVGGDEPDEFLQIKSLVLDGPAALDGKMETGDVIVSVNDTCVLGHTHAQVVKIFQSIPIGSS

SEQ ID NO: 340 - protein, 72 aa, Homo sapiens
RKSTRGFGFTVVGGDEPDEFLQIKSLVLDGPAALDGKMETGDVIVSVNDTCVLGHTHAQVVKIFQSIPIGAS

SEQ ID NO: 341 - protein, 72 aa, Homo sapiens
RKSSRGFGFTVVGGDEPGEFLQIKSLVLDGPAALDGKMETGDVIVSVNDTCVLGHTHAQVVKIFQSIPIGAS

SEQ ID NO: 342 - protein, 72 aa, Homo sapiens
RKSSRGFGFTVVGGDEPDEFLQIKSLALDGPAALDGKMETGDVIVSVNDTCVLGHTHAQVVKIFQSIPIGAS

SEQ ID NO: 343 - protein, 72 aa, Homo sapiens
RKSSRGFGFTVVGGDEPDEFLQIKSLVLDGPAALAGKMETGDVIVSVNDTCVLGHTHAQVVKIFQSIPIGAS

SEQ ID NO: 344 - protein, 72 aa, Homo sapiens
RKSSRGFGFTVVGGDEPDEFLQIKSLVLDGPAALDGKMETADVIVSVNDTCVLGHTHAQVVKIFQSIPIGAS

SEQ ID NO: 345 - protein, 72 aa, Homo sapiens
RKSSRGFGFTVVGGDEPDEFLQIKSLVLDGPAALDGKMETGDVIVSVNDTAVLGHTHAQVVKIFQSIPIGAS

SEQ ID NO: 346 - protein, 72 aa, Homo sapiens
RKSSRGFGFTVVGGDEPDEFLQIKSLVLDGPAALDGKMETGDVIVSVNDTCVLGHTHAQAVKIFQSIPIGAS

SEQ ID NO: 347 - protein, 72 aa, Homo sapiens
RKSSRGFGFTVVGGDEPDEFLQIKSLVLDGPAALDGKMETGDVIVSVNDTCVLGHTHAQVVKIFQSIAIGAS

SEQ ID NO: 348 - protein, 72 aa, Homo sapiens
RKSSRGFGFTVVGGDEPDEFLQIKSLVLDGPAALDGKMETGDVIVSVNDTCVLGHTHAQVVKIFQSIPIGAA

SEQ ID NO: 349 - protein, 72 aa, Homo sapiens
RKSSSGFGFTVVGGDEPDEFLQIKSLVLDGPAALDGKMETGDVIVSVNDTCVLGHTHAQVVKIFQSIPIGAS

SEQ ID NO: 350 - protein, 72 aa, Homo sapiens
RKSSRGFGFTVVGGLEPDEFLQIKSLVLDGPAALDGKMETGDVIVSVNDTCVLGHTHAQVVKIFQSIPIGAS

SEQ ID NO: 351 - protein, 72 aa, Homo sapiens
RKSSRGFGFTVVGGDEPDEFLQITSLVLDGPAALDGKMETGDVIVSVNDTCVLGHTHAQVVKIFQSIPIGAS

SEQ ID NO: 352 - protein, 72 aa, Homo sapiens
RKSSRGFGFTVVGGDEPDEFLQIKSLVLDGPAGLDGKMETGDVIVSVNDTCVLGHTHAQVVKIFQSIPIGAS

SEQ ID NO: 353 - protein, 72 aa, Homo sapiens
RKSSRGFGFTVVGGDEPDEFLQIKSLVLDGPAALDGKMETSDVIVSVNDTCVLGHTHAQVVKIFQSIPIGAS

SEQ ID NO: 354 - protein, 72 aa, Homo sapiens
RKSSRGFGFTVVGGDEPDEFLQIKSLVLDGPAALDGKMETGDVIVSVKDTCVLGHTHAQVVKIFQSIPIGAS

SEQ ID NO: 355 - protein, 72 aa, Homo sapiens
RKSSRGFGFTVVGGDEPDEFLQIKSLVLDGPAALDGKMETGDVIVSVNDTCVLFHTHAQVVKIFQSIPIGAS

SEQ ID NO: 356 - protein, 72 aa, Homo sapiens
RKSSRGFGFTVVGGDEPDEFLQIKSLVLDGPAALDGKMETGDVIVSVNDTCVLGHTHAQNVKIFQSIPIGAS

SEQ ID NO: 357 - protein, 72 aa, Homo sapiens
RKSSRGFGFTVVGGDEPDEFLQIKSLVLDGPAALDGKMETGDVIVSVNDTCVLGHTHAQVVKIFQSIPISAS